SEQUENCE LISTING (2 sequences)

SEQ ID NO: 1 - protein, 218 aa, synthetic construct
RSIGTTKLRLVEFSAFLEQQRDPDSYNKHLFVHIGHANHSYSDPLLESVDIRQIYDKFPEKKGGLKELFGKGPQNAFFLVKFWADLNCNIQDDAGAFYGVTSQYESSENMTVTCSTKVCSFGKQVVEKVETEYARFENGRFVYRINRSPMCEYMINFIHKLKHLPEKYMMNSVLENFTILLVVTNRDTQETLLCMACVFEVSNSEHGAQHHIYRLVKD

SEQ ID NO: 2 - protein, 51 aa, synthetic construct
AGHQIVHVRGDSETDLEALFNAVMNPKTANVPQTVPMRLRKLPDSFFKPPE

(19) United States
(12) Patent Application Publication
(10) Pub. No.: US 2025/0154127 A1
(43) Pub. Date: May 15, 2025

(54) BICYCLIC HETEROARYL COMPOUNDS AND USES THEREOF

(71) Applicant: ETERN BIOPHARMA (SHANGHAI) CO., LTD., Shanghai (CN)

(72) Inventors: Qiangang ZHENG, Shanghai (CN); Zhenting GAO, Shanghai (CN); Qinglong ZENG, Shanghai (CN); Jing LI, Shanghai (CN); Ming XU, Shanghai (CN); Xiaofeng ZHONG, Shanghai (CN); Xin GUO, Shanghai (CN); Jidong ZHU, Shanghai (CN)

(21) Appl. No.: 18/837,121

(22) PCT Filed: Feb. 7, 2023
(86) PCT No.: PCT/CN2023/074835

§ 371 Date: Aug. 8, 2024

(30) Foreign Application Priority Data
Feb. 8, 2022 (WO) PCT/CN2022/075544
Nov. 17, 2022 (WO) PCT/CN2022/132667

(51) Int. Cl. C07D 401/14; A61K 31/4439; A61K 31/444; A61K 31/4545; A61K 31/497; A61K 31/501; A61K 31/506; A61K 31/5377; C07D 405/14; C07D 471/04; C07D 487/04; C07D 491/107

(52) U.S. Cl. CPC C07D 401/14; A61K 31/4439; A61K 31/444; A61K 31/4545; A61K 31/497; A61K 31/501; A61K 31/506; A61K 31/5377; C07D 405/14; C07D 471/04; C07D 487/04; C07D 491/107

(57) ABSTRACT

Provided are bicyclic heteroaryl compounds of Formula (I), methods of making such compounds, pharmaceutical compositions and medicaments comprising such compounds, and methods of using such compounds in treating cancer and other diseases.

### CROSS-REFERENCE

This application claims the benefit International Application No. PCT/CN2022/075544, filed Feb. 8, 2022, and International Application No. PCT/CN2022/132667, filed Nov. 17, 2022, which are incorporated herein by reference in their entirety.

### BACKGROUND OF THE INVENTION

The Hippo signaling pathway is involved in restraining cell proliferation and promoting apoptosis. As many cancers are marked by unchecked cell division, this signaling pathway is of interest in the study of new therapeutics for the treatment of cancer and other hyperproliferative diseases and disorders.

### SUMMARY OF THE INVENTION

In one aspect, described herein is a compound that has the structure of Formula (I), or a pharmaceutically acceptable salt or solvate thereof:

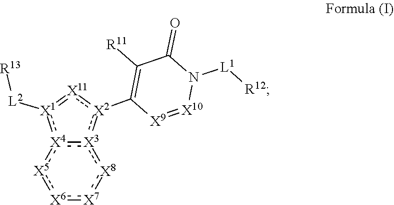

wherein:
X¹ is C, C(R¹), or N;
X² is C, C(R²), or N;
X³ is C, C(R³), or N;
X⁴ is C, C(R⁴), or N;
X⁵ is C(R⁵), C(R⁵)(R^{5a}), N(R^{5b}), or N;
X⁶ is C(R⁶), C(R⁶)(R^{6a}), N(R^{6b}), or N;
X⁷ is C(R⁷), C(R⁷)(R^{7a}), N(R^{7b}), or N;
X⁸ is C(R⁸), C(R⁸)(R^{8a}), N(R^{8b}), or N;
X⁹ is C(R⁹) or N;
X¹⁰ is C(R¹⁰) or N;
X¹¹ is C(R¹⁶), C(R¹⁶)(R^{16a}), C(O), N(R^{16b}), or N;
L¹ and L² are independently selected from a bond and C₁₋₆alkylene optionally substituted with one, two, or three groups selected from R^{14a};
R¹, R², R³, and R⁴ are independently selected from hydrogen and C₁₋₆alkyl;
R⁵, R^{5a}, R⁶, R^{6a}, R⁷, R^{7a}, R⁸, R^{8a}, R¹⁶, and R^{16a} are independently selected from hydrogen, halogen, —CN, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR²⁰, —SR²⁰, —SF₅, —N(R²⁰)(R^{21a}), —C(O)OR²⁰, —OC(O)N(R²⁰)(R²¹), —N(R²²)C(O)N(R²⁰)(R²¹), —N(R²²)C(O)OR²³, —N(R²²)S(O)₂R²³, —C(O)R²³, —S(O)R²³, —OC(O)R²³, —C(O)N(R²⁰)(R²¹), —C(O)C(O)N(R²⁰)(R²¹), —N(R²²)C(O)R²³, —S(O)₂R²³, —S(O)₂N(R²⁰)(R²¹)—, —N═S(═O)(R²³)₂, —S(═O)(═NH)N(R²⁰)(R²¹), —S(═O)(═NH)C(R²⁰)(R²¹), —S(═O)(═NR²³)R²³, —CH₂C(O)N(R²⁰)(R²¹), —CH₂N(R²²)C(O)R²³, —CH₂S(O)₂R²³, and —CH₂S(O)₂N(R²⁰)(R²¹), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three groups selected from R^{14b};
R^{5b}, R^{6b}, R^{7b}, R^{8b}, and R^{16b} are independently selected from hydrogen, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —C(O)OR²⁰, —OC(O)N(R²⁰)(R²¹), —C(O)R²³, —C(O)N(R²⁰)(R²¹), and —S(O)₂R², wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three groups selected from R^{14c};
R⁹, R¹⁰, and R¹¹ are independently selected from hydrogen, halogen, and C₁₋₆alkyl;
R¹² is selected from C₁₋₆alkyl, C₁₋₆alkoxyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl, wherein C₁₋₆alkyl, C₁₋₆alkoxyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three groups selected from R^{15a};
R¹³ is selected from C₁₋₆alkyl, C₁₋₆alkoxyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl, wherein C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three groups selected from R^{15b};
each R^{14a}, R^{14b}, and R^{14c} are each independently selected from halogen, oxo, —CN, C₁₋₆alkyl, C₁₋₆haloalkyl, C₂₋₆alkenyl, C₂₋₆alkynyl, C₃₋₁₀cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR²⁰, —SR²⁰, —SF₅, —N(R²⁰)(R²¹), —C(O)OR²⁰, —OC(O)N(R²⁰)(R²¹), —N(R²²)C(O)N(R²⁰)(R²¹), —N(R²²)C(O)OR²³, —N(R²²)S(O)₂R²³, —C(O)R²³, —S(O)R²³, —OC(O)R²³, —C(O)N(R²⁰)(R²¹), —C(O)C(O)N(R²⁰)(R²¹), —N(R²²)C(O)R²³, —S(O)₂R²³, —S(O)₂N(R²⁰)(R²¹)—, —N═S(═O)(R²³)₂, —S(═O)(═NH)N(R²⁰)(R²¹), —S(═O)(═NH)C(R²⁰)(R²¹), —S(═O)(═NR²³)R²³, —CH₂C(O)N(R²⁰)(R²¹), —CH₂N(R²²)C(O)R²³, —CH₂S(O)₂R²³, —CH₂S(O)₂N(R²⁰)(R²¹), wherein C₁₋₆alkyl, C₁₋₆haloalkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²⁰, —SR²⁰, —SF₅, —N(R²⁰)(R²¹), —C(O)OR²⁰, —OC(O)N(R²⁰)(R²¹), —N(R²²)C(O)N(R²⁰)(R²¹), —N(R²²)C(O)OR²³, —N(R²)S(O)₂R²³, —C(O)R²³, —S(O)R²³, —OC(O)R²³, —C(O)N(R²⁰)(R²¹), —C(O)C(O)N(R²⁰)(R²¹), —N(R²²)C(O)R²³, —S(O)₂R²³, —S(O)₂N(R²⁰)(R²¹)—, —N═S(═O)(R²³)₂, —S(═O)(═NH)N(R²⁰)(R²¹), —S(═O)(═NH)C(R²⁰)(R²¹), —S(═O)(═NR²³)R²³, —CH₂C(O)N(R²⁰)(R²¹), —CH₂N(R²²)C(O)R²³, —CH₂S(O)₂R²³, and —CH₂S(O)₂N(R²⁰)(R²¹);
each R^{15a} is independently selected from halogen, oxo, —CN, C₁₋₆alkyl, C₁₋₆haloalkyl, C₂₋₆alkenyl, C₂₋₆alkynyl, C₃₋₁₀cycloalkyl, —CH₂—C₃₋₆cycloalkyl, C₂₋₉heterocycloalkyl, —CH₂—C₂₋₉heterocycloalkyl, —NH—C₃₋₉cycloalkyl, C₆₋₁₀aryl, —CH₂—C₆₋₁₀aryl, C₁₋₉heteroaryl, —CH₂—C₁₋₉heteroaryl, —OR²⁰, —SR²⁰, —SF₅, —N(R²⁰)(R²¹), —C(O)OR²⁰, —OC(O)N(R²⁰)(R²¹), —N(R²²)C(O)N(R²⁰)(R²¹), —N(R²²)C(O)OR²³, —N(R²)S(O)₂R²³, —C(O)R²³, —S(O)R²³, —OC(O)R²³, —C(O)N(R²⁰)(R²¹), —C(O)C(O)N(R²⁰)(R²¹), —N(R²²)C(O)R²³, —S(O)₂R²³, —S(O)₂N(R²⁰)(R²¹)—, —N═S(═O)(R²³)₂, —S(═O)(═NH)N(R²⁰)(R²¹), —S(═O)(═NH)C(R²⁰)(R²¹), —S(═O)(═NR²³)R²³, —CH₂C(O)N(R²⁰)(R²¹), —CH₂N(R²²)C(O)R²³, —CH₂S(O)₂R²³, —CH₂S(O)₂N(R²⁰)(R²¹), wherein C₁₋₆alkyl, C₁₋₆haloalkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²⁰, —SR²⁰, —SF₅, —N(R²⁰)(R²¹), —C(O)OR²⁰, —OC(O)N(R²⁰)(R²¹), —N(R²²)C(O)N(R²⁰)(R²¹), —N(R²²)C(O)OR²³, —N(R²²)S(O)₂R²³, —C(O)R²³, —S(O)R²³, —OC(O)R²³, —C(O)N(R²⁰)(R²¹), —C(O)C(O)N(R²⁰)(R²¹), —N(R²²)C(O)R²³, —S(O)₂R²³, —S(O)₂N(R²⁰)(R²¹)—, —N═S(═O)(R²³)₂, —S(═O)(═NH)N(R²⁰)(R²¹), —S(═O)(═NH)C(R²⁰)(R²¹), —S(═O)(═NR²³)R²³, —CH₂C(O)N(R²⁰)(R²¹), —CH₂N(R²²)C(O)R²³, —CH₂S(O)₂R²³, and —CH₂S(O)₂N(R²⁰)(R²¹); or two R^{15a} are combined to form a C₃₋₆cycloalkyl or C₂₋₉heterocycloalkyl, wherein C₃₋₆cycloalkyl and C₂₋₉heterocycloalkyl are optionally substituted with one, two, or three groups independently selected from halogen, oxo, C₁₋₆alkyl, C₁₋₆haloalkyl, and C₁₋₆alkoxy;
each R^{15b} is independently selected from halogen, oxo, —CN, C₁₋₆alkyl, C₁₋₆haloalkyl, C₂₋₆alkenyl, C₂₋₆alkynyl, C₃₋₁₀cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR²⁰, —SR²⁰, —SF₅, —N(R²⁰)(R²¹), —C(O)OR²⁰, —OC(O)N(R²⁰)(R²¹), —N(R²²)C(O)N(R²⁰)(R²¹), —N(R²²)C(O)OR²³, —N(R²²)S(O)₂R²³, —C(O)R²³, —S(O)R²³, —OC(O)R²³, —C(O)N(R²⁰)(R²¹), —C(O)C(O)N(R²⁰)(R²¹), —N(R²²)C(O)R²³, —S(O)₂R²³, —S(O)₂N(R²⁰)(R²¹)—, —N═S(═O)(R²³)₂, —S(═O)(═NH)N(R²⁰)(R²¹), —S(═O)(═NH)C(R²⁰)(R²¹), —S(═O)(═NR²³)R²³,
—CH₂C(O) N(R²⁰)(R²¹), —CH₂N(R²²)C(O)R²³, —CH₂S(O)₂R²³, —CH₂S(O)₂N(R²⁰)(R²¹), wherein C₁₋₆alkyl, C₁₋₆haloalkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²⁰, —SR²⁰, —SF₅, —N(R²⁰)(R²¹), —C(O)OR²⁰, —OC(O)N(R²⁰)(R²¹), —N(R²²)C(O)N(R²⁰)(R²¹), —N(R²²)C(O)OR²³, —N(R²)S(O)₂R²³, —C(O)R²³, —S(O)R²³, —OC(O)R²³, —C(O)N(R²⁰)(R²¹), —C(O)C(O)N(R²⁰)(R²¹), —N(R²²)C(O)R²³, —S(O)₂R²³, —S(O)₂N(R²⁰)(R²¹)—, —N═S(═O)(R²³)₂, —S(═O)(═NH)N(R²⁰)(R²¹), —S(═O)(═NH)C(R²⁰)(R²¹), —S(═O)(═NR²³)R²³, —CH₂C(O)N(R²⁰)(R²¹), —CH₂N(R²²)C(O)R²³, —CH₂S(O)₂R²³, and —CH₂S(O)₂N(R²⁰)(R²¹); or two R^{15b} are combined to form a C₃₋₆cycloalkyl or C₂₋₉heterocycloalkyl, wherein C₃₋₆cycloalkyl and C₂₋₉heterocycloalkyl are optionally substituted with one, two, or three groups independently selected from halogen, oxo, C₁₋₆alkyl, C₁₋₆haloalkyl, and C₁₋₆alkoxy;
each R²⁰ is independently selected from hydrogen, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R²¹ is independently selected from hydrogen, C₁₋₆alkyl, and C₁₋₆haloalkyl; or R²⁰ and R²¹, together with the nitrogen to which they are attached, form a C₂₋₉heterocycloalkyl;
each R^{21a} is independently selected from C₁₋₆alkyl and C₁₋₆haloalkyl;
each R²² is independently selected from hydrogen, C₁₋₆alkyl, and C₁₋₆haloalkyl;
each R²³ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl; and
[symbol] indicates a single or double bond such that all valences are satisfied.

In some embodiments is a compound of Formula (I), or a pharmaceutically acceptable salt or solvate thereof, wherein X⁹ is C(R⁹). In some embodiments is a compound of Formula (I), or a pharmaceutically acceptable salt or solvate thereof, wherein R⁹ is hydrogen. In some embodiments is a compound of Formula (I), or a pharmaceutically acceptable salt or solvate thereof, wherein X¹⁰ is C(R¹⁰). In some embodiments is a compound of Formula (I), or a pharmaceutically acceptable salt or solvate thereof, wherein R¹⁰ is hydrogen. In some embodiments is a compound of Formula (I), or a pharmaceutically acceptable salt or solvate thereof, wherein R¹¹ is hydrogen.

In another aspect, described herein is a compound that has the structure of Formula (II), or a pharmaceutically acceptable salt or solvate thereof:

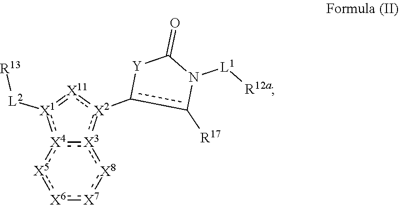

wherein:
X¹ is C, C(R¹), or N;
X² is C, C(R²), or N;
X³ is C, C(R³), or N;
X⁴ is C, C(R⁴), or N;
X⁵ is C(R⁵), C(R⁵)(R^{5a}), N(R^{5b}), or N;
X⁶ is C(R⁶), C(R⁶)(R^{6a}), N(R^{6b}), or N;
X⁷ is C(R⁷), C(R⁷)(R^{7a}), N(R^{7b}), or N;
X⁸ is C(R⁸), C(R⁸)(R^{8a}), N(R^{8b}), or N;
X¹¹ is C(R¹⁶), C(R¹⁶)(R^{16a}), C(O), N(R^{16b}), or N;
Y is N(R¹⁸) or O;
L¹ and L² are independently selected from a bond and C₁₋₆alkylene optionally substituted with one, two, or three groups selected from R^{14a};
R¹, R², R³, and R⁴ are independently selected from hydrogen and C₁₋₆alkyl;
R⁵, R^{5a}, R⁶, R^{6a}, R⁷, R^{7a}, R⁸, R^{8a}, R¹⁶, and R^{16a} are independently selected from hydrogen, halogen, —CN, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR²⁰, —SR²⁰, —SF₅, —N(R²⁰)(R^{21a}), —C(O)OR²⁰, —OC(O)N(R²⁰)(R²¹), —N(R²²)C(O)N(R²⁰)(R²¹), —N(R²²)C(O)OR²³, —N(R²²)S(O)₂R²³, —C(O)R²³, —S(O)R²³, —OC(O)R²³, —C(O)N(R²⁰)(R²¹), —C(O)C(O)N(R²⁰)(R²¹), —N(R²²)C(O)R²³, —S(O)₂R²³, —S(O)₂N(R²⁰)(R²¹)—, —N═S(═O)(R²³)₂, —S(═O)(═NH)N(R²⁰)(R²¹), —S(═O)(═NH)C(R²⁰)(R²¹), —S(═O)(═NR²³)R²³, —CH₂C(O)N(R²⁰)(R²¹), —CH₂N(R²²)C(O)R²³, —CH₂S(O)₂R²³, and —CH₂S(O)₂N(R²⁰)(R²¹), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three groups selected from R^{14b};
R^{5b}, R^{6b}, R^{7b}, R^{8b}, and R^{16b} are independently selected from hydrogen, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —C(O)OR²⁰, —OC(O)N(R²⁰)(R²¹), —C(O)R²³, —C(O)N(R²⁰)(R²¹), and —S(O)₂R²³, wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three groups selected from R^{14c};
R^{12a} is selected from C₃₋₁₀cycloalkyl, C₂₋₉heterocycloalkyl, C₆₋₁₀aryl, and C₁₋₉heteroaryl, wherein C₃₋₁₀cycloalkyl, C₂₋₉heterocycloalkyl, C₆₋₁₀aryl, and C₁₋₉heteroaryl are optionally substituted with one, two, or three groups selected from R^{15a};
R¹³ is selected from C₁₋₆alkyl, C₁₋₆alkoxyl, C₁₋₆haloalkyl, C₂₋₄alkenyl, C₂₋₆alkynyl, C₃₋₁₀cycloalkyl, C₂₋₉heterocycloalkyl, C₆₋₁₀aryl, and C₁₋₉heteroaryl, wherein C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three groups selected from R^{15b};
each R^{14a}, R^{14b}, and R^{14c} are each independently selected from halogen, oxo, —CN, C₁₋₆alkyl, C₁₋₆haloalkyl, C₂₋₆alkenyl, C₂₋₆alkynyl, C₃₋₁₀cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR²⁰, —SR²⁰, —SF₅, —N(R²⁰)(R²¹), —C(O)OR²⁰, —OC(O)N(R²⁰)(R²¹), —N(R²²)C(O)N(R²⁰)(R²¹), —N(R²²)C(O)OR²³, —N(R²²)S(O)₂R²³, —C(O)R²³, —S(O)R²³, —OC(O)R²³, —C(O)N(R²⁰)(R²¹), —C(O)C(O)N(R²⁰)(R²¹), —N(R²²)C(O)R²³, —S(O)₂R²³, —S(O)₂N(R²⁰)(R²¹)—, —N═S(═O)(R²³)₂, —S(═O)(═NH)N(R²⁰)(R²¹), —S(═O)(═NH)C(R²⁰)(R²¹), —S(═O)(═NR²³)R²³, —CH₂C(O)N(R²⁰)(R²¹), —CH₂N(R²²)C(O)R²³, —CH₂S(O)₂R²³, —CH₂S(O)₂N(R²⁰)(R²¹), wherein C₁₋₆alkyl, C₁₋₆haloalkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²⁰, —SR²⁰, —SF₅, —N(R²⁰)(R²¹), —C(O)OR²⁰, —OC(O)N(R²⁰)(R²¹), —N(R²²)C(O)N(R²⁰)(R²¹), —N(R²²)C(O)OR²³, —N(R²²)S(O)₂R²³, —C(O)R²³, —S(O)R²³, —OC(O)R²³, —C(O)N(R²⁰)(R²¹), —C(O)C(O)N(R²⁰)(R²¹), —N(R²²)C(O)R²³, —S(O)₂R²³, —S(O)₂N(R²⁰)(R²¹)—, —N═S(═O)(R²³)₂, —S(═O)(═NH)N(R²⁰)(R²¹), —S(═O)(═NH)C(R²⁰)(R²¹), —S(═O)(═NR²³)R²³, —CH₂C(O)N(R²⁰)(R²¹), —CH₂N(R²²)C(O)R²³, —CH₂S(O)₂R²³, and —CH₂S(O)₂N(R²⁰)(R²¹);
each R^{15a} is independently selected from halogen, oxo, —CN, C₁₋₆alkyl, C₁₋₆haloalkyl, C₂₋₆alkenyl, C₂₋₆alkynyl, C₃₋₁₀cycloalkyl, —CH₂—C₃₋₆cycloalkyl, —NH—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR²⁰, —SR²⁰, —SF₅, —N(R²⁰)(R²¹), —C(O)OR²⁰, —OC(O)N(R²⁰)(R²¹), —N(R²²)C(O)N(R²⁰)(R²¹), —N(R²²)C(O)OR²³, —N(R²²)S(O)₂R²³, —C(O)R²³, —S(O)R²³, —OC(O)R²³, —C(O)N(R²⁰)(R²¹), —C(O)C(O)N(R²⁰)(R²¹), —N(R²²)C(O)R²³, —S(O)₂R²³, —S(O)₂N(R²⁰)(R²¹)—, —N═S(═O)(R²³)₂, —S(═O)(═NH)N(R²⁰)(R²¹), —S(═O)(═NH)C(R²⁰)(R²¹), —S(═O)(═NR²³)R²³, —CH₂C(O)N(R²⁰)(R²¹), —CH₂N(R²²)C(O)R²³, —CH₂S(O)₂R²³, —CH₂S(O)₂N(R²⁰)(R²¹), wherein C₁₋₆alkyl, C₁₋₆haloalkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²⁰, —SR²⁰, —SF₅, —N(R²⁰)(R²¹), —C(O)OR²⁰, —OC(O)N(R²⁰)(R²¹), —N(R²²)C(O)N(R²⁰)(R²¹), —N(R²²)C(O)OR²³, —N(R²²)S(O)₂R²³, —C(O)R²³, —S(O)R²³, —OC(O)R²³, —C(O)N(R²⁰)(R²¹), —C(O)C(O)N(R²⁰)(R²¹), —N(R²²)C(O)R²³, —S(O)₂R²³, —S(O)₂N(R²⁰)(R²¹)—, —N═S(═O)(R²³)₂, —S(═O)(═NH)N(R²⁰)(R²¹), —S(═O)(═NH)C(R²⁰)(R²¹), —S(═O)(═NR²³)R²³, —CH₂C(O)N(R²⁰)(R²¹), —CH₂N(R²²)C(O)R²³, —CH₂S(O)₂R²³, and —CH₂S(O)₂N(R²⁰)(R²¹); or two R^{15a} are combined to form a C₃₋₆cycloalkyl or C₂₋₉heterocycloalkyl, wherein C₃₋₆cycloalkyl and C₂₋₉heterocycloalkyl are optionally substituted with one, two, or three groups independently selected from halogen, oxo, C₁₋₆alkyl, C₁₋₆haloalkyl, and C₁₋₆alkoxy;
each R^{15b} is independently selected from halogen, oxo, —CN, C₁₋₆alkyl, C₁₋₆haloalkyl, C₂₋₆alkenyl, C₂₋₆alkynyl, C₃₋₁₀cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR²⁰, —SR²⁰, —SF₅, —N(R²⁰)(R²¹), —C(O)OR²⁰, —OC(O)N(R²⁰)(R²¹), —N(R²²)C(O)N(R²⁰)(R²¹), —N(R²²)C(O)OR²³, —N(R²²)S(O)₂R²³, —C(O)R²³, —S(O)R²³, —OC(O)R²³, —C(O)N(R²⁰)(R²¹), —C(O)C(O)N(R²⁰)(R²¹), —N(R²²)C(O)R²³, —S(O)₂R²³, —S(O)₂N(R²⁰)(R²¹)—, —N═S(═O)(R²³)₂, —S(═O)(═NH)N(R²⁰)(R²¹), —S(═O)(═NH)C(R²⁰)(R²¹), —S(═O)(═NR²³)R²³, —CH₂C(O)N(R²⁰)(R²¹), —CH₂N(R²)C(O)R²³, —CH₂S(O)₂R²³, —CH₂S(O)₂N(R²⁰)(R²¹), wherein C₁₋₆alkyl, C₁₋₆haloalkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²⁰, —SR²⁰, —SF₅, —N(R²⁰)(R²¹), —C(O)OR²⁰, —OC(O)N(R²⁰)(R²¹), —N(R²²)C(O)N(R²⁰)(R²¹), —N(R²²)C(O)OR²³, —N(R²²)S(O)₂R²³, —C(O)R²³, —S(O)R²³, —OC(O)R²³, —C(O)N(R²⁰)(R²¹), —C(O)C(O)N(R²⁰)(R²¹), —N(R²²)C(O)R²³, —S(O)₂R²³, —S(O)₂N(R²⁰)(R²¹)—, —N═S(═O)(R²³)₂, —S(═O)(═NH)N(R²⁰)(R²¹), —S(═O)(═NH)C(R²⁰)(R²¹), —S(═O)(═NR²³)R²³, —CH₂C(O)N(R²⁰)(R²¹), —CH₂N(R²²)C(O)R²³, —CH₂S(O)₂R²³, and —CH₂S(O)₂N(R²⁰)(R²¹); or two R^{15b} are combined to form a C₃₋₆cycloalkyl or C₂₋₉heterocycloalkyl, wherein C₃₋₆cycloalkyl and C₂₋₉heterocycloalkyl are optionally substituted with one, two, or three groups independently selected from halogen, oxo, C₁₋₆alkyl, C₁₋₆haloalkyl, and C₁₋₆alkoxy;
R¹⁷ is selected from hydrogen, halogen, and C₁₋₆alkyl;
R¹⁸ is selected from hydrogen and C₁₋₆alkyl;
each R²⁰ is independently selected from hydrogen, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R²¹ is independently selected from hydrogen, C₁₋₆alkyl, and C₁₋₆haloalkyl; or R²⁰ and R²¹, together with the nitrogen to which they are attached, form a C₂₋₉heterocycloalkyl;
each R^{21a} is independently selected from C₁₋₆alkyl and C₁₋₆haloalkyl;
each R²² is independently selected from hydrogen, C₁₋₆alkyl, and C₁₋₆haloalkyl;
each R²³ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl; and
[symbol] indicates a single or double bond such that all valences are satisfied.

In some embodiments is a compound of Formula (II), or a pharmaceutically acceptable salt or solvate thereof, wherein Y is N(R¹⁸). In some embodiments is a compound of Formula (II), or a pharmaceutically acceptable salt or solvate thereof, wherein Y is O. In some embodiments is a compound of Formula (II), or a pharmaceutically acceptable salt or solvate thereof, wherein R¹⁷ is hydrogen.

In some embodiments is a compound of Formula (I) or (II), or a pharmaceutically acceptable salt or solvate thereof, wherein X¹¹ is N. In some embodiments is a compound of Formula (I) or (II), or a pharmaceutically acceptable salt or solvate thereof, wherein X¹¹ is C(R¹⁶). In some embodiments is a compound of Formula (I) or (II), or a pharmaceutically acceptable salt or solvate thereof, wherein X¹¹ is C(H). In some embodiments is a compound of Formula (I) or (II), or a pharmaceutically acceptable salt or solvate thereof, wherein X¹¹ is C(O). In some embodiments is a compound of Formula (I) or (II), or a pharmaceutically acceptable salt or solvate thereof, wherein X¹ is N and X² is C. In some embodiments is a compound of Formula (I) or (II), or a pharmaceutically acceptable salt or solvate thereof, wherein X¹ is C and X² is N. In some embodiments is a compound of Formula (I) or (II), or a pharmaceutically acceptable salt or solvate thereof, wherein X³ is C and X⁴ is C. In some embodiments is a compound of Formula (I) or (II), or a pharmaceutically acceptable salt or solvate thereof, wherein X³ is N and X⁴ is C. In some embodiments is a compound of Formula (I) or (II), or a pharmaceutically acceptable salt or solvate thereof, wherein X³ is C and X⁴ is N. In some embodiments is a compound of Formula (I) or (II), or a pharmaceutically acceptable salt or solvate thereof, wherein X⁵ is C(R⁵) or N; X⁶ is C(R⁶) or N; X⁷ is C(R⁷) or N; and X⁸ is C(R⁸) or N.

In some embodiments is a compound of Formula (I), or a pharmaceutically acceptable salt or solvate thereof, having the structure of Formula (Ia):

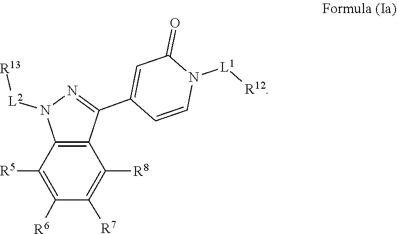

In some embodiments is a compound of Formula (I), or a pharmaceutically acceptable salt or solvate thereof, having the structure of Formula (Ib):

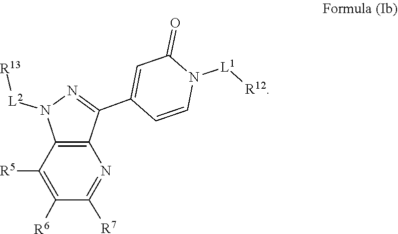

In some embodiments is a compound of Formula (I), or a pharmaceutically acceptable salt or solvate thereof, having the structure of Formula (Ic):

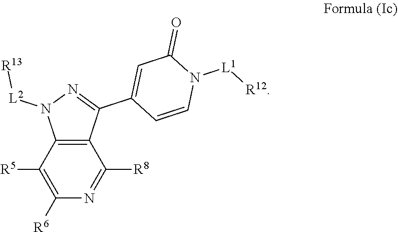

In some embodiments is a compound of Formula (I), or a pharmaceutically acceptable salt or solvate thereof, having the structure of Formula (Id):

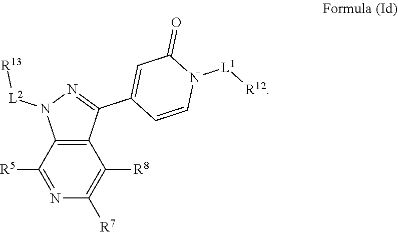

In some embodiments is a compound of Formula (I), or a pharmaceutically acceptable salt or solvate thereof, having the structure of Formula (Ie):

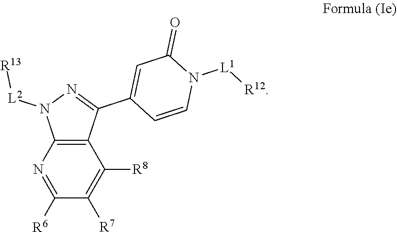

In some embodiments is a compound of Formula (I), or a pharmaceutically acceptable salt or solvate thereof, having the structure of Formula (If):

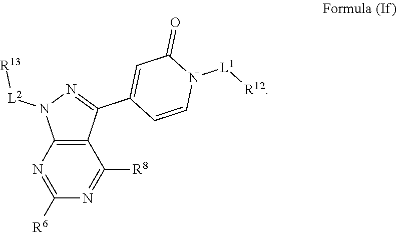

In some embodiments is a compound of Formula (I), or a pharmaceutically acceptable salt or solvate thereof, having the structure of Formula (Ig):

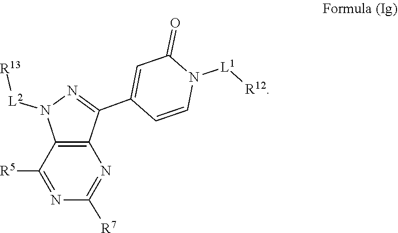

In some embodiments is a compound of Formula (I), or a pharmaceutically acceptable salt or solvate thereof, having the structure of Formula (Ih):

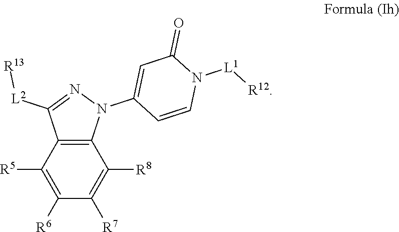

In some embodiments is a compound of Formula (I), or a pharmaceutically acceptable salt or solvate thereof, having the structure of Formula (Ii):

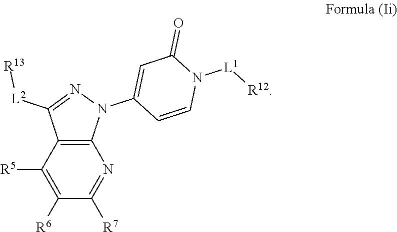

In some embodiments is a compound of Formula (I), or a pharmaceutically acceptable salt or solvate thereof, having the structure of Formula (Ij):

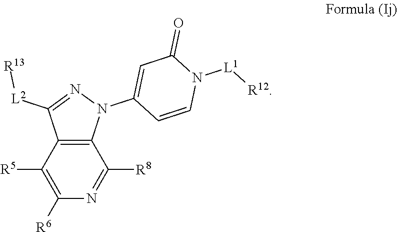

In some embodiments is a compound of Formula (I), or a pharmaceutically acceptable salt or solvate thereof, having the structure of Formula (Ik):

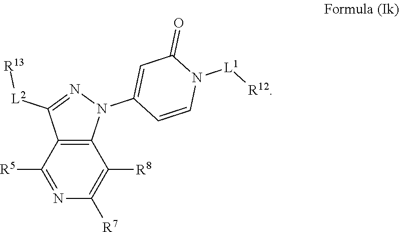

In some embodiments is a compound of Formula (I), or a pharmaceutically acceptable salt or solvate thereof, having the structure of Formula (Il):

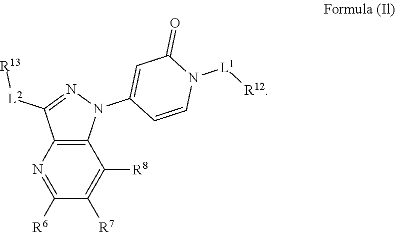

Formula (II). In some embodiments is a compound of Formula (I), or a pharmaceutically acceptable salt or solvate thereof, having the structure of Formula (Im):

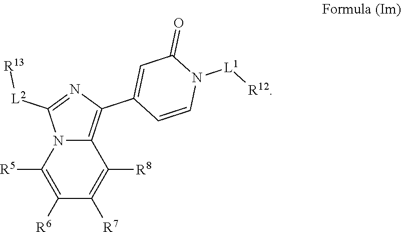

In some embodiments is a compound of Formula (I), or a pharmaceutically acceptable salt or solvate thereof, having the structure of Formula (In):

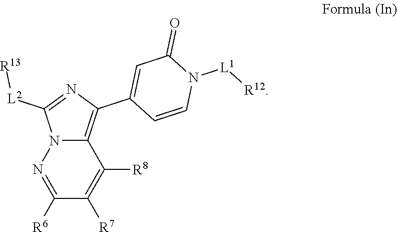

In some embodiments is a compound of Formula (I), or a pharmaceutically acceptable salt or solvate thereof, having the structure of Formula (Io):

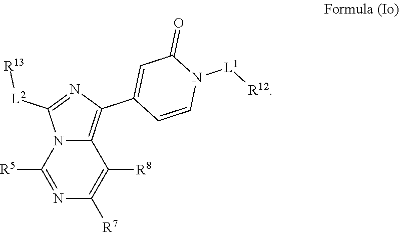

In some embodiments is a compound of Formula (I), or a pharmaceutically acceptable salt or solvate thereof, having the structure of Formula (Ip):

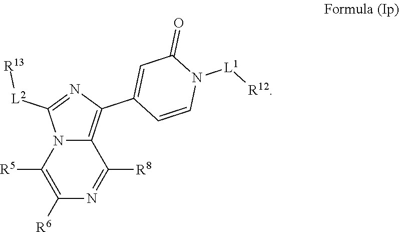

In some embodiments is a compound of Formula (I), or a pharmaceutically acceptable salt or solvate thereof, having the structure of Formula (Iq):

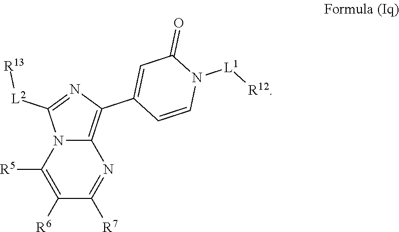

In some embodiments is a compound of Formula (I), or a pharmaceutically acceptable salt or solvate thereof, having the structure of Formula (Ir):

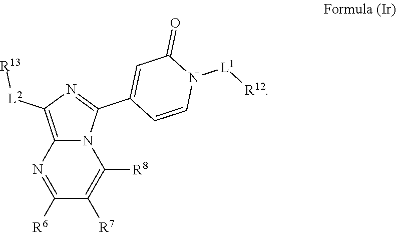

In some embodiments is a compound of Formula (I), or a pharmaceutically acceptable salt or solvate thereof, having the structure of Formula (Is):

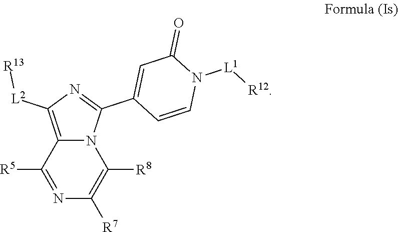

In some embodiments is a compound of Formula (I), or a pharmaceutically acceptable salt or solvate thereof, having the structure of Formula (It):

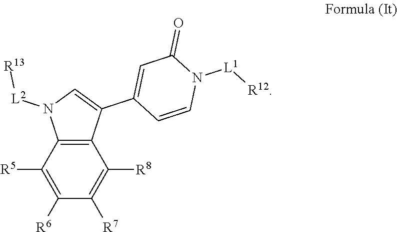

In some embodiments is a compound of Formula (I), or a pharmaceutically acceptable salt or solvate thereof, having the structure of Formula (Iu):

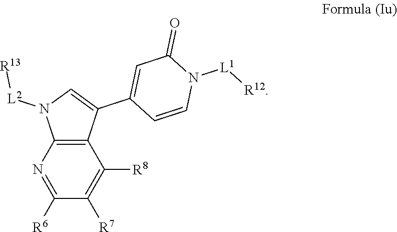

In some embodiments is a compound of Formula (I), or a pharmaceutically acceptable salt or solvate thereof, having the structure of Formula (Iv):

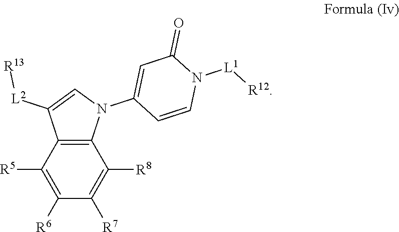

In some embodiments is a compound of Formula (I), or a pharmaceutically acceptable salt or solvate thereof, having the structure of Formula (Iw):

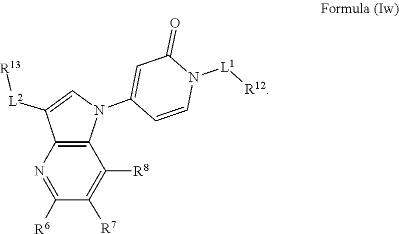

In some embodiments is a compound of Formula (I), or a pharmaceutically acceptable salt or solvate thereof, having the structure of Formula (Ix):

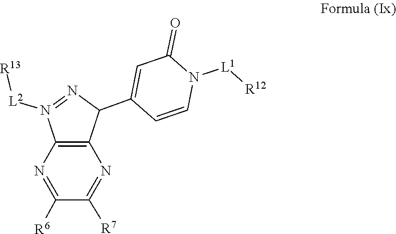

In some embodiments is a compound of Formula (I), or a pharmaceutically acceptable salt or solvate thereof, having the structure of Formula (Iy):

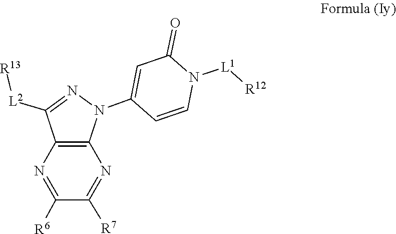

In some embodiments is a compound of Formula (II), or a pharmaceutically acceptable salt or solvate thereof, having the structure of Formula (IIa):

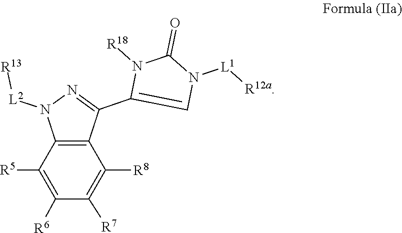

In some embodiments is a compound of Formula (II), or a pharmaceutically acceptable salt or solvate thereof, having the structure of Formula (IIb):

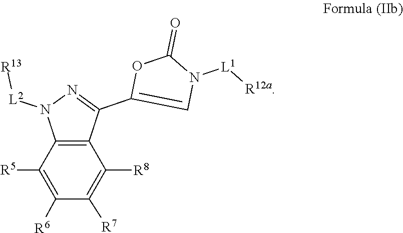

In some embodiments is a compound of Formula (I), (Ia), (Ib), (Ic), (Id), (Ig), (Ih), (Ii), (Ij), (Ik), (Im), (Io), (Ip), (Iq), (Is), (It), (Iv), (II), (IIa), or (IIb), or a pharmaceutically acceptable salt or solvate thereof, wherein R⁵ is selected from hydrogen, halogen, —CN, C₁₋₆alkyl, C₁₋₆haloalkyl, —OR²⁰, —C(O)OR²⁰, —C(O)R²³, —C(O)N(R²⁰)(R²¹), —S(O)₂R²³, and —S(O)₂N(R²⁰)(R²¹)—, wherein C₁₋₆alkyl is optionally substituted with one, two, or three groups selected from R^{14b}. In some embodiments is a compound of Formula (I), (Ia), (Ib), (Ic), (Id), (Ig), (Ih), (Ii), (Ij), (Ik), (Im), (Io), (Ip), (Iq), (Is), (It), (Iv), (II), (IIa), or (IIb), or a pharmaceutically acceptable salt or solvate thereof, wherein R⁵ is selected from hydrogen, halogen, —CN, C₁₋₆alkyl, —OR²⁰, and —C(O)N(R²⁰)(R²¹), wherein C₁₋₆alkyl is optionally substituted with one, two, or three groups selected from R^{14b}, and wherein R²⁰ is independently selected from hydrogen and C₁₋₆alkyl. In some embodiments is a compound of Formula (I), (Ia), (Ib), (Ic), (Id), (Ig), (Ih), (Ii), (Ij), (Ik), (Im), (Io), (Ip), (Iq), (Is), (It), (Iv), (II), (IIa), or (IIb), or a pharmaceutically acceptable salt or solvate thereof, wherein R⁵ is hydrogen. In some embodiments is a compound of Formula (I), (Ia), (Ib), (Ic), (Id), (Ig), (Ih), (Ii), (Ij), (Ik), (Im), (Io), (Ip), (Iq), (Is), (It), (Iv), (II), (IIa), or (IIb), or a pharmaceutically acceptable salt or solvate thereof, wherein R⁵ is halogen. In some embodiments is a compound of Formula (I), (Ia), (Ib), (Ic), (Id), (Ig), (Ih), (Ii), (Ij), (Ik), (Im), (Io), (Ip), (Iq), (Is), (It), (Iv), (II), (IIa), or (IIb), or a pharmaceutically acceptable salt or solvate thereof, wherein R⁵ is F, or Cl. In some embodiments is a compound of Formula (I), (Ia), (Ib), (Ic), (Id), (Ig), (Ih), (Ii), (Ij), (Ik), (Im), (Io), (Ip), (Iq), (Is), (It), (Iv), (II), (IIa), or (IIb), or a pharmaceutically acceptable salt or solvate thereof, wherein R⁵ is F.

In some embodiments is a compound of Formula (I), (Ia), (Ib), (Ic), (Ie), (If), (Ih), (Ii), (Ij), (Il), (Im), (In), (Ip), (Iq), (Ir), (It), (Iu), (Iv), (Iw), (Ix), (Iy), (II), (IIa), or (IIb), or a pharmaceutically acceptable salt or solvate thereof, wherein R⁶ is selected from hydrogen, halogen, —CN, C₁₋₆alkyl, C₁₋₆haloalkyl, —OR²⁰, —C(O)OR²⁰, —C(O)R²³, —C(O)N(R²⁰)(R²¹), —S(O)₂R²³, and —S(O)₂N(R²⁰)(R²¹)—, wherein C₁₋₆alkyl is optionally substituted with one, two, or three groups selected from R^{14b}. In some embodiments is a compound of Formula (I), (Ia), (Ib), (Ic), (Ie), (If), (Ih), (Ii), (Ij), (Il), (Im), (In), (Ip), (Iq), (Ir), (It), (Iu), (Iv), (Iw), (Ix), (Iy), (II), (IIa), or (IIb), or a pharmaceutically acceptable salt or solvate thereof, wherein R⁶ is selected from hydrogen, halogen, —CN, C₁₋₆alkyl, —OR²⁰, and —C(O)N(R²⁰)(R²¹), wherein C₁₋₆alkyl is optionally substituted with one, two, or three groups selected from R^{14b}, and wherein R²⁰ is independently selected from hydrogen and C₁₋₆alkyl. In some embodiments is a compound of Formula (I), (Ia), (Ib), (Ic), (Ie), (If), (Ih), (Ii), (Ij), (Il), (Im), (In), (Ip), (Iq), (Ir), (It), (Iu), (Iv), (Iw), (Ix), (Iy), (II), (IIa), or (IIb), or a pharmaceutically acceptable salt or solvate thereof, wherein R⁶ is hydrogen. In some embodiments is a compound of Formula (I), (Ia), (Ib), (Ic), (Ie), (If), (Ih), (Ii), (Ij), (Il), (Im), (In), (Ip), (Iq), (Ir), (It), (Iu), (Iv), (Iw), (Ix), (Iy), (II), (IIa), or (IIb), or a pharmaceutically acceptable salt or solvate thereof, wherein R⁶ is halogen. In some embodiments is a compound of Formula (I), (Ia), (Ib), (Ic), (Ie), (If), (Ih), (Ii), (Ij), (Il), (Im), (In), (Ip), (Iq), (Ir), (It), (Iu), (Iv), (Iw), (Ix), (Iy), (II), (IIa), or (IIb), or a pharmaceutically acceptable salt or solvate thereof, wherein R⁶ is F, or Cl. In some embodiments is a compound of Formula (I), (Ia), (Ib), (Ic), (Ie), (If), (Ih), (Ii), (Ij), (Il), (Im), (In), (Ip), (Iq), (Ir), (It), (Iu), (Iv), (Iw), (Ix), (Iy), (II), (IIa), or (IIb), or a pharmaceutically acceptable salt or solvate thereof, wherein R⁶ is F.

In some embodiments is a compound of Formula (I), (Ia), (Ib), (Id), (Ie), (Ig), (Ih), (Ii), (Ik), (Il), (Im), (In), (Io), (Iq), (Ir), (Is), (It), (Iu), (Iv), (Iw), (Ix), (Iy), (II), (IIa), or (IIb), or a pharmaceutically acceptable salt or solvate thereof, wherein R⁷ is selected from hydrogen, halogen, —CN, C₁₋₆alkyl, C₁₋₆haloalkyl, —OR²⁰, —C(O)OR²⁰, —C(O)R²³, —C(O)N(R²⁰)(R²¹), —S(O)₂R²³, and —S(O)₂N(R²⁰)(R²¹)—, wherein C₁₋₆alkyl is optionally substituted with one, two, or three groups selected from R^{14b}. In some embodiments is a compound of Formula (I), (Ia), (Ib), (Id), (Ie), (Ig), (Ih), (Ii), (Ik), (Il), (Im), (In), (Io), (Iq), (Ir), (Is), (It), (Iu), (Iv), (Iw), (Ix), (Iy), (II), (IIa), or (Jib), or a pharmaceutically acceptable salt or solvate thereof, wherein R⁷ is selected from hydrogen, halogen, —CN, C₁₋₆alkyl, —OR²⁰, and —C(O)N(R²⁰)(R²¹), wherein C₁₋₆alkyl is optionally substituted with one, two, or three groups selected from R^{14b}, and wherein R²⁰ is independently selected from hydrogen and C₁₋₆alkyl. In some embodiments is a compound of Formula (I), (Ia), (Ib), (Id), (Ie), (Ig), (Ih), (Ii), (Ik), (Il), (Im), (In), (Io), (Iq), (Ir), (Is), (It), (Iu), (Iv), (Iw), (Ix), (Iy), (II), (IIa), or (IIb), or a pharmaceutically acceptable salt or solvate thereof, wherein R⁷ is hydrogen. In some embodiments is a compound of Formula (I), (Ia), (Ib), (Id), (Ie), (Ig), (Ih), (Ii), (Ik), (Il), (Im), (In), (Io), (Iq), (Ir), (Is), (It), (Iu), (Iv), (Iw), (Ix), (Iy), (II), (IIa), or (IIb), or a pharmaceutically acceptable salt or solvate thereof, wherein R⁷ is halogen. In some embodiments is a compound of Formula (I), (Ia), (Ib), (Id), (Ie), (Ig), (Ih), (Ii), (Ik), (Il), (Im), (In), (Io), (Iq), (Ir), (Is), (It), (Iu), (Iv), (Iw), (Ix), (Iy), (II), (IIa), or (IIb), or a pharmaceutically acceptable salt or solvate thereof, wherein R⁷ is F, or Cl. In some embodiments is a compound of Formula (I), (Ia), (Ib), (Id), (Ie), (Ig), (Ih), (Ii), (Ik), (Il), (Im), (In), (Io), (Iq), (Ir), (Is), (It), (Iu), (Iv), (Iw), (Ix), (Iy), (II), (IIa), or (IIb), or a pharmaceutically acceptable salt or solvate thereof, wherein R⁷ is F.

In some embodiments is a compound of Formula (I), (Ia), (Ic), (Id), (Ie), (If), (Ih), (Ij), (Ik), (Il), (Im), (In), (Io), (Ip), (Ir), (Is), (It), (Iu), (Iv), (Iw), (II), (IIa), or (IIb), or a pharmaceutically acceptable salt or solvate thereof, wherein R⁸ is selected from hydrogen, halogen, —CN, C₁₋₆alkyl, C₁₋₆haloalkyl, —OR²⁰, —C(O)OR²⁰, —C(O)R²³, —C(O)N(R²⁰)(R²¹), —S(O)₂R²³, and —S(O)₂N(R²⁰)(R²¹)—, wherein C₁₋₆alkyl is optionally substituted with one, two, or three groups selected from R^{14b}. In some embodiments is a compound of Formula (I), (Ia), (Ic), (Id), (Ie), (If), (Ih), (Ij), (Ik), (Il), (Im), (In), (Io), (Ip), (Ir), (Is), (It), (Iu), (Iv), (Iw), (II), (IIa), or (IIb), or a pharmaceutically acceptable salt or solvate thereof, wherein R⁸ is selected from hydrogen, halogen, —CN, C₁₋₆alkyl, —OR²⁰, and —C(O)N(R²⁰)(R²¹), wherein C₁₋₆alkyl is optionally substituted with one, two, or three groups selected from R^{14b}, and wherein R²⁰ is independently selected from hydrogen and C₁₋₆alkyl. In some embodiments is a compound of Formula (I), (Ia), (Ic), (Id), (Ie), (If), (Ih), (Ij), (Ik), (Il), (Im), (In), (Io), (Ip), (Ir), (Is), (It), (Iu), (Iv), (Iw), (II), (IIa), or (IIb), or a pharmaceutically acceptable salt or solvate thereof, wherein R⁸ is hydrogen. In some embodiments is a compound of Formula (I), (Ia), (Ic), (Id), (Ie), (If), (Ih), (Ij), (Ik), (Il), (Im), (In), (Io), (Ip), (Ir), (Is), (It), (Iu), (Iv), (Iw), (II), (IIa), or (IIb), or a pharmaceutically acceptable salt or solvate thereof, wherein R⁸ is halogen. In some embodiments is a compound of Formula (I), (Ia), (Ic), (Id), (Ie), (If), (Ih), (Ij), (Ik), (Ii), (Im), (In), (Io), (Ip), (Ir), (Is), (It), (Iu), (Iv), (Iw), (II), (IIa), or (IIb), or a pharmaceutically acceptable salt or solvate thereof, wherein R⁸ is F, or Cl. In some embodiments is a compound of Formula (I), (Ia), (Ic), (Id), (Ie), (If), (Ih), (Ij), (Ik), (Il), (Im), (In), (Io), (Ip), (Ir), (Is), (It), (Iu), (Iv), (Iw), (II), (IIa), or (IIb), or a pharmaceutically acceptable salt or solvate thereof, wherein R⁸ is F.

In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), (Iy), (II), (IIa), or (IIb), or a pharmaceutically acceptable salt or solvate thereof, wherein L² is a bond. In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), (Iy), (II), (IIa), or (IIb), or a pharmaceutically acceptable salt or solvate thereof, wherein L² is C₁₋₆alkylene optionally substituted with one, two, or three groups selected from R^{14a}. In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), (Iy), (II), (IIa), or (IIb), or a pharmaceutically acceptable salt or solvate thereof, wherein R¹³ is selected from C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl, wherein C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three groups selected from R^{15b}. In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), (Iy), (II), (IIa), or (IIb), or a pharmaceutically acceptable salt or solvate thereof, wherein R¹³ is selected from C₃₋₁₀cycloalkyl, C₂₋₉heterocycloalkyl, C₆₋₁₀aryl, and C₁₋₉heteroaryl, wherein C₃₋₁₀cycloalkyl, C₂₋₉heterocycloalkyl, C₆₋₁₀aryl, and C₁₋₉heteroaryl are optionally substituted with one, two, or three groups selected from R^{15b}. In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), (Iy), (II), (IIa), or (IIb), or a pharmaceutically acceptable salt or solvate thereof, wherein R¹³ is C₆₋₁₀aryl optionally substituted with one, two, or three groups selected from R^{15b}. In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), (Iy), (II), (IIa), or (IIb), or a pharmaceutically acceptable salt or solvate thereof, wherein R¹³ is phenyl substituted with one, two, or three groups selected from R^{15b}. In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), (Iy), (II), (IIa), or (IIb), or a pharmaceutically acceptable salt or solvate thereof, wherein R¹³ is C₁₋₉heteroaryl optionally substituted with one, two, or three groups selected from R^{15b}. In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), (Iy), (II), (IIa), or (IIb), or a pharmaceutically acceptable salt or solvate thereof, wherein R¹³ is C₁₋₉heteroaryl optionally substituted with one, two, or three groups selected from R^{15b}, wherein C₁₋₉heteroaryl is selected from pyridyl, pyrimidinyl, pyrazinyl, pyridazinyl, thienyl, furanyl, pyrrolyl, oxazolyl, thiazolyl, pyrazolyl, indazolyl, and imidazolyl. In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), (Iy), (II), (IIa), or (IIb), or a pharmaceutically acceptable salt or solvate thereof, wherein R¹³ is C₃₋₁₀cycloalkyl optionally substituted with one, two, or three groups selected from R^{15b}. In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), (Iy), (II), (IIa), or (IIb), or a pharmaceutically acceptable salt or solvate thereof, wherein R¹³ is C₂₋₉heterocycloalkyl optionally substituted with one, two, or three groups selected from R^{15b}. In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), (Iy), (II), (IIa), or (IIb), or a pharmaceutically acceptable salt or solvate thereof, wherein each R^{15b} is independently selected from halogen, C₁₋₆alkyl, C₁₋₆haloalkyl, and —OR²⁰, wherein R²⁰ is selected from C₁₋₆alkyl and C₁₋₆haloalkyl. In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), (Iy), (II), (IIa), or (IIb), or a pharmaceutically acceptable salt or solvate thereof, wherein L¹ is C₁₋₆alkylene optionally substituted with one, two, or three groups selected from R^{14a}. In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), (Iy), (II), (IIa), or (IIb), or a pharmaceutically acceptable salt or solvate thereof, wherein L¹ is unsubstituted C₁₋₆alkylene. In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), (Iy), (II), (IIa), or (IIb), or a pharmaceutically acceptable salt or solvate thereof, wherein L¹ is a bond. In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), (Iy), (II), (IIa), or (IIb), or a pharmaceutically acceptable salt or solvate thereof, wherein R¹² is selected from C₂₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl, wherein C₂₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three groups selected from R^{15a}. In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), or (Iy), or a pharmaceutically acceptable salt or solvate thereof, wherein R¹² is C₁₋₉heteroaryl optionally substituted with one, two, or three groups selected from R^{15a}. In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), or (Iy), or a pharmaceutically acceptable salt or solvate thereof, wherein R¹² is C₁₋₉heteroaryl optionally substituted with one, two, or three groups selected from R^{15a}, wherein C₁₋₉heteroaryl is selected from pyridyl, pyrimidinyl, pyrazinyl, pyridazinyl, thienyl, furanyl, pyrrolyl, oxazolyl, thiazolyl, pyrazolyl, indazolyl, and imidazolyl. In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), or (Iy), or a pharmaceutically acceptable salt or solvate thereof, wherein R¹² is C₂₋₉heterocycloalkyl optionally substituted with one, two, or three groups selected from R^{15a}. In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), or (Iy), or a pharmaceutically acceptable salt or solvate thereof, wherein each R^{15a} is independently selected from halogen, oxo, —CN, C₁₋₆alkyl, C₁₋₆haloalkyl, C₂₋₆alkenyl, C₂₋₆alkynyl, C₃₋₁₀cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR²⁰, —SR²⁰, —SF₅, —N(R²⁰)(R²¹), —C(O)OR²⁰, —OC(O)N(R²⁰)(R²¹), —N(R²²)C(O)N(R²⁰)(R²¹), —N(R²²)C(O)OR²³, —N(R²²)S(O)₂R²³, —C(O)R²³, —S(O)R²³, —OC(O)R²³, —C(O)N(R²⁰)(R²¹), —C(O)C(O)N(R²⁰)(R²¹), —N(R²²)C(O)R²³, —S(O)₂R²³, —S(O)₂N(R²⁰)(R²¹)—, —N═S(═O)(R²³)₂, —S(═O)(═NH)N(R²⁰)(R²¹), —S(═O)(═NH)C(R²⁰)(R²¹), —S(═O)(═NR²³)R²³, —CH₂C(O)N(R²⁰)(R²¹), —CH₂N(R²²)C(O)R²³, —CH₂S(O)₂R²³, —CH₂S(O)₂N(R²⁰)(R²¹), wherein C₁₋₆alkyl, C₁₋₆haloalkyl, C₂₋₆alkenyl, C₂₋₆alkynyl, C₃₋₆cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²⁰, —SR²⁰, —SF₅, —N(R²⁰)(R²¹), —C(O)OR²⁰, —OC(O)N(R²⁰)(R²¹), —N(R²²)C(O)N(R²⁰)(R²¹), —N(R²²)C(O)OR²³, —N(R²²)S(O)₂R²³, —C(O)R²³, —S(O)R¹³, —OC(O)R²³, —C(O)N(R²⁰)(R²¹), —C(O)C(O)N(R²⁰)(R²¹), —N(R²²)C(O)R²³, —S(O)₂R²³, —S(O)₂N(R²⁰)(R²¹)—, —N═S(═O)(R²³)₂, —S(═O)(═NH)N(R²⁰)(R²¹), —S(═O)(═NH)C(R²⁰)(R²¹), —S(═O)(═NR²³)R²³, —CH₂C(O)N(R²⁰)(R²¹), —CH₂N(R²²)C(O)R²³, —CH₂S(O)₂R²³, and —CH₂S(O)₂N(R²⁰)(R²¹); or two R^{15a} are combined to form a C₃₋₆cycloalkyl or C₂₋₉heterocycloalkyl, wherein C₃₋₆cycloalkyl and C₂₋₉heterocycloalkyl are optionally substituted with one, two, or three groups independently selected from halogen, oxo, C₁₋₆alkyl, C₁₋₆haloalkyl, and C₁₋₆alkoxy. In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), or (Iy), or a pharmaceutically acceptable salt or solvate thereof, wherein each R¹⁵, is independently selected from halogen, —CN, C₁₋₆alkyl, and C₁₋₆haloalkyl, C₃₋₁₀cycloalkyl, —CH₂—C₃₋₆cycloalkyl, C₂₋₉heterocycloalkyl, —CH₂—C₂₋₉heterocycloalkyl, —NH—C₃₋₉cycloalkyl, C₆₋₁₀aryl, —CH₂—C₆₋₁₀aryl, C₁₋₉heteroaryl, —CH₂—C₁₋₉heteroaryl, —OR²⁰, —SR²⁰, —SF₅, —N(R²⁰)(R²¹), —C(O)OR²⁰, —OC(O)N(R²⁰)(R²¹), —N(R²²)C(O)N(R²⁰)(R²¹), —N(R²²)C(O)OR²³, —N(R²²)S(O)₂R²³, —C(O)R²³, —S(O)R²³, —OC(O)R²³, —C(O)N(R²⁰)(R²¹), —C(O)C(O)N(R²⁰)(R²¹), —N(R²²)C(O)R²³, —S(O)₂R²³, —S(O)₂N(R²⁰)(R²¹)—, —N═S(═O)(R²³)₂, —S(═O)(═NH)N(R²⁰)(R²¹), —S(═O)(═NH)C(R²⁰)(R²¹), —S(═O)(═NR²³)R²³, —CH₂C(O)N(R²⁰)(R²¹), —CH₂N(R²²)C(O)R²³, —CH₂S(O)₂R²¹, —CH₂S(O)₂N(R²⁰)(R²¹), wherein C₁₋₆alkyl, C₁₋₆haloalkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²⁰, —SR²⁰, —SF₅, —N(R²⁰)(R²¹), —C(O)OR²⁰, —OC(O)N(R²⁰)(R²¹), —N(R²²)C(O)N(R²⁰)(R²¹), —N(R²²)C(O)OR²³, —N(R²²)S(O)₂R²³, —C(O)R²³, —S(O)R²³, —OC(O)R²³, —C(O)N(R²⁰)(R²¹), —C(O)C(O)N(R²⁰)(R²¹), —N(R²²)C(O)R²³, —S(O)₂R²³, —S(O)₂N(R²⁰)(R²¹)—, —N═S(═O)(R²³)₂, —S(═O)(═NH)N(R²⁰)(R²¹), —S(═O)(═NH)C(R²⁰)(R²¹), —S(═O)(═NR²³)R²³, —CH₂C(O)N(R²⁰)(R²¹), —CH₂N(R²²)C(O)R²³, —CH₂S(O)₂R²³, and —CH₂S(O)₂N(R²⁰)(R²¹); or two R^{15a} are combined to form a C₃₋₆cycloalkyl or C₂₋₉heterocycloalkyl, wherein C₃₋₆cycloalkyl and C₂₋₉heterocycloalkyl are optionally substituted with one, two, or three groups independently selected from halogen, oxo, C₁₋₆alkyl, C₁₋₆haloalkyl, and C₁₋₆alkoxy; each R²⁰ is independently selected from hydrogen, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), or (Iy), or a pharmaceutically acceptable salt or solvate thereof, wherein each R^{14a}, R^{14b}, and R^{14c} are each independently selected from halogen, oxo, —CN, C₁₋₆alkyl, C₁₋₆haloalkyl, C₂₋₆alkenyl, C₂₋₆alkynyl, C₃₋₁₀cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR²⁰, —SR²⁰, —SF₅, —N(R²⁰)(R²¹), —C(O)OR²⁰, —OC(O)N(R²⁰)(R²¹), —N(R²²)C(O)N(R²⁰)(R²¹), —N(R²²)C(O)OR²³, —N(R²²)S(O)₂R²³, —C(O)R²³, —S(O)R²³, —OC(O)R²³, —C(O)N(R²⁰)(R²¹), —C(O)C(O)N(R²⁰)(R²¹), —N(R²²)C(O)R²³, —S(O)₂R²³, —S(O)₂N(R²⁰)(R²¹)—, —N═S(═O)(R²³)₂, —S(═O)(═NH)N(R²⁰)(R²¹), —S(═O)(═NH)C(R²⁰)(R²¹), —S(═O)(═NR²³)R²³, —CH₂C(O)N(R²⁰)(R²¹), —CH₂N(R²²)C(O)R²³, —CH₂S(O)₂R²³, —CH₂S(O)₂N(R²⁰)(R²¹), wherein C₁₋₆alkyl, C₁₋₆haloalkyl, C₂₋₆alkenyl, C₂₋₆alkynyl, C₃₋₆cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²⁰, —SR²⁰, —SF₅, —N(R²⁰)(R²¹), —C(O)OR²⁰, —OC(O)N(R²⁰)(R²¹), —N(R²²)C(O)N(R²⁰)(R²¹), —N(R²²)C(O)OR²³, —N(R²²)S(O)₂R²³, —C(O)R²³, —S(O)R²³, —OC(O)R²³, —C(O)N(R²⁰)(R²¹), —C(O)C(O)N(R²⁰)(R²¹), —N(R²²)C(O)R²³, —S(O)₂R²³, —S(O)₂N(R²⁰)(R²¹)—, —N═S(═O)(R²³)₂, —S(═O)(═NH)N(R²⁰)(R²¹), —S(═O)(═NH)C(R²⁰)(R²¹), —S(═O)(═NR²³)R²³, —CH₂C(O)N(R²⁰)(R²¹), —CH₂N(R²²)C(O)R²³, —CH₂S(O)₂R²³, and —CH₂S(O)₂N(R²⁰)(R²¹);
In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), or (Iy), or a pharmaceutically acceptable salt or solvate thereof, wherein each R^{15b} is independently selected from halogen, oxo, —CN, C₁₋₆alkyl, C₁₋₆haloalkyl, C₂₋₆alkenyl, C₂₋₆alkynyl, C₃₋₁₀cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR²⁰, —SR²⁰, —SF₅, —N(R²⁰)(R²¹), —C(O)OR²⁰, —OC(O)N(R²⁰)(R²¹), —N(R²²)C(O)N(R²⁰)(R²¹), —N(R²²)C(O)OR²³, —N(R²²)S(O)₂R²³, —C(O)R²³, —S(O)R²³, —OC(O)R²³, —C(O)N(R²⁰)(R²¹), —C(O)C(O)N(R²⁰)(R²¹), —N(R²²)C(O)R²³, —S(O)₂R²³, —S(O)₂N(R²⁰)(R²¹)—, —N═S(═O)(R²³)₂, —S(═O)(═NH)N(R²⁰)(R²¹), —S(═O)(═NH)C(R²⁰)(R²¹), —S(═O)(═NR²³)R²³, —CH₂C(O) N(R²⁰)(R²¹), —CH₂N(R²²)C(O)R²³, —CH₂S(O)₂R²³, —CH₂S(O)₂N(R²⁰)(R²¹), wherein C₁₋₆alkyl, C₁₋₆haloalkyl, C₂₋₆alkenyl, C₂₋₆alkynyl, C₃₋₆cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²⁰, —SR²⁰, —SF₅, —N(R²⁰)(R²¹), —C(O)OR²⁰, —OC(O)N(R²⁰)(R²¹), —N(R²²)C(O)N(R²⁰)(R²¹), —N(R²²)C(O)OR²³, —N(R²²)S(O)₂R²³, —C(O)R²³, —S(O)R²³, —OC(O)R²³, —C(O)N(R²⁰)(R²¹), —C(O)C(O)N(R²⁰)(R²¹), —N(R²²)C(O)R²³, —S(O)₂R²³, —S(O)₂N(R²⁰)(R²¹)—, —N═S(═O)(R²³)₂, —S(═O)(═NH)N(R²⁰)(R²¹), —S(═O)(═NH)C(R²⁰)(R²¹), —S(═O)(═NR²)R²³, —CH₂C(O)N(R²⁰)(R²¹), —CH₂N(R²²)C(O)R²³, —CH₂S(O)₂R²³, and —CH₂S(O)₂N(R²⁰)(R²¹); or two R^{15b} are combined to form a C₃₋₆cycloalkyl or C₂₋₉heterocycloalkyl, wherein C₃₋₆cycloalkyl and C₂₋₉heterocycloalkyl are optionally substituted with one, two, or three groups independently selected from halogen, oxo, C₁₋₆alkyl, C₁₋₆haloalkyl, and C₁₋₆alkoxy
each R²¹ is independently selected from hydrogen, C₁₋₆alkyl, and C₁₋₆haloalkyl; or R²⁰ and R²¹, together with the nitrogen to which they are attached, form a C₂₋₉heterocycloalkyl;
each R^{21a} is independently selected from C₁₋₆alkyl and C₁₋₆haloalkyl;
each R²² is independently selected from hydrogen, C₁₋₆alkyl, and C₁₋₆haloalkyl;
each R²³ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl; and

In some embodiments is a compound of Formula (II), (IIa), or (IIb), or a pharmaceutically acceptable salt or solvate thereof, wherein R^{12a} is C₁₋₉heteroaryl optionally substituted with one, two, or three groups selected from R^{15a}. In some embodiments is a compound of Formula (II), (IIa), or (IIb), or a pharmaceutically acceptable salt or solvate thereof, wherein R^{12a} is C₁₋₉heteroaryl optionally substituted with one, two, or three groups selected from R^{15a}, wherein C₁₋₉heteroaryl is selected from pyridyl, pyrimidinyl, pyrazinyl, pyridazinyl, thienyl, furanyl, pyrrolyl, oxazolyl, thiazolyl, pyrazolyl, indazolyl, and imidazolyl. In some embodiments is a compound of Formula (II), (IIa), or (IIb), or a pharmaceutically acceptable salt or solvate thereof, wherein R^{12a} is C₂₋₉heterocycloalkyl optionally substituted with one, two, or three groups selected from R^{15a}. In some embodiments is a compound of Formula (II), (IIa), or (IIb), or a pharmaceutically acceptable salt or solvate thereof, wherein each R^{15a} is independently selected from halogen, —CN, C₁₋₆alkyl, and C₁₋₆haloalkyl.

In another aspect, described herein is a pharmaceutical composition comprising a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), (Iy), (II), (IIa), or (IIb), or a pharmaceutically acceptable salt or solvate thereof, and a pharmaceutically acceptable excipient.

In another aspect, described herein is a method of inhibition of the Hippo pathway in a subject in need thereof comprising administering to the subject a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), (Iy), (II), (IIa), or (IIb), or a pharmaceutically acceptable salt or solvate thereof. In some embodiments is a method of inhibition of YAP-TEAD and/or TAZ-TEAD interaction in a subject in need thereof comprising administering to the patient a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), (Iy), (II), (IIa), or (IIb), or a pharmaceutically acceptable salt or solvate thereof.

In another aspect, described herein is a method of treating a disease or condition affected by the inhibition of YAP-TEAD and/or TAZ-TEAD interaction in a subject in need thereof comprising administering to the subject a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), (Iy), (II), (IIa), or (IIb), or a pharmaceutically acceptable salt or solvate thereof.

In another aspect, described herein is a method of treating cancer in a subject in need thereof comprising administering to the subject a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), (Iy), (II), (IIa), or (IIb), or a pharmaceutically acceptable salt or solvate thereof. In some embodiments is a method of treating cancer in a subject in need thereof comprising administering to the subject a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), (Iy), (II), (IIa), or (IIb), or a pharmaceutically acceptable salt or solvate thereof, wherein the cancer is selected from breast cancer, lung cancer, liver cancer, ovarian cancer, squamous cancer, renal cancer, gastric cancer, medulloblastoma, colon cancer, and pancreatic cancer.

In another aspect, described herein is a method of treating cardiovascular disease in a subject in need thereof comprising administering to the subject a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), (Iy), (II), (IIa), or (IIb), or a pharmaceutically acceptable salt or solvate thereof.

In another aspect, described herein is a method of treating fibrosis in a subject in need thereof comprising administering to the subject a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), (Iy), (II), (IIa), or (IIb), or a pharmaceutically acceptable salt or solvate thereof. In some embodiments is a method of treating fibrosis in a subject in need thereof comprising administering to the subject a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), (Iy), (II), (IIa), or (IIb), or a pharmaceutically acceptable salt or solvate thereof, wherein the fibrosis is liver fibrosis, kidney fibrosis and lung fibrosis.

Any combination of the groups described above for the various variables is contemplated herein. Throughout the specification, groups and substituents thereof are chosen by one skilled in the field to provide stable moieties and compounds.

Other objects, features and advantages of the compounds, methods and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### DETAILED DESCRIPTION OF THE INVENTION

Proper function of the Hippo pathway involves a cascade of kinases in the cytoplasm which results in the phosphorylation of two transcriptional co activators, YAP (Yes-associated protein) and TAZ (Transcription co-activator with PDZ binding motif), as well as their sequestration in the cytoplasm and eventually their degradation. Non-phosphorylated, activated YAP/TAZ co-activators are translocated into the cell nucleus, binding and activating Transcriptional enhanced associate domain (TEAD) transcription factor family (TEAD1-4) which plays a role in the development of a wide variety of cancer types and hyperproliferative diseases. Therefore, inhibition of YAP, TAZ, TEAD, and YAP-TEAD or TAZ-TEAD protein-protein interaction is an interesting target for preventing and/or treating cancer and other hyperproliferative disorders and diseases associated with the dysfunction of the Hippo pathway.

### Certain Terminology

Unless otherwise stated, the following terms used in this application have the definitions given below. The use of the term “including” as well as other forms, such as “include”, “includes” and “included” is not limiting. The section headings used herein are for organizational purposes only and are not to be construed as limiting the subject matter described.

As used herein, C₁-Cₓ includes C₁-C₂, C₁-C₃ . . . C₁-Cₓ. By way of example only, a group designated as “C₁-C₄” indicates that there are one to four carbon atoms in the moiety, i.e. groups containing 1 carbon atom, 2 carbon atoms, 3 carbon atoms or 4 carbon atoms. Thus, by way of example only, “C₁-C₄ alkyl” indicates that there are one to four carbon atoms in the alkyl group, i.e., the alkyl group is selected from among methyl, ethyl, propyl, iso-propyl, n-butyl, iso-butyl, sec-butyl, and t-butyl.

An “alkyl” group refers to an aliphatic hydrocarbon group. The alkyl group is branched or straight chain. In some embodiments, the “alkyl” group has 1 to 10 carbon atoms, i.e. a C₁-C₁₀alkyl. Whenever it appears herein, a numerical range such as “1 to 10” refers to each integer in the given range; e.g., “1 to 10 carbon atoms” means that the alkyl group consist of 1 carbon atom, 2 carbon atoms, 3 carbon atoms, 4 carbon atoms, 5 carbon atoms, 6 carbon atoms, etc., up to and including 10 carbon atoms, although the present definition also covers the occurrence of the term “alkyl” where no numerical range is designated. In some embodiments, an alkyl is a C₁-C₆alkyl. In one aspect, the alkyl is methyl, ethyl, propyl, iso-propyl, n-butyl, iso-butyl, sec-butyl, or t-butyl. Typical alkyl groups include, but are in no way limited to, methyl, ethyl, propyl, isopropyl, butyl, isobutyl, sec-butyl, tertiary butyl, pentyl, neopentyl, or hexyl.

An “alkylene” group refers to a divalent alkyl radical. Any of the above mentioned monovalent alkyl groups may be an alkylene by abstraction of a second hydrogen atom from the alkyl. In some embodiments, an alkylene is a C₁-C₆alkylene. In other embodiments, an alkylene is a C₁-C₄alkylene. In certain embodiments, an alkylene comprises one to four carbon atoms (e.g., C₁-C₄ alkylene). In other embodiments, an alkylene comprises one to three carbon atoms (e.g., C₁-C₃ alkylene). In other embodiments, an alkylene comprises one to two carbon atoms (e.g., C₁-C₂ alkylene). In other embodiments, an alkylene comprises one carbon atom (e.g., C₁ alkylene). In other embodiments, an alkylene comprises two carbon atoms (e.g., C₂ alkylene). In other embodiments, an alkylene comprises two to four carbon atoms (e.g., C₂-C₄ alkylene). Typical alkylene groups include, but are not limited to, —CH₂—, —CH(CH₃)—, —C(CH₃)₂—, —CH₂CH₂—, —CH₂CH(CH₃)—, —CH₂C(CH₃)₂—, —CH₂CH₂CH₂—, —CH₂CH₂CH₂CH₂—, and the like.

“Deuteroalkyl” refers to an alkyl group where 1 or more hydrogen atoms of an alkyl are replaced with deuterium.

The term “alkenyl” refers to a type of alkyl group in which at least one carbon-carbon double bond is present. In one embodiment, an alkenyl group has the formula —C(R)═CR₂, wherein R refers to the remaining portions of the alkenyl group, which may be the same or different. In some embodiments, R is H or an alkyl. In some embodiments, an alkenyl is selected from ethenyl (i.e., vinyl), propenyl (i.e., allyl), butenyl, pentenyl, pentadienyl, and the like. Non-limiting examples of an alkenyl group include —CH═CH₂, —C(CH₃)═CH₂, —CH═CHCH₃, —C(CH₃)═CHCH₃, and —CH₂CH═CH₂.

The term “alkynyl” refers to a type of alkyl group in which at least one carbon-carbon triple bond is present. In one embodiment, an alkenyl group has the formula —C≡C—R, wherein R refers to the remaining portions of the alkynyl group. In some embodiments, R is H or an alkyl. In some embodiments, an alkynyl is selected from ethynyl, propynyl, butynyl, pentynyl, hexynyl, and the like. Non-limiting examples of an alkynyl group include —C≡CH, —C≡CCH₃—C≡CCH₂CH₃, —CH₂C≡CH.

An “alkoxy” group refers to a (alkyl)O— group, where alkyl is as defined herein.

The term “alkylamine” refers to the —N(alkyl)ₓH_{y} group, where x is 0 and y is 2, or where x is 1 and y is 1, or where x is 2 and y is 0.

The term “aromatic” refers to a planar ring having a delocalized π-electron system containing 4n+2π electrons, where n is an integer. The term “aromatic” includes both carbocyclic aryl (“aryl”, e.g., phenyl) and heterocyclic aryl (or “heteroaryl” or “heteroaromatic”) groups (e.g., pyridine). The term includes monocyclic or fused-ring polycyclic (i.e., rings which share adjacent pairs of carbon or nitrogen atoms) groups.

The term “carbocyclic” or “carbocycle” refers to a ring or ring system where the atoms forming the backbone of the ring are all carbon atoms. The term thus distinguishes carbocyclic from “heterocyclic” rings or “heterocycles” in which the ring backbone contains at least one atom which is different from carbon. In some embodiments, at least one of the two rings of a bicyclic carbocycle is aromatic. In some embodiments, both rings of a bicyclic carbocycle are aromatic. Carbocycle includes cycloalkyl and aryl.

As used herein, the term “aryl” refers to an aromatic ring wherein each of the atoms forming the ring is a carbon atom. In one aspect, aryl is phenyl or a naphthyl. In some embodiments, an aryl is a phenyl. In some embodiments, an aryl is a C₆-C₁₀aryl. Depending on the structure, an aryl group is a monoradical or a diradical (i.e., an arylene group).

The term “cycloalkyl” refers to a monocyclic or polycyclic aliphatic, non-aromatic radical, wherein each of the atoms forming the ring (i.e. skeletal atoms) is a carbon atom. In some embodiments, cycloalkyls are spirocyclic or bridged compounds. In some embodiments, cycloalkyls are optionally fused with an aromatic ring, and the point of attachment is at a carbon that is not an aromatic ring carbon atom. Cycloalkyl groups include groups having from 3 to 10 ring atoms. In some embodiments, cycloalkyl groups are selected from among cyclopropyl, cyclobutyl, cyclopentyl, cyclopentenyl, cyclohexyl, cyclohexenyl, cycloheptyl, cyclooctyl, spiro[2.2]pentyl, norbornyl and bicyclo[1.1.1]pentyl. In some embodiments, a cycloalkyl is a C₃-C₆cycloalkyl. In some embodiments, a cycloalkyl is a monocyclic cycloalkyl. Monocyclic cycloalkyls include, but are not limited to, cyclopropyl, cyclobutyl, cyclopentyl, cyclohexyl, cycloheptyl, and cyclooctyl. Polycyclic cycloalkyls include, for example, adamantyl, norbornyl (i.e., bicyclo[2.2.2]octyl and bicyclo[2.2.1]heptanyl), norbornenyl, decalinyl, 7,7-dimethyl-bicyclo[2.2.1]heptanyl, and the like.

The term “halo” or, alternatively, “halogen” or “halide” means fluoro, chloro, bromo or iodo. In some embodiments, halo is fluoro, chloro, or bromo.

The term “haloalkyl” refers to an alkyl in which one or more hydrogen atoms are replaced by a halogen atom. In one aspect, a fluoroalkyl is a C₁-C₆fluoroalkyl.

The term “fluoroalkyl” refers to an alkyl in which one or more hydrogen atoms are replaced by a fluorine atom. In one aspect, a fluoroalkyl is a C₁-C₆fluoroalkyl. In some embodiments, a fluoroalkyl is selected from trifluoromethyl, difluoromethyl, fluoromethyl, 2,2,2-trifluoroethyl, 1-fluoromethyl-2-fluoroethyl, and the like.

The term “heteroalkyl” refers to an alkyl group in which one or more skeletal atoms of the alkyl are selected from an atom other than carbon, e.g., oxygen, nitrogen (e.g. —NH—, —N(alkyl)-, sulfur, or combinations thereof. A heteroalkyl is attached to the rest of the molecule at a carbon atom of the heteroalkyl. In one aspect, a heteroalkyl is a C₁-C₆heteroalkyl.

The term “heteroalkylene” refers to a divalent heteroalkyl radical.

The term “heterocycle” or “heterocyclic” refers to heteroaromatic rings (also known as heteroaryls) and heterocycloalkyl rings (also known as heteroalicyclic groups) containing one to four heteroatoms in the ring(s), where each heteroatom in the ring(s) is selected from O, S and N, wherein each heterocyclic group has from 3 to 10 atoms in its ring system, and with the proviso that any ring does not contain two adjacent O or S atoms. In some embodiments, heterocycles are monocyclic, bicyclic, polycyclic, spirocyclic or bridged compounds. Non-aromatic heterocyclic groups (also known as heterocycloalkyls) include rings having 3 to 10 atoms in its ring system and aromatic heterocyclic groups include rings having 5 to 10 atoms in its ring system. The heterocyclic groups include benzo-fused ring systems. Examples of non-aromatic heterocyclic groups are pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, pyrrolin-2-yl, pyrrolin-3-yl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 2-azabicyclo[2.2.2]octanyl, 3-azabicyclo[3.2.1]octanyl, 5-azabicyclo[2.1.1]hexanyl, 6-azabicyclo[3.1.1]heptanyl, 7-azabicyclo[2.2.1]heptanyl, 8-azabicyclo[3.2.1]octanyl, 3H-indolyl, indolin-2-onyl, isoindolin-1-onyl, isoindoline-1,3-dionyl, 3,4-dihydroisoquinolin-1(2H)-onyl, 3,4-dihydroquinolin-2(1H)-onyl, isoindoline-1,3-dithionyl, benzo[d]oxazol-2(3H)-onyl, 1H-benzo[d]imidazol-2(3H)-onyl, benzo[d]thiazol-2(3H)-onyl, and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups are either C-attached (or C-linked) or N-attached where such is possible. For instance, a group derived from pyrrole includes both pyrrol-1-yl (N-attached) or pyrrol-3-yl (C-attached). Further, a group derived from imidazole includes imidazol-1-yl or imidazol-3-yl (both N-attached) or imidazol-2-yl, imidazol-4-yl or imidazol-5-yl (all C-attached). The heterocyclic groups include benzo-fused ring systems. Non-aromatic heterocycles are optionally substituted with one or two oxo (═O) moieties, such as pyrrolidin-2-one. In some embodiments, at least one of the two rings of a bicyclic heterocycle is aromatic. In some embodiments, both rings of a bicyclic heterocycle are aromatic.

The terms “heteroaryl” or, alternatively, “heteroaromatic” refers to an aryl group that includes one or more ring heteroatoms selected from nitrogen, oxygen and sulfur. Illustrative examples of heteroaryl groups include monocyclic heteroaryls and bicyclic heteroaryls. Monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Bicyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, a heteroaryl contains 0-4 N atoms in the ring. In some embodiments, a heteroaryl contains 1-4 N atoms in the ring. In some embodiments, a heteroaryl contains 0-4 N atoms, 0-1 O atoms, and 0-1 S atoms in the ring. In some embodiments, a heteroaryl contains 1-4 N atoms, 0-1 O atoms, and 0-1 S atoms in the ring. In some embodiments, heteroaryl is a C₁-C₉heteroaryl. In some embodiments, monocyclic heteroaryl is a C₁-C₅heteroaryl. In some embodiments, monocyclic heteroaryl is a 5-membered or 6-membered heteroaryl. In some embodiments, bicyclic heteroaryl is a C₆-C₉heteroaryl.

A “heterocycloalkyl” or “heteroalicyclic” group refers to a cycloalkyl group that includes at least one heteroatom selected from nitrogen, oxygen and sulfur. In some embodiments, a heterocycloalkyl is fused with an aryl or heteroaryl. In some embodiments, the heterocycloalkyl is oxazolidinonyl, pyrrolidinyl, tetrahydrofuranyl, tetrahydrothienyl, tetrahydropyranyl, tetrahydrothiopyranyl, piperidinyl, morpholinyl, thiomorpholinyl, piperazinyl, piperidin-2-onyl, pyrrolidine-2,5-dithionyl, pyrrolidine-2,5-dionyl, pyrrolidinonyl, imidazolidinyl, imidazolidin-2-onyl, or thiazolidin-2-onyl. The term heteroalicyclic also includes all ring forms of the carbohydrates, including but not limited to the monosaccharides, the disaccharides and the oligosaccharides. In one aspect, a heterocycloalkyl is a C₂-C₁₀heterocycloalkyl. In another aspect, a heterocycloalkyl is a C₄-C₁₀heterocycloalkyl. In some embodiments, a heterocycloalkyl contains 0-2 N atoms in the ring. In some embodiments, a heterocycloalkyl contains 0-2 N atoms, 0-2 O atoms and 0-1 S atoms in the ring.

The term “oxo” refers to the ═O radical.

The term “bond” or “single bond” refers to a chemical bond between two atoms, or two moieties when the atoms joined by the bond are considered to be part of larger substructure. In one aspect, when a group described herein is a bond, the referenced group is absent thereby allowing a bond to be formed between the remaining identified groups.

The term “moiety” refers to a specific segment or functional group of a molecule. Chemical moieties are often recognized chemical entities embedded in or appended to a molecule.

The term “optionally substituted” or “substituted” means that the referenced group is optionally substituted with one or more additional group(s) individually and independently selected from D, halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, alkenyl, alkynyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from D, halogen, —CN, —NH₂, —NH(CH₃), —N(CH₃)₂, —OH, —CO₂H, —CO₂(C₁-C₄alkyl), —C(═O)NH₂, —C(═O)NH(C₁-C₄alkyl), —C(═O)N(C₁-C₄alkyl)₂, —S(═O)₂NH₂, —S(═O)₂NH(C₁-C₄alkyl), —S(═O)₂N(C₁-C₄alkyl)₂, C₁-C₄alkyl, C₃-C₆cycloalkyl, C₁-C₄fluoroalkyl, C₁-C₄heteroalkyl, C₁-C₄alkoxy, C₁-C₄fluoroalkoxy, —SC₁-C₄alkyl, —S(═O)C₁-C₄alkyl, and —S(═O)₂C₁-C₄alkyl. In some embodiments, optional substituents are independently selected from D, halogen, —CN, —NH₂, —OH, —NH(CH₃), —N(CH₃)₂, —CH₃, —CH₂CH₃, —CF₃, —OCH₃, and —OCF₃.

In some embodiments, substituted groups are substituted with one or two of the preceding groups. In some embodiments, an optional substituent on an aliphatic carbon atom (acyclic or cyclic) includes oxo (═O).

The term “acceptable” with respect to a formulation, composition or ingredient, as used herein, means having no persistent detrimental effect on the general health of the subject being treated.

The term “modulate” as used herein, means to interact with a target either directly or indirectly so as to alter the activity of the target, including, by way of example only, to enhance the activity of the target, to inhibit the activity of the target, to limit the activity of the target, or to extend the activity of the target.

The terms “administer” “administering”, “administration” and the like, as used herein, refer to the methods that may be used to enable delivery of compounds or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Those of skill in the art are familiar with administration techniques that can be employed with the compounds and methods described herein. In some embodiments, the compounds and compositions described herein are administered orally.

The terms “co-administration” or the like, as used herein, are meant to encompass administration of the selected therapeutic agents to a single patient and are intended to include treatment regimens in which the agents are administered by the same or different route of administration or at the same or different time.

The terms “effective amount” or “therapeutically effective amount” as used herein, refer to a sufficient amount of an agent or a compound being administered, which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is optionally determined using techniques, such as a dose escalation study.

The terms “enhance” or “enhancing” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount” as used herein, refers to an amount adequate to enhance the effect of another therapeutic agent in a desired system.

The term “pharmaceutical combination” as used herein, means a product that results from the mixing or combining of more than one active ingredient and includes both fixed and non-fixed combinations of the active ingredients. The term “fixed combination” means that the active ingredients, e.g. a compound described herein, or a pharmaceutically acceptable salt thereof, and a co-agent, are both administered to a patient simultaneously in the form of a single entity or dosage. The term “non-fixed combination” means that the active ingredients, e.g. a compound described herein, or a pharmaceutically acceptable salt thereof, and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The terms “kit” and “article of manufacture” are used as synonyms.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

The terms “treat” “treating” or “treatment” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

### Compounds

The compounds of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), (Iy), (II), (IIa), or (IIb), described herein, including pharmaceutically acceptable salts, active metabolites and pharmaceutically acceptable solvates thereof, are inhibitors of YAP-TEAD and/or inhibitors of TAZ-TEAD protein-protein interaction. In some embodiments, the compounds of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), (Iy), (II), (IIa), or (IIb), described herein, including pharmaceutically acceptable salts and solvates thereof, are inhibitors of YAP-TEAD and inhibitors of TAZ-TEAD protein-protein interaction. In some embodiments, the compounds of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), (Iy), (II), (IIa), or (IIb), described herein, including pharmaceutically acceptable salts and solvates thereof, are inhibitors of YAP-TEAD. In some embodiments, the compounds of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), (Iy), (II), (IIa), or (IIb), described herein, including pharmaceutically acceptable salts and solvates thereof, are inhibitors of TAZ-TEAD protein-protein interaction.

In some embodiments, described herein is a compound of Formula (I), or a pharmaceutically acceptable salt or solvate thereof:

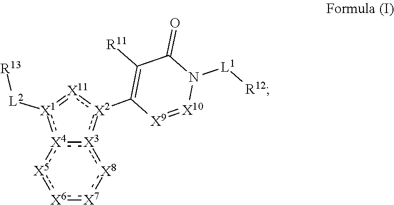

wherein,
X¹ is C, C(R¹), or N;
X² is C, C(R²), or N;
X³ is C, C(R³), or N;
X⁴ is C, C(R⁴), or N;
X⁵ is C(R⁵), C(R⁵)(R^{5a}), N(R^{5b}), or N;
X⁶ is C(R⁶), C(R⁶)(R^{6a}), N(R^{6b}), or N;
X⁷ is C(R⁷), C(R⁷)(R^{7a}), N(R^{7b}), or N;
X⁸ is C(R⁸), C(R⁸)(R^{8a}), N(R^{8b}), or N;
X⁹ is C(R⁹) or N;
X¹⁰ is C(R¹⁰) or N;
X¹¹ is C(R¹⁶), C(R¹⁶)(R^{16a}), C(O), N(R^{16b}), or N;
L¹ and L² are independently selected from a bond and C₁₋₆alkylene optionally substituted with one, two, or three groups selected from R^{14a};
R¹, R², R³, and R⁴ are independently selected from hydrogen and C₁₋₆alkyl;
R⁵, R^{5a}, R⁶, R^{6a}, R⁷, R^{7a}, R⁸, R^{8a}, R¹⁶, and R^{16a} are independently selected from hydrogen, halogen, —CN, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR²⁰, —SR²⁰, —SF₅, —N(R²⁰)(R^{21a}), —C(O)OR²⁰, —OC(O)N(R²⁰)(R²¹), —N(R²²)C(O)N(R²⁰)(R²¹), —N(R²²)C(O)OR²³, —N(R²²)S(O)₂R²³, —C(O)R²³, —S(O)R²³, —OC(O)R²³, —C(O)N(R²⁰)(R²¹), —C(O)C(O)N(R²⁰)(R²¹), —N(R²²)C(O)R²³, —S(O)₂R²³, —S(O)₂N(R²⁰)(R²¹)—, —N═S(═O)(R²³)₂, —S(═O)(═NH)N(R²⁰)(R²¹), —S(═O)(═NH)C(R²⁰)(R²¹), —S(═O)(═NR²³)R²³, —CH₂C(O)N(R²⁰)(R²¹), —CH₂N(R²²)C(O)R²³, —CH₂S(O)₂R²³, and —CH₂S(O)₂N(R²⁰)(R²¹), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three groups selected from R^{14b};
R^{5b}, R^{6b}, R^{7b}, R^{8b}, and R^{16b} are independently selected from hydrogen, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —C(O)OR²⁰, —OC(O)N(R²⁰)(R²¹), —C(O)R²³, —C(O)N(R²⁰)(R²¹), and —S(O)₂R²³, wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three groups selected from R^{14c};
R⁹, R¹⁰, and R¹¹ are independently selected from hydrogen, halogen, and C₁₋₆alkyl;
R¹² is selected from C₁₋₆alkyl, C₁₋₆alkoxyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three groups selected from R^{15a};
R¹³ is selected from C₁₋₆alkyl, C₁₋₆alkoxyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl, wherein C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three groups selected from
each R^{14a}, R^{14b}, and R^{14c} are each independently selected from halogen, oxo, —CN, C₁₋₆alkyl, C₁₋₆haloalkyl, C₂₋₆alkenyl, C₂₋₆alkynyl, C₃₋₁₀cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR²⁰, —SR²⁰, —SF₅, —N(R²⁰)(R²¹), —C(O)OR²⁰, —OC(O)N(R²⁰)(R²¹), —N(R²²)C(O)N(R²⁰)(R²¹), —N(R²²)C(O)OR²³, —N(R²²)S(O)₂R²³, —C(O)R²³, —S(O)R²³, —OC(O)R²³, —C(O)N(R²⁰)(R²¹), —C(O)C(O)N(R²⁰)(R²¹), —N(R²²)C(O)R²³, —S(O)₂R²³, —S(O)₂N(R²⁰)(R²¹)—, —N═S(═O)(R²³)₂, —S(═O)(═NH)N(R²⁰)(R²¹), —S(═O)(═NH)C(R²⁰)(R²¹), —S(═O)(═NR²³)R²³, —CH₂C(O)N(R²⁰)(R²¹), —CH₂N(R²²)C(O)R²³, —CH₂S(O)₂R²³, —CH₂S(O)₂N(R²⁰)(R²¹), wherein C₁₋₆alkyl, C₁₋₆haloalkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²⁰, —SR²⁰, —SF₅, —N(R²⁰)(R²¹), —C(O)OR²⁰, —OC(O)N(R²⁰)(R²¹), —N(R²²)C(O)N(R²⁰)(R²¹), —N(R²²)C(O)OR²³, —N(R²²)S(O)₂R²³, —C(O)R²³, —S(O)R²³, —OC(O)R²³, —C(O)N(R²⁰)(R²¹), —C(O)C(O)N(R²⁰)(R²¹), —N(R²²)C(O)R²³, —S(O)₂R²³, —S(O)₂N(R²⁰)(R²¹)—, —N═S(═O)(R²³)₂, —S(═O)(═NH)N(R²⁰)(R²¹), —S(═O)(═NH)C(R²⁰)(R²¹), —S(═O)(═NR²³)R²³, —CH₂C(O)N(R²⁰)(R²¹), —CH₂N(R²²)C(O)R²³, —CH₂S(O)₂R²³, and —CH₂S(O)₂N(R²⁰)(R²¹);
each R^{15a} is independently selected from halogen, oxo, —CN, C₁₋₆alkyl, C₁₋₆alkoxyl, C₁₋₆haloalkyl, C₂₋₆alkenyl, C₂₋₆alkynyl, C₃₋₁₀cycloalkyl, —CH₂—C₃₋₆cycloalkyl, —NH—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR²⁰, —SR²⁰, —SF₅, —N(R²⁰)(R²¹), —C(O)OR²⁰, —OC(O)N(R²⁰)(R²¹), —N(R²²)C(O)N(R²⁰)(R²¹), —N(R²²)C(O)OR²³, —N(R²²)S(O)₂R²³, —C(O)R²³, —S(O)R²³, —OC(O)R²³, —C(O)N(R²⁰)(R²¹), —C(O)C(O)N(R²⁰)(R²¹), —N(R²²)C(O)R²³, —S(O)₂R²³, —S(O)₂N(R²⁰)(R²¹)—, —N═S(═O)(R²³)₂, —S(═O)(═NH)N(R²⁰)(R²¹), —S(═O)(═NH)C(R²⁰)(R²¹), —S(═O)(═NR²³)R²³, —CH₂C(O)N(R²⁰)(R²¹), —CH₂N(R²²)C(O)R²³, —CH₂S(O)₂R²³, —CH₂S(O)₂N(R²⁰)(R²¹), wherein C₁₋₆alkyl, C₁₋₆haloalkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²⁰, —SR²⁰, —SF₅, —N(R²⁰)(R²¹), —C(O)OR²⁰, —OC(O)N(R²⁰)(R²¹), —N(R²²)C(O)N(R²⁰)(R²¹), —N(R²²)C(O)OR²³, —N(R²²)S(O)₂R²³, —C(O)R²³, —S(O)R²³, —OC(O)R²³, —C(O)N(R²⁰)(R²¹), —C(O)C(O)N(R²⁰)(R²¹), —N(R²²)C(O)R²³, —S(O)₂R²³, —S(O)₂N(R²⁰)(R²¹)—, —N═S(═O)(R²³)₂, —S(═O)(═NH)N(R²⁰)(R²¹), —S(═O)(═NH)C(R²⁰)(R²¹), —S(═O)(═NR²³)R²³, —CH₂C(O)N(R²⁰)(R²¹), —CH₂N(R²²)C(O)R²³, —CH₂S(O)₂R²³, and —CH₂S(O)₂N(R²⁰)(R²¹); or two R^{15a} are combined to form a C₃₋₆cycloalkyl or C₂₋₉heterocycloalkyl, wherein C₃₋₆cycloalkyl and C₂₋₉heterocycloalkyl are optionally substituted with one, two, or three groups independently selected from halogen, oxo, C₁₋₆alkyl, C₁₋₆haloalkyl, and C₁₋₆alkoxy;
each R^{15b} is independently selected from halogen, oxo, —CN, C₁₋₆alkyl, C₁₋₆haloalkyl, C₂₋₆alkenyl, C₂₋₆alkynyl, C₃₋₁₀cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR²⁰, —SR²⁰, —SF₅, —N(R²⁰)(R²¹), —C(O)OR²⁰, —OC(O)N(R²⁰)(R²¹), —N(R²²)C(O)N(R²⁰)(R²¹), —N(R²²)C(O)OR²³, —N(R²²)S(O)₂R²³, —C(O)R²³, —S(O)R²³, —OC(O)R²³, —C(O)N(R²⁰)(R²¹), —C(O)C(O)N(R²⁰)(R²¹), —N(R²²)C(O)R²³, —S(O)₂R²³, —S(O)₂N(R²⁰)(R²¹)—, —N═S(═O)(R²³)₂, —S(═O)(═NH)N(R²⁰)(R²¹), —S(═O)(═NH)C(R²⁰)(R²¹), —S(═O)(═NR²²)R²³, —CH₂C(O) N(R²⁰)(R²¹), —CH₂N(R²)C(O)R²³, —CH₂S(O)₂R²³, —CH₂S(O)₂N(R²⁰)(R²¹), wherein C₁₋₆alkyl, C₁₋₆haloalkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₆heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²⁰, —SR²⁰, —SF₅, —N(R²⁰)(R²¹), —C(O)OR²⁰, —OC(O)N(R²⁰)(R²¹), —N(R²²)C(O)N(R²⁰)(R²¹), —N(R²²)C(O)OR²³, —N(R²²)S(O)₂R²³, —C(O)R²³, —S(O)R²³, —OC(O)R²³, —C(O)N(R²⁰)(R²¹), —C(O)C(O)N(R²⁰)(R²¹), —N(R²²)C(O)R²³, —S(O)₂R²³, —S(O)₂N(R²⁰)(R²¹)—, —N═S(═O)(R²³)₂, —S(═O)(═NH)N(R²⁰)(R²¹), —S(═O)(═NH)C(R²⁰)(R²¹), —S(═O)(═NR²³)R²³, —CH₂C(O)N(R²⁰)(R²¹), —CH₂N(R²²)C(O)R²³, —CH₂S(O)₂R²³, and —CH₂S(O)₂N(R²⁰)(R²¹); or two R^{15b} are combined to form a C₃₋₆cycloalkyl or C₂₋₉heterocycloalkyl, wherein C₃₋₆cycloalkyl and C₂₋₉heterocycloalkyl are optionally substituted with one, two, or three groups independently selected from halogen, oxo, C₁₋₆alkyl, C₁₋₆haloalkyl, and C₁₋₆alkoxy;
each R²⁰ is independently selected from hydrogen, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R²¹ is independently selected from hydrogen, C₁₋₆alkyl, and C₁₋₆haloalkyl; or R²⁰ and R²¹, together with the nitrogen to which they are attached, form a C₂₋₉heterocycloalkyl;
each R^{21a} is independently selected from C₁₋₆alkyl and C₁₋₆haloalkyl;
each R²² is independently selected from hydrogen, C₁₋₆alkyl, and C₁₋₆haloalkyl;
each R²³ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₆heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl; and
[symbol] indicates a single or double bond such that all valences are satisfied.

For any and all of the embodiments, substituents are selected from among a subset of the listed alternatives. For example, in some embodiments is a compound of Formula (I), or a pharmaceutically acceptable salt or solvate thereof, wherein X⁹ is C(R⁹). In some embodiments is a compound of Formula (I), or a pharmaceutically acceptable salt or solvate thereof, wherein R⁹ is hydrogen. In some embodiments is a compound of Formula (I), or a pharmaceutically acceptable salt or solvate thereof, wherein R⁹ is C₁₋₆alkyl. In some embodiments is a compound of Formula (I), or a pharmaceutically acceptable salt or solvate thereof, wherein R⁹ is —CH₃.

In some embodiments is a compound of Formula (I), or a pharmaceutically acceptable salt or solvate thereof, wherein X¹⁰ is C(R¹⁰). In some embodiments is a compound of Formula (I), or a pharmaceutically acceptable salt or solvate thereof, wherein R¹⁰ is hydrogen. In some embodiments is a compound of Formula (I), or a pharmaceutically acceptable salt or solvate thereof, wherein R¹⁰ is C₁₋₆alkyl. In some embodiments is a compound of Formula (I), or a pharmaceutically acceptable salt or solvate thereof, wherein R¹⁰ is —CH₃.

In some embodiments is a compound of Formula (I), or a pharmaceutically acceptable salt or solvate thereof, wherein R⁹ and R¹⁰ are hydrogen.

In some embodiments is a compound of Formula (I), or a pharmaceutically acceptable salt or solvate thereof, wherein R¹¹ is hydrogen. In some embodiments is a compound of Formula (I), or a pharmaceutically acceptable salt or solvate thereof, wherein R¹¹ is C₁₋₆alkyl. In some embodiments is a compound of Formula (I), or a pharmaceutically acceptable salt or solvate thereof, wherein R¹¹ is —CH₃.

In some embodiments is a compound of Formula (I), or a pharmaceutically acceptable salt or solvate thereof, wherein R⁹, R¹⁰, and R¹¹ are hydrogen.

In some embodiments is a compound of Formula (I), or a pharmaceutically acceptable salt or solvate thereof, wherein X¹¹ is N. In some embodiments is a compound of Formula (I), or a pharmaceutically acceptable salt or solvate thereof, wherein X¹¹ is C(R¹⁶). In some embodiments is a compound of Formula (I), or a pharmaceutically acceptable salt or solvate thereof, wherein X¹¹ is C(H). In some embodiments is a compound of Formula (I), or a pharmaceutically acceptable salt or solvate thereof, wherein X¹¹ is C(O).

In some embodiments is a compound of Formula (I), or a pharmaceutically acceptable salt or solvate thereof, wherein X¹ is C. In some embodiments is a compound of Formula (I), or a pharmaceutically acceptable salt or solvate thereof, wherein X¹ is N. In some embodiments is a compound of Formula (I), or a pharmaceutically acceptable salt or solvate thereof, wherein X¹ is C(R¹). In some embodiments is a compound of Formula (I), or a pharmaceutically acceptable salt or solvate thereof, wherein X¹ is C(H).

In some embodiments is a compound of Formula (I), or a pharmaceutically acceptable salt or solvate thereof, wherein X² is C. In some embodiments is a compound of Formula (I), or a pharmaceutically acceptable salt or solvate thereof, wherein X² is N. In some embodiments is a compound of Formula (I), or a pharmaceutically acceptable salt or solvate thereof, wherein X² is C(R²). In some embodiments is a compound of Formula (I), or a pharmaceutically acceptable salt or solvate thereof, wherein X² is C(H).

In some embodiments is a compound of Formula (I), or a pharmaceutically acceptable salt or solvate thereof, wherein X¹ is N and X² is C. In some embodiments is a compound of Formula (I), or a pharmaceutically acceptable salt or solvate thereof, wherein X¹ is C and X² is N.

In some embodiments is a compound of Formula (I), or a pharmaceutically acceptable salt or solvate thereof, wherein X³ is C. In some embodiments is a compound of Formula (I), or a pharmaceutically acceptable salt or solvate thereof, wherein X³ is N. In some embodiments is a compound of Formula (I), or a pharmaceutically acceptable salt or solvate thereof, wherein X³ is C(R³). In some embodiments is a compound of Formula (I), or a pharmaceutically acceptable salt or solvate thereof, wherein X³ is C(H).

In some embodiments is a compound of Formula (I), or a pharmaceutically acceptable salt or solvate thereof, wherein X⁴ is C. In some embodiments is a compound of Formula (I), or a pharmaceutically acceptable salt or solvate thereof, wherein X⁴ is N. In some embodiments is a compound of Formula (I), or a pharmaceutically acceptable salt or solvate thereof, wherein X⁴ is C(R⁴). In some embodiments is a compound of Formula (I), or a pharmaceutically acceptable salt or solvate thereof, wherein X⁴ is C(H).

In some embodiments is a compound of Formula (I), or a pharmaceutically acceptable salt or solvate thereof, wherein X³ is C and X⁴ is C. In some embodiments is a compound of Formula (I), or a pharmaceutically acceptable salt or solvate thereof, wherein X³ is N and X⁴ is C. In some embodiments is a compound of Formula (I), or a pharmaceutically acceptable salt or solvate thereof, wherein X³ is C and X⁴ is N.

In some embodiments is a compound of Formula (I), or a pharmaceutically acceptable salt or solvate thereof, wherein X⁵ is C(R⁵) or N; X⁶ is C(R⁶) or N; X⁷ is C(R⁷) or N; and X⁸ is C(R⁸) or N. In some embodiments is a compound of Formula (I), or a pharmaceutically acceptable salt or solvate thereof, wherein X⁵ is C(R⁵); X⁶ is C(R⁶); X⁷ is C(R⁷); and X⁸ is C(R⁸). In some embodiments is a compound of Formula (I), or a pharmaceutically acceptable salt or solvate thereof, wherein X⁵ is N; X⁶ is C(R⁶); X⁷ is C(R⁷); and X⁸ is C(R⁸). In some embodiments is a compound of Formula (I), or a pharmaceutically acceptable salt or solvate thereof, wherein X⁵ is C(R⁵); X⁶ is N; X⁷ is C(R⁷); and X⁸ is C(R⁸). In some embodiments is a compound of Formula (I), or a pharmaceutically acceptable salt or solvate thereof, wherein X⁵ is C(R⁵); X⁶ is C(R⁶); X⁷ is N; and X⁸ is C(R⁵). In some embodiments is a compound of Formula (I), or a pharmaceutically acceptable salt or solvate thereof, wherein X⁵ is C(R⁵); X⁶ is C(R⁶); X⁷ is C(R⁷); and X⁸ is N. In some embodiments is a compound of Formula (I), or a pharmaceutically acceptable salt or solvate thereof, wherein X⁵ is N; X⁶ is C(R⁶); X⁷ is N; and X⁸ is C(R⁸). In some embodiments is a compound of Formula (I), or a pharmaceutically acceptable salt or solvate thereof, wherein X⁵ is C(R⁵); X⁶ is N; X⁷ is C(R⁷); and X₈ is N.

In some embodiments is a compound of Formula (I), or a pharmaceutically acceptable salt or solvate thereof, wherein R¹² is selected from C₁₋₆alkyl, C₃₋₁₀cycloalkyl, C₂₋₉heterocycloalkyl, C₆₋₁₀aryl, and C₁₋₉heteroaryl, wherein C₁₋₆alkyl, C₃₋₁₀cycloalkyl, C₂₋₉heterocycloalkyl, C₆₋₁₀aryl, and C₁₋₉heteroaryl are optionally substituted with one, two, or three groups selected from R^{15a}.

In some embodiments is a compound of Formula (I), or a pharmaceutically acceptable salt or solvate thereof, wherein R¹² is selected from C₁₋₆alkyl, C₅₋₆cycloalkyl, C₃₋₇heterocycloalkyl, C₆₋₁₀aryl, and C₃₋₇heteroaryl, wherein C₁₋₆alkyl, C₅₋₆cycloalkyl, C₃₋₇heterocycloalkyl, C₆₋₁₀aryl, and C₃₋₇heteroaryl are optionally substituted with one, two, or three groups selected from R^{15a}.

In some embodiments is a compound of Formula (I), or a pharmaceutically acceptable salt or solvate thereof, wherein R^{15a} is independently selected from halogen, oxo, —CN, C₁₋₆alkyl, C₁₋₆alkoxyl, C₁₋₆haloalkyl, C₃₋₁₀cycloalkyl, —CH₂—C₃₋₆cycloalkyl, —NH—C₃₋₆cycloalkyl, C₃₋₇heterocycloalkyl, —CH₂—C₂₋₉heterocycloalkyl, C₁₋₉heteroaryl, —CH₂—C₁₋₉heteroaryl, —OR²⁰, —N(R²⁰)(R²¹), —OC(O)R²³, wherein C₁₋₆alkyl, C₁₋₆alkoxyl, C₁₋₆haloalkyl, C₃₋₁₀cycloalkyl, —CH₂—C₃₋₆cycloalkyl, —NH—C₃₋₆cycloalkyl, C₃₋₇heterocycloalkyl, —CH₂—C₂₋₉heterocycloalkyl, C₁₋₉heteroaryl, —CH₂—C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²⁰, and —N(R²⁰)(R²¹); or two R^{15a} are combined to form a C₃₋₆cycloalkyl or C₂₋₉heterocycloalkyl, wherein C₃₋₆cycloalkyl and C₂₋₉heterocycloalkyl are optionally substituted with one, two, or three groups independently selected from halogen, oxo, C₁₋₆alkyl, C₁₋₆haloalkyl, and C₁₋₆alkoxy.

In some embodiments is a compound of Formula (I), or a pharmaceutically acceptable salt or solvate thereof, wherein R^{15a} is independently selected from halogen, oxo, —CN, C₁₋₆alkyl, C₁₋₆alkoxyl, C₁₋₆haloalkyl, —NH—C₃₋₆cycloalkyl, C₃₋₇heterocycloalkyl, —OR²⁰, and —N(R²⁰)(R²¹), wherein C₁₋₆alkyl, C₁₋₆alkoxyl, C₁₋₆haloalkyl, —NH—C₃₋₆cycloalkyl, C₃₋₇heterocycloalkyl, are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²⁰, and —N(R²⁰)(R²¹), or two R^{15a} are combined to form a C₃₋₆cycloalkyl or C₂₋₉heterocycloalkyl, wherein C₃₋₆cycloalkyl and C₂₋₉heterocycloalkyl are optionally substituted with one, two, or three groups independently selected from halogen, oxo, C₁₋₆alkyl, C₁₋₆haloalkyl, and C₁₋₆alkoxy.

In some embodiments is a compound of Formula (I), or a pharmaceutically acceptable salt or solvate thereof, wherein R^{15a} is independently selected from —C₃₋₇heterocycloalkyl, —NH—C₃₋₆cycloalkyl, —OR²⁰, and —N(R²⁰)(R²¹), wherein —C₃₋₇heterocycloalkyl and —NH—C₃₋₆cycloalkyl, are optionally substituted with one, two, or three groups independently selected from halogen, C₁₋₆alkyl, and —OR²⁰.

In some embodiments is a compound of Formula (I), or a pharmaceutically acceptable salt or solvate thereof, wherein R^{15a} is independently selected from —OR²⁰, and —N(R²⁰)(R²¹), optionally, wherein two R^{15a} are combined to form a C₃₋₆cycloalkyl or C₂₋₉heterocycloalkyl, wherein C₃₋₆cycloalkyl and C₂₋₉heterocycloalkyl are optionally substituted with one, two, or three groups independently selected from halogen, and —OR²⁰.

In some embodiments is a compound of Formula (I), or a pharmaceutically acceptable salt or solvate thereof, wherein R¹³ is selected from C₁₋₆alkyl, C₃₋₁₀cycloalkyl, C₂₋₉heterocycloalkyl, C₆₋₁₀aryl, and C₁₋₉heteroaryl, wherein C₁₋₆alkyl, C₃₋₁₀cycloalkyl, C₂₋₉heterocycloalkyl, C₆₋₁₀aryl, and C₁₋₉heteroaryl are optionally substituted with one, two, or three groups selected from R^{15b}.

In some embodiments is a compound of Formula (I), or a pharmaceutically acceptable salt or solvate thereof, wherein R¹³ is selected from C₁₋₆alkyl, C₅₋₆cycloalkyl, C₃₋₇heterocycloalkyl, C₆₋₁₀aryl, and C₃₋₇heteroaryl, wherein C₁₋₆alkyl, C₅₋₆cycloalkyl, C₃₋₇heterocycloalkyl, C₆₋₁₀aryl, and C₃₋₇heteroaryl are optionally substituted with one, two, or three groups selected from R^{15b}.

In some embodiments is a compound of Formula (I), or a pharmaceutically acceptable salt or solvate thereof, wherein R^{15b} is independently selected from halogen, oxo, —CN, C₁₋₆alkyl, C₁₋₆haloalkyl, C₂₋₆alkenyl, C₂₋₆alkynyl, C₃₋₁₀cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, and —OR²⁰, wherein C₁₋₆alkyl, C₁₋₆haloalkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₂₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, and —OR²⁰; or two R^{15b} are combined to form a C₃₋₆cycloalkyl or C₂₋₉heterocycloalkyl, wherein C₃₋₆cycloalkyl and C₂₋₉heterocycloalkyl are optionally substituted with one, two, or three groups independently selected from halogen, oxo, C₁₋₆alkyl, C₁₋₆haloalkyl, and C₁₋₆alkoxy.

In some embodiments is a compound of Formula (I), or a pharmaceutically acceptable salt or solvate thereof, wherein R^{15b} is independently selected from halogen, oxo, —CN, C₁₋₆alkyl, C₁₋₆haloalkyl, C₃₋₆cycloalkyl, —CH₂—C₃₋₆cycloalkyl, C₆₋₁₀aryl, —CH₂—C₆₋₁₀aryl, and —OR²⁰, wherein C₁₋₆alkyl, C₁₋₆haloalkyl, C₃₋₆cycloalkyl, —CH₂—C₃₋₆cycloalkyl, C₆₋₁₀aryl, and —CH₂—C₆₋₁₀aryl, are optionally substituted with one, two, or three groups independently selected from halogen, oxo, —CN, C₁₋₆alkyl, C₁₋₆haloalkyl, C₁₋₆alkoxy, C₁₋₆haloalkoxy, and —OR²⁰.

In some embodiments is a compound of Formula (I), or a pharmaceutically acceptable salt or solvate thereof, wherein R^{15b} is independently selected from halogen, C₁₋₆alkyl, C₁₋₆haloalkyl, C₃₋₆cycloalkyl, and —OR²⁰, wherein C₁₋₆alkyl, C₁₋₆haloalkyl, and C₃₋₆cycloalkyl, are optionally substituted with one, two, or three groups independently selected from halogen, oxo, —CN, C₁₋₆alkyl, C₁₋₆haloalkyl, C₁₋₆alkoxy, C₁₋₆haloalkoxy, and —OR²⁰.

In some embodiments is a compound of Formula (I), or a pharmaceutically acceptable salt or solvate thereof, wherein R^{15b} is C₁₋₆haloalkyl, or —OR²⁰.

In some embodiments is a compound of Formula (I), or a pharmaceutically acceptable salt or solvate thereof, wherein each R²⁰ is independently selected from hydrogen, C₁₋₆alkyl, and C₃₋₆cycloalkyl, wherein C₁₋₆alkyl, C₃₋₆cycloalkyl, is optionally substituted with one, two, or three groups selected from halogen, hydroxy, C₁₋₆alkyl, and C₁₋₆alkoxy, C₁₋₉heteroaryl.

In some embodiments is a compound of Formula (I), or a pharmaceutically acceptable salt or solvate thereof, wherein each R²⁰ is independently selected from hydrogen, —CH₃, —CH₂CH₂OH, —CH₂CH₂OCH₃, C₃cycloalkyl and cyclobutyl, wherein cyclobutyl is optionally substituted with —OCH₃.

In some embodiments is a compound of Formula (I), or a pharmaceutically acceptable salt or solvate thereof, wherein each R²¹ is independently selected from hydrogen, and C₁₋₆alkyl; or R²⁰ and R²¹, together with the nitrogen to which they are attached, form a C₂₋₉heterocycloalkyl.

In some embodiments is a compound of Formula (I), or a pharmaceutically acceptable salt or solvate thereof, wherein each R²¹ is independently selected from hydrogen, —CH₃, and C₁₋₆alkyl; or R²⁰ and R²¹ together with the nitrogen to which they are attached, form azetidinyl, pyrrolidinyl, morpholino,

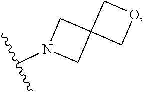 [SMILES: *N1CC2(COC2)C1]

or piperidinyl, wherein azetidinyl, pyrrolidinyl, morpholino,

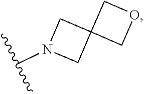 [SMILES: *N1CC2(COC2)C1]

and piperidinyl are optionally substituted with hydroxy, or C₁₋₆alkyl.

In some embodiments is a compound of Formula (I), or a pharmaceutically acceptable salt or solvate thereof, having the structure of Formula (Ia):

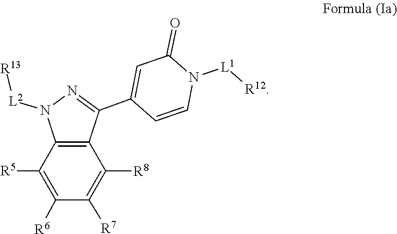

In some embodiments is a compound of Formula (I), or a pharmaceutically acceptable salt or solvate thereof, having the structure of Formula (Ib):

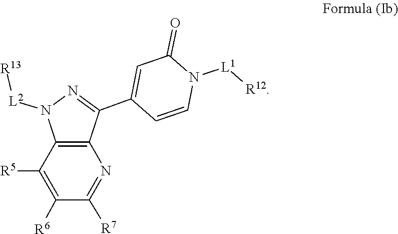

In some embodiments is a compound of Formula (I), or a pharmaceutically acceptable salt or solvate thereof, having the structure of Formula (Ic):

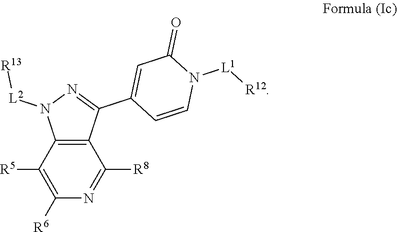

In some embodiments is a compound of Formula (I), or a pharmaceutically acceptable salt or solvate thereof, having the structure of Formula (Id):

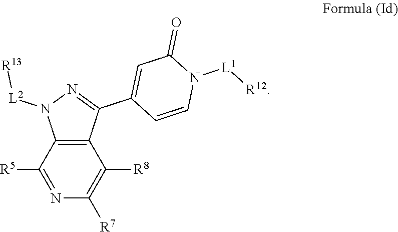

In some embodiments is a compound of Formula (I), or a pharmaceutically acceptable salt or solvate thereof, having the structure of Formula (Ie):

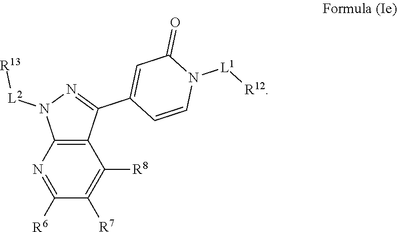

In some embodiments is a compound of Formula (I), or a pharmaceutically acceptable salt or solvate thereof, having the structure of Formula (If):

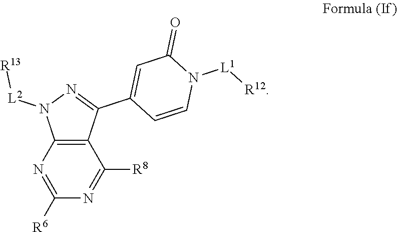

In some embodiments is a compound of Formula (I), or a pharmaceutically acceptable salt or solvate thereof, having the structure of Formula (Ig):

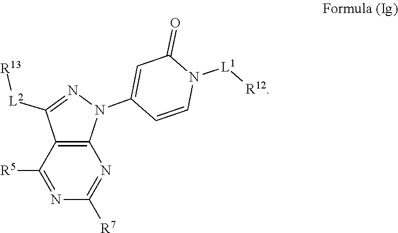

In some embodiments is a compound of Formula (I), or a pharmaceutically acceptable salt or solvate thereof, having the structure of Formula (Ih):

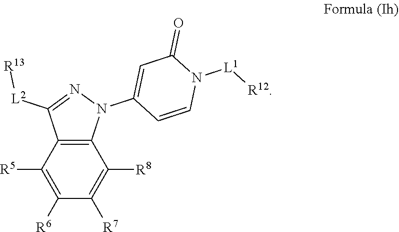

In some embodiments is a compound of Formula (I), or a pharmaceutically acceptable salt or solvate thereof, having the structure of Formula (Ii):

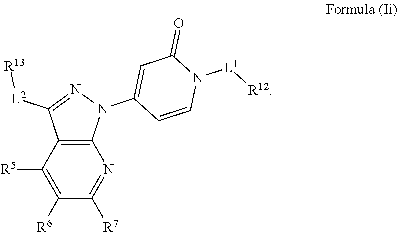

In some embodiments is a compound of Formula (I), or a pharmaceutically acceptable salt or solvate thereof, having the structure of Formula (Ij):

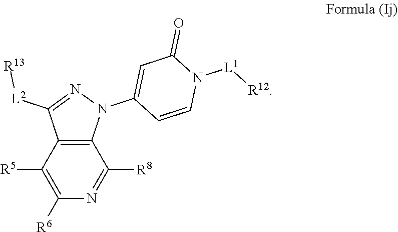

In some embodiments is a compound of Formula (I), or a pharmaceutically acceptable salt or solvate thereof, having the structure of Formula (Ik):

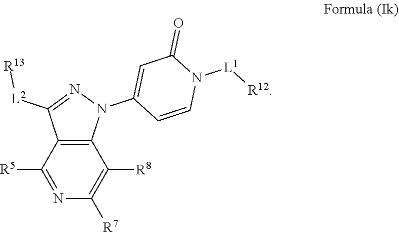

In some embodiments is a compound of Formula (I), or a pharmaceutically acceptable salt or solvate thereof, having the structure of Formula (Il):

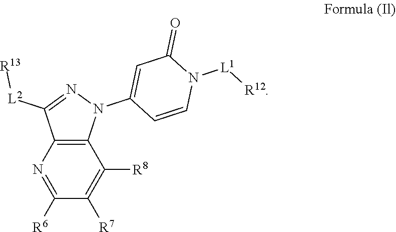

In some embodiments is a compound of Formula (I), or a pharmaceutically acceptable salt or solvate thereof, having the structure of Formula (Im):

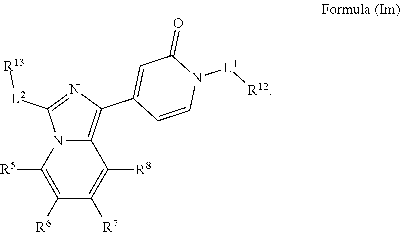

In some embodiments is a compound of Formula (I), or a pharmaceutically acceptable salt or solvate thereof, having the structure of Formula (In):

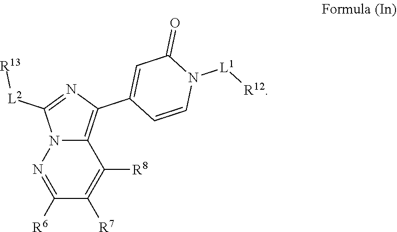

In some embodiments is a compound of Formula (I), or a pharmaceutically acceptable salt or solvate thereof, having the structure of Formula (Io):

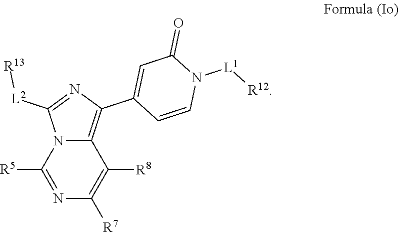

In some embodiments is a compound of Formula (I), or a pharmaceutically acceptable salt or solvate thereof, having the structure of Formula (Ip):

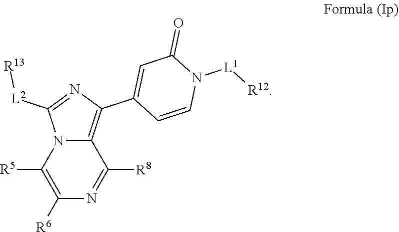

In some embodiments is a compound of Formula (I), or a pharmaceutically acceptable salt or solvate thereof, having the structure of Formula (Iq):

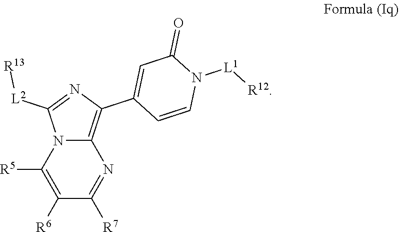

In some embodiments is a compound of Formula (I), or a pharmaceutically acceptable salt or solvate thereof, having the structure of Formula (Ir):

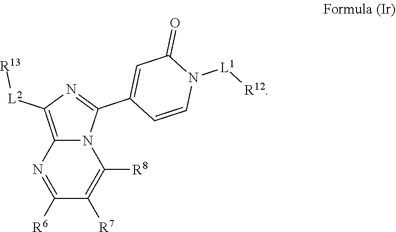

In some embodiments is a compound of Formula (I), or a pharmaceutically acceptable salt or solvate thereof, having the structure of Formula (Is):

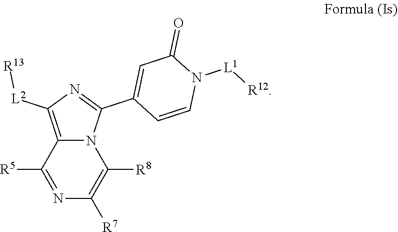

In some embodiments is a compound of Formula (I), or a pharmaceutically acceptable salt or solvate thereof, having the structure of Formula (It):

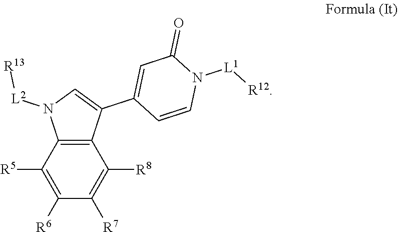

In some embodiments is a compound of Formula (I), or a pharmaceutically acceptable salt or solvate thereof, having the structure of Formula (Iu):

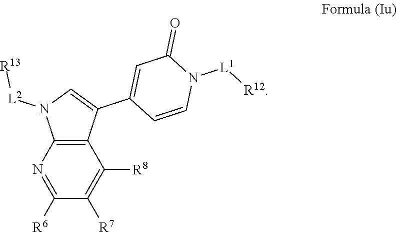

In some embodiments is a compound of Formula (I), or a pharmaceutically acceptable salt or solvate thereof, having the structure of Formula (Iv):

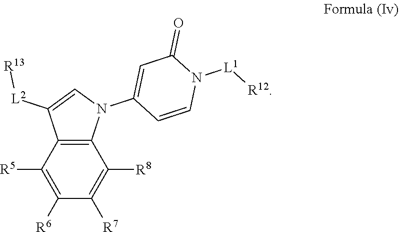

In some embodiments is a compound of Formula (I), or a pharmaceutically acceptable salt or solvate thereof, having the structure of Formula (Iw):

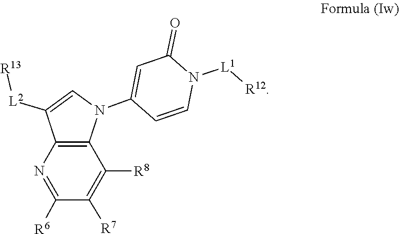

In some embodiments is a compound of Formula (I), or a pharmaceutically acceptable salt or solvate thereof, having the structure of Formula (Ix):

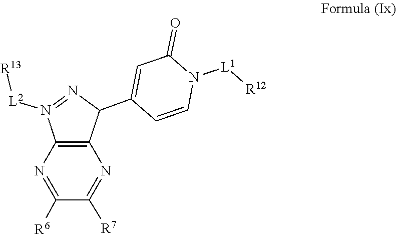

In some embodiments is a compound of Formula (I), or a pharmaceutically acceptable salt or solvate thereof, having the structure of Formula (Iy):

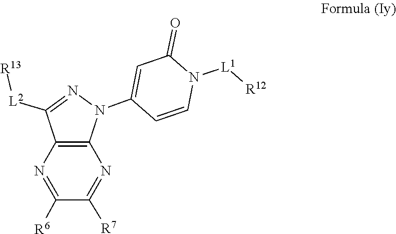

In some embodiments is a compound of Formula (I), (Ia), (Ib), (Ic), (Id), (Ig), (Ih), (Ii), (Ij), (Ik), (Im), (Io), (Ip), (Iq), (Is), (It), or (Iv), or a pharmaceutically acceptable salt or solvate thereof, wherein R⁵ is selected from hydrogen, halogen, —CN, C₁₋₆alkyl, C₁₋₆haloalkyl, —OR²⁰, —C(O)OR²⁰, —C(O)R²³, —C(O)N(R²⁰)(R²¹), —S(O)₂R²³, and —S(O)₂N(R²⁰)(R²¹)—, wherein C₁₋₆alkyl is optionally substituted with one, two, or three groups selected from R^{14b}. In some embodiments is a compound of Formula (I), (Ia), (Ib), (Ic), (Id), (Ig), (Ih), (Ii), (Ij), (Ik), (Im), (Io), (Ip), (Iq), (Is), (It), or (Iv), or a pharmaceutically acceptable salt or solvate thereof, wherein R⁵ is selected from hydrogen, halogen, —CN, C₁₋₆alkyl, —OR²⁰, and —C(O)N(R²⁰)(R²¹), wherein C₁₋₆alkyl is optionally substituted with one, two, or three groups selected from R^{14b}, and wherein R²⁰ is independently selected from hydrogen and C₁₋₆alkyl. In some embodiments is a compound of Formula (I), (Ia), (Ib), (Ic), (Id), (Ig), (Ih), (Ii), (Ij), (Ik), (Im), (Io), (Ip), (Iq), (Is), (It), or (Iv), or a pharmaceutically acceptable salt or solvate thereof, wherein R⁵ is hydrogen. In some embodiments is a compound of Formula (I), (Ia), (Ib), (Ic), (Id), (Ig), (Ih), (Ii), (Ij), (Ik), (Im), (Io), (Ip), (Iq), (Is), (It), or (Iv), or a pharmaceutically acceptable salt or solvate thereof, wherein R⁵ is halogen. In some embodiments is a compound of Formula (I), (Ia), (Ib), (Ic), (Id), (Ig), (Ih), (Ii), (Ij), (Ik), (Im), (Io), (Ip), (Iq), (Is), (It), or (Iv), or a pharmaceutically acceptable salt or solvate thereof, wherein R⁵ is F, or Cl. In some embodiments is a compound of Formula (I), (Ia), (Ib), (Ic), (Id), (Ig), (Ih), (Ii), (Ij), (Ik), (Im), (Io), (Ip), (Iq), (Is), (It), (Iv), (II), (IIa), or (IIb), or a pharmaceutically acceptable salt or solvate thereof, wherein R⁵ is F. In some embodiments is a compound of Formula (I), (Ia), (Ib), (Ic), (Id), (Ig), (Ih), (Ii), (Ij), (Ik), (Im), (Io), (Ip), (Iq), (Is), (It), or (Iv), or a pharmaceutically acceptable salt or solvate thereof, wherein R⁵ is —CN. In some embodiments is a compound of Formula (I), (Ia), (Ib), (Ic), (Id), (Ig), (Ih), (Ii), (Ij), (Ik), (Im), (Io), (Ip), (Iq), (Is), (It), or (Iv), or a pharmaceutically acceptable salt or solvate thereof, wherein R⁵ is C₁₋₆alkyl optionally substituted with one, two, or three groups selected from R^{14b}. In some embodiments is a compound of Formula (I), (Ia), (Ib), (Ic), (Id), (Ig), (Ih), (Ii), (Ij), (Ik), (Im), (Io), (Ip), (Iq), (Is), (It), or (Iv), or a pharmaceutically acceptable salt or solvate thereof, wherein R⁵ is C₁₋₆alkyl substituted with one, two, or three groups selected from R^{14b}. In some embodiments is a compound of Formula (I), (Ia), (Ib), (Ic), (Id), (Ig), (Ih), (Ii), (Ij), (Ik), (Im), (Io), (Ip), (Iq), (Is), (It), or (Iv), or a pharmaceutically acceptable salt or solvate thereof, wherein R⁵ is unsubstituted C₁₋₆alkyl. In some embodiments is a compound of Formula (I), (Ia), (Ib), (Ic), (Id), (Ig), (Ih), (Ii), (Ij), (Ik), (Im), (Io), (Ip), (Iq), (Is), (It), or (Iv), or a pharmaceutically acceptable salt or solvate thereof, wherein R⁵ is —OR²⁰, wherein R²⁰ is independently selected from hydrogen and C₁₋₆alkyl. In some embodiments is a compound of Formula (I), (Ia), (Ib), (Ic), (Id), (Ig), (Ih), (Ii), (Ij), (Ik), (Im), (Io), (Ip), (Iq), (Is), (It), or (Iv), or a pharmaceutically acceptable salt or solvate thereof, wherein R⁵ is —C(O)N(R²⁰)(R²¹). In some embodiments is a compound of Formula (I), (Ia), (Ib), (Ic), (Id), (Ig), (Ih), (Ii), (Ij), (Ik), (Im), (Io), (Ip), (Iq), (Is), (It), or (Iv), or a pharmaceutically acceptable salt or solvate thereof, wherein R⁵ is —C(O)NH₂.

In some embodiments is a compound of Formula (I), (Ia), (Ib), (Ic), (Ie), (If), (Ih), (Ii), (Ij), (Il), (Im), (In), (Ip), (Iq), (Ir), (It), (Iu), (Iv), or (Iw), or a pharmaceutically acceptable salt or solvate thereof, wherein R⁶ is selected from hydrogen, halogen, —CN, C₁₋₆alkyl, C₁₋₆haloalkyl, —OR²⁰, —C(O)OR²⁰, —C(O)R²³, —C(O)N(R²⁰)(R²¹), —S(O)₂R²¹, and —S(O)₂N(R²⁰)(R²¹)—, wherein C₁₋₆alkyl is optionally substituted with one, two, or three groups selected from R^{14b}. In some embodiments is a compound of Formula (I), (Ia), (Ib), (Ic), (Ie), (If), (Ih), (Ii), (Ij), (Il), (Im), (In), (Ip), (Iq), (Ir), (It), (Iu), (Iv), (Iw), (Ix), or (Iy), or a pharmaceutically acceptable salt or solvate thereof, wherein R⁶ is selected from hydrogen, halogen, —CN, C₁₋₆alkyl, —OR²⁰, and —C(O)N(R²⁰)(R²¹), wherein C₁₋₆alkyl is optionally substituted with one, two, or three groups selected from R^{14b}, and wherein R²⁰ is independently selected from hydrogen and C₁₋₆alkyl. In some embodiments is a compound of Formula (I), (Ia), (Ib), (Ic), (Ie), (If), (Ih), (Ii), (Ij), (Il), (Im), (In), (Ip), (Iq), (Ir), (It), (Iu), (Iv), (Iw), (Ix), or (Iy), or a pharmaceutically acceptable salt or solvate thereof, wherein R⁶ is hydrogen. In some embodiments is a compound of Formula (I), (Ia), (Ib), (Ic), (Ie), (If), (Ih), (Ii), (Ij), (Il), (Im), (In), (Ip), (Iq), (Ir), (It), (Iu), (Iv), (Iw), (Ix), or (Iy), or a pharmaceutically acceptable salt or solvate thereof, wherein R⁶ is halogen. In some embodiments is a compound of Formula (I), (Ia), (Ib), (Ic), (Ie), (If), (Ih), (Ii), (Ij), (Il), (Im), (In), (Ip), (Iq), (Ir), (It), (Iu), (Iv), (Iw), (Ix), or (Iy), or a pharmaceutically acceptable salt or solvate thereof, wherein R⁶ is —CN. In some embodiments is a compound of Formula (I), (Ia), (Ib), (Ic), (Ie), (If), (Ih), (Ii), (Ij), (Il), (Im), (In), (Ip), (Iq), (Ir), (It), (Iu), (Iv), (Iw), (Ix), or (Iy), or a pharmaceutically acceptable salt or solvate thereof, wherein R⁶ is C₁₋₆alkyl optionally substituted with one, two, or three groups selected from R^{14b}. In some embodiments is a compound of Formula (I), (Ia), (Ib), (Ic), (Ie), (If), (Ih), (Ii), (Ij), (Il), (Im), (In), (Ip), (Iq), (Ir), (It), (Iu), (Iv), (Iw), (Ix), or (Iy), or a pharmaceutically acceptable salt or solvate thereof, wherein R⁶ is C₁₋₆alkyl substituted with one, two, or three groups selected from R^{14b}. In some embodiments is a compound of Formula (I), (Ia), (Ib), (Ic), (Ie), (If), (Ih), (Ii), (Ij), (Il), (Im), (In), (Ip), (Iq), (Ir), (It), (Iu), (Iv), (Iw), (Ix), or (Iy), or a pharmaceutically acceptable salt or solvate thereof, wherein R⁶ is unsubstituted C₁₋₆alkyl. In some embodiments is a compound of Formula (I), (Ia), (Ib), (Ic), (Ie), (If), (Ih), (Ii), (Ij), (Il), (Im), (In), (Ip), (Iq), (Ir), (It), (Iu), (Iv), (Iw), (Ix), or (Iy), or a pharmaceutically acceptable salt or solvate thereof, wherein R⁶ is —OR²⁰, wherein R²⁰ is independently selected from hydrogen and C₁₋₆alkyl. In some embodiments is a compound of Formula (I), (Ia), (Ib), (Ic), (Ie), (If), (Ih), (Ii), (Ij), (Il), (Im), (In), (Ip), (Iq), (Ir), (It), (Iu), (Iv), (Iw), (Ix), or (Iy), or a pharmaceutically acceptable salt or solvate thereof, wherein R⁶ is —C(O)N(R²⁰)(R²¹). In some embodiments is a compound of Formula (I), (Ia), (Ib), (Ic), (Ie), (If), (Ih), (Ii), (Ij), (Il), (Im), (In), (Ip), (Iq), (Ir), (It), (Iu), (Iv), (Iw), (Ix), or (Iy), or a pharmaceutically acceptable salt or solvate thereof, wherein R⁶ is —C(O)NH₂.

In some embodiments is a compound of Formula (I), (Ia), (Ib), (Id), (Ie), (Ig), (Ih), (Ii), (Ik), (Il), (Im), (In), (Io), (Iq), (Ir), (Is), (It), (Iu), (Iv), (Iw), (Ix), or (Iy), or a pharmaceutically acceptable salt or solvate thereof, wherein R⁷ is selected from hydrogen, halogen, —CN, C₁₋₆alkyl, C₁₋₆haloalkyl, —OR²⁰, —C(O)OR²⁰, —C(O)R²³, —C(O)N(R²⁰)(R²¹), —S(O)₂R²³, and —S(O)₂N(R²⁰)(R²¹)—, wherein C₁₋₆alkyl is optionally substituted with one, two, or three groups selected from R^{14b}. In some embodiments is a compound of Formula (I), (Ia), (Ib), (Id), (Ie), (Ig), (Ih), (Ii), (Ik), (Il), (Im), (In), (Io), (Iq), (Ir), (Is), (It), (Iu), (Iv), (Iw), (Ix), or (Iy), or a pharmaceutically acceptable salt or solvate thereof, wherein R⁷ is selected from hydrogen, halogen, —CN, C₁₋₆alkyl, —OR²⁰, and —C(O)N(R²⁰)(R²¹), wherein C₁₋₆alkyl is optionally substituted with one, two, or three groups selected from R^{14b}, and wherein R²⁰ is independently selected from hydrogen and C₁₋₆alkyl. In some embodiments is a compound of Formula (I), (Ia), (Ib), (Id), (Ie), (Ig), (Ih), (Ii), (Ik), (Il), (Im), (In), (Io), (Iq), (Ir), (Is), (It), (Iu), (Iv), (Iw), (Ix), or (Iy), or a pharmaceutically acceptable salt or solvate thereof, wherein R⁷ is hydrogen. In some embodiments is a compound of Formula (I), (Ia), (Ib), (Id), (Ie), (Ig), (Ih), (Ii), (Ik), (Il), (Im), (In), (Io), (Iq), (Ir), (Is), (It), (Iu), (Iv), (Iw), (Ix), or (Iy), or a pharmaceutically acceptable salt or solvate thereof, wherein R⁷ is halogen. In some embodiments is a compound of Formula (I), (Ia), (Ib), (Id), (Ie), (Ig), (Ih), (Ii), (Ik), (Il), (Im), (In), (Io), (Iq), (Ir), (Is), (It), (Iu), (Iv), (Iw), (Ix), or (Iy), or a pharmaceutically acceptable salt or solvate thereof, wherein R⁷ is —CN. In some embodiments is a compound of Formula (I), (Ia), (Ib), (Id), (Ie), (Ig), (Ih), (Ii), (Ik), (Il), (Im), (In), (Io), (Iq), (Ir), (Is), (It), (Iu), (Iv), (Iw), (Ix), or (Iy), or a pharmaceutically acceptable salt or solvate thereof, wherein R⁷ is C₁₋₆alkyl optionally substituted with one, two, or three groups selected from R^{14b}. In some embodiments is a compound of Formula (I), (Ia), (Ib), (Id), (Ie), (Ig), (Ih), (Ii), (Ik), (Il), (Im), (In), (Io), (Iq), (Ir), (Is), (It), (Iu), (Iv), (Iw), (Ix), or (Iy), or a pharmaceutically acceptable salt or solvate thereof, wherein R⁷ is C₁₋₆alkyl substituted with one, two, or three groups selected from R^{14b}. In some embodiments is a compound of Formula (I), (Ia), (Ib), (Id), (Ie), (Ig), (Ih), (Ii), (Ik), (Il), (Im), (In), (Io), (Iq), (Ir), (Is), (It), (Iu), (Iv), (Iw), (Ix), or (Iy), or a pharmaceutically acceptable salt or solvate thereof, wherein R⁷ is unsubstituted C₁₋₆alkyl. In some embodiments is a compound of Formula (I), (Ia), (Ib), (Id), (Ie), (Ig), (Ih), (Ii), (Ik), (Il), (Im), (In), (Io), (Iq), (Ir), (Is), (It), (Iu), (Iv), (Iw), (Ix), or (Iy), or a pharmaceutically acceptable salt or solvate thereof, wherein R⁷ is —OR²⁰, wherein R²⁰ is independently selected from hydrogen and C₁₋₆alkyl. In some embodiments is a compound of Formula (I), (Ia), (Ib), (Id), (Ie), (Ig), (Ih), (Ii), (Ik), (Il), (Im), (In), (Io), (Iq), (Ir), (Is), (It), (Iu), (Iv), (Iw), (Ix), or (Iy), or a pharmaceutically acceptable salt or solvate thereof, wherein R⁷ is —C(O)N(R²⁰)(R²¹). In some embodiments is a compound of Formula (I), (Ia), (Ib), (Id), (Ie), (Ig), (Ih), (Ii), (Ik), (Il), (Im), (In), (Io), (Iq), (Ir), (Is), (It), (Iu), (Iv), (Iw), (Ix), or (Iy), or a pharmaceutically acceptable salt or solvate thereof, wherein R⁷ is —C(O)NH₂.

In some embodiments is a compound of Formula (I), (Ia), (Ic), (Id), (Ie), (If), (Ih), (Ij), (Ik), (Il), (Im), (In), (Io), (Ip), (Ir), (Is), (It), (Iu), (Iv), or (Iw), or a pharmaceutically acceptable salt or solvate thereof, wherein R⁸ is selected from hydrogen, halogen, —CN, C₁₋₆alkyl, C₁₋₆haloalkyl, —OR²⁰, —C(O)OR²⁰, —C(O)R²³, —C(O)N(R²⁰)(R²¹), —S(O)₂R²³, and —S(O)₂N(R²⁰)(R²¹)—, wherein C₁₋₆alkyl is optionally substituted with one, two, or three groups selected from R^{14b}. In some embodiments is a compound of Formula (I), (Ia), (Ic), (Id), (Ie), (If), (Ih), (Ij), (Ik), (Il), (Im), (In), (Io), (Ip), (Ir), (Is), (It), (Iu), (Iv), or (Iw), or a pharmaceutically acceptable salt or solvate thereof, wherein R⁸ is selected from hydrogen, halogen, —CN, C₁₋₆alkyl, —OR²⁰, and —C(O)N(R²⁰)(R²¹), wherein C₁₋₆alkyl is optionally substituted with one, two, or three groups selected from R^{14b}, and wherein R²⁰ is independently selected from hydrogen and C₁₋₆alkyl. In some embodiments is a compound of Formula (I), (Ia), (Ic), (Id), (Ie), (If), (Ih), (Ij), (Ik), (Il), (Im), (In), (Io), (Ip), (Ir), (Is), (It), (Iu), (Iv), or (Iw), or a pharmaceutically acceptable salt or solvate thereof, wherein R⁸ is hydrogen. In some embodiments is a compound of Formula (I), (Ia), (Ic), (Id), (Ie), (If), (Ih), (Ij), (Ik), (Il), (Im), (In), (Io), (Ip), (Ir), (Is), (It), (Iu), (Iv), or (Iw), or a pharmaceutically acceptable salt or solvate thereof, wherein R⁸ is halogen. In some embodiments is a compound of Formula (I), (Ia), (Ic), (Id), (Ie), (If), (Ih), (Ij), (Ik), (Il), (Im), (In), (Io), (Ip), (Ir), (Is), (It), (Iu), (Iv), or (Iw), or a pharmaceutically acceptable salt or solvate thereof, wherein R⁸ is —CN. In some embodiments is a compound of Formula (I), (Ia), (Ic), (Id), (Ie), (If), (Ih), (Ij), (Ik), (Il), (Im), (In), (Io), (Ip), (Ir), (Is), (It), (Iu), (Iv), or (Iw), or a pharmaceutically acceptable salt or solvate thereof, wherein R⁸ is C₁₋₆alkyl optionally substituted with one, two, or three groups selected from R^{14b}. In some embodiments is a compound of Formula (I), (Ia), (Ic), (Id), (Ie), (If), (Ih), (Ij), (Ik), (Ii), (Im), (In), (Io), (Ip), (Ir), (Is), (It), (Iu), (Iv), or (Iw), or a pharmaceutically acceptable salt or solvate thereof, wherein R⁸ is C₁₋₆alkyl substituted with one, two, or three groups selected from R^{14b}. In some embodiments is a compound of Formula (I), (Ia), (Ic), (Id), (Ie), (If), (Ih), (Ij), (Ik), (Il), (Im), (In), (Io), (Ip), (Ir), (Is), (It), (Iu), (Iv), or (Iw), or a pharmaceutically acceptable salt or solvate thereof, wherein R⁸ is unsubstituted C₁₋₆alkyl. In some embodiments is a compound of Formula (I), (Ia), (Ic), (Id), (Ie), (If), (Ih), (Ij), (Ik), (Il), (Im), (In), (Io), (Ip), (Ir), (Is), (It), (Iu), (Iv), or (Iw), or a pharmaceutically acceptable salt or solvate thereof, wherein R⁸ is —OR²⁰, wherein R²⁰ is independently selected from hydrogen and C₁₋₆alkyl. In some embodiments is a compound of Formula (I), (Ia), (Ic), (Id), (Ie), (If), (Ih), (Ij), (Ik), (Il), (Im), (In), (Io), (Ip), (Ir), (Is), (It), (Iu), (Iv), or (Iw), or a pharmaceutically acceptable salt or solvate thereof, wherein R⁸ is —C(O)N(R²⁰)(R²¹). In some embodiments is a compound of Formula (I), (Ia), (Ic), (Id), (Ie), (If), (Ih), (Ij), (Ik), (Il), (Im), (In), (Io), (Ip), (Ir), (Is), (It), (Iu), (Iv), or (Iw), or a pharmaceutically acceptable salt or solvate thereof, wherein R⁸ is —C(O)NH₂.

In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), or (Iy), or a pharmaceutically acceptable salt or solvate thereof, wherein L² is a bond. In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), or (Iy), or a pharmaceutically acceptable salt or solvate thereof, wherein L² is C₁₋₆alkylene optionally substituted with one, two, or three groups selected from R^{14a}. In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), or (Iy), or a pharmaceutically acceptable salt or solvate thereof, wherein L² is unsubstituted C₁₋₆alkylene. In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), or (Iw), or a pharmaceutically acceptable salt or solvate thereof, wherein L² is —CH₂—.

In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), or (Iy), or a pharmaceutically acceptable salt or solvate thereof, wherein R¹³ is selected from C₃₋₁₀cycloalkyl, C₂₋₉heterocycloalkyl, C₆₋₁₀aryl, and C₁₋₉heteroaryl, wherein C₃₋₁₀cycloalkyl, C₂₋₉heterocycloalkyl, C₆₋₁₀aryl, and C₁₋₉heteroaryl are optionally substituted with one, two, or three groups selected from R^{15b}. In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), or (Iy), or a pharmaceutically acceptable salt or solvate thereof, wherein R¹³ is C₆₋₁₀aryl optionally substituted with one, two, or three groups selected from R^{15b}. In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), or (Iy), or a pharmaceutically acceptable salt or solvate thereof, wherein R¹³ is phenyl optionally substituted with one, two, or three groups selected from R^{15b}. In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), or (Iy), or a pharmaceutically acceptable salt or solvate thereof, wherein R¹³ is phenyl substituted with one, two, or three groups selected from R^{15b}. In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), or (Iy), or a pharmaceutically acceptable salt or solvate thereof, wherein R¹³ is phenyl substituted with one R^{15b} group. In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), or (Iy), or a pharmaceutically acceptable salt or solvate thereof, wherein R¹³ is C₁₋₉heteroaryl optionally substituted with one, two, or three groups selected from R^{15b}. In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), or (Iy), or a pharmaceutically acceptable salt or solvate thereof, wherein R¹³ is C₁₋₉heteroaryl optionally substituted with one, two, or three groups selected from R^{15b}, wherein C₁₋₉heteroaryl is selected from pyridyl, pyrimidinyl, pyrazinyl, pyridazinyl, thienyl, furanyl, pyrrolyl, oxazolyl, thiazolyl, pyrazolyl, indazolyl, and imidazolyl. In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), or (Iy), or a pharmaceutically acceptable salt or solvate thereof, wherein R¹³ is C₁₋₉heteroaryl substituted with one, two, or three groups selected from R^{15b}, wherein C₁₋₉heteroaryl is selected from pyridyl, pyrimidinyl, pyrazinyl, pyridazinyl, thienyl, furanyl, pyrrolyl, oxazolyl, thiazolyl, pyrazolyl, indazolyl, and imidazolyl. In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), or (Iy), or a pharmaceutically acceptable salt or solvate thereof, wherein R¹³ is C₁₋₉heteroaryl substituted with one R^{15b} group, wherein C₁₋₉heteroaryl is selected from pyridyl, pyrimidinyl, pyrazinyl, pyridazinyl, thienyl, furanyl, pyrrolyl, oxazolyl, thiazolyl, pyrazolyl, indazolyl, and imidazolyl. In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), or (Iy), or a pharmaceutically acceptable salt or solvate thereof, wherein R¹³ is C₃₋₁₀cycloalkyl optionally substituted with one, two, or three groups selected from R^{15b}. In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), or (Iy), or a pharmaceutically acceptable salt or solvate thereof, wherein R¹³ is C₂₋₉heterocycloalkyl optionally substituted with one, two, or three groups selected from R^{15b}.

In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), or (Iy), or a pharmaceutically acceptable salt or solvate thereof, wherein each R^{15b} is independently selected from halogen, C₁₋₆alkyl, C₁₋₆haloalkyl, and —OR²⁰, wherein R²⁰ is selected from C₁₋₆alkyl and C₁₋₆haloalkyl. In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), or (Iy), or a pharmaceutically acceptable salt or solvate thereof, wherein each R^{15b} is halogen. In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), or (Iy), or a pharmaceutically acceptable salt or solvate thereof, wherein each R^{15b} is C₁₋₆haloalkyl. In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), or (Iy), or a pharmaceutically acceptable salt or solvate thereof, wherein each R^{15b} is —CF₃. In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), or (Iy), or a pharmaceutically acceptable salt or solvate thereof, wherein each R^{15b} is C₁₋₆alkyl. In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), or (Iy), or a pharmaceutically acceptable salt or solvate thereof, wherein each R^{15b} is —CH₃. In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), or (Iy), or a pharmaceutically acceptable salt or solvate thereof, wherein each R^{15b} is —OR²⁰, wherein R²⁰ is selected from C₁₋₆alkyl and C₁₋₆haloalkyl.

In some embodiments is a compound of Formula (I), (Ia), (Ib), (Ic), (Id), (Ie), (If), (Ig), (Ih), (Ii), (Ij), (Ik), (Ii), (Im), (In), (Io), (Ip), (Iq), (Ir), (Is), (It), (Iu), (Iv), (Iw), (Ix), or (Iy), or a pharmaceutically acceptable salt or solvate thereof, wherein L¹ is C₁₋₆alkylene optionally substituted with one, two, or three groups selected from R^{14a}. In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), or (Iy), or a pharmaceutically acceptable salt or solvate thereof, wherein L¹ is unsubstituted C₁₋₆alkylene. In some embodiments is a compound of Formula (I), (Ia), (Ib), (Ic), (Id), (Ie), (If), (Ig), (Ih), (Ii), (Ij), (Ik), (It), (Im), (In), (Io), (Ip), (Iq), (Ir), (Is), (It), (Iu), (Iv), (Iw), (Ix), or (Iy), or a pharmaceutically acceptable salt or solvate thereof, wherein L¹ is —CH₂—. In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), or (Iy), or a pharmaceutically acceptable salt or solvate thereof, wherein L¹ is a bond.

In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), or (Iy), or a pharmaceutically acceptable salt or solvate thereof, wherein R¹² is selected from C₁₋₆alkyl, C₃₋₁₀cycloalkyl, C₂₋₉heterocycloalkyl, C₆₋₁₀aryl, and C₁₋₆heteroaryl, wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three groups selected from R^{15a}. In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), or (Iy), or a pharmaceutically acceptable salt or solvate thereof, wherein R¹² is C₁₋₉heteroaryl optionally substituted with one, two, or three groups selected from R^{15a}. In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), or (Iy), or a pharmaceutically acceptable salt or solvate thereof, wherein R¹² is C₁₋₉heteroaryl optionally substituted with one, two, or three groups selected from R^{15a}, wherein C₁₋₉heteroaryl is selected from pyridyl, pyrimidinyl, pyrazinyl, pyridazinyl, thienyl, furanyl, pyrrolyl, oxazolyl, thiazolyl, pyrazolyl, indazolyl, and imidazolyl. In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), or (Iy), or a pharmaceutically acceptable salt or solvate thereof, wherein R¹² is C₁₋₉heteroaryl optionally substituted with one, two, or three groups selected from R^{15a}, wherein C₁₋₉heteroaryl is selected from pyridyl, pyrimidinyl, pyrazinyl, pyridazinyl, and pyrazolyl. In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), or (Iy), or a pharmaceutically acceptable salt or solvate thereof, wherein R¹² is C₁₋₉heteroaryl optionally substituted with one, two, or three groups selected from R^{15a}, wherein C₁₋₉heteroaryl is selected from pyridyl, pyrimidinyl, and pyrazolyl. In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), or (Iy), or a pharmaceutically acceptable salt or solvate thereof, wherein R¹² is pyridyl optionally substituted with one, two, or three groups selected from R^{15a}. In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), or (Iy), or a pharmaceutically acceptable salt or solvate thereof, wherein R¹² is unsubstituted pyridyl. In some embodiments is a compound of Formula (I), (Ia), (Ib), (Ic), (Id), (Ie), (If), (Ig), (Ih), (Ii), (Ij), (Ik), (Ii), (Im), (In), (Io), (Ip), (Iq), (Ir), (Is), (It), (Iu), (Iv), (Iw), (Ix), or (Iy), or a pharmaceutically acceptable salt or solvate thereof, wherein R¹² is pyrimidinyl optionally substituted with one, two, or three groups selected from R^{15a}. In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), or (Iy), or a pharmaceutically acceptable salt or solvate thereof, wherein R¹² is unsubstituted pyrimidinyl. In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), or (Iw), or a pharmaceutically acceptable salt or solvate thereof, wherein R¹² is pyrazolyl optionally substituted with one, two, or three groups selected from R^{15a}. In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), or (Iy), or a pharmaceutically acceptable salt or solvate thereof, wherein R¹² is unsubstituted pyrazolyl. In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), or (Iy), or a pharmaceutically acceptable salt or solvate thereof, wherein R¹² is C₂₋₉heterocycloalkyl optionally substituted with one, two, or three groups selected from R^{15a}. In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), or (Iy), or a pharmaceutically acceptable salt or solvate thereof, wherein R¹² is C₁₋₆alkyl optionally substituted with one, two, or three groups selected from R^{15a}. In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), or (Iy), or a pharmaceutically acceptable salt or solvate thereof, wherein R¹² is unsubstituted C₁₋₆alkyl.

In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), or (Iy), or a pharmaceutically acceptable salt or solvate thereof, wherein each R^{15a} is independently selected from halogen, —CN, C₁₋₆alkyl, C₁₋₆alkoxyl, and C₁₋₆haloalkyl. In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), or (Iy), or a pharmaceutically acceptable salt or solvate thereof, wherein each R^{15a} is halogen. In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), or (Iy), or a pharmaceutically acceptable salt or solvate thereof, wherein each R^{15a} is C₁₋₆haloalkyl. In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), or (Iy), or a pharmaceutically acceptable salt or solvate thereof, wherein each R^{15a} is —CF₃. In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), or (Iy), or a pharmaceutically acceptable salt or solvate thereof, wherein each R^{15a} is C₁₋₆alkyl. In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), or (Iy), or a pharmaceutically acceptable salt or solvate thereof, wherein each R^{15a} is —CH₃. In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), or (Iy), or a pharmaceutically acceptable salt or solvate thereof, wherein each R^{15a} is —CN. In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), or (Iy), or a pharmaceutically acceptable salt or solvate thereof, wherein each R^{15a} is —N(R²⁰)(R²¹).

In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), or (Iw), or a pharmaceutically acceptable salt or solvate thereof, wherein each R^{15a} is independently selected from —NH₂, —NH(CH₃), —N(CH₃)₂, —N(CH₃)(CH₂)₂OH, —NH(CH₂)₂OCH₃,

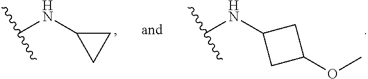

In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), or (Iw), or a pharmaceutically acceptable salt or solvate thereof, wherein each R^{15a} is —NH₂. In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), or (Iy), or a pharmaceutically acceptable salt or solvate thereof, wherein each R^{15a} is —NH(CH₃). In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), or (Iy), or a pharmaceutically acceptable salt or solvate thereof, wherein each R^{15a} is —N(CH₃)₂. In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), or (Iy), or a pharmaceutically acceptable salt or solvate thereof, wherein each R^{15a} is —N(CH₃)(CH₂)₂OH. In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), or (Iy), or a pharmaceutically acceptable salt or solvate thereof, wherein each R^{15a} is —NH(CH₂)₂OCH₃. In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), or (Iy), or a pharmaceutically acceptable salt or solvate thereof, wherein each R^{15a} is

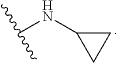 [SMILES: *NC1CC1]

In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), or (Iy), or a pharmaceutically acceptable salt or solvate thereof, wherein each R^{15a} is

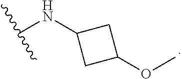 [SMILES: *NC1CC(OC)C1]

In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), or (Iy), or a pharmaceutically acceptable salt or solvate thereof, wherein each R^{15a} is —OR²⁰, wherein R²⁰ is independently selected from hydrogen, C₁₋₆alkyl, and C₃₋₆cycloalkyl, wherein C₁₋₆alkyl, and C₃₋₆cycloalkyl, is optionally substituted with one, two, or three groups selected from halogen, hydroxy, C₁₋₆alkyl, and C₁₋₆alkoxy, C₁₋₉heteroaryl. In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), or (Iy), or a pharmaceutically acceptable salt or solvate thereof, wherein each R^{15a} is —OR²⁰, wherein R²⁰ is independently selected from hydrogen, —CH₃, —CH₂CH₂OH, —CH₂CH₂OCH₃, cyclopropyl and cyclobutyl, wherein cyclopropyl and cyclobutyl, is optionally substituted with —OCH₃. In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), or (Iy), or a pharmaceutically acceptable salt or solvate thereof, wherein each R^{15a} is independently selected from —OH, —OCH₃, —OCH₂CH₂OH, and —OCH₂CH₂OCH₃.

In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), or (Iy), or a pharmaceutically acceptable salt or solvate thereof, wherein each R^{15a} is independently selected from —OR²⁰, and —N(R²⁰)(R²¹), optionally, wherein two R^{15a} are combined to form a C₃₋₆cycloalkyl or C₂₋₉heterocycloalkyl, wherein C₃₋₆cycloalkyl and C₂₋₉heterocycloalkyl are optionally substituted with one, two, or three groups independently selected from halogen, and —OR²⁰.

In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), or (Iy), or a pharmaceutically acceptable salt or solvate thereof, wherein each R^{15a} is —N(R²⁰)(R²¹). In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), or (Iy), or a pharmaceutically acceptable salt or solvate thereof, wherein each R^{15a} is —N(R²⁰)(R²¹), wherein R²⁰ and R²¹, together with the nitrogen to which they are attached, form a C₂₋₉heterocycloalkyl.

In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), or (Iy), or a pharmaceutically acceptable salt or solvate thereof, wherein each R^{15a} is —N(R²⁰)(R²¹), wherein R²⁰ and R²¹, together with the nitrogen to which they are attached, form a C₂₋₉heterocycloalkyl, wherein C₂₋₉heterocycloalkyl is optionally substituted with hydroxy or C₁₋₆alkyl. In some embodiments is a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), or (Iy), or a pharmaceutically acceptable salt or solvate thereof, wherein each R^{15a} is azetidinyl, pyrrolidinyl, morpholino,

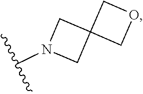 [SMILES: *N1CC2(COC2)C1]

or piperidinyl, wherein azetidinyl, pyrrolidinyl, morpholino,

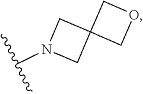 [SMILES: *N1CC2(COC2)C1]

and piperidinyl are optionally substituted with hydroxy.

In some embodiments, described herein is a compound of Formula (II), or a pharmaceutically acceptable salt or solvate thereof:

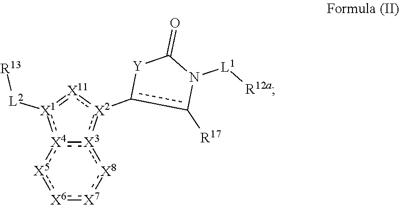

wherein,
X¹ is C, C(R¹), or N;
X² is C, C(R²), or N;
X³ is C, C(R³), or N;
X⁴ is C, C(R⁴), or N;
X⁵ is C(R⁵), C(R⁵)(R₅ₐ), N(R^{5b}), or N;
X⁶ is C(R⁶), C(R⁶)(R^{6a}), N(R^{6b}), or N;
X⁷ is C(R⁷), C(R⁷)(R^{7a}), N(R^{7b}), or N;
X⁸ is C(R⁸), C(R⁸)(R^{8a}), N(R^{8b}), or N;
X¹¹ is C(R¹⁶), C(R¹⁶)(R^{16a}), C(O), N(R^{16b}), or N;
Y is N(R¹⁸) or O;
L¹ and L² are independently selected from a bond and C₁₋₆alkylene optionally substituted with one, two, or three groups selected from R^{14a};
R¹, R², R³, and R⁴ are independently selected from hydrogen and C₁₋₆alkyl;
R⁵, R^{5a}, R⁶, R^{6a}, R⁷, R^{7a}, R⁸, R^{8a}, R¹⁶, and R^{16a} are independently selected from hydrogen, halogen, —CN, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR²⁰, —SR²⁰, —SF₅, —N(R²⁰)(R^{21a}), —C(O)OR²⁰, —OC(O)N(R²⁰)(R²¹), —N(R²²)C(O)N(R²⁰)(R²¹), —N(R²²)C(O)OR²³, —N(R²²)S(O)₂R²³, —C(O)R²³, —S(O)R²³, —OC(O)R²³, —C(O)N(R²⁰)(R²¹), —C(O)C(O)N(R²⁰)(R²¹), —N(R²²)C(O)R²³, —S(O)₂R²³, —S(O)₂N(R²⁰)(R²¹)—, —N═S(═O)(R²³)₂, —S(═O)(═NH)N(R²⁰)(R²¹), —S(═O)(═NH)C(R²⁰)(R²¹), —S(═O)(═NR²³)R²³, —CH₂C(O)N(R²⁰)(R²¹), —CH₂N(R²²)C(O)R²³, —CH₂S(O)₂R²³, and —CH₂S(O)₂N(R²⁰)(R²¹), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three groups selected from R^{14b};
R^{5b}, R^{6b}, R^{7b}, R^{8b}, and R^{16b} are independently selected from hydrogen, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —C(O)OR²⁰, —OC(O)N(R²⁰)(R²¹), —C(O)R²³, —C(O)N(R²⁰)(R²¹), and —S(O)₂R²³, wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three groups selected from R^{14c};
R^{12a} is selected from C₃₋₁₀cycloalkyl, C₂₋₉heterocycloalkyl, C₆₋₁₀aryl, and C₁₋₉heteroaryl, wherein C₃₋₁₀cycloalkyl, C₂₋₉heterocycloalkyl, C₆₋₁₀aryl, and C₁₋₉heteroaryl are optionally substituted with one, two, or three groups selected from R^{15a};
R¹³ is selected from C₁₋₆alkyl, C₁₋₆alkoxyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl, wherein C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three groups selected from R^{15b};
each R^{14a}, R^{14b}, and R^{14c} are each independently selected from halogen, oxo, —CN, C₁₋₆alkyl, C₁₋₆haloalkyl, C₂₋₆alkenyl, C₂₋₆alkynyl, C₃₋₁₀cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR²⁰, —SR²⁰, —SF₅, —N(R²⁰)(R²¹), —C(O)OR²⁰, —OC(O)N(R²⁰)(R²¹), —N(R²²)C(O)N(R²⁰)(R²¹), —N(R²²)C(O)OR²³, —N(R²²)S(O)₂R²³, —C(O)R²³, —S(O)R²³, —OC(O)R²³, —C(O)N(R²⁰)(R²¹), —C(O)C(O)N(R²⁰)(R²¹), —N(R²²)C(O)R²³, —S(O)₂R²³, —S(O)₂N(R²⁰)(R²¹)—, —N═S(═O)(R²³)₂, —S(═O)(═NH)N(R²⁰)(R²¹), —S(═O)(═NH)C(R²⁰)(R²¹), —S(═O)(═NR²³)R²³, —CH₂C(O)N(R²⁰)(R²¹), —CH₂N(R²³)C(O)R²³, —CH₂S(O)₂R²³, —CH₂S(O)₂N(R²⁰)(R²¹), wherein C₁₋₆alkyl, C₁₋₆haloalkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²⁰, —SR²⁰, —SF₅, —N(R²⁰)(R²¹), —C(O)OR²⁰, —OC(O)N(R²⁰)(R²¹), —N(R²²)C(O)N(R²⁰)(R²¹), —N(R²²)C(O)OR²³, —N(R²²)S(O)₂R²³, —C(O)R²³, —S(O)R²³, —OC(O)R²³, —C(O)N(R²⁰)(R²¹), —C(O)C(O)N(R²⁰)(R²¹), —N(R²²)C(O)R²³, —S(O)₂R²³, —S(O)₂N(R²⁰)(R²¹)—, —N═S(═O)(R²³)₂, —S(═O)(═NH)N(R²⁰)(R²¹), —S(═O)(═NH)C(R²⁰)(R²¹), —S(═O)(═NR²³)R²³, —CH₂C(O)N(R²⁰)(R²¹), —CH₂N(R²²)C(O)R²³, —CH₂S(O)₂R²³, and —CH₂S(O)₂N(R²⁰)(R²¹);
each R^{15a} is independently selected from halogen, oxo, —CN, C₁₋₆alkyl, C₁₋₆haloalkyl, C₂₋₆alkenyl, C₂₋₆alkynyl, C₃₋₁₀cycloalkyl, —CH₂—C₃₋₆cycloalkyl, C₂₋₉heterocycloalkyl, —CH₂—C₂₋₉heterocycloalkyl, —NH—C₃₋₉cycloalkyl, C₆₋₁₀aryl, —CH₂—C₆₋₁₀aryl, C₁₋₉heteroaryl, —CH₂—C₁₋₉heteroaryl, —OR²⁰, —SR²⁰, —SF₅, —N(R²⁰)(R²¹), —C(O)OR²⁰, —OC(O)N(R²⁰)(R²¹), —N(R²²)C(O)N(R²⁰)(R²¹), —N(R²²)C(O)OR²³, —N(R²²)S(O)₂R²³, —C(O)R²³, —S(O)R²³, —OC(O)R²³, —C(O)N(R²⁰)(R²¹), —C(O)C(O)N(R²⁰)(R²¹), —N(R²²)C(O)R²³, —S(O)₂R²³, —S(O)₂N(R²⁰)(R²¹)—, —N═S(═O)(R²³)₂, —S(═O)(═NH)N(R²⁰)(R²¹), —S(═O)(═NH)C(R²⁰)(R²¹), —S(═O)(═NR²³)R²³, —CH₂C(O)N(R²⁰)(R²¹), —CH₂N(R²²)C(O)R²³, —CH₂S(O)₂R²³, —CH₂S(O)₂N(R²⁰)(R²¹), wherein C₁₋₆alkyl, C₁₋₆haloalkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²⁰, —SR²⁰, —SF₅, —N(R²⁰)(R²¹), —C(O)OR²⁰, —OC(O)N(R²⁰)(R²¹), —N(R²²)C(O)N(R²⁰)(R²¹), —N(R²²)C(O)OR²³, —N(R²²)S(O)₂R²³, —C(O)R²³, —S(O)R²³, —OC(O)R²³, —C(O)N(R²⁰)(R²¹), —C(O)C(O)N(R²⁰)(R²¹), —N(R²²)C(O)R²³, —S(O)₂R²³, —S(O)₂N(R²⁰)(R²¹)—, —N═S(═O)(R²³)₂, —S(═O)(═NH)N(R²⁰)(R²¹), —S(═O)(═NH)C(R²⁰)(R²¹), —S(═O)(═NR²³)R²³, —CH₂C(O)N(R²⁰)(R²¹), —CH₂N(R²²)C(O)R²³, —CH₂S(O)₂R²³, and —CH₂S(O)₂N(R²⁰)(R²¹); or two R^{15a} are combined to form a C₃₋₆cycloalkyl or C₂₋₉heterocycloalkyl, wherein C₃₋₆cycloalkyl and C₂₋₉heterocycloalkyl are optionally substituted with one, two, or three groups independently selected from halogen, oxo, C₁₋₆alkyl, C₁₋₆haloalkyl, and C₁₋₆alkoxy;
each R^{15b} is independently selected from halogen, oxo, —CN, C₁₋₆alkyl, C₁₋₆haloalkyl, C₂₋₆alkenyl, C₂₋₆alkynyl, C₃₋₁₀cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR²⁰, —SR²⁰, —SF₅, —N(R²⁰)(R²¹), —C(O)OR²⁰, —OC(O)N(R²⁰)(R²¹), —N(R²²)C(O)N(R²⁰)(R²¹), —N(R²²)C(O)OR²³, —N(R²²)S(O)₂R²³, —C(O)R²³, —S(O)R²³, —OC(O)R²³, —C(O)N(R²⁰)(R²¹), —C(O)C(O)N(R²⁰)(R²¹), —N(R²²)C(O)R²³, —S(O)₂R²³, —S(O)₂N(R²⁰)(R²¹)—, —N═S(═O)(R²³)₂, —S(═O)(═NH)N(R²⁰)(R²¹), —S(═O)(═NH)C(R²⁰)(R²¹), —S(═O)(═NR²³)R²³, —CH₂C(O)N(R²⁰)(R²¹), —CH₂N(R²²)C(O)R²³, —CH₂S(O)₂R²³, —CH₂S(O)₂N(R²⁰)(R²¹), wherein C₁₋₆alkyl, C₁₋₆haloalkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²⁰, —SR²⁰, —SF₅, —N(R²⁰)(R²¹), —C(O)OR²⁰, —OC(O)N(R²⁰)(R²¹), —N(R²²)C(O)N(R²⁰)(R²¹), —N(R²²)C(O)OR²³, —N(R²²)S(O)₂R²³, —C(O)R²³, —S(O)R²³, —OC(O)R²³, —C(O)N(R²⁰)(R²¹), —C(O)C(O)N(R²⁰)(R²¹), —N(R²²)C(O)R²³, —S(O)₂R²³, —S(O)₂N(R²⁰)(R²¹)—, —N═S(═O)(R²³)₂, —S(═O)(═NH)N(R²⁰)(R²¹), —S(═O)(═NH)C(R²⁰)(R²¹), —S(═O)(═NR²³)R²³, —CH₂C(O)N(R²⁰)(R²¹), —CH₂N(R²²)C(O)R²³, —CH₂S(O)₂R²³, and —CH₂S(O)₂N(R²⁰)(R²¹); or two R^{15b} are combined to form a C₃₋₆cycloalkyl or C₂₋₉heterocycloalkyl, wherein C₃₋₆cycloalkyl and C₂₋₉heterocycloalkyl are optionally substituted with one, two, or three groups independently selected from halogen, oxo, C₁₋₆alkyl, C₁₋₆haloalkyl, and C₁₋₆alkoxy;
R¹⁷ is selected from hydrogen, halogen, and C₁₋₆alkyl;
R¹⁸ is selected from hydrogen and C₁₋₆alkyl;
each R²⁰ is independently selected from hydrogen, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R²¹ is independently selected from hydrogen, C₁₋₆alkyl, and C₁₋₆haloalkyl; or R²⁰ and R²¹, together with the nitrogen to which they are attached, form a C₂₋₉heterocycloalkyl;
each R^{21a} is independently selected from C₁₋₆alkyl and C₁₋₆haloalkyl;
each R²² is independently selected from hydrogen, C₁₋₆alkyl, and C₁₋₆haloalkyl;
each R²³ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl; and
[symbol] indicates a single or double bond such that all valences are satisfied.

In some embodiments is a compound of Formula (II), or a pharmaceutically acceptable salt or solvate thereof, wherein Y is N(R¹⁸). In some embodiments is a compound of Formula (II), or a pharmaceutically acceptable salt or solvate thereof, wherein Y is N(H). In some embodiments is a compound of Formula (II), or a pharmaceutically acceptable salt or solvate thereof, wherein Y is N(R¹⁸) and R¹⁸ is C₁₋₆alkyl. In some embodiments is a compound of Formula (II), or a pharmaceutically acceptable salt or solvate thereof, wherein Y is N(CH₃). In some embodiments is a compound of Formula (II), or a pharmaceutically acceptable salt or solvate thereof, wherein Y is O.

In some embodiments is a compound of Formula (II), or a pharmaceutically acceptable salt or solvate thereof, wherein R¹⁷ is hydrogen. In some embodiments is a compound of Formula (II), or a pharmaceutically acceptable salt or solvate thereof, wherein R¹⁷ is C₁₋₆alkyl. In some embodiments is a compound of Formula (II), or a pharmaceutically acceptable salt or solvate thereof, wherein R¹⁷ is —CH₃.

In some embodiments is a compound of Formula (II), or a pharmaceutically acceptable salt or solvate thereof, wherein X¹¹ is N. In some embodiments is a compound of Formula (II), or a pharmaceutically acceptable salt or solvate thereof, wherein X¹¹ is C(R¹⁶). In some embodiments is a compound of Formula (II), or a pharmaceutically acceptable salt or solvate thereof, wherein X¹¹ is C(H). In some embodiments is a compound of Formula (II), or a pharmaceutically acceptable salt or solvate thereof, wherein X¹¹ is C(O).

In some embodiments is a compound of Formula (II), or a pharmaceutically acceptable salt or solvate thereof, wherein X¹ is C. In some embodiments is a compound of Formula (II), or a pharmaceutically acceptable salt or solvate thereof, wherein X¹ is N. In some embodiments is a compound of Formula (II), or a pharmaceutically acceptable salt or solvate thereof, wherein X is C(R¹). In some embodiments is a compound of Formula (II), or a pharmaceutically acceptable salt or solvate thereof, wherein X¹ is C(H).

In some embodiments is a compound of Formula (II), or a pharmaceutically acceptable salt or solvate thereof, wherein X² is C. In some embodiments is a compound of Formula (II), or a pharmaceutically acceptable salt or solvate thereof, wherein X² is N. In some embodiments is a compound of Formula (II), or a pharmaceutically acceptable salt or solvate thereof, wherein X² is C(R²). In some embodiments is a compound of Formula (II), or a pharmaceutically acceptable salt or solvate thereof, wherein X² is C(H).

In some embodiments is a compound of Formula (II), or a pharmaceutically acceptable salt or solvate thereof, wherein X¹ is N and X² is C. In some embodiments is a compound of Formula (II), or a pharmaceutically acceptable salt or solvate thereof, wherein X¹ is C and X² is N.

In some embodiments is a compound of Formula (II), or a pharmaceutically acceptable salt or solvate thereof, wherein X³ is C. In some embodiments is a compound of Formula (II), or a pharmaceutically acceptable salt or solvate thereof, wherein X³ is N. In some embodiments is a compound of Formula (II), or a pharmaceutically acceptable salt or solvate thereof, wherein X³ is C(R³). In some embodiments is a compound of Formula (II), or a pharmaceutically acceptable salt or solvate thereof, wherein X³ is C(H).

In some embodiments is a compound of Formula (II), or a pharmaceutically acceptable salt or solvate thereof, wherein X⁴ is C. In some embodiments is a compound of Formula (II), or a pharmaceutically acceptable salt or solvate thereof, wherein X⁴ is N. In some embodiments is a compound of Formula (II), or a pharmaceutically acceptable salt or solvate thereof, wherein X⁴ is C(R⁴). In some embodiments is a compound of Formula (II), or a pharmaceutically acceptable salt or solvate thereof, wherein X⁴ is C(H).

In some embodiments is a compound of Formula (II), or a pharmaceutically acceptable salt or solvate thereof, wherein X³ is C and X⁴ is C. In some embodiments is a compound of Formula (II), or a pharmaceutically acceptable salt or solvate thereof, wherein X³ is N and X⁴ is C. In some embodiments is a compound of Formula (II), or a pharmaceutically acceptable salt or solvate thereof, wherein X³ is C and X⁴ is N.

In some embodiments is a compound of Formula (II), or a pharmaceutically acceptable salt or solvate thereof, wherein X⁵ is C(R⁵) or N; X⁶ is C(R⁶) or N; X⁷ is C(R⁷) or N; and X⁸ is C(R⁸) or N. In some embodiments is a compound of Formula (II), or a pharmaceutically acceptable salt or solvate thereof, wherein X⁵ is C(R⁵); X⁶ is C(R⁶); X⁷ is C(R⁷); and X⁸ is C(R⁸). In some embodiments is a compound of Formula (II), or a pharmaceutically acceptable salt or solvate thereof, wherein X⁵ is N; X⁶ is C(R⁶); X⁷ is C(R⁷); and X⁸ is C(R⁸). In some embodiments is a compound of Formula (II), or a pharmaceutically acceptable salt or solvate thereof, wherein X⁵ is C(R⁵); X⁶ is N; X⁷ is C(R⁷); and X⁸ is C(R⁸). In some embodiments is a compound of Formula (II), or a pharmaceutically acceptable salt or solvate thereof, wherein X⁵ is C(R⁵); X⁶ is C(R⁶); X⁷ is N; and X⁸ is C(R⁸). In some embodiments is a compound of Formula (II), or a pharmaceutically acceptable salt or solvate thereof, wherein X⁵ is C(R⁵); X⁶ is C(R⁶) X⁷ is C(R⁷); and X⁸ is N. In some embodiments is a compound of Formula (II), or a pharmaceutically acceptable salt or solvate thereof, wherein X⁵ is N; X⁶ is C(R⁶); X⁷ is N; and X⁸ is C(R⁸). In some embodiments is a compound of Formula (II), or a pharmaceutically acceptable salt or solvate thereof, wherein X⁵ is C(R⁵); X⁶ is N; X⁷ is C(R⁷); and X⁸ is N.

In some embodiments is a compound of Formula (II), or a pharmaceutically acceptable salt or solvate thereof, having the structure of Formula (IIa):

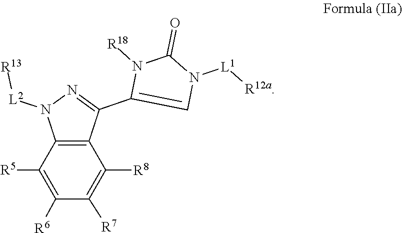

In some embodiments is a compound of Formula (II), or a pharmaceutically acceptable salt or solvate thereof, having the structure of Formula (IIb):

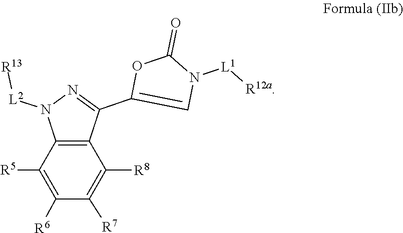

In some embodiments is a compound of Formula (II), (IIa), or (IIb), or a pharmaceutically acceptable salt or solvate thereof, wherein R⁵ is selected from hydrogen, halogen, —CN, C₁₋₆alkyl, C₁₋₆haloalkyl, —OR²⁰, —C(O)OR²⁰, —C(O)R²³, —C(O)N(R²⁰)(R²¹), —S(O)₂R²³, and —S(O)₂N(R²⁰)(R²¹)—, wherein C₁₋₆alkyl is optionally substituted with one, two, or three groups selected from R^{14b}. In some embodiments is a compound of Formula (II), (IIa), or (IIb), or a pharmaceutically acceptable salt or solvate thereof, wherein R⁵ is selected from hydrogen, halogen, —CN, C₁₋₆alkyl, —OR²⁰, and —C(O)N(R²⁰)(R²¹), wherein C₁₋₆alkyl is optionally substituted with one, two, or three groups selected from R^{14b}, and wherein R²⁰ is independently selected from hydrogen and C₁₋₆alkyl. In some embodiments is a compound of Formula (II), (IIa), or (IIb), or a pharmaceutically acceptable salt or solvate thereof, wherein R⁵ is hydrogen. In some embodiments is a compound of Formula (II), (IIa), or (IIb), or a pharmaceutically acceptable salt or solvate thereof, wherein R⁵ is halogen. In some embodiments is a compound of Formula (II), (IIa), or (IIb), or a pharmaceutically acceptable salt or solvate thereof, wherein R⁵ is F, or Cl. In some embodiments is a compound of Formula (II), (IIa), or (IIb), or a pharmaceutically acceptable salt or solvate thereof, wherein R⁵ is F. In some embodiments is a compound of Formula (II), (IIa), or (IIb), or a pharmaceutically acceptable salt or solvate thereof, wherein R⁵ is —CN. In some embodiments is a compound of Formula (II), (IIa), or (IIb), or a pharmaceutically acceptable salt or solvate thereof, wherein R⁵ is C₁₋₆alkyl optionally substituted with one, two, or three groups selected from R^{14b}. In some embodiments is a compound of Formula (II), (IIa), or (IIb), or a pharmaceutically acceptable salt or solvate thereof, wherein R⁵ is C₁₋₆alkyl substituted with one, two, or three groups selected from R^{14b}. In some embodiments is a compound of Formula (II), (IIa), or (IIb), or a pharmaceutically acceptable salt or solvate thereof, wherein R⁵ is unsubstituted C₁₋₆alkyl. In some embodiments is a compound of Formula (II), (IIa), or (IIb), or a pharmaceutically acceptable salt or solvate thereof, wherein R⁵ is —OR²⁰, wherein R²⁰ is independently selected from hydrogen and C₁₋₆alkyl. In some embodiments is a compound of Formula (II), (IIa), or (IIb), or a pharmaceutically acceptable salt or solvate thereof, wherein R⁵ is —C(O)N(R²⁰)(R²¹). In some embodiments is a compound of Formula (II), (IIa), or (IIb), or a pharmaceutically acceptable salt or solvate thereof, wherein R⁵ is —C(O)NH₂.

In some embodiments is a compound of Formula (II), (IIa), or (IIb), or a pharmaceutically acceptable salt or solvate thereof, wherein R⁶ is selected from hydrogen, halogen, —CN, C₁₋₆alkyl, C₁₋₆haloalkyl, —OR²⁰, —C(O)OR²⁰, —C(O)R²³, —C(O)N(R²⁰)(R²¹), —S(O)₂R²³, and —S(O)₂N(R²⁰)(R²¹)—, wherein C₁₋₆alkyl is optionally substituted with one, two, or three groups selected from R^{14b}. In some embodiments is a compound of Formula (II), (IIa), or (IIb), or a pharmaceutically acceptable salt or solvate thereof, wherein R⁶ is selected from hydrogen, halogen, —CN, C₁₋₆alkyl, —OR²⁰, and —C(O)N(R²⁰)(R²¹), wherein C₁₋₆alkyl is optionally substituted with one, two, or three groups selected from R^{14b}, and wherein R²⁰ is independently selected from hydrogen and C₁₋₆alkyl. In some embodiments is a compound of Formula (II), (IIa), or (IIb), or a pharmaceutically acceptable salt or solvate thereof, wherein R⁶ is hydrogen. In some embodiments is a compound of Formula (II), (IIa), or (IIb), or a pharmaceutically acceptable salt or solvate thereof, wherein R⁶ is halogen. In some embodiments is a compound of Formula (II), (IIa), or (IIb), or a pharmaceutically acceptable salt or solvate thereof, wherein R⁶ is —CN. In some embodiments is a compound of Formula (II), (IIa), or (IIb), or a pharmaceutically acceptable salt or solvate thereof, wherein R⁶ is C₁₋₆alkyl optionally substituted with one, two, or three groups selected from R^{14b}. In some embodiments is a compound of Formula (II), (IIa), or (IIb), or a pharmaceutically acceptable salt or solvate thereof, wherein R⁶ is C₁₋₆alkyl substituted with one, two, or three groups selected from R^{14b}. In some embodiments is a compound of Formula (II), (IIa), or (IIb), or a pharmaceutically acceptable salt or solvate thereof, wherein R⁶ is unsubstituted C₁₋₆alkyl. In some embodiments is a compound of Formula (II), (IIa), or (IIb), or a pharmaceutically acceptable salt or solvate thereof, wherein R⁶ is —OR²⁰, wherein R²⁰ is independently selected from hydrogen and C₁₋₆alkyl. In some embodiments is a compound of Formula (II), (IIa), or (IIb), or a pharmaceutically acceptable salt or solvate thereof, wherein R⁶ is —C(O)N(R²⁰)(R²¹). In some embodiments is a compound of Formula (II), (IIa), or (IIb), or a pharmaceutically acceptable salt or solvate thereof, wherein R⁶ is —C(O)NH₂.

In some embodiments is a compound of Formula (II), (IIa), or (IIb), or a pharmaceutically acceptable salt or solvate thereof, wherein R⁷ is selected from hydrogen, halogen, —CN, C₁₋₆alkyl, C₁₋₆haloalkyl, —OR²⁰, —C(O)OR²⁰, —C(O)R²³, —C(O)N(R²⁰)(R²¹), —S(O)₂R²³, and —S(O)₂N(R²⁰)(R²¹)—, wherein C₁₋₆alkyl is optionally substituted with one, two, or three groups selected from R^{14b}. In some embodiments is a compound of Formula (II), (IIa), or (IIb), or a pharmaceutically acceptable salt or solvate thereof, wherein R⁷ is selected from hydrogen, halogen, —CN, C₁₋₆alkyl, —OR²⁰, and —C(O)N(R²⁰)(R²¹), wherein C₁₋₆alkyl is optionally substituted with one, two, or three groups selected from R^{14b}, and wherein R²⁰ is independently selected from hydrogen and C₁₋₆alkyl. In some embodiments is a compound of Formula (II), (IIa), or (IIb), or a pharmaceutically acceptable salt or solvate thereof, wherein R⁷ is hydrogen. In some embodiments is a compound of Formula (II), (IIa), or (IIb), or a pharmaceutically acceptable salt or solvate thereof, wherein R⁷ is halogen. In some embodiments is a compound of Formula (II), (IIa), or (IIb), or a pharmaceutically acceptable salt or solvate thereof, wherein R⁷ is —CN. In some embodiments is a compound of Formula (II), (IIa), or (IIb), or a pharmaceutically acceptable salt or solvate thereof, wherein R⁷ is C₁₋₆alkyl optionally substituted with one, two, or three groups selected from R^{14b}. In some embodiments is a compound of Formula (II), (IIa), or (IIb), or a pharmaceutically acceptable salt or solvate thereof, wherein R⁷ is C₁₋₆alkyl substituted with one, two, or three groups selected from R^{14b}. In some embodiments is a compound of Formula (II), (IIa), or (IIb), or a pharmaceutically acceptable salt or solvate thereof, wherein R⁷ is unsubstituted C₁₋₆alkyl. In some embodiments is a compound of Formula (II), (IIa), or (IIb), or a pharmaceutically acceptable salt or solvate thereof, wherein R⁷ is —OR²⁰, wherein R²⁰ is independently selected from hydrogen and C₁₋₆alkyl. In some embodiments is a compound of Formula (II), (IIa), or (IIb), or a pharmaceutically acceptable salt or solvate thereof, wherein R⁷ is —C(O)N(R²⁰)(R²¹). In some embodiments is a compound of Formula (II), (IIa), or (IIb), or a pharmaceutically acceptable salt or solvate thereof, wherein R⁷ is —C(O)NH₂.

In some embodiments is a compound of Formula (II), (IIa), or (IIb), or a pharmaceutically acceptable salt or solvate thereof, wherein R⁸ is selected from hydrogen, halogen, —CN, C₁₋₆alkyl, C₁₋₆haloalkyl, —OR²⁰, —C(O)OR²⁰, —C(O)R²³, —C(O)N(R²⁰)(R²¹), —S(O)₂R²³, and —S(O)₂N(R²⁰)(R²¹)—, wherein C₁₋₆alkyl is optionally substituted with one, two, or three groups selected from R^{14b}. In some embodiments is a compound of Formula (II), (IIa), or (IIb), or a pharmaceutically acceptable salt or solvate thereof, wherein R⁸ is selected from hydrogen, halogen, —CN, C₁₋₆alkyl, —OR²⁰, and —C(O)N(R²⁰)(R²¹), wherein C₁₋₆alkyl is optionally substituted with one, two, or three groups selected from R^{14b}, and wherein R²⁰ is independently selected from hydrogen and C₁₋₆alkyl. In some embodiments is a compound of Formula (II), (IIa), or (IIb), or a pharmaceutically acceptable salt or solvate thereof, wherein R⁸ is hydrogen.

In some embodiments is a compound of Formula (II), (IIa), or (IIb), or a pharmaceutically acceptable salt or solvate thereof, wherein R⁸ is halogen. In some embodiments is a compound of Formula (II), (IIa), or (IIb), or a pharmaceutically acceptable salt or solvate thereof, wherein R⁸ is —CN. In some embodiments is a compound of Formula (II), (IIa), or (IIb), or a pharmaceutically acceptable salt or solvate thereof, wherein R⁸ is C₁₋₆alkyl optionally substituted with one, two, or three groups selected from R^{14b}. In some embodiments is a compound of Formula (II), (IIa), or (IIb), or a pharmaceutically acceptable salt or solvate thereof, wherein R⁸ is C₁₋₆alkyl substituted with one, two, or three groups selected from R^{14b}. In some embodiments is a compound of Formula (II), (IIa), or (IIb), or a pharmaceutically acceptable salt or solvate thereof, wherein R⁸ is unsubstituted C₁₋₆alkyl. In some embodiments is a compound of Formula (II), (IIa), or (IIb), or a pharmaceutically acceptable salt or solvate thereof, wherein R⁸ is —OR²⁰, wherein R²⁰ is independently selected from hydrogen and C₁₋₆alkyl. In some embodiments is a compound of Formula (II), (IIa), or (IIb), or a pharmaceutically acceptable salt or solvate thereof, wherein R⁸ is —C(O)N(R²⁰)(R²¹). In some embodiments is a compound of Formula (II), (IIa), or (IIb), or a pharmaceutically acceptable salt or solvate thereof, wherein R⁸ is —C(O)NH₂.

In some embodiments is a compound of Formula (II), (IIa), or (IIb), or a pharmaceutically acceptable salt or solvate thereof, wherein R¹³ is selected from C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl, wherein C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three groups selected from R^{15b}. In some embodiments is a compound of Formula (II), (IIa), or (IIb), or a pharmaceutically acceptable salt or solvate thereof, wherein each R^{14a}, R^{14b}, and R^{14c} are each independently selected from halogen, oxo, —CN, C₁₋₆alkyl, C₁₋₆haloalkyl, C₂₋₆alkenyl, C₂₋₆alkynyl, C₃₋₁₀cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR²⁰, —SR²⁰, —SF₅, —N(R²⁰)(R²¹), —C(O)OR²⁰, —OC(O)N(R²⁰)(R²¹), —N(R²²)C(O)N(R²⁰)(R²¹), —N(R²²)C(O)OR²³, —N(R²²)S(O)₂R²³, —C(O)R²³, —S(O)R²³, —OC(O)R²³, —C(O)N(R²⁰)(R²¹), —C(O)C(O)N(R²⁰)(R²¹), —N(R²²)C(O)R²³, —S(O)₂R²³, —S(O)₂N(R²⁰)(R²¹)—, —N═S(═O)(R²³)₂, —S(═O)(═NH)N(R²⁰)(R²¹), —S(═O)(═NH)C(R²⁰)(R²¹), —S(═O)(═NR²³)R²³, —CH₂C(O)N(R²⁰)(R²¹), —CH₂N(R²²)C(O)R²³, —CH₂S(O)₂R²³, —CH₂S(O)₂N(R²⁰)(R²¹), wherein C₁₋₆alkyl, C₁₋₆haloalkyl, C₂₋₆alkenyl, C₂₋₆alkynyl, C₃₋₆cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²⁰, —SR²⁰, —SF₅, —N(R²⁰)(R²¹), —C(O)OR²⁰, —OC(O)N(R²⁰)(R²¹), —N(R²²)C(O)N(R²⁰)(R²¹), —N(R²²)C(O)OR²³, —N(R²²)S(O)₂R²³, —C(O)R²³, —S(O)R²³, —OC(O)R²³, —C(O)N(R²⁰)(R²¹), —C(O)C(O)N(R²⁰)(R²¹), —N(R²²)C(O)R²³, —S(O)₂R²³, —S(O)₂N(R²⁰)(R²¹)—, —N═S(═O)(R²³)₂, —S(═O)(═NH)N(R²⁰)(R²¹), —S(═O)(═NH)C(R²⁰)(R²¹), —S(═O)(═NR²³)R²³, —CH₂C(O)N(R²⁰)(R²¹), —CH₂N(R²²)C(O)R²³, —CH₂S(O)₂R²³, and —CH₂S(O)₂N(R²⁰)(R²¹). In some embodiments is a compound of Formula (II), (IIa), or (IIb), or a pharmaceutically acceptable salt or solvate thereof, wherein each R^{15a} is independently selected from halogen, oxo, —CN, C₁₋₆alkyl, C₁₋₆haloalkyl, C₂₋₆alkenyl, C₂₋₆alkynyl, C₃₋₁₀cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR²⁰, —SR²⁰, —SF₅, —N(R²⁰)(R²¹), —C(O)OR²⁰, —OC(O)N(R²⁰)(R²¹), —N(R²²)C(O)N(R²⁰)(R²¹), —N(R²²)C(O)OR²³, —N(R²²)S(O)₂R²³, —C(O)R²³, —S(O)R²³, —OC(O)R²³, —C(O)N(R²⁰)(R²¹), —C(O)C(O)N(R²⁰)(R²¹), —N(R²²)C(O)R²³, —S(O)₂R²³, —S(O)₂N(R²⁰)(R²¹)—, —N═S(═O)(R²³)₂, —S(═O)(═NH)N(R²⁰)(R²¹), —S(═O)(═NH)C(R²⁰)(R²¹), —S(═O)(═NR²³)R²³, —CH₂C(O)N(R²⁰)(R²¹), —CH₂N(R²²)C(O)R²³, —CH₂S(O)₂R²³, —CH₂S(O)₂N(R²⁰)(R²¹), wherein C₁₋₆alkyl, C₁₋₆haloalkyl, C₂₋₆alkenyl, C₂₋₆alkynyl, C₃₋₆cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²⁰, —SR²⁰, —SF₅, —N(R²⁰)(R²¹), —C(O)OR²⁰, —OC(O)N(R²⁰)(R²¹), —N(R²²)C(O)N(R²⁰)(R²¹), —N(R²²)C(O)OR²³, —N(R²²)S(O)₂R²³, —C(O)R²³, —S(O)R²³, —OC(O)R²³, —C(O)N(R²⁰)(R²¹), —C(O)C(O)N(R²⁰)(R²¹), —N(R²²)C(O)R²³, —S(O)₂R²³, —S(O)₂N(R²⁰)(R²¹)—, —N═S(═O)(R²³)₂, —S(═O)(═NH)N(R²⁰)(R²¹), —S(═O)(═NH)C(R²⁰)(R²¹), —S(═O)(═NR²³)R²³, —CH₂C(O)N(R²⁰)(R²¹), —CH₂N(R²²)C(O)R²³, —CH₂S(O)₂R²³, and —CH₂S(O)₂N(R²⁰)(R²¹); or two R^{15a} are combined to form a C₃₋₆cycloalkyl or C₂₋₉heterocycloalkyl, wherein C₃₋₆cycloalkyl and C₂₋₉heterocycloalkyl are optionally substituted with one, two, or three groups independently selected from halogen, oxo, C₁₋₆alkyl, C₁₋₆haloalkyl, and C₁₋₆alkoxy.

In some embodiments is a compound of Formula (II), (IIa), or (IIb), or a pharmaceutically acceptable salt or solvate thereof, wherein L² is a bond. In some embodiments is a compound of Formula (II), (IIa), or (IIb), or a pharmaceutically acceptable salt or solvate thereof, wherein L² is C₁₋₆alkylene optionally substituted with one, two, or three groups selected from R^{14a}. In some embodiments is a compound of Formula (II), (IIa), or (IIb), or a pharmaceutically acceptable salt or solvate thereof, wherein L² is unsubstituted C₁₋₆alkylene. In some embodiments is a compound of Formula (II), (IIa), or (IIb), or a pharmaceutically acceptable salt or solvate thereof, wherein L² is —CH₂—.

In some embodiments is a compound of Formula (II), (IIa), or (IIb), or a pharmaceutically acceptable salt or solvate thereof, wherein R¹³ is selected from C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl, wherein C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three groups selected from R^{15b}. In some embodiments is a compound of Formula (II), (IIa), or (IIb), or a pharmaceutically acceptable salt or solvate thereof, wherein R¹³ is selected from C₃₋₁₀cycloalkyl, C₂₋₉heterocycloalkyl, C₆₋₁₀aryl, and C₁₋₉heteroaryl, wherein C₃₋₁₀cycloalkyl, C₂₋₉heterocycloalkyl, C₆₋₁₀aryl, and C₁₋₉heteroaryl are optionally substituted with one, two, or three groups selected from R^{15b}. In some embodiments is a compound of Formula (II), (IIa), or (IIb), or a pharmaceutically acceptable salt or solvate thereof, wherein R¹³ is C₆₋₁₀aryl optionally substituted with one, two, or three groups selected from R^{15b}. In some embodiments is a compound of Formula (II), (IIa), or (IIb), or a pharmaceutically acceptable salt or solvate thereof, wherein R¹³ is phenyl optionally substituted with one, two, or three groups selected from R^{15b}. In some embodiments is a compound of Formula (II), (IIa), or (IIb), or a pharmaceutically acceptable salt or solvate thereof, wherein R¹³ is phenyl substituted with one, two, or three groups selected from R^{15b}. In some embodiments is a compound of Formula (II), (IIa), or (IIb), or a pharmaceutically acceptable salt or solvate thereof, wherein R¹³ is phenyl substituted with one R^{15b} group. In some embodiments is a compound of Formula (II), (IIa), or (IIb), or a pharmaceutically acceptable salt or solvate thereof, wherein R¹³ is C₁₋₉heteroaryl optionally substituted with one, two, or three groups selected from R^{15b}. In some embodiments is a compound of Formula (II), (IIa), or (IIb), or a pharmaceutically acceptable salt or solvate thereof, wherein R¹³ is C₁₋₉heteroaryl optionally substituted with one, two, or three groups selected from R^{15b}, wherein C₁₋₉heteroaryl is selected from pyridyl, pyrimidinyl, pyrazinyl, pyridazinyl, thienyl, furanyl, pyrrolyl, oxazolyl, thiazolyl, pyrazolyl, and imidazolyl. In some embodiments is a compound of Formula (II), (IIa), or (IIb), or a pharmaceutically acceptable salt or solvate thereof, wherein R¹³ is C₁₋₉heteroaryl optionally substituted with one, two, or three groups selected from R^{15b}, wherein C₁₋₉heteroaryl is selected from pyridyl, pyrimidinyl, pyrazinyl, pyridazinyl, thienyl, furanyl, pyrrolyl, oxazolyl, thiazolyl, pyrazolyl, indazolyl, and imidazolyl. In some embodiments is a compound of Formula (II), (IIa), or (IIb), or a pharmaceutically acceptable salt or solvate thereof, wherein R¹³ is C₁₋₉heteroaryl substituted with one, two, or three groups selected from R^{15b}, wherein C₁₋₉heteroaryl is selected from pyridyl, pyrimidinyl, pyrazinyl, pyridazinyl, thienyl, furanyl, pyrrolyl, oxazolyl, thiazolyl, pyrazolyl, and imidazolyl. In some embodiments is a compound of Formula (II), (IIa), or (IIb), or a pharmaceutically acceptable salt or solvate thereof, wherein R¹³ is C₁₋₉heteroaryl substituted with one, two, or three groups selected from R^{15b}, wherein C₁₋₉heteroaryl is selected from pyridyl, pyrimidinyl, pyrazinyl, pyridazinyl, thienyl, furanyl, pyrrolyl, oxazolyl, thiazolyl, pyrazolyl, indazolyl, and imidazolyl. In some embodiments is a compound of Formula (II), (IIa), or (IIb), or a pharmaceutically acceptable salt or solvate thereof, wherein R¹³ is C₁₋₉heteroaryl substituted with one R^{15b} group, wherein C₁₋₉heteroaryl is selected from pyridyl, pyrimidinyl, pyrazinyl, pyridazinyl, thienyl, furanyl, pyrrolyl, oxazolyl, thiazolyl, pyrazolyl, and imidazolyl. In some embodiments is a compound of Formula (II), (IIa), or (IIb), or a pharmaceutically acceptable salt or solvate thereof, wherein R¹³ is C₁₋₉heteroaryl substituted with one R^{15b} group, wherein C₁₋₉heteroaryl is selected from pyridyl, pyrimidinyl, pyrazinyl, pyridazinyl, thienyl, furanyl, pyrrolyl, oxazolyl, thiazolyl, pyrazolyl, indazolyl, and imidazolyl. In some embodiments is a compound of Formula (II), (IIa), or (IIb), or a pharmaceutically acceptable salt or solvate thereof, wherein R¹³ is C₃₋₁₀cycloalkyl optionally substituted with one, two, or three groups selected from R^{15b}. In some embodiments is a compound of Formula (II), (IIa), or (IIb), or a pharmaceutically acceptable salt or solvate thereof, wherein R¹³ is C₂₋₉heterocycloalkyl optionally substituted with one, two, or three groups selected from R^{15b}.

In some embodiments is a compound of Formula (II), (IIa), or (IIb), or a pharmaceutically acceptable salt or solvate thereof, wherein each R^{15b} is independently selected from halogen, C₁₋₆alkyl, C₁₋₆haloalkyl, and —OR²⁰, wherein R²⁰ is selected from C₁₋₆alkyl and C₁₋₆haloalkyl. In some embodiments is a compound of Formula (II), (IIa), or (IIb), or a pharmaceutically acceptable salt or solvate thereof, wherein each R^{15b} is halogen. In some embodiments is a compound of Formula (II), (IIa), or (IIb), or a pharmaceutically acceptable salt or solvate thereof, wherein each R^{15b} is C₁₋₆haloalkyl. In some embodiments is a compound of Formula (II), (IIa), or (IIb), or a pharmaceutically acceptable salt or solvate thereof, wherein each R^{15b} is —CF₃. In some embodiments is a compound of Formula (II), (IIa), or (IIb), or a pharmaceutically acceptable salt or solvate thereof, wherein each R^{15b} is C₁₋₆alkyl. In some embodiments is a compound of Formula (II), (IIa), or (IIb), or a pharmaceutically acceptable salt or solvate thereof, wherein each R^{15b} is —CH₃. In some embodiments is a compound of Formula (II), (IIa), or (IIb), or a pharmaceutically acceptable salt or solvate thereof, wherein each R^{15b} is —OR²⁰, wherein R²⁰ is selected from C₁₋₆alkyl and C₁₋₆haloalkyl.

In some embodiments is a compound of Formula (II), (IIa), or (IIb), or a pharmaceutically acceptable salt or solvate thereof, wherein L¹ is C₁₋₆alkylene optionally substituted with one, two, or three groups selected from R^{14a}. In some embodiments is a compound of Formula (II), (IIa), or (IIb), or a pharmaceutically acceptable salt or solvate thereof, wherein L¹ is unsubstituted C₁₋₆alkylene. In some embodiments is a compound of Formula (II), (IIa), or (IIb), or a pharmaceutically acceptable salt or solvate thereof, wherein L¹ is —CH₂—. In some embodiments is a compound of Formula (II), (IIa), or (IIb), or a pharmaceutically acceptable salt or solvate thereof, wherein L¹ is a bond.

In some embodiments is a compound of Formula (II), (IIa), or (IIb), or a pharmaceutically acceptable salt or solvate thereof, wherein R^{12a} is C₁₋₉heteroaryl optionally substituted with one, two, or three groups selected from R^{15a}. In some embodiments is a compound of Formula (II), (IIa), or (IIb), or a pharmaceutically acceptable salt or solvate thereof, wherein R¹², is C₁₋₉heteroaryl optionally substituted with one, two, or three groups selected from R^{15a}, wherein C₁₋₉heteroaryl is selected from pyridyl, pyrimidinyl, pyrazinyl, pyridazinyl, thienyl, furanyl, pyrrolyl, oxazolyl, thiazolyl, pyrazolyl, indazolyl, and imidazolyl. In some embodiments is a compound of Formula (II), (IIa), or (IIb), or a pharmaceutically acceptable salt or solvate thereof, wherein R^{12a} is C₁₋₉heteroaryl optionally substituted with one, two, or three groups selected from R^{15a}, wherein C₁₋₆heteroaryl is selected from pyridyl, pyrimidinyl, pyrazinyl, pyridazinyl, thienyl, furanyl, pyrrolyl, oxazolyl, thiazolyl, pyrazolyl, and imidazolyl. In some embodiments is a compound of Formula (II), (IIa), or (IIb), or a pharmaceutically acceptable salt or solvate thereof, wherein R^{12a} is C₁₋₉heteroaryl optionally substituted with one, two, or three groups selected from R^{15a}, wherein C₁₋₉heteroaryl is selected from pyridyl, pyrimidinyl, pyrazinyl, pyridazinyl, and pyrazolyl. In some embodiments is a compound of Formula (II), (IIa), or (IIb), or a pharmaceutically acceptable salt or solvate thereof, wherein R^{12a} is C₁₋₉heteroaryl optionally substituted with one, two, or three groups selected from R^{15a}, wherein C₁₋₉heteroaryl is selected from pyridyl, pyrimidinyl, and pyrazolyl. In some embodiments is a compound of Formula (II), (IIa), or (IIb), or a pharmaceutically acceptable salt or solvate thereof, wherein R^{12a} is pyridyl optionally substituted with one, two, or three groups selected from R^{15a}. In some embodiments is a compound of Formula (II), (IIa), or (IIb), or a pharmaceutically acceptable salt or solvate thereof, wherein R^{12a} is unsubstituted pyridyl. In some embodiments is a compound of Formula (II), (IIa), or (IIb), or a pharmaceutically acceptable salt or solvate thereof, wherein R^{12a} is pyrimidinyl optionally substituted with one, two, or three groups selected from R^{15a}. In some embodiments is a compound of Formula (II), (IIa), or (IIb), or a pharmaceutically acceptable salt or solvate thereof, wherein R^{12a} is unsubstituted pyrimidinyl. In some embodiments is a compound of Formula (II), (IIa), or (IIb), or a pharmaceutically acceptable salt or solvate thereof, wherein R^{12a} is pyrazolyl optionally substituted with one, two, or three groups selected from R^{15a}. In some embodiments is a compound of Formula (II), (IIa), or (IIb), or a pharmaceutically acceptable salt or solvate thereof, wherein R^{12a} is unsubstituted pyrazolyl. In some embodiments is a compound of Formula (II), (IIa), or (IIb), or a pharmaceutically acceptable salt or solvate thereof, wherein R^{12a} is C₂₋₉heterocycloalkyl optionally substituted with one, two, or three groups selected from R^{15a}. In some embodiments is a compound of Formula (II), (IIa), or (IIb), or a pharmaceutically acceptable salt or solvate thereof, wherein R^{12a} is C₁₋₆alkyl optionally substituted with one, two, or three groups selected from R^{15a}. In some embodiments is a compound of Formula (II), (IIa), or (IIb), or a pharmaceutically acceptable salt or solvate thereof, wherein R^{12a} is unsubstituted C₁₋₆alkyl. In some embodiments is a compound of Formula (II), (IIa), or (IIb), or a pharmaceutically acceptable salt or solvate thereof, wherein R^{12a} is C₂₋₆alkyl optionally substituted with one, two, or three groups selected from R^{15a}. In some embodiments is a compound of Formula (II), (IIa), or (IIb), or a pharmaceutically acceptable salt or solvate thereof, wherein R^{12a} is unsubstituted C₂₋₆alkyl.

In some embodiments is a compound of Formula (II), (IIa), or (IIb), or a pharmaceutically acceptable salt or solvate thereof, wherein each R^{15a} is independently selected from halogen, —CN, C₁₋₆alkyl, and C₁₋₆haloalkyl. In some embodiments is a compound of Formula (II), (IIa), or (IIb), or a pharmaceutically acceptable salt or solvate thereof, wherein each R^{15a} is halogen. In some embodiments is a compound of Formula (II), (IIa), or (IIb), or a pharmaceutically acceptable salt or solvate thereof, wherein each R^{15a} is C₁₋₆haloalkyl. In some embodiments is a compound of Formula (II), (IIa), or (IIb), or a pharmaceutically acceptable salt or solvate thereof, wherein each R^{15a} is —CF₃. In some embodiments is a compound of Formula (II), (IIa), or (IIb), or a pharmaceutically acceptable salt or solvate thereof, wherein each R^{15a} is C₁₋₆alkyl. In some embodiments is a compound of Formula (II), (IIa), or (IIb), or a pharmaceutically acceptable salt or solvate thereof, wherein each R^{15a} is —CH₃. In some embodiments is a compound of Formula (II), (IIa), or (IIb), or a pharmaceutically acceptable salt or solvate thereof, wherein each R^{15a} is —CN. In some embodiments is a compound of Formula (II), (IIa), or (IIb), or a pharmaceutically acceptable salt or solvate thereof, wherein each R^{15a} is —N(R²⁰)(R²¹). In some embodiments is a compound of Formula (II), (IIa), or (IIb), or a pharmaceutically acceptable salt or solvate thereof, wherein each R^{15a} is —NH₂.

Any combination of the groups described above for the various variables is contemplated herein. Throughout the specification, groups and substituents thereof are chosen by one skilled in the field to provide stable moieties and compounds.

In some embodiments is a compound, or a pharmaceutically acceptable salt or solvate thereof, selected from:

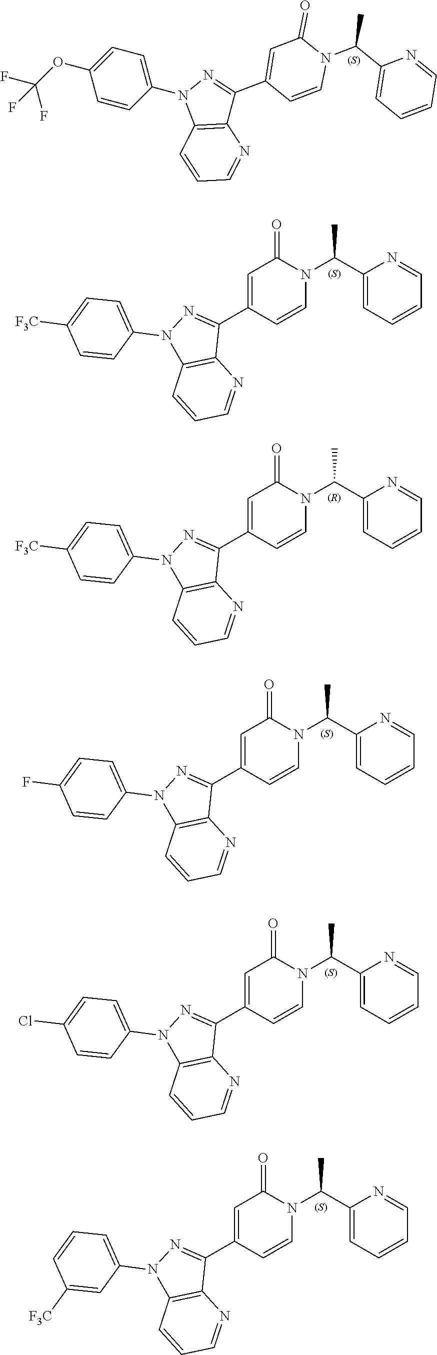

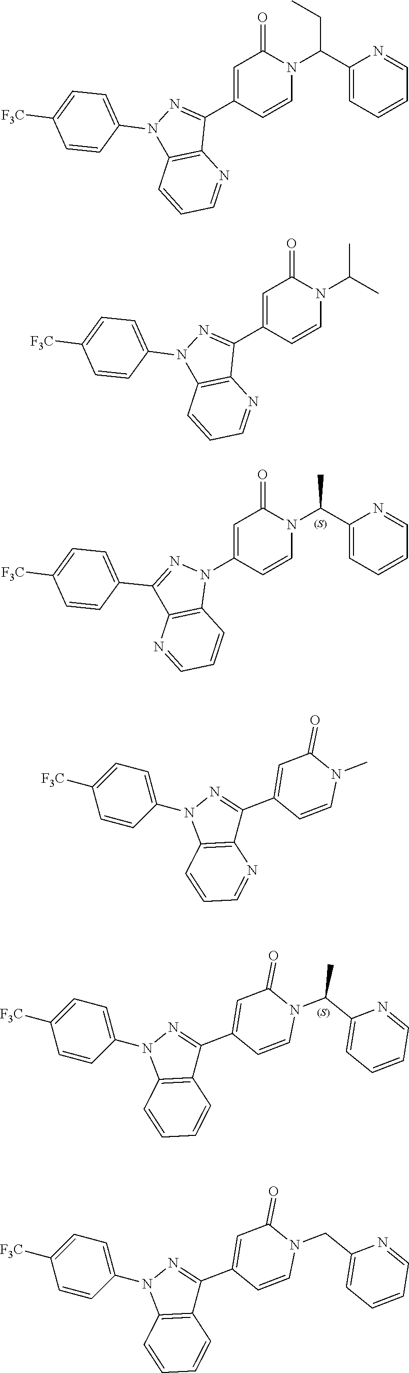

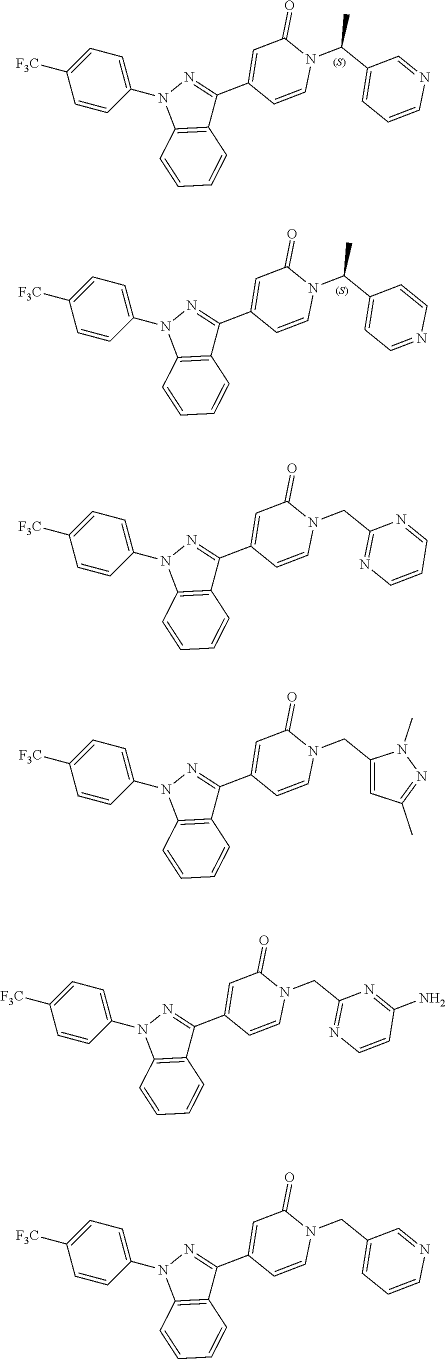

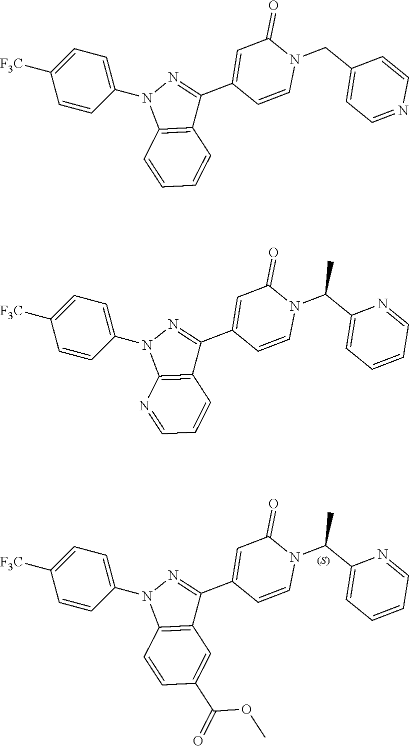

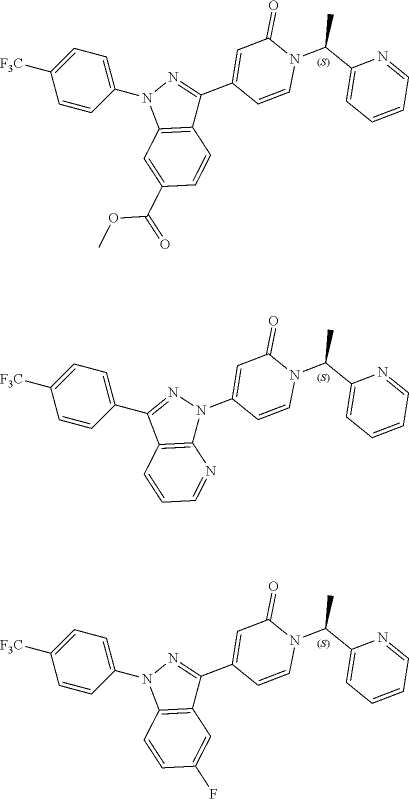

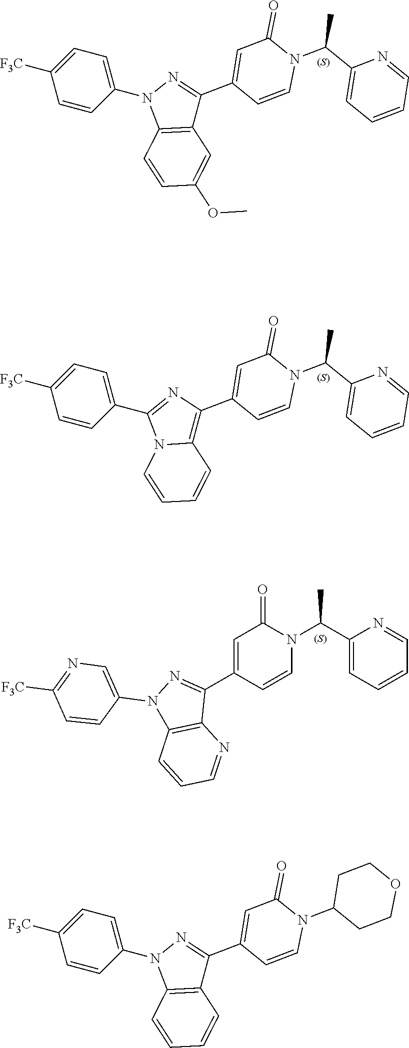

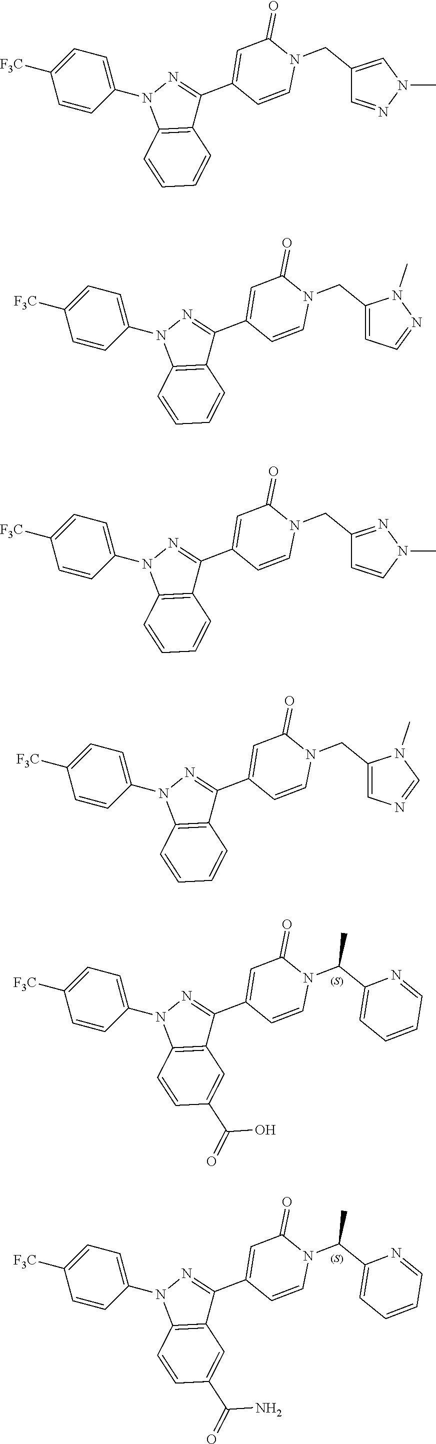

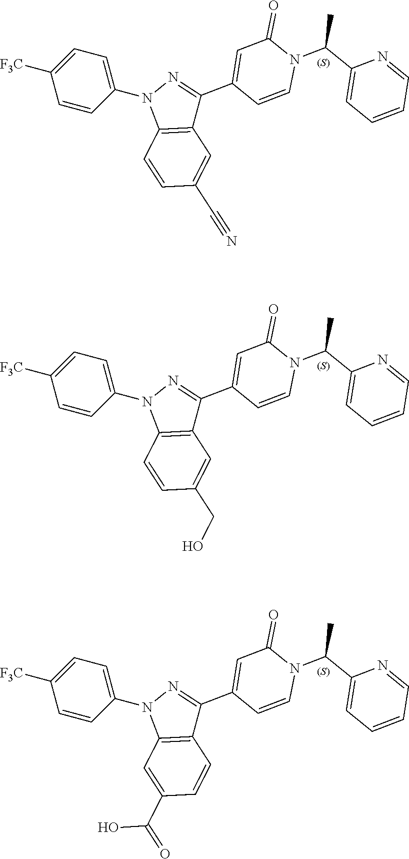

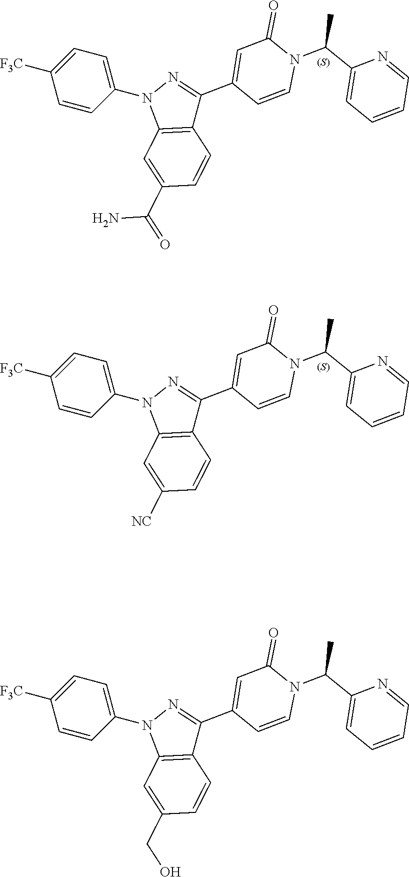

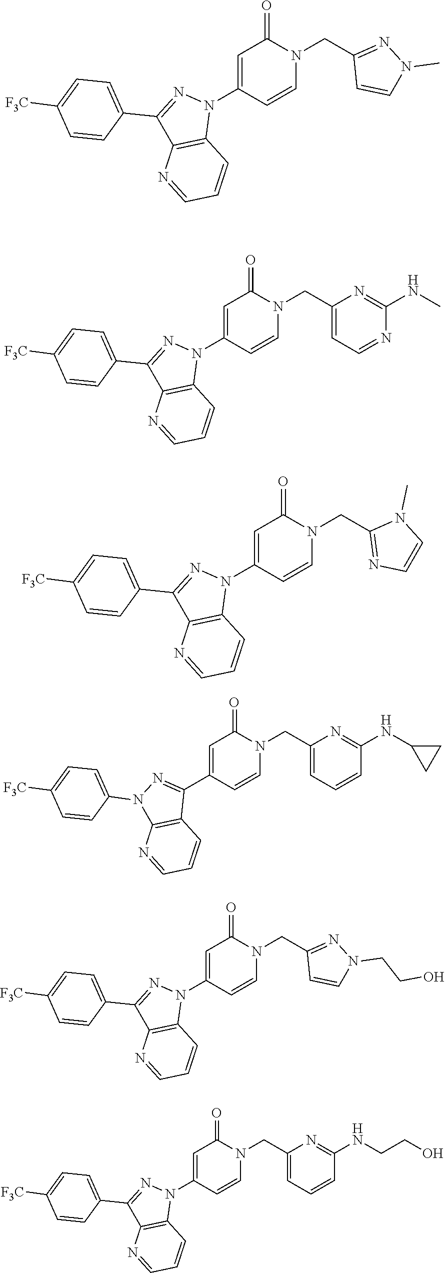

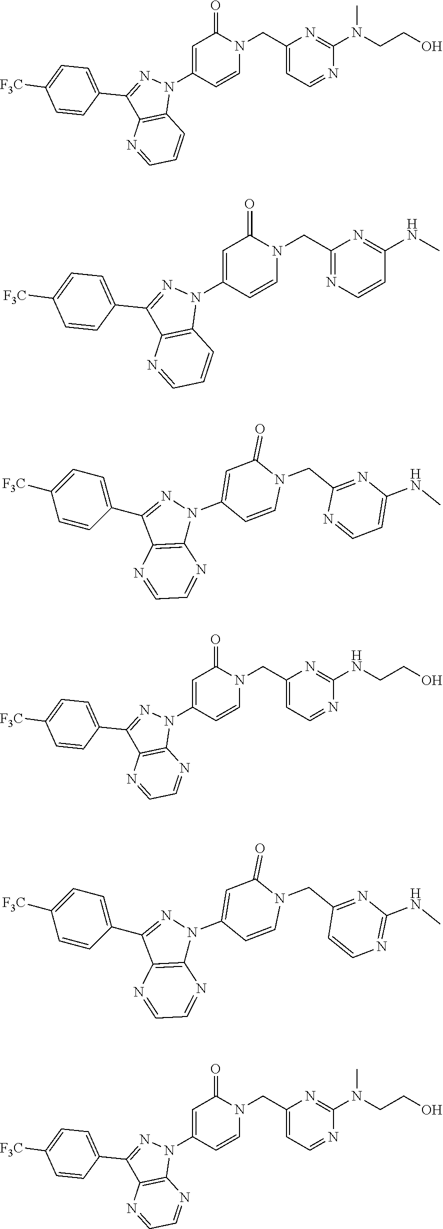 [SMILES: CN(CCO)c1nccc(Cn2ccc(-n3nc(-c4ccc(C(F)(F)F)cc4)c4ncccc43)cc2=O)n1 ; CNc1ccnc(Cn2ccc(-n3nc(-c4ccc(C(F)(F)F)cc4)c4ncccc43)cc2=O)n1 ; CNc1ccnc(Cn2ccc(-n3nc(-c4ccc(C(F)(F)F)cc4)c4nccnc43)cc2=O)n1 ; O=c1cc(-n2nc(-c3ccc(C(F)(F)F)cc3)c3nccnc32)ccn1Cc1ccnc(NCCO)n1 ; CNc1nccc(Cn2ccc(-n3nc(-c4ccc(C(F)(F)F)cc4)c4nccnc43)cc2=O)n1 ; CN(CCO)c1nccc(Cn2ccc(-n3nc(-c4ccc(C(F)(F)F)cc4)c4nccnc43)cc2=O)n1]

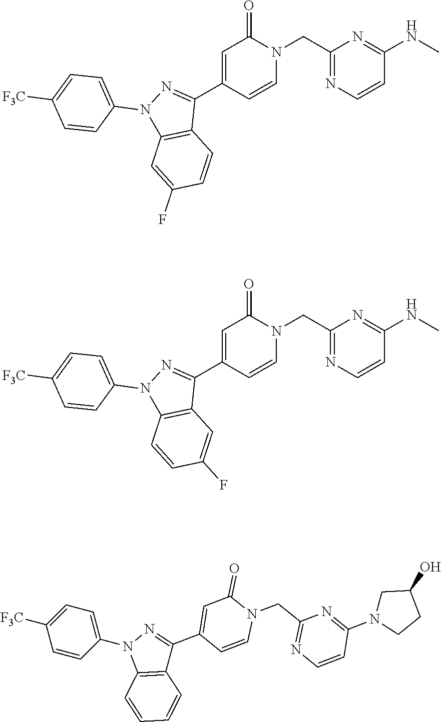 [SMILES: CNc1ccnc(Cn2ccc(-c3nn(-c4ccc(C(F)(F)F)cc4)c4cc(F)ccc34)cc2=O)n1 ; CNc1ccnc(Cn2ccc(-c3nn(-c4ccc(C(F)(F)F)cc4)c4ccc(F)cc34)cc2=O)n1 ; O=c1cc(-c2nn(-c3ccc(C(F)(F)F)cc3)c3ccccc23)ccn1Cc1nccc(N2CC[C@H](O)C2)n1]

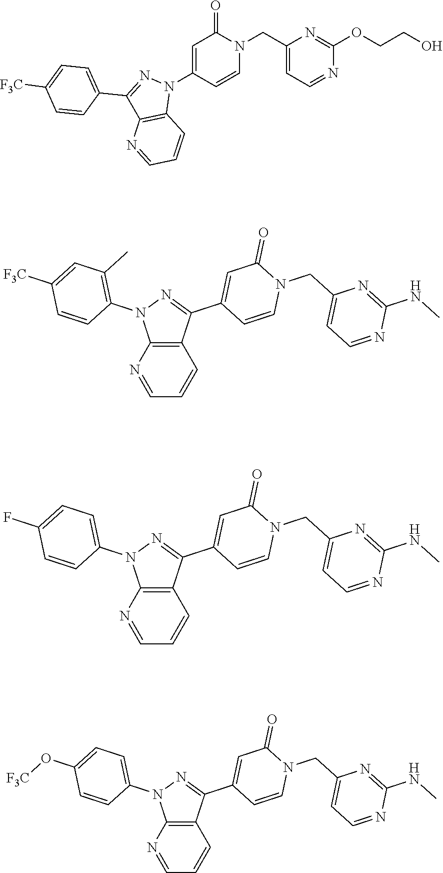 [SMILES: O=c1cc(-n2nc(-c3ccc(C(F)(F)F)cc3)c3ncccc32)ccn1Cc1ccnc(OCCO)n1 ; CNc1nccc(Cn2ccc(-c3nn(-c4ccc(C(F)(F)F)cc4C)c4ncccc34)cc2=O)n1 ; CNc1nccc(Cn2ccc(-c3nn(-c4ccc(F)cc4)c4ncccc34)cc2=O)n1 ; CNc1nccc(Cn2ccc(-c3nn(-c4ccc(OC(F)(F)F)cc4)c4ncccc34)cc2=O)n1]

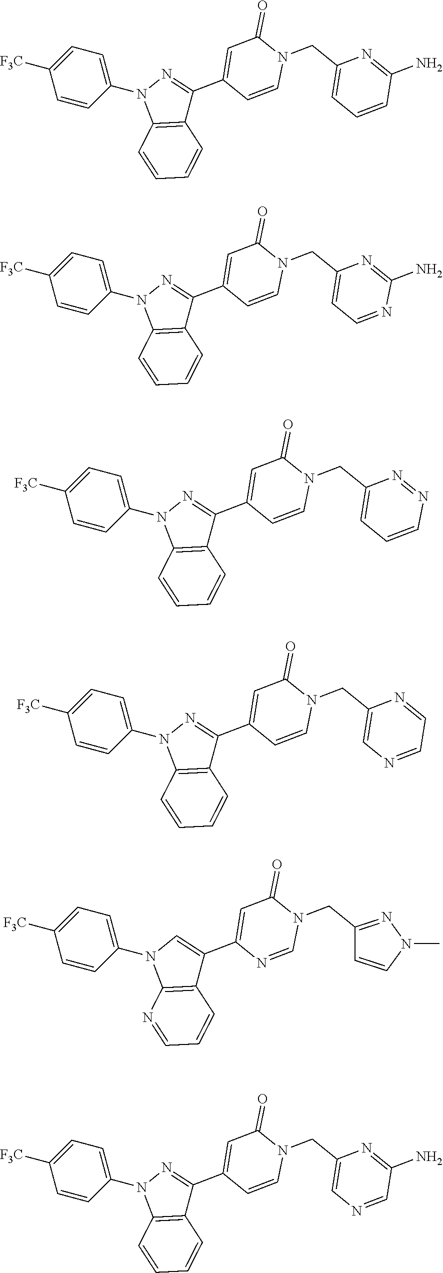 [SMILES: Nc1cccc(Cn2ccc(-c3nn(-c4ccc(C(F)(F)F)cc4)c4ccccc34)cc2=O)n1 ; Nc1nccc(Cn2ccc(-c3nn(-c4ccc(C(F)(F)F)cc4)c4ccccc34)cc2=O)n1 ; O=c1cc(-c2nn(-c3ccc(C(F)(F)F)cc3)c3ccccc23)ccn1Cc1cccnn1 ; O=c1cc(-c2nn(-c3ccc(C(F)(F)F)cc3)c3ccccc23)ccn1Cc1cnccn1 ; Cn1ccc(Cn2cnc(-c3cn(-c4ccc(C(F)(F)F)cc4)c4ncccc34)cc2=O)n1 ; Nc1cncc(Cn2ccc(-c3nn(-c4ccc(C(F)(F)F)cc4)c4ccccc34)cc2=O)n1]

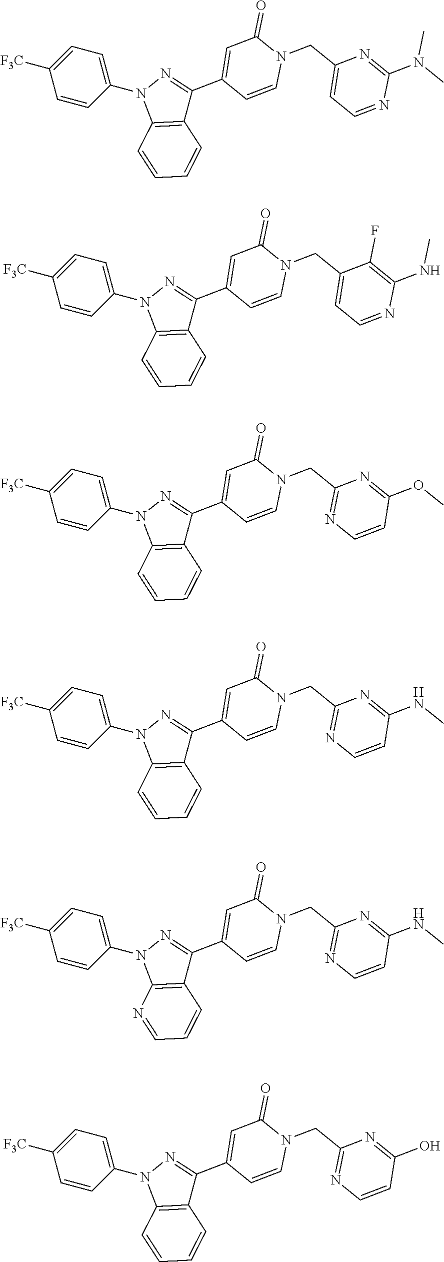 [SMILES: CN(C)c1nccc(Cn2ccc(-c3nn(-c4ccc(C(F)(F)F)cc4)c4ccccc34)cc2=O)n1 ; CNc1nccc(Cn2ccc(-c3nn(-c4ccc(C(F)(F)F)cc4)c4ccccc34)cc2=O)c1F ; COc1ccnc(Cn2ccc(-c3nn(-c4ccc(C(F)(F)F)cc4)c4ccccc34)cc2=O)n1 ; CNc1ccnc(Cn2ccc(-c3nn(-c4ccc(C(F)(F)F)cc4)c4ccccc34)cc2=O)n1 ; CNc1ccnc(Cn2ccc(-c3nn(-c4ccc(C(F)(F)F)cc4)c4ncccc34)cc2=O)n1 ; O=c1cc(-c2nn(-c3ccc(C(F)(F)F)cc3)c3ccccc23)ccn1Cc1nccc(O)n1]

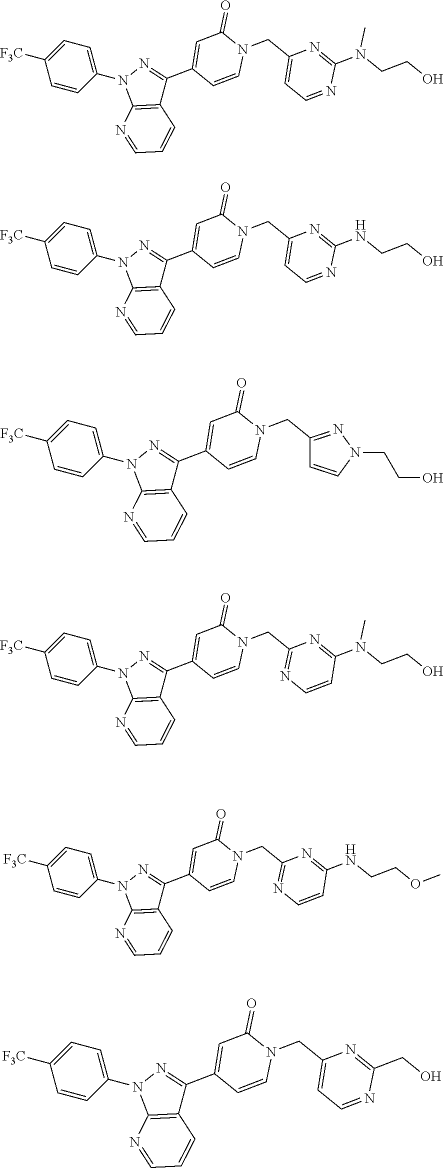 [SMILES: CN(CCO)c1nccc(Cn2ccc(-c3nn(-c4ccc(C(F)(F)F)cc4)c4ncccc34)cc2=O)n1 ; O=c1cc(-c2nn(-c3ccc(C(F)(F)F)cc3)c3ncccc23)ccn1Cc1ccnc(NCCO)n1 ; O=c1cc(-c2nn(-c3ccc(C(F)(F)F)cc3)c3ncccc23)ccn1Cc1ccn(CCO)n1 ; CN(CCO)c1ccnc(Cn2ccc(-c3nn(-c4ccc(C(F)(F)F)cc4)c4ncccc34)cc2=O)n1 ; COCCNc1ccnc(Cn2ccc(-c3nn(-c4ccc(C(F)(F)F)cc4)c4ncccc34)cc2=O)n1 ; O=c1cc(-c2nn(-c3ccc(C(F)(F)F)cc3)c3ncccc23)ccn1Cc1ccnc(CO)n1]

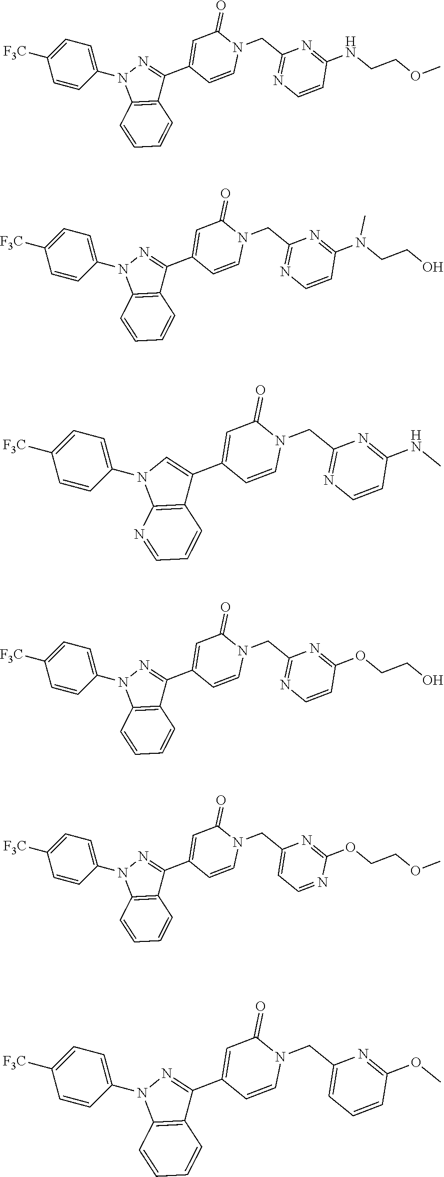 [SMILES: COCCNc1ccnc(Cn2ccc(-c3nn(-c4ccc(C(F)(F)F)cc4)c4ccccc34)cc2=O)n1 ; CN(CCO)c1ccnc(Cn2ccc(-c3nn(-c4ccc(C(F)(F)F)cc4)c4ccccc34)cc2=O)n1 ; CNc1ccnc(Cn2ccc(-c3cn(-c4ccc(C(F)(F)F)cc4)c4ncccc34)cc2=O)n1 ; O=c1cc(-c2nn(-c3ccc(C(F)(F)F)cc3)c3ccccc23)ccn1Cc1nccc(OCCO)n1 ; COCCOc1nccc(Cn2ccc(-c3nn(-c4ccc(C(F)(F)F)cc4)c4ccccc34)cc2=O)n1 ; COc1cccc(Cn2ccc(-c3nn(-c4ccc(C(F)(F)F)cc4)c4ccccc34)cc2=O)n1]

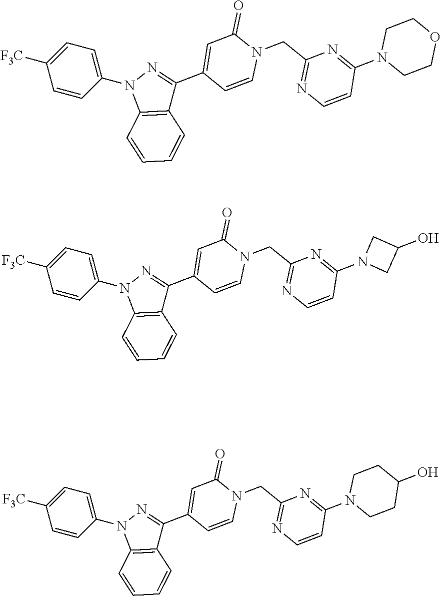 [SMILES: O=c1cc(-c2nn(-c3ccc(C(F)(F)F)cc3)c3ccccc23)ccn1Cc1nccc(N2CCOCC2)n1 ; O=c1cc(-c2nn(-c3ccc(C(F)(F)F)cc3)c3ccccc23)ccn1Cc1nccc(N2CC(O)C2)n1 ; O=c1cc(-c2nn(-c3ccc(C(F)(F)F)cc3)c3ccccc23)ccn1Cc1nccc(N2CCC(O)CC2)n1]

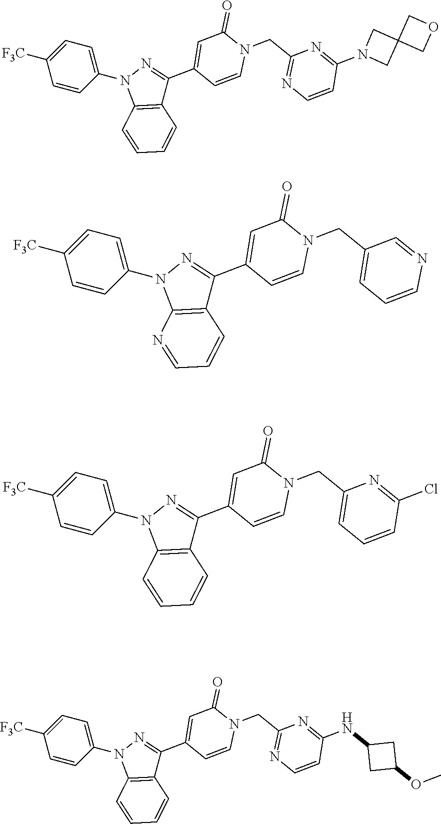 [SMILES: O=c1cc(-c2nn(-c3ccc(C(F)(F)F)cc3)c3ccccc23)ccn1Cc1nccc(N2CC3(COC3)C2)n1 ; O=c1cc(-c2nn(-c3ccc(C(F)(F)F)cc3)c3ncccc23)ccn1Cc1cccnc1 ; O=c1cc(-c2nn(-c3ccc(C(F)(F)F)cc3)c3ccccc23)ccn1Cc1cccc(Cl)n1 ; CO[C@H]1C[C@@H](Nc2ccnc(Cn3ccc(-c4nn(-c5ccc(C(F)(F)F)cc5)c5ccccc45)cc3=O)n2)C1]

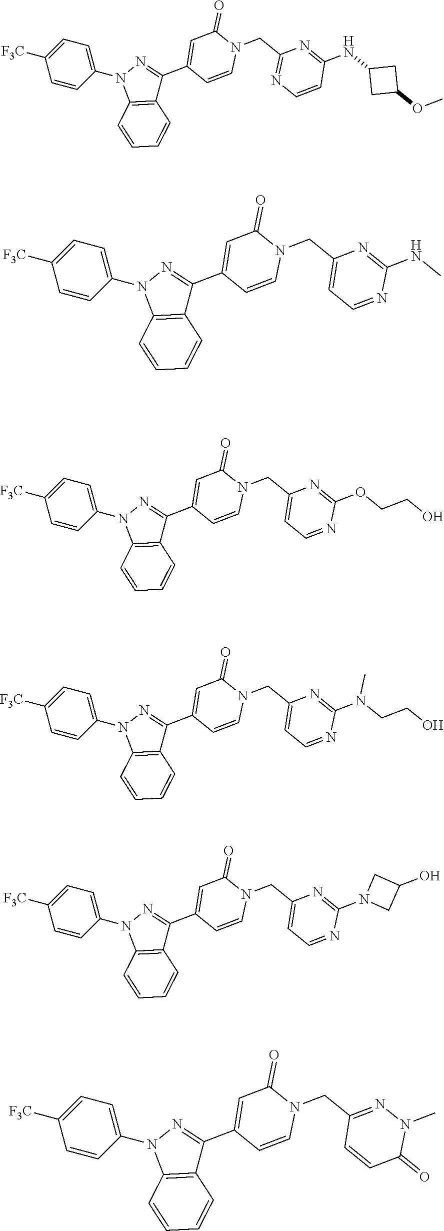 [SMILES: CO[C@H]1C[C@H](Nc2ccnc(Cn3ccc(-c4nn(-c5ccc(C(F)(F)F)cc5)c5ccccc45)cc3=O)n2)C1 ; CNc1nccc(Cn2ccc(-c3nn(-c4ccc(C(F)(F)F)cc4)c4ccccc34)cc2=O)n1 ; O=c1cc(-c2nn(-c3ccc(C(F)(F)F)cc3)c3ccccc23)ccn1Cc1ccnc(OCCO)n1 ; CN(CCO)c1nccc(Cn2ccc(-c3nn(-c4ccc(C(F)(F)F)cc4)c4ccccc34)cc2=O)n1 ; O=c1cc(-c2nn(-c3ccc(C(F)(F)F)cc3)c3ccccc23)ccn1Cc1ccnc(N2CC(O)C2)n1 ; Cn1nc(Cn2ccc(-c3nn(-c4ccc(C(F)(F)F)cc4)c4ccccc34)cc2=O)ccc1=O]

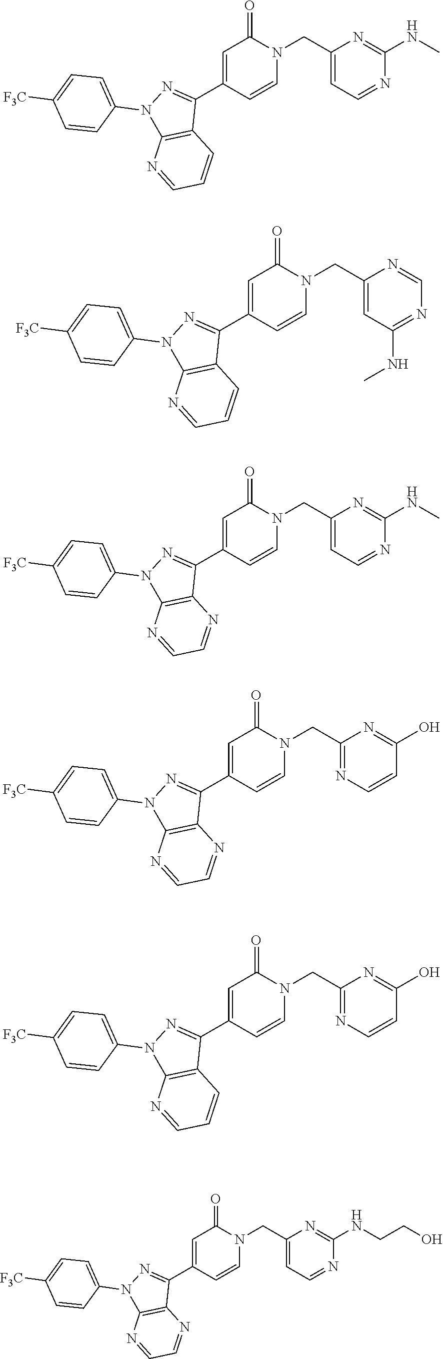 [SMILES: CNc1nccc(Cn2ccc(-c3nn(-c4ccc(C(F)(F)F)cc4)c4ncccc34)cc2=O)n1 ; CNc1cc(Cn2ccc(-c3nn(-c4ccc(C(F)(F)F)cc4)c4ncccc34)cc2=O)ncn1 ; CNc1nccc(Cn2ccc(-c3nn(-c4ccc(C(F)(F)F)cc4)c4nccnc34)cc2=O)n1 ; O=c1cc(-c2nn(-c3ccc(C(F)(F)F)cc3)c3nccnc23)ccn1Cc1nccc(O)n1 ; O=c1cc(-c2nn(-c3ccc(C(F)(F)F)cc3)c3ncccc23)ccn1Cc1nccc(O)n1 ; O=c1cc(-c2nn(-c3ccc(C(F)(F)F)cc3)c3nccnc23)ccn1Cc1ccnc(NCCO)n1]

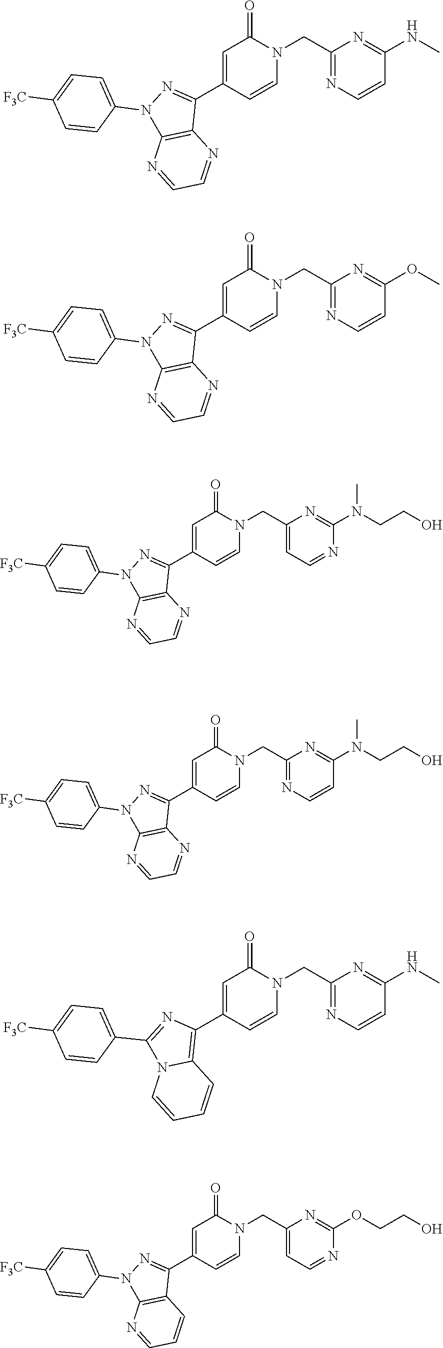 [SMILES: CNc1ccnc(Cn2ccc(-c3nn(-c4ccc(C(F)(F)F)cc4)c4nccnc34)cc2=O)n1 ; COc1ccnc(Cn2ccc(-c3nn(-c4ccc(C(F)(F)F)cc4)c4nccnc34)cc2=O)n1 ; CN(CCO)c1nccc(Cn2ccc(-c3nn(-c4ccc(C(F)(F)F)cc4)c4nccnc34)cc2=O)n1 ; CN(CCO)c1ccnc(Cn2ccc(-c3nn(-c4ccc(C(F)(F)F)cc4)c4nccnc34)cc2=O)n1 ; CNc1ccnc(Cn2ccc(-c3nc(-c4ccc(C(F)(F)F)cc4)n4ccccc34)cc2=O)n1 ; O=c1cc(-c2nn(-c3ccc(C(F)(F)F)cc3)c3ncccc23)ccn1Cc1ccnc(OCCO)n1]

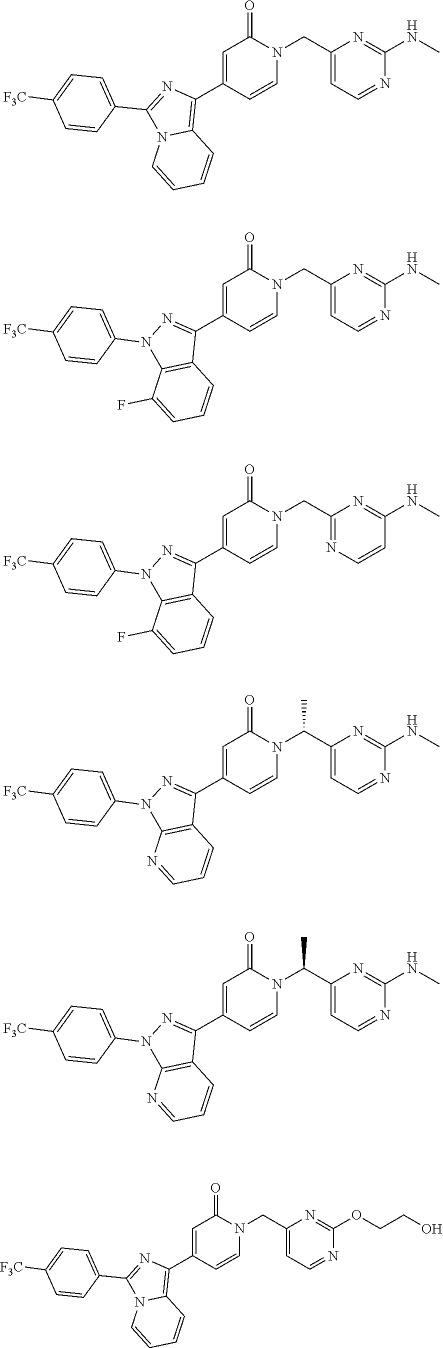 [SMILES: CNc1nccc(Cn2ccc(-c3nc(-c4ccc(C(F)(F)F)cc4)n4ccccc34)cc2=O)n1 ; CNc1nccc(Cn2ccc(-c3nn(-c4ccc(C(F)(F)F)cc4)c4c(F)cccc34)cc2=O)n1 ; CNc1ccnc(Cn2ccc(-c3nn(-c4ccc(C(F)(F)F)cc4)c4c(F)cccc34)cc2=O)n1 ; CNc1nccc([C@@H](C)n2ccc(-c3nn(-c4ccc(C(F)(F)F)cc4)c4ncccc34)cc2=O)n1 ; CNc1nccc([C@H](C)n2ccc(-c3nn(-c4ccc(C(F)(F)F)cc4)c4ncccc34)cc2=O)n1 ; O=c1cc(-c2nc(-c3ccc(C(F)(F)F)cc3)n3ccccc23)ccn1Cc1ccnc(OCCO)n1]

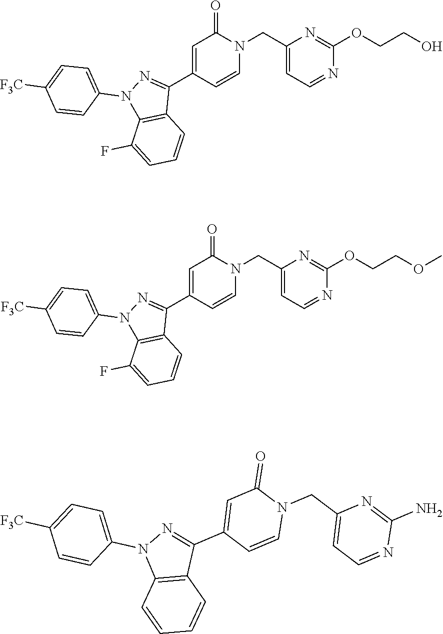 [SMILES: O=c1cc(-c2nn(-c3ccc(C(F)(F)F)cc3)c3c(F)cccc23)ccn1Cc1ccnc(OCCO)n1 ; COCCOc1nccc(Cn2ccc(-c3nn(-c4ccc(C(F)(F)F)cc4)c4c(F)cccc34)cc2=O)n1 ; Nc1nccc(Cn2ccc(-c3nn(-c4ccc(C(F)(F)F)cc4)c4ccccc34)cc2=O)n1]

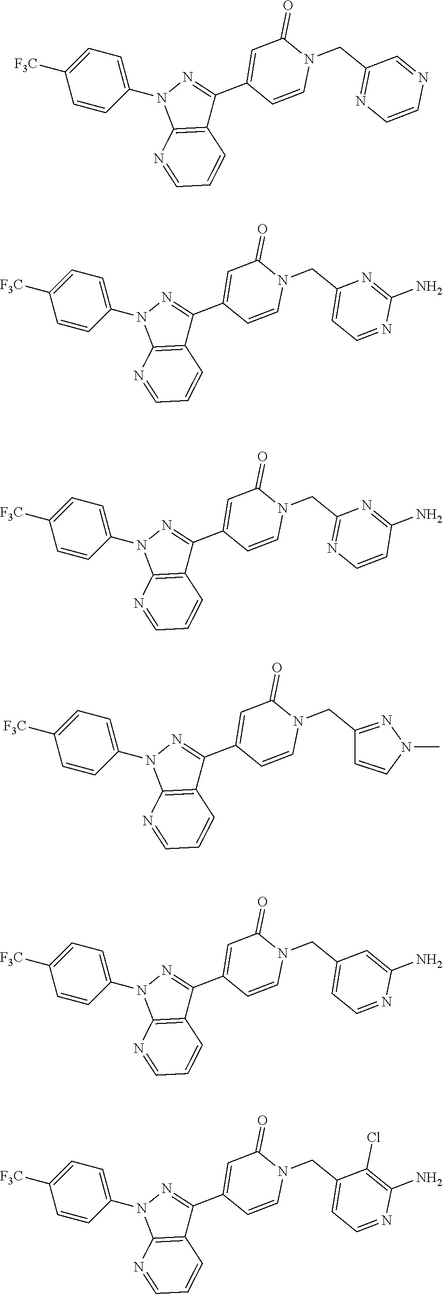 [SMILES: O=c1cc(-c2nn(-c3ccc(C(F)(F)F)cc3)c3ncccc23)ccn1Cc1cnccn1 ; Nc1nccc(Cn2ccc(-c3nn(-c4ccc(C(F)(F)F)cc4)c4ncccc34)cc2=O)n1 ; Nc1ccnc(Cn2ccc(-c3nn(-c4ccc(C(F)(F)F)cc4)c4ncccc34)cc2=O)n1 ; Cn1ccc(Cn2ccc(-c3nn(-c4ccc(C(F)(F)F)cc4)c4ncccc34)cc2=O)n1 ; Nc1cc(Cn2ccc(-c3nn(-c4ccc(C(F)(F)F)cc4)c4ncccc34)cc2=O)ccn1 ; Nc1nccc(Cn2ccc(-c3nn(-c4ccc(C(F)(F)F)cc4)c4ncccc34)cc2=O)c1Cl]

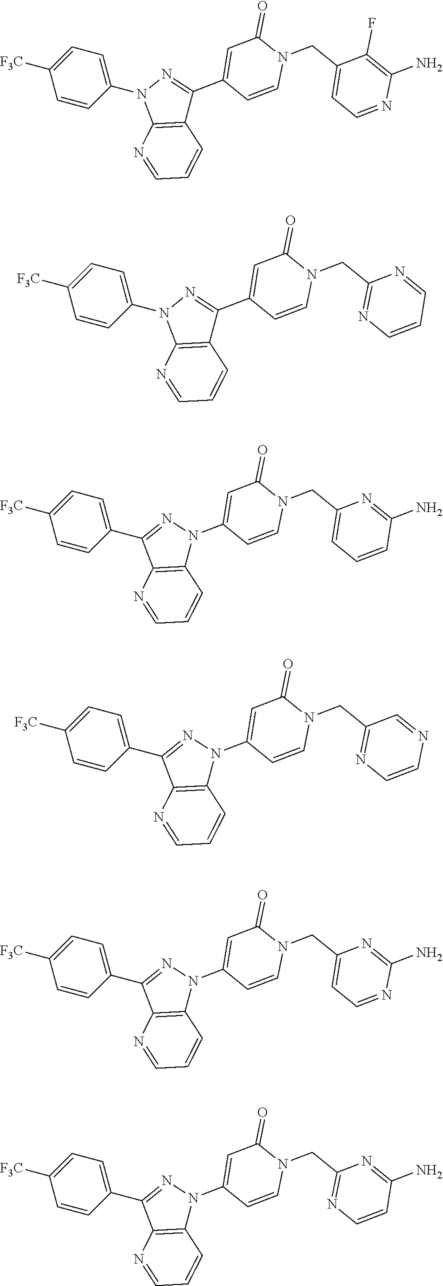 [SMILES: Nc1nccc(Cn2ccc(-c3nn(-c4ccc(C(F)(F)F)cc4)c4ncccc34)cc2=O)c1F ; O=c1cc(-c2nn(-c3ccc(C(F)(F)F)cc3)c3ncccc23)ccn1Cc1ncccn1 ; Nc1cccc(Cn2ccc(-n3nc(-c4ccc(C(F)(F)F)cc4)c4ncccc43)cc2=O)n1 ; O=c1cc(-n2nc(-c3ccc(C(F)(F)F)cc3)c3ncccc32)ccn1Cc1cnccn1 ; Nc1nccc(Cn2ccc(-n3nc(-c4ccc(C(F)(F)F)cc4)c4ncccc43)cc2=O)n1 ; Nc1ccnc(Cn2ccc(-n3nc(-c4ccc(C(F)(F)F)cc4)c4ncccc43)cc2=O)n1]

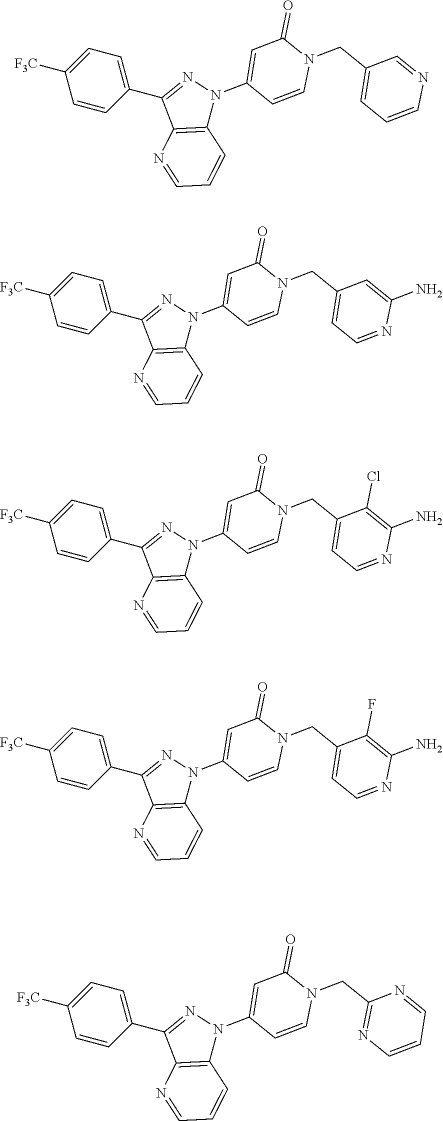 [SMILES: O=c1cc(-n2nc(-c3ccc(C(F)(F)F)cc3)c3ncccc32)ccn1Cc1cccnc1 ; Nc1cc(Cn2ccc(-n3nc(-c4ccc(C(F)(F)F)cc4)c4ncccc43)cc2=O)ccn1 ; Nc1nccc(Cn2ccc(-n3nc(-c4ccc(C(F)(F)F)cc4)c4ncccc43)cc2=O)c1Cl ; Nc1nccc(Cn2ccc(-n3nc(-c4ccc(C(F)(F)F)cc4)c4ncccc43)cc2=O)c1F ; O=c1cc(-n2nc(-c3ccc(C(F)(F)F)cc3)c3ncccc32)ccn1Cc1ncccn1]

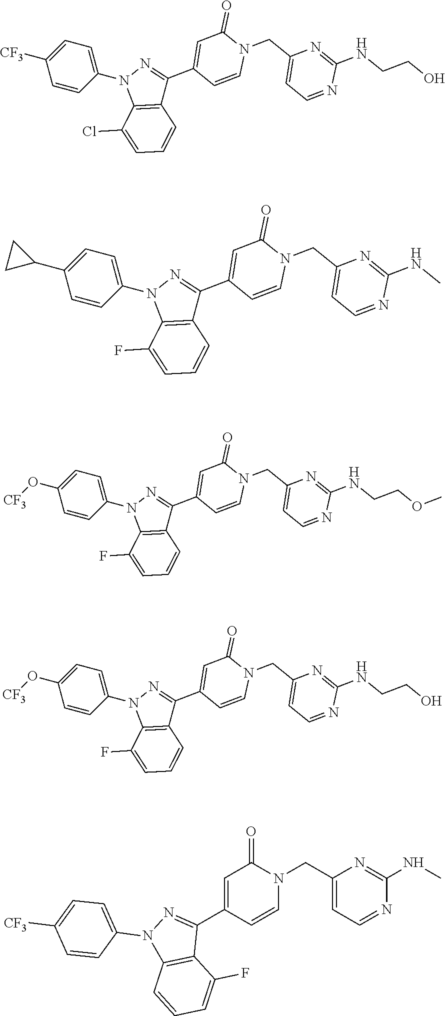 [SMILES: O=c1cc(-c2nn(-c3ccc(C(F)(F)F)cc3)c3c(Cl)cccc23)ccn1Cc1ccnc(NCCO)n1 ; CNc1nccc(Cn2ccc(-c3nn(-c4ccc(C5CC5)cc4)c4c(F)cccc34)cc2=O)n1 ; COCCNc1nccc(Cn2ccc(-c3nn(-c4ccc(OC(F)(F)F)cc4)c4c(F)cccc34)cc2=O)n1 ; O=c1cc(-c2nn(-c3ccc(OC(F)(F)F)cc3)c3c(F)cccc23)ccn1Cc1ccnc(NCCO)n1 ; CNc1nccc(Cn2ccc(-c3nn(-c4ccc(C(F)(F)F)cc4)c4cccc(F)c34)cc2=O)n1]

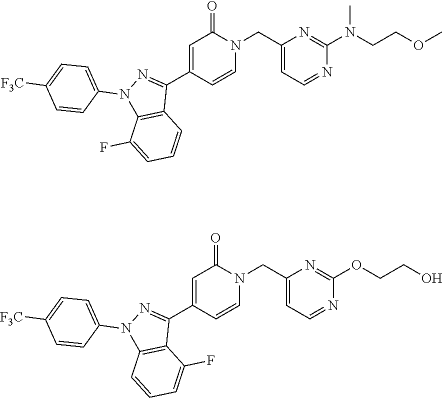 [SMILES: COCCN(C)c1nccc(Cn2ccc(-c3nn(-c4ccc(C(F)(F)F)cc4)c4c(F)cccc34)cc2=O)n1 ; O=c1cc(-c2nn(-c3ccc(C(F)(F)F)cc3)c3cccc(F)c23)ccn1Cc1ccnc(OCCO)n1]

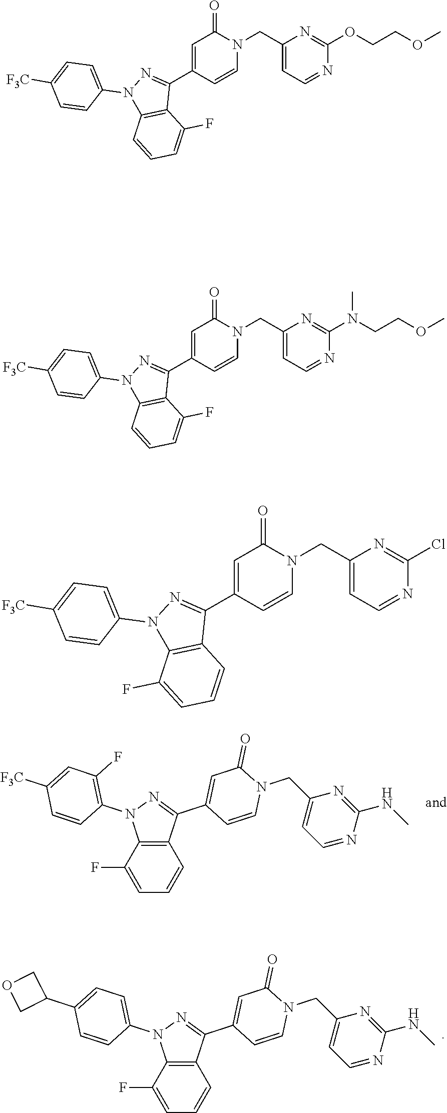 [SMILES: COCCOc1nccc(Cn2ccc(-c3nn(-c4ccc(C(F)(F)F)cc4)c4cccc(F)c34)cc2=O)n1 ; COCCN(C)c1nccc(Cn2ccc(-c3nn(-c4ccc(C(F)(F)F)cc4)c4cccc(F)c34)cc2=O)n1 ; O=c1cc(-c2nn(-c3ccc(C(F)(F)F)cc3)c3c(F)cccc23)ccn1Cc1ccnc(Cl)n1 ; CNc1nccc(Cn2ccc(-c3nn(-c4ccc(C(F)(F)F)cc4F)c4c(F)cccc34)cc2=O)n1 ; CNc1nccc(Cn2ccc(-c3nn(-c4ccc(C5COC5)cc4)c4c(F)cccc34)cc2=O)n1]

In some embodiments, compounds described herein are in the form of pharmaceutically acceptable salts. As well, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

“Pharmaceutically acceptable” as used herein, refers a material, such as a carrier or diluent, which does not abrogate the biological activity or properties of the compound, and is relatively nontoxic, i.e., the material is administered to an individual without causing undesirable biological effects or interacting in a deleterious manner with any of the components of the composition in which it is contained.

The term “pharmaceutically acceptable salt” refers to a form of a therapeutically active agent that consists of a cationic form of the therapeutically active agent in combination with a suitable anion, or in alternative embodiments, an anionic form of the therapeutically active agent in combination with a suitable cation. Handbook of Pharmaceutical Salts: Properties, Selection and Use. International Union of Pure and Applied Chemistry, Wiley-VCH 2002. S. M. Berge, L. D. Bighley, D. C. Monkhouse, J. Pharm. Sci. 1977, 66, 1-19. P. H. Stahl and C. G. Wermuth, editors, Handbook of Pharmaceutical Salts: Properties, Selection and Use, Weinheim/Zürich: Wiley-VCH/VHCA, 2002. Pharmaceutical salts typically are more soluble and more rapidly soluble in stomach and intestinal juices than non-ionic species and so are useful in solid dosage forms. Furthermore, because their solubility often is a function of pH, selective dissolution in one or another part of the digestive tract is possible, and this capability can be manipulated as one aspect of delayed and sustained release behaviors. Also, because the salt-forming molecule can be in equilibrium with a neutral form, passage through biological membranes can be adjusted.

In some embodiments, pharmaceutically acceptable salts are obtained by reacting a compound described herein with an acid to provide a “pharmaceutically acceptable acid addition salt”. In some embodiments, the compound described herein (i.e. free base form) is basic and is reacted with an organic acid or an inorganic acid. Inorganic acids include, but are not limited to, hydrochloric acid, hydrobromic acid, sulfuric acid, phosphoric acid, nitric acid, and metaphosphoric acid. Organic acids include, but are not limited to, 1-hydroxy-2-naphthoic acid; 2,2-dichloroacetic acid; 2-hydroxyethanesulfonic acid; 2-oxoglutaric acid; 4-acetamidobenzoic acid; 4-aminosalicylic acid; acetic acid; adipic acid; ascorbic acid (L); aspartic acid (L); benzenesulfonic acid; benzoic acid; camphoric acid (+); camphor-10-sulfonic acid (+); capric acid (decanoic acid); caproic acid (hexanoic acid); caprylic acid (octanoic acid); carbonic acid; cinnamic acid; citric acid; cyclamic acid; dodecylsulfuric acid; ethane-1,2-disulfonic acid; ethanesulfonic acid; formic acid; fumaric acid; galactaric acid; gentisic acid; glucoheptonic acid (D); gluconic acid (D); glucuronic acid (D); glutamic acid; glutaric acid; glycerophosphoric acid; glycolic acid; hippuric acid; isobutyric acid; lactic acid (DL); lactobionic acid; lauric acid; maleic acid; malic acid (−L); malonic acid; mandelic acid (DL); methanesulfonic acid; monomethyl fumarate, naphthalene-1,5-disulfonic acid; naphthalene-2-sulfonic acid; nicotinic acid; oleic acid; oxalic acid; palmitic acid; pamoic acid; phosphoric acid; proprionic acid; pyroglutamic acid (−L); salicylic acid; sebacic acid; stearic acid; succinic acid; sulfuric acid; tartaric acid (+L); thiocyanic acid; toluenesulfonic acid (p); and undecylenic acid.

In some embodiments, a compound described herein is prepared as a chloride salt, sulfate salt, bromide salt, mesylate salt, maleate salt, citrate salt or phosphate salt.

In some embodiments, pharmaceutically acceptable salts are obtained by reacting a compound described herein with a base to provide a “pharmaceutically acceptable base addition salt.”

In some embodiments, the compound described herein is acidic and is reacted with a base. In such situations, an acidic proton of the compound described herein is replaced by a metal ion, e.g., lithium, sodium, potassium, magnesium, calcium, or an aluminum ion. In some cases, compounds described herein coordinate with an organic base, such as, but not limited to, ethanolamine, diethanolamine, triethanolamine, tromethamine, meglumine, N-methylglucamine, dicyclohexylamine, tris(hydroxymethyl)methylamine. In other cases, compounds described herein form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, potassium carbonate, sodium hydroxide, lithium hydroxide, and the like. In some embodiments, the compounds provided herein are prepared as a sodium salt, calcium salt, potassium salt, magnesium salt, meglumine salt, N-methylglucamine salt or ammonium salt.

It should be understood that a reference to a pharmaceutically acceptable salt includes the solvent addition forms. In some embodiments, solvates contain either stoichiometric or non-stoichiometric amounts of a solvent, and are formed during the process of isolating or purifying the compound with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. In addition, the compounds provided herein optionally exist in unsolvated as well as solvated forms.

The methods and formulations described herein include the use of N-oxides (if appropriate), crystalline forms (also known as polymorphs), or pharmaceutically acceptable salts of compounds described herein, as well as active metabolites of these compounds having the same type of activity.

In some embodiments, sites on the organic radicals (e.g. alkyl groups, aromatic rings) of compounds described herein are susceptible to various metabolic reactions. Incorporation of appropriate substituents on the organic radicals will reduce, minimize or eliminate this metabolic pathway. In specific embodiments, the appropriate substituent to decrease or eliminate the susceptibility of the aromatic ring to metabolic reactions is, by way of example only, a halogen, deuterium, an alkyl group, a haloalkyl group, or a deuteroalkyl group.

In another embodiment, the compounds described herein are labeled isotopically (e.g. with a radioisotope) or by another other means, including, but not limited to, the use of chromophores or fluorescent moieties, bioluminescent labels, or chemiluminescent labels.

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, fluorine and chlorine, such as, for example, ²H, ³H, ¹³C, ¹⁴C, ⁵N, ¹⁸O, ¹⁷O, ³⁵S, ¹⁸F, ³⁶Cl. In one aspect, isotopically-labeled compounds described herein, for example those into which radioactive isotopes such as ³H and ¹⁴C are incorporated, are useful in drug and/or substrate tissue distribution assays. In one aspect, substitution with isotopes such as deuterium affords certain therapeutic advantages resulting from greater metabolic stability, such as, for example, increased in vivo half-life or reduced dosage requirements. In some embodiments, one or more hydrogen atoms of the compounds described herein is replaced with deuterium.

In some embodiments, the compounds described herein possess one or more stereocenters and each stereocenter exists independently in either the R or S configuration. The compounds presented herein include all diastereomeric, enantiomeric, atropisomers, and epimeric forms as well as the appropriate mixtures thereof. The compounds and methods provided herein include all cis, trans, syn, anti, entgegen (E), and zusammen (Z) isomers as well as the appropriate mixtures thereof.

Individual stereoisomers are obtained, if desired, by methods such as, stereoselective synthesis and/or the separation of stereoisomers by chiral chromatographic columns. In certain embodiments, compounds described herein are prepared as their individual stereoisomers by reacting a racemic mixture of the compound with an optically active resolving agent to form a pair of diastereoisomeric compounds/salts, separating the diastereomers and recovering the optically pure enantiomers. In some embodiments, resolution of enantiomers is carried out using covalent diastereomeric derivatives of the compounds described herein. In another embodiment, diastereomers are separated by separation/resolution techniques based upon differences in solubility. In other embodiments, separation of stereoisomers is performed by chromatography or by the forming diastereomeric salts and separation by recrystallization, or chromatography, or any combination thereof. Jean Jacques, Andre Collet, Samuel H. Wilen, “Enantiomers, Racemates and Resolutions”, John Wiley and Sons, Inc., 1981. In some embodiments, stereoisomers are obtained by stereoselective synthesis.

### Synthesis of Compounds

Compounds described herein are synthesized using standard synthetic techniques or using methods known in the art in combination with methods described herein.

Unless otherwise indicated, conventional methods of mass spectroscopy, NMR, HPLC, protein chemistry, biochemistry, recombinant DNA techniques and pharmacology are employed.

Compounds are prepared using standard organic chemistry techniques such as those described in, for example, March's Advanced Organic Chemistry, 6^{th} Edition, John Wiley and Sons, Inc. Alternative reaction conditions for the synthetic transformations described herein may be employed such as variation of solvent, reaction temperature, reaction time, as well as different chemical reagents and other reaction conditions. The starting materials are available from commercial sources or are readily prepared.

Suitable reference books and treatise that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatise that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

In some embodiments, compounds are prepared as described in the Examples.

### Methods

In some embodiments, described herein is a method of inhibition of the Hippo pathway in a subject in need thereof comprising administering to the subject a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), (Iy), (II), (IIa), or (IIb), or a pharmaceutically acceptable salt or solvate thereof.

In some embodiments, described herein is a method of inhibition of YAP-TEAD and/or TAZ-TEAD interaction in a subject in need thereof comprising administering to the subject a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), (Iy), (II), (IIa), or (IIb), or a pharmaceutically acceptable salt or solvate thereof. In some embodiments, described herein is a method of inhibition of YAP-TEAD and TAZ-TEAD interaction in a subject in need thereof comprising administering to the subject a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), (Iy), (II), (IIa), or (IIb), or a pharmaceutically acceptable salt or solvate thereof.

In some embodiments, described herein is a method of inhibition of YAP-TEAD in a subject in need thereof comprising administering to the subject a compound of Formula (I), (Ia), (Ib), (Ic), (Id), (Ie), (If), (Ig), (Ih), (Ii), (Ij), (Ik), (Il), (Im), (In), (o), (Ip), (Iq), (Ir), (Is), (It), (Iu), (Iv), (Iw), (Ix), (Iy), (II), (IIa), or (IIb), or a pharmaceutically acceptable salt or solvate thereof.

In some embodiments, described herein is a method of inhibition of TAZ-TEAD interaction in a subject in need thereof comprising administering to the subject a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), (Iy), (II), (IIa), or (IIb), or a pharmaceutically acceptable salt or solvate thereof.

In some embodiments, described herein is a method of treating a disease or condition affected by the inhibition of YAP-TEAD and/or TAZ-TEAD interaction in a subject in need thereof comprising administering to the subject a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), (Iy), (II), (IIa), or (IIb), or a pharmaceutically acceptable salt or solvate thereof. In some embodiments, described herein is a method of treating a disease or condition affected by the inhibition of YAP-TEAD and TAZ-TEAD interaction in a subject in need thereof comprising administering to the subject a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), (Iy), (II), (IIa), or (IIb), or a pharmaceutically acceptable salt or solvate thereof. In some embodiments, described herein is a method of treating a disease or condition affected by the inhibition of YAP-TEAD in a subject in need thereof comprising administering to the subject a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), (Iy), (II), (IIa), or (IIb), or a pharmaceutically acceptable salt or solvate thereof. In some embodiments, described herein is a method of treating a disease or condition affected by the inhibition of TAZ-TEAD interaction in a subject in need thereof comprising administering to the subject a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), (Iy), (II), (IIa), or (IIb), or a pharmaceutically acceptable salt or solvate thereof.

In some embodiments, described herein is a method of treating cancer in a subject in need thereof comprising administering to the subject a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), (Iy), (II), (IIa), or (IIb), or a pharmaceutically acceptable salt or solvate thereof. In some embodiments, described herein is a method of treating cancer in a subject in need thereof comprising administering to the subject a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Ix), (Iy), (Iw), (II), (IIa), or (IIb), or a pharmaceutically acceptable salt or solvate thereof, wherein the cancer is selected from breast cancer, lung cancer, liver cancer, ovarian cancer, squamous cancer, renal cancer, gastric cancer, medulloblastoma, colon cancer, and pancreatic cancer. In some embodiments, the cancer is breast cancer. In some embodiments, the cancer is lung cancer. In some embodiments, the cancer is liver cancer. In some embodiments, the cancer is ovarian cancer. In some embodiments, the cancer is squamous cancer. In some embodiments, the cancer is renal cancer. In some embodiments, the cancer is gastric cancer. In some embodiments, the cancer is medulloblastoma. In some embodiments, the cancer is colon cancer. In some embodiments, the cancer is pancreatic cancer.

In some embodiments, described herein is a method of treating a hyperproliferative disease and disorder in a subject in need thereof comprising administering to the subject a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), (Iy), (II), (IIa), or (IIb), or a pharmaceutically acceptable salt or solvate thereof.

In some embodiments, described herein is a method of treating cardiovascular disease in a subject in need thereof comprising administering to the subject a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), (Iy), (II), (IIa), or (IIb), or a pharmaceutically acceptable salt or solvate thereof.

In some embodiments, described herein is a method of treating fibrosis in a subject in need thereof comprising administering to the subject a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), (Iy), (II), (IIa), or (IIb), or a pharmaceutically acceptable salt or solvate thereof. In some embodiments, described herein is a method of treating fibrosis in a subject in need thereof comprising administering to the subject a compound of Formula (I), (Ia), (Ib), (Ic), (Id), (Ie), (If), (Ig), (Ih), (Ii), (Ij), (Ik), (Il), (Im), (In), (Io), (Ip), (Iq), (Ir), (Is), (It), (Iu), (Iv), (Iw), (Ix), (Iy), (II), (IIa), or (IIb), or a pharmaceutically acceptable salt or solvate thereof, wherein the fibrosis is liver fibrosis, kidney fibrosis and lung fibrosis.

### Pharmaceutical Compositions

In some embodiments, the compounds described herein are formulated into pharmaceutical compositions. Pharmaceutical compositions are formulated in a conventional manner using one or more pharmaceutically acceptable inactive ingredients that facilitate processing of the active compounds into preparations that are used pharmaceutically. Proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

In some embodiments, the compounds described herein are administered either alone or in combination with pharmaceutically acceptable carriers, excipients or diluents, in a pharmaceutical composition.

Administration of the compounds and compositions described herein can be affected by any method that enables delivery of the compounds to the site of action. These methods include, though are not limited to delivery via enteral routes (including oral, gastric or duodenal feeding tube, rectal suppository and rectal enema), parenteral routes (injection or infusion, including intraarterial, intracardiac, intradermal, intraduodenal, intramedullary, intramuscular, intraosseous, intraperitoneal, intrathecal, intravascular, intravenous, intravitreal, epidural and subcutaneous), inhalational, transdermal, transmucosal, sublingual, buccal and topical (including epicutaneous, dermal, enema, eye drops, ear drops, intranasal, vaginal) administration, although the most suitable route may depend upon for example the condition and disorder of the recipient.

In some embodiments, pharmaceutical compositions suitable for oral administration are presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. In some embodiments, the active ingredient is presented as a bolus, electuary or paste.

Pharmaceutical compositions which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. In some embodiments, the tablets are coated or scored and are formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In some embodiments, stabilizers are added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or Dragee coatings for identification or to characterize different combinations of active compound doses.

In some embodiments, pharmaceutical compositions are formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The compositions may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Pharmaceutical compositions for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical compositions may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example, as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

Pharmaceutical compositions may also be formulated in rectal compositions such as suppositories or retention enemas, e.g., containing conventional suppository bases such as cocoa butter, polyethylene glycol, or other glycerides.

Pharmaceutical compositions may be administered topically, that is by non-systemic administration. This includes the application of a compound of the present invention externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Pharmaceutical compositions suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient may comprise, for topical administration, from 0.001% to 10% w/w, for instance from 1% to 2% by weight of the formulation.

Pharmaceutical compositions for administration by inhalation are conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, pharmaceutical preparations may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

In some embodiments, a compound disclosed herein is formulated in such a manner that delivery of the compound to a particular region of the gastrointestinal tract is achieved. For example, a compound disclosed herein is formulated for oral delivery with bioadhesive polymers, pH-sensitive coatings, time dependent, biodegradable polymers, microflora activated systems, and the like, in order to effect delivering of the compound to a particular region of the gastrointestinal tract.

In some embodiments, a compound disclosed herein is formulated to provide a controlled release of the compound. Controlled release refers to the release of the compound described herein from a dosage form in which it is incorporated according to a desired profile over an extended period of time. Controlled release profiles include, for example, sustained release, prolonged release, pulsatile release, and delayed release profiles. In contrast to immediate release compositions, controlled release compositions allow delivery of an agent to a subject over an extended period of time according to a predetermined profile. Such release rates can provide therapeutically effective levels of agent for an extended period of time and thereby provide a longer period of pharmacologic response while minimizing side effects as compared to conventional rapid release dosage forms. Such longer periods of response provide for many inherent benefits that are not achieved with the corresponding short acting, immediate release preparations.

Approaches to deliver the intact therapeutic compound to the particular regions of the gastrointestinal tract (e.g. such as the colon), include:
(i) Coating with polymers: The intact molecule can be delivered to the colon without absorbing at the upper part of the intestine by coating of the drug molecule with the suitable polymers, which degrade only in the colon.
(ii) Coating with pH-sensitive polymers: The majority of enteric and colon targeted delivery systems are based on the coating of tablets or pellets, which are filled into conventional hard gelatin capsules. Most commonly used pH-dependent coating polymers are methacrylic acid copolymers, commonly known as Eudragit® S, more specifically Eudragit® L and Eudragit® S. Eudragit® L100 and S 100 are copolymers of methacrylic acid and methyl methacrylate.
(iii) Coating with biodegradable polymers;
(iv) Embedding in matrices;
(v) Embedding in biodegradable matrices and hydrogels;
(vi) Embedding in pH-sensitive matrices;
(vii) Timed release systems;
(viii) Redox-sensitive polymers;
(ix) Bioadhesive systems;
(x) Coating with microparticles;
(xi) Osmotic controlled drug delivery;

Another approach towards colon-targeted drug delivery or controlled-release systems includes embedding the drug in polymer matrices to trap it and release it in the colon. These matrices can be pH-sensitive or biodegradable. Matrix-Based Systems, such as multi-matrix (MMX)-based delayed-release tablets, ensure the drug release in the colon.

Additional pharmaceutical approaches to targeted delivery of therapeutics to particular regions of the gastrointestinal tract are known. Chourasia M K, Jain S K, Pharmaceutical approaches to colon targeted drug delivery systems, J Pharm Sci. 2003 January-April; 6(1):33-66. Patel M, Shah T, Amin A. Therapeutic opportunities in colon-specific drug-delivery systems Crit Rev Ther Drug Carrier Syst. 2007; 24(2):147-202. Kumar P, Mishra B. Colon targeted drug delivery systems—an overview. Curr Drug Deliv. 2008 July; 5(3):186-98. Van den Mooter G. Colon drug delivery. Expert Opin Drug Deliv. 2006 January; 3(1):111-25. Seth Amidon, Jack E. Brown, and Vivek S. Dave, Colon-Targeted Oral Drug Delivery Systems: Design Trends and Approaches, AAPS PharmSciTech. 2015 August; 16(4): 731-741.

It should be understood that in addition to the ingredients particularly mentioned above, the compounds and compositions described herein may include other agents conventional in the art having regard to the type of formulation in question, for example those suitable for oral administration may include flavoring agents.

### Methods of Dosing and Treatment Regimens

In one embodiment, the compounds described herein, or a pharmaceutically acceptable salt thereof, are used in the preparation of medicaments for the treatment of diseases or conditions in a mammal that would benefit from inhibition of YAP-TEAD and/or TAZ-TEAD interaction. Methods for treating any of the diseases or conditions described herein in a mammal in need of such treatment, involves administration of pharmaceutical compositions that include at least one compound described herein or a pharmaceutically acceptable salt, active metabolite, prodrug, or pharmaceutically acceptable solvate thereof, in therapeutically effective amounts to said mammal.

In certain embodiments, the compositions containing the compound(s) described herein are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation and/or dose ranging clinical trial.

In prophylactic applications, compositions containing the compounds described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. When used in patients, effective amounts for this use will depend on the severity and course of the disease, disorder or condition, previous therapy, the patient's health status and response to the drugs, and the judgment of the treating physician. In one aspect, prophylactic treatments include administering to a mammal, who previously experienced at least one symptom of the disease being treated and is currently in remission, a pharmaceutical composition comprising a compound described herein, or a pharmaceutically acceptable salt thereof, in order to prevent a return of the symptoms of the disease or condition.

In certain embodiments, wherein the patient's condition does not improve, upon the doctor's discretion, the compounds are administered chronically, that is, for an extended period of time, including throughout the duration of the patient's life in order to ameliorate or otherwise control or limit the symptoms of the patient's disease or condition.

In certain embodiments, wherein a patient's status does improve, the dose of drug being administered is temporarily reduced or temporarily suspended for a certain length of time (i.e., a “drug holiday”). In specific embodiments, the length of the drug holiday is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug holiday is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

Once improvement of the patient's conditions has occurred, a maintenance dose is administered if necessary. Subsequently, in specific embodiments, the dosage or the frequency of administration, or both, is reduced, as a function of the symptoms, to a level at which the improved disease, disorder or condition is retained. In certain embodiments, however, the patient requires intermittent treatment on a long-term basis upon any recurrence of symptoms.

The amount of a given agent that corresponds to such an amount varies depending upon factors such as the particular compound, disease condition and its severity, the identity (e.g., weight, sex) of the subject or host in need of treatment, but nevertheless is determined according to the particular circumstances surrounding the case, including, e.g., the specific agent being administered, the route of administration, the condition being treated, and the subject or host being treated.

In general, however, doses employed for adult human treatment are typically in the range of 0.01 mg-5000 mg per day. In one aspect, doses employed for adult human treatment are from about 1 mg to about 1000 mg per day. In one embodiment, the desired dose is conveniently presented in a single dose or in divided doses administered simultaneously or at appropriate intervals, for example as two, three, four or more sub-doses per day.

In one embodiment, the daily dosages appropriate for the compound described herein, or a pharmaceutically acceptable salt thereof, are from about 0.01 to about 50 mg/kg per body weight. In some embodiments, the daily dosage or the amount of active in the dosage form are lower or higher than the ranges indicated herein, based on a number of variables in regard to an individual treatment regime. In various embodiments, the daily and unit dosages are altered depending on a number of variables including, but not limited to, the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens are determined by standard pharmaceutical procedures in cell cultures or experimental animals, including, but not limited to, the determination of the LD₅₀ and the ED₅₀. The dose ratio between the toxic and therapeutic effects is the therapeutic index and it is expressed as the ratio between LD₅₀ and ED₅₀. In certain embodiments, the data obtained from cell culture assays and animal studies are used in formulating the therapeutically effective daily dosage range and/or the therapeutically effective unit dosage amount for use in mammals, including humans. In some embodiments, the daily dosage amount of the compounds described herein lies within a range of circulating concentrations that include the ED₅₀ with minimal toxicity. In certain embodiments, the daily dosage range and/or the unit dosage amount varies within this range depending upon the dosage form employed and the route of administration utilized.

In any of the aforementioned aspects are further embodiments in which the effective amount of the compound described herein, or a pharmaceutically acceptable salt thereof, is: (a) systemically administered to the mammal; and/or (b) administered orally to the mammal; and/or (c) intravenously administered to the mammal; and/or (d) administered by injection to the mammal; and/or (e) administered topically to the mammal; and/or (f) administered non-systemically or locally to the mammal.

In any of the aforementioned aspects are further embodiments comprising single administrations of the effective amount of the compound, including further embodiments in which (i) the compound is administered once a day; or (ii) the compound is administered to the mammal multiple times over the span of one day.

In any of the aforementioned aspects are further embodiments comprising multiple administrations of the effective amount of the compound, including further embodiments in which (i) the compound is administered continuously or intermittently: as in a single dose; (ii) the time between multiple administrations is every 6 hours; (iii) the compound is administered to the mammal every 8 hours; (iv) the compound is administered to the mammal every 12 hours; (v) the compound is administered to the mammal every 24 hours. In further or alternative embodiments, the method comprises a drug holiday, wherein the administration of the compound is temporarily suspended or the dose of the compound being administered is temporarily reduced; at the end of the drug holiday, dosing of the compound is resumed. In one embodiment, the length of the drug holiday varies from 2 days to 1 year.

In certain instances, it is appropriate to administer at least one compound described herein, or a pharmaceutically acceptable salt thereof, in combination with one or more other therapeutic agents.

In one embodiment, the therapeutic effectiveness of one of the compounds described herein is enhanced by administration of an adjuvant (i.e., by itself the adjuvant has minimal therapeutic benefit, but in combination with another therapeutic agent, the overall therapeutic benefit to the patient is enhanced). Or, in some embodiments, the benefit experienced by a patient is increased by administering one of the compounds described herein with another agent (which also includes a therapeutic regimen) that also has therapeutic benefit.

In one specific embodiment, a compound described herein, or a pharmaceutically acceptable salt thereof, is co-administered with a second therapeutic agent, wherein the compound described herein, or a pharmaceutically acceptable salt thereof, and the second therapeutic agent modulate different aspects of the disease, disorder or condition being treated, thereby providing a greater overall benefit than administration of either therapeutic agent alone.

In any case, regardless of the disease, disorder or condition being treated, the overall benefit experienced by the patient may be additive of the two therapeutic agents or the patient may experience a synergistic benefit.

In certain embodiments, different therapeutically-effective dosages of the compounds disclosed herein will be utilized in formulating pharmaceutical composition and/or in treatment regimens when the compounds disclosed herein are administered in combination with one or more additional agent, such as an additional therapeutically effective drug, an adjuvant or the like. Therapeutically-effective dosages of drugs and other agents for use in combination treatment regimens is optionally determined by means similar to those set forth hereinabove for the actives themselves. Furthermore, the methods of prevention/treatment described herein encompasses the use of metronomic dosing, i.e., providing more frequent, lower doses in order to minimize toxic side effects. In some embodiments, a combination treatment regimen encompasses treatment regimens in which administration of a compound described herein, or a pharmaceutically acceptable salt thereof, is initiated prior to, during, or after treatment with a second agent described herein, and continues until any time during treatment with the second agent or after termination of treatment with the second agent. It also includes treatments in which a compound described herein, or a pharmaceutically acceptable salt thereof, and the second agent being used in combination are administered simultaneously or at different times and/or at decreasing or increasing intervals during the treatment period. Combination treatment further includes periodic treatments that start and stop at various times to assist with the clinical management of the patient.

It is understood that the dosage regimen to treat, prevent, or ameliorate the condition(s) for which relief is sought, is modified in accordance with a variety of factors (e.g. the disease, disorder or condition from which the subject suffers; the age, weight, sex, diet, and medical condition of the subject). Thus, in some instances, the dosage regimen actually employed varies and, in some embodiments, deviates from the dosage regimens set forth herein.

For combination therapies described herein, dosages of the co-administered compounds vary depending on the type of co-drug employed, on the specific drug employed, on the disease or condition being treated and so forth. In additional embodiments, when co-administered with one or more other therapeutic agents, the compound provided herein is administered either simultaneously with the one or more other therapeutic agents, or sequentially.

In combination therapies, the multiple therapeutic agents (one of which is one of the compounds described herein) are administered in any order or even simultaneously. If administration is simultaneous, the multiple therapeutic agents are, by way of example only, provided in a single, unified form, or in multiple forms (e.g., as a single pill or as two separate pills).

The compounds described herein, or a pharmaceutically acceptable salt thereof, as well as combination therapies, are administered before, during or after the occurrence of a disease or condition, and the timing of administering the composition containing a compound varies. Thus, in one embodiment, the compounds described herein are used as a prophylactic and are administered continuously to subjects with a propensity to develop conditions or diseases in order to prevent the occurrence of the disease or condition. In another embodiment, the compounds and compositions are administered to a subject during or as soon as possible after the onset of the symptoms. In specific embodiments, a compound described herein is administered as soon as is practicable after the onset of a disease or condition is detected or suspected, and for a length of time necessary for the treatment of the disease. In some embodiments, the length required for treatment varies, and the treatment length is adjusted to suit the specific needs of each subject. For example, in specific embodiments, a compound described herein or a formulation containing the compound is administered for at least 2 weeks, about 1 month to about 5 years.

### EXAMPLES

The following examples are provided for illustrative purposes only and not to limit the scope of the claims provided herein.

As used above, and throughout the description of the invention, the following abbreviations, unless otherwise indicated, shall be understood to have the following meanings:
ACN or MeCN acetonitrile
AcOH acetic acid
Ac acetyl
BINAP 2,2′-bis(diphenylphosphino)-1,1′-binaphthalene
Bn benzyl
BOC or Boc tert-butyl carbamate
t-Bu tert-butyl
Cy cyclohexyl
DBA or dba dibenzylideneacetone
DCE dichloroethane (ClCH₂CH₂Cl)
DCM dichloromethane (CH₂Cl₂)
DIPEA or DIEA diisopropylethylamine
DMAP 4-(N,N-dimethylamino)pyridine
DME 1,2-dimethoxyethane
DMF N,N-dimethylformamide
DMA N,N-dimethylacetamide
DMSO dimethylsulfoxide
Dppf or dppf 1,1′-bis(diphenylphosphino)ferrocene
EEDQ 2-Ethoxy-1-ethoxycarbonyl-1,2-dihydroquinoline
eq equivalent(s)
Et ethyl
Et₂O diethyl ether
EtOH ethanol
EtOAc ethyl acetate
HATU 1-[bis(dimethylamino)methylene]-1H-1,2,3-triazolo[4,5-b]pyridinium 3-oxid hexafluorophosphate
HMPA hexamethylphosphoramide
HPLC high performance liquid chromatography
KHMDS potassium bis(trimethylsilyl)amide
NaHMDS sodium bis(trimethylsilyl)amide
LiHMDS lithium bis(trimethylsilyl)amide
LAH lithium aluminum anhydride
LCMS liquid chromatography mass spectrometry
Me methyl
MeOH methanol
MS mass spectroscopy
Ms mesyl
NBS N-bromosuccinimide
NMM N-methyl-morpholine
NMP N-methyl-pyrrolidin-2-one
NMR nuclear magnetic resonance
PCC pyridinium chlorochromate
Ph phenyl
PPTS pyridium p-toluenesulfonate
iPr/i-Pr iso-propyl
TBS tert-butyldimethylsilyl
RP-HPLC reverse phase-high pressure liquid chromatography
TFA trifluoroacetic acid
TEA triethylamine
THF tetrahydrofuran
TLC thin layer chromatography

### Chemistry Examples

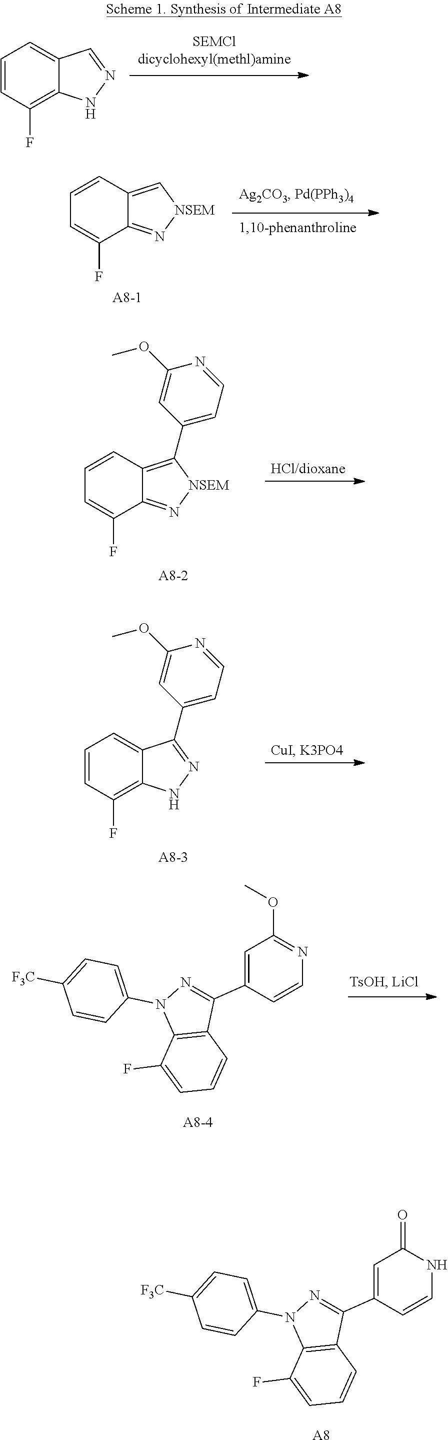

Step 1: To a solution of 7-fluoro-1H-indazole (3 g, 22.04 mmol) and dicyclohexyl(methyl)amine (5.17 g, 26.45 mmol) in THF (50 mL) was added 1-chloro-5,5-dimethyl-2-oxa-5-silahexane (4.41 g, 26.45 mmol) at 25° C. The yellow solution was stirred at 25° C. for 12 h. The mixture was quenched with saturated sodium bicarbonate aqueous solution (50 mL) and extracted with EtOAc (30 mL*3). The combined organic layer was washed with brine (50 mL), dried and filtered. The filtrate was concentrated under reduced pressure. The residue was purified by combi flash (6% EtOAc in Heptane) to give compound A8-1 (5.8 g, 21.77 mmol, 98.81%) as yellow oil.

LC-MS (ESI) m/z: 267.1 [M+H]⁺.

Step 2: To a mixture of compound A8-1 (5.8 g, 21.77 mmol) and 4-iodo-2-methoxypyridine (5.37 g, 22.86 mmol) in Water (100 mL) were added Pd(PPh₃)₄ (2.52 g, 2.18 mmol), 1,10-phenanthroline (0.78 g, 4.36 mmol) and silver carbonate (9.01 g, 32.66 mmol). The mixture was stirred at 100° C. for 18 h under N₂ atmosphere. The mixture was cooled to RT, diluted with water (60 mL) and EtOAc (60 mL). The mixture was filtered, the filtrate was extracted with EtOAc (30 mL*2). The combined organic layer was washed with brine, dried over anhydrous Na₂SO₄, filtered and concentrated under reduced pressure. The residue was purified by silica gel (6% EtOAc in Heptane) to give compound A8-2 (6.5 g, 17.4 mmol, 79.95%) as a colourless oil.

LC-MS (ESI) m/z: 374.1 [M+H]⁺.

Step 3: To a solution of compound A8-2 (5 g, 13.39 mmol) in MeOH (50 mL) was added HCL/dioxane (20 mL, 4.0 M), and the mixture was stirred at 20° C. for 1 h. The mixture was concentrated under reduced pressure. the residue was adjusted pH to 8 with saturated sodium bicarbonate aqueous solution, then the mixture was extracted with EtOAc (100 mL×2). The combined layer was washed with brine, dried over anhydrous Na₂SO₄, filtered and concentrated under reduced pressure to give compound A8-3 (3.2 g, 13.05 mmol, 97.46%) as a white solid.

LC-MS (ESI) m/z: 244.1 [M+H]⁺.

Step 4: To a solution of compound A8-3 (800 mg, 3.29 mmol) in DMF (10 mL) was added 1-iodo-4-(trifluoromethyl)benzene (890 mg, 3.45 mmol), CuI (375 mg, 1.97 mmol), N,N′-DiMethylethylenediaMine (347 mg, 3.95 mmol) and K₃PO₄ (1.39 g, 6.58 mmol). The mixture was stirred at 130° C. for 14 h under N2 atmosphere. The mixture was diluted with water (60 mL) and EtOAc (60 mL). The mixture was filtered, the filtrate was extracted with EtOAc (50 mL>2). The combined organic layer was washed with brine, dried over anhydrous Na₂SO₄, filtered and concentrated under reduced pressure. The residue was purified by silica gel (16% EtOAc in Heptane) to give compound A8-4 (950 mg, 2.45 mmol, 74.57%) as a white solid.

LC-MS (ESI) m/z: 388.1 [M+H]⁺.

Step 5: To a solution of compound A8-4 (950 mg, 2.45 mmol) in DMF (8 mL) was added p-Toluenesulfonic acid monohydrate (2.33 g, 12.25 mmol) and LiCl (519 mg, 12.25 mmol). The mixture was stirred at 120° C. for 1 h. The mixture was cooled to RT and added water (60 mL) dropwise. The mixture was stirred at RT for 0.5 h. The mixture was filtered, the solid was washed with water twice. The solid was dried to afford compound A8 (900 mg, 2.41 mmol, 98.30%) as a white solid.

LC-MS (ESI) m/z: 374.1 [M+H]⁺.

¹H-NMR (400 MHz, DMSO-d₆) δ 11.81 (s, 1H), 8.04-7.95 (m, 5H), 7.55 (d, J=6.8 Hz, 1H), 7.52-7.46 (m, 1H), 7.45-7.39 (m, 1H), 6.96 (d, J=1.2 Hz, 1H), 6.81 (dd, J=6.8, 1.7 Hz, 1H).

The following intermediates listed below were synthesized according to the procedure of A8.

| Intermediate | | |
|---|---|---|
| No. | Structure | Analytical data |
| A1 | 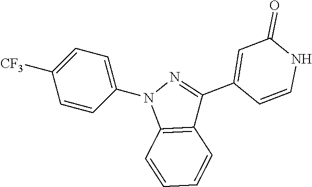 [SMILES: O=c1cc(-c2nn(-c3ccc(C(F)(F)F)cc3)c3ccccc23)cc[nH]1] | LCMS: m/z 356.1 [M + H]⁺. |
| A2 | 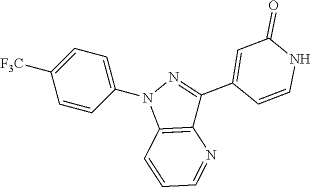 [SMILES: O=c1cc(-c2nn(-c3ccc(C(F)(F)F)cc3)c3cccnc23)cc[nH]1] | LCMS: m/z 357.1 [M + H]⁺. |
| A3 | 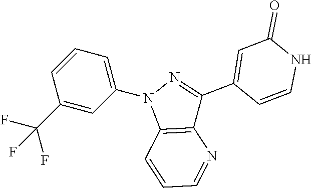 [SMILES: O=c1cc(-c2nn(-c3cccc(C(F)(F)F)c3)c3cccnc23)cc[nH]1] | LCMS: m/z 357.1 [M + H]⁺. |
| A4 | 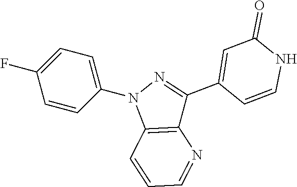 [SMILES: O=c1cc(-c2nn(-c3ccc(F)cc3)c3cccnc23)cc[nH]1] | LCMS: m/z 307.1 [M + H]⁺. |
| A5 | 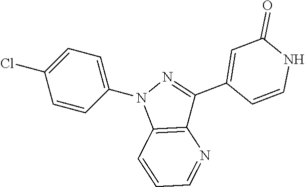 [SMILES: O=c1cc(-c2nn(-c3ccc(Cl)cc3)c3cccnc23)cc[nH]1] | LCMS: m/z 323.1 [M + H]⁺. |
| A6 | 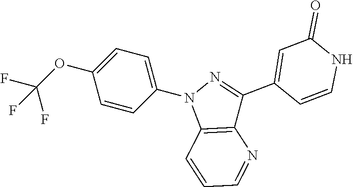 [SMILES: O=c1cc(-c2nn(-c3ccc(OC(F)(F)F)cc3)c3cccnc23)cc[nH]1] | LCMS: m/z 373.1 [M + H]⁺. |
| A7 | 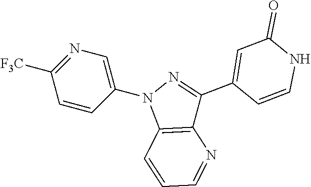 [SMILES: O=c1cc(-c2nn(-c3ccc(C(F)(F)F)nc3)c3cccnc23)cc[nH]1] | LCMS: m/z 358.1 [M + H]⁺. |
| A8 | 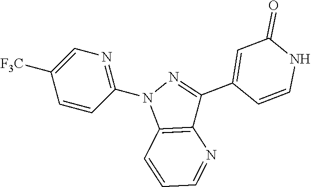 [SMILES: O=c1cc(-c2nn(-c3ccc(C(F)(F)F)cn3)c3cccnc23)cc[nH]1] | LCMS: m/z 358.1 [M + H]⁺. |
| A9 | 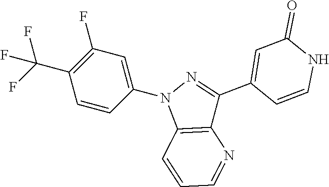 [SMILES: O=c1cc(-c2nn(-c3ccc(C(F)(F)F)c(F)c3)c3cccnc23)cc[nH]1] | LCMS: m/z 375.1 [M + H]⁺. |
| A10 | 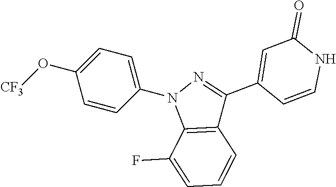 [SMILES: O=c1cc(-c2nn(-c3ccc(OC(F)(F)F)cc3)c3c(F)cccc23)cc[nH]1] | LCMS: m/z 390.1 [M + H]⁺. |
| A11 | 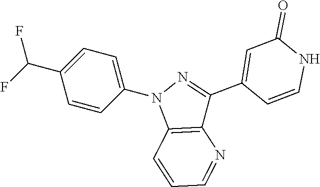 [SMILES: O=c1cc(-c2nn(-c3ccc(C(F)F)cc3)c3cccnc23)cc[nH]1] | LCMS: m/z 339.1 [M + H]⁺. |
| A12 | 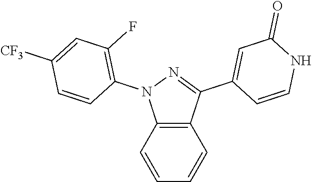 [SMILES: O=c1cc(-c2nn(-c3ccc(C(F)(F)F)cc3F)c3ccccc23)cc[nH]1] | LCMS: m/z 374.1 [M + H]⁺. |
| A13 | 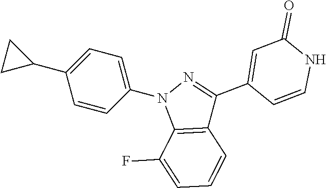 [SMILES: O=c1cc(-c2nn(-c3ccc(C4CC4)cc3)c3c(F)cccc23)cc[nH]1] | LCMS: m/z 346.1 [M + H]⁺. |
| A14 | 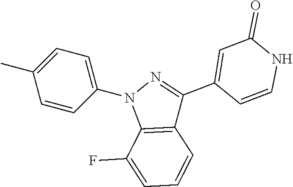 [SMILES: Cc1ccc(-n2nc(-c3cc[nH]c(=O)c3)c3cccc(F)c32)cc1] | LCMS: m/z 320.1 [M + H]⁺. |
| A15 | 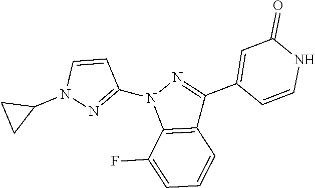 [SMILES: O=c1cc(-c2nn(-c3ccn(C4CC4)n3)c3c(F)cccc23)cc[nH]1] | LCMS: m/z 336.1 [M + H]⁺. |
| A16 | 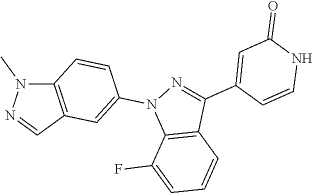 [SMILES: Cn1ncc2cc(-n3nc(-c4cc[nH]c(=O)c4)c4cccc(F)c43)ccc21] | LCMS: m/z 360.1 [M + H]⁺. |
| A17 | 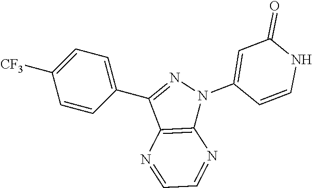 [SMILES: O=c1cc(-n2nc(-c3ccc(C(F)(F)F)cc3)c3nccnc32)cc[nH]1] | LCMS: m/z 358.1 [M + H]⁺. |
| A18 | 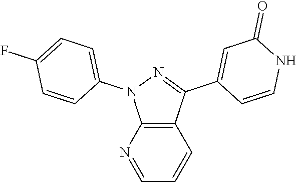 [SMILES: O=c1cc(-c2nn(-c3ccc(F)cc3)c3ncccc23)cc[nH]1] | LCMS: m/z 307.1 [M + H]⁺. |
| A19 | 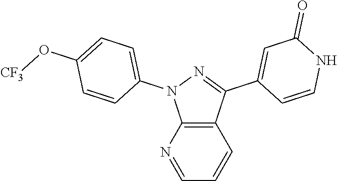 [SMILES: O=c1cc(-c2nn(-c3ccc(OC(F)(F)F)cc3)c3ncccc23)cc[nH]1] | LCMS: m/z 373.1 [M + H]⁺. |
| A20 | 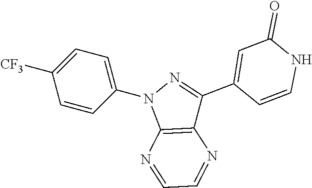 [SMILES: O=c1cc(-c2nn(-c3ccc(C(F)(F)F)cc3)c3nccnc23)cc[nH]1] | LCMS: m/z 358.1 [M + H]⁺. |
| A21 | 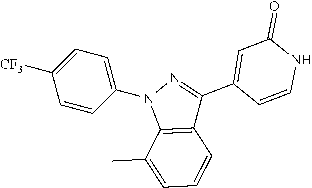 [SMILES: Cc1cccc2c(-c3cc[nH]c(=O)c3)nn(-c3ccc(C(F)(F)F)cc3)c12] | LCMS: m/z 370.1 [M + H]⁺. |
| A22 | 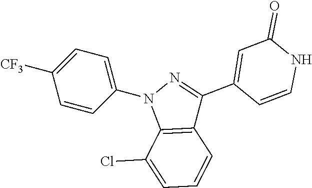 [SMILES: O=c1cc(-c2nn(-c3ccc(C(F)(F)F)cc3)c3c(Cl)cccc23)cc[nH]1] | LCMS: m/z 390.1 [M + H]⁺. |
| A23 | 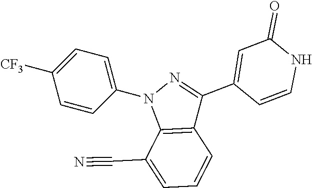 [SMILES: N#Cc1cccc2c(-c3cc[nH]c(=O)c3)nn(-c3ccc(C(F)(F)F)cc3)c12] | LCMS: m/z 381.1 [M + H]⁺. |
| A24 | 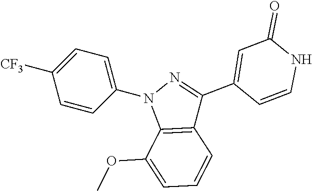 [SMILES: COc1cccc2c(-c3cc[nH]c(=O)c3)nn(-c3ccc(C(F)(F)F)cc3)c12] | LCMS: m/z 386.1 [M + H]⁺. |
| A27 | 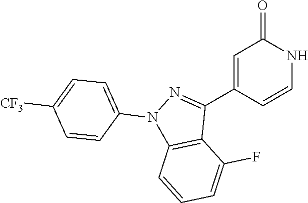 [SMILES: O=c1cc(-c2nn(-c3ccc(C(F)(F)F)cc3)c3cccc(F)c23)cc[nH]1] | LCMS: m/z 374.1 [M + H]⁺. |
| A28 | 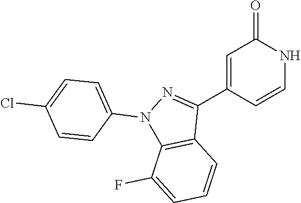 [SMILES: O=c1cc(-c2nn(-c3ccc(Cl)cc3)c3c(F)cccc23)cc[nH]1] | LCMS: m/z 340.1 [M + H]⁺. |
| B1 | 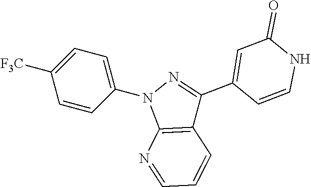 [SMILES: O=c1cc(-c2nn(-c3ccc(C(F)(F)F)cc3)c3ncccc23)cc[nH]1] | LCMS: m/z 357.1 [M + H]⁺. |
| B2 | 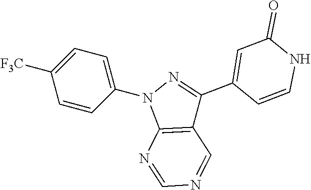 [SMILES: O=c1cc(-c2nn(-c3ccc(C(F)(F)F)cc3)c3ncncc23)cc[nH]1] | LCMS: m/z 358.1 [M + H]⁺. |
| B3 | 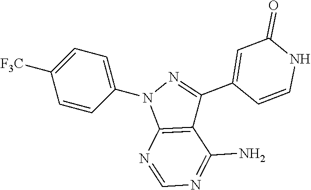 [SMILES: Nc1ncnc2c1c(-c1cc[nH]c(=O)c1)nn2-c1ccc(C(F)(F)F)cc1] | LCMS: m/z 373.1 [M + H]⁺. |
| B6 | 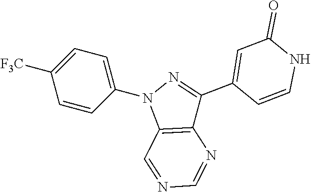 [SMILES: O=c1cc(-c2nn(-c3ccc(C(F)(F)F)cc3)c3cncnc23)cc[nH]1] | LCMS: m/z 358.1 [M + H]⁺. |
| B7 | 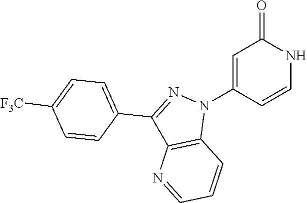 [SMILES: O=c1cc(-n2nc(-c3ccc(C(F)(F)F)cc3)c3ncccc32)cc[nH]1] | LCMS: m/z 357.1 [M + H]⁺. |
| B8 | 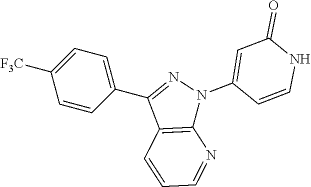 [SMILES: O=c1cc(-n2nc(-c3ccc(C(F)(F)F)cc3)c3cccnc32)cc[nH]1] | LCMS: m/z 357.1 [M + H]⁺. |
| B9 | 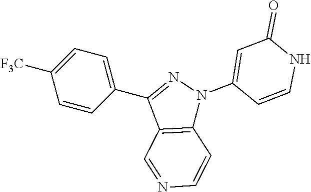 [SMILES: O=c1cc(-n2nc(-c3ccc(C(F)(F)F)cc3)c3cnccc32)cc[nH]1] | LCMS: m/z 357.1 [M + H]⁺. |
| B12 | 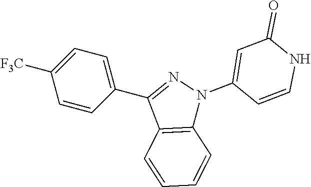 [SMILES: O=c1cc(-n2nc(-c3ccc(C(F)(F)F)cc3)c3ccccc32)cc[nH]1] | LCMS: m/z 356.1 [M + H]⁺. |
| B27 | 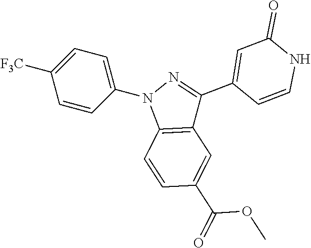 [SMILES: COC(=O)c1ccc2c(c1)c(-c1cc[nH]c(=O)c1)nn2-c1ccc(C(F)(F)F)cc1] | LCMS: m/z 414.1 [M + H]⁺. |
| B24 | 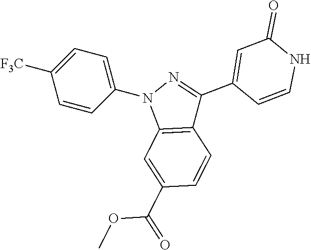 [SMILES: COC(=O)c1ccc2c(-c3cc[nH]c(=O)c3)nn(-c3ccc(C(F)(F)F)cc3)c2c1] | LCMS: m/z 414.1 [M + H]⁺. |
| C1 | 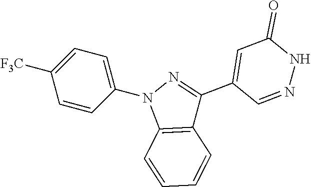 [SMILES: O=c1cc(-c2nn(-c3ccc(C(F)(F)F)cc3)c3ccccc23)cn[nH]1] | LCMS: m/z 357.1 [M + H]⁺. |
| C3 | 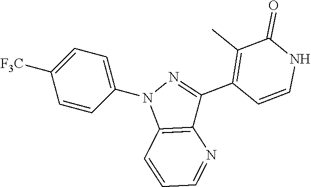 [SMILES: Cc1c(-c2nn(-c3ccc(C(F)(F)F)cc3)c3cccnc23)cc[nH]c1=O] | LCMS: m/z 371.1 [M + H]⁺. |

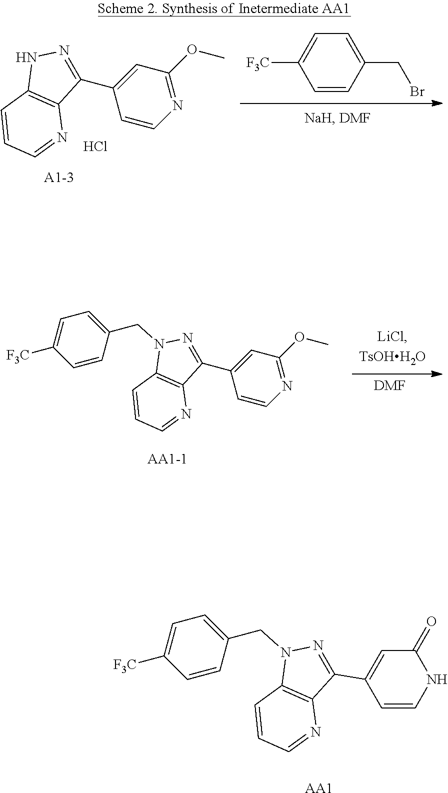

Step 1. To a solution of compound A1-3 (100 mg, 0.381 mmol, prepared by the method described in Scheme 1) in DMF (4 mL) were added NaH (46 mg, 1.14 mmol, 60% in mineral oil) and 1-(bromomethyl)-4-(trifluoromethyl)benzene (109 mg, 0.457 mmol), and the mixture was stirred at 20° C. for 12 h. The mixture was diluted with water (15 mL). The mixture was extracted with EtOAc (20 mL×2). The combined organic layer was washed with brine, dried over anhydrous Na₂SO₄, filtered and concentrated under reduced pressure. The residue was purified by flash column chromatography on silica gel (gradient elution, 0-25% EtOAc/PE) to afford compound AA1-1 (85 mg, 58.1%) as Colorless oil.

LC-MS (ESI) m/z: 385.1 [M+H]⁺.

Step 2. To a solution of compound AA1-1 (85 mg, 0.221 mmol) in DMF (3 mL) were added LiCl (47 mg, 1.11 mmol) and p-toluenesulfonic acid monohydrate (148 mg, 0.781 mmol), and the mixture was stirred at 120° C. for 1 h. The mixture was diluted with water (15 mL). The mixture was extracted with EtOAc (20 mL×2). The combined organic layer was washed with brine, dried over anhydrous Na₂SO₄, filtered and concentrated under reduced pressure to afford compound AA1 (80 mg, 97.7%) as a white solid.

LC-MS (ESI) m/z: 371.1 [M+H]⁺.

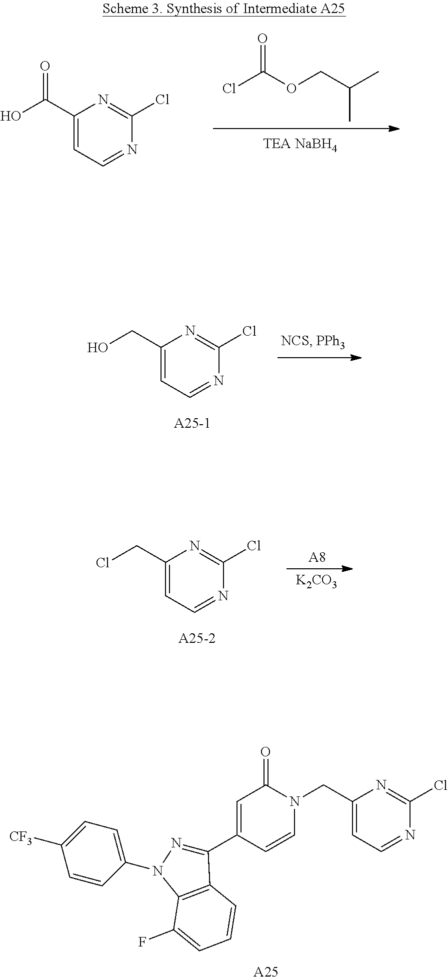

Step 1: To a solution of 2-chloropyrimidine-4-carboxylic acid (10 g, 63.08 mmol) and TEA (7.02 g, 69.38 mmol) in THF (100 mL) was slowly added Isobutyl Chloroformate (9.48 g, 69.38 mmol). The mixture was stirred at 25° C. for 1 h. The formed precipitate was filtered off and to obtained clear solution was slowly added a solution of NaBH₄ (4.77 g, 126.15 mmol) in water (20 mL). The reaction mixture was stirred at 25° C. for 30 min. Then the mixture was added water (100 mL) and extracted with EtOAc (40 mL*3). Combined organic fractions were washed with brine (60 mL) and dried over anhydrous Na₂SO₄. After evaporation of the solvents under reduced pressure, the residue was purified by combi flash (40% EtOAc/Heptane) to give compound A25-1 (2 g, 13.86 mmol, 21.93%) as white solid.

LC-MS: m/z=144.9 [M+H]⁺.

Step 2: To a solution of compound A25-1 (1.8 g, 12.452 mmol) and PPh₃ (3.27 g, 12.45 mmol) in DCM (40 mL) was added NCS (1.66 g, 12.45 mmol) slowly at 0° C. The mixture was stirred at 20° C. for 2 h. The mixture was diluted with water and extracted with EtOAc. The combined organic layer was washed with brine, dried over anhydrous Na₂SO₄, filtered and concentrated under reduced pressure. The residue was purified by flash column chromatography on silica gel (Gradient elution, 10% EtOAc/Heptane) to give compound A25-2 (1.8 g, 11.04 mmol, 88.69%) as a colorless oil.

LC-MS: m/z=163.0 [M+H]⁺.

Step 3: To a solution of compound A8 (500 mg, 1.34 mmol) in DMF (10 mL) was added compound A25-2 (327 mg, 2.00 mmol) and K₂CO₃ (370 mg, 2.68 mmol). The mixture was stirred at 40° C. for 15 h. The mixture was diluted with water and extracted with EtOAc twice. The combined organic layer was washed with brine, dried over anhydrous Na₂SO₄, filtered and concentrated under reduced pressure. The residue was purified by flash column chromatography on silica gel (Gradient elution, 4% MeOH in DCM) to give compound A25 (470 mg, 0.94 mmol, 70.20%) as a white solid.

LC-MS: m/z=500.1 [M+H]⁺.

The following intermediates listed below were synthesized according to the procedure of A25:

| Intermediate | | |
|---|---|---|
| No. | Structure | Analytical data |
| A26 | 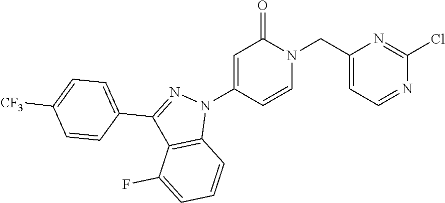 [SMILES: O=c1cc(-n2nc(-c3ccc(C(F)(F)F)cc3)c3c(F)cccc32)ccn1Cc1ccnc(Cl)n1] | LCMS: m/z 500.1 [M + H]⁺. |

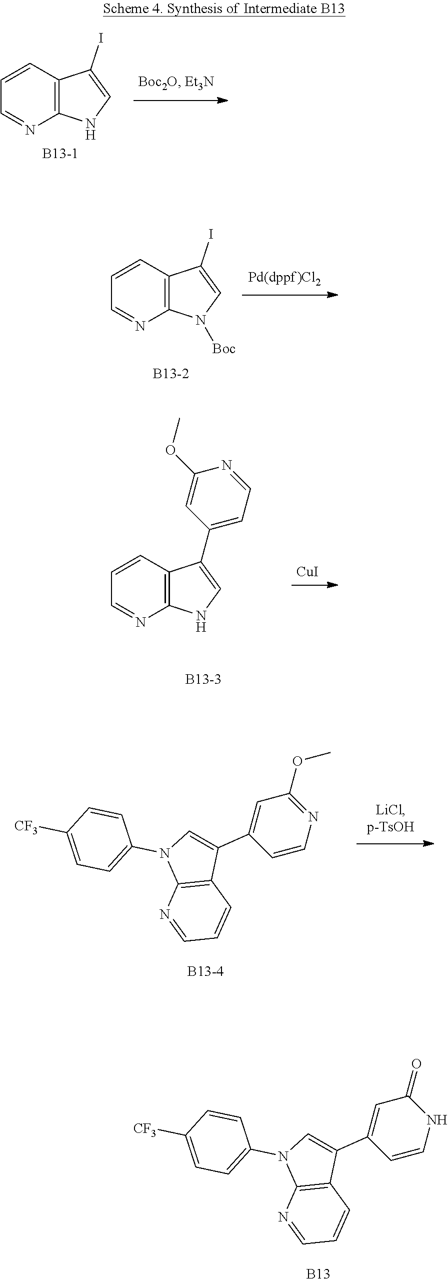

Step 1: To a solution of B13-1 (1 g, 4.11 mmol), DMAP (0.05 g, 0.41 mmol) and Et₃N (0.86 mL, 6.17 mmol) in THF (20 mL) was added Boc₂O (1.06 mL, 4.94 mmol). The yellow solution was stirred at 25° C. for 12 h. TLC showed the reaction was completed. The mixture was concentrated under reduced pressure. The residue was purified by combi flash (0-20% EtOAc/Petroleum ether) to afford B13-2 (1.35 g, 3.93 mmol, 95.62%) as yellow oil.

LC-MS (ESI) m/z: 345.1 [M+H]⁺

Step 2: To a suspension of B13-2 (100 mg, 0.29 mmol), (2-methoxypyridin-4-yl)bornanediol (66.66 mg, 0.44 mmol) and K₃PO₄ (123.35 mg, 0.58 mmol) in dioxane (2 mL) and H₂O (0.2 mL) was added Pd(dppf)Cl₂·CH₂Cl₂ (23.73 mg, 0.03 mmol). The red suspension was stirred at 100° C. for 12 h under nitrogen protection. TLC showed the reaction was completed. The mixture was concentrated under reduced pressure. The residue was purified by combi flash (0-50% EtOAc/Petroleum ether) to afford B13-3 (45 mg, 0.20 mmol, 68.65%) as yellow solid.

LC-MS (ESI) m/z: 226.1 [M+H]⁺

Step 3: A suspension of B13-3 (45 mg, 0.20 mmol), 4-iodo-1-(trifluoromethyl)benzene (0.04 mL, 0.30 mmol), CuI (22.83 mg, 0.12 mmol), N,N′-dimethylethylenediamine (21.13 mg, 0.240 mmol) and K₃PO₄ (84.81 mg, 0.40 mmol) in DMF (2 mL) was stirred at 130° C. for 12 h under nitrogen protection. TLC showed the reaction was completed. The mixture was extracted with EtOAc (15 mL*3) and water (20 mL), the combined organic layer was washed with brine (20 mL), dried and filtered. The filtrate was concentrated under reduced pressure. The residue was purified by combi flash (0-7% EtOAc/Petroleum ether) to afford B13-4 (60 mg, 0.162 mmol, 81.31%) as yellow solid.

LC-MS (ESI) m/z: 370.1 [M+H]⁺

Step 4: According to the step 5 of A8, B13 was synthesized.

LC-MS (ESI) m/z: 356.1 [M+H]⁺

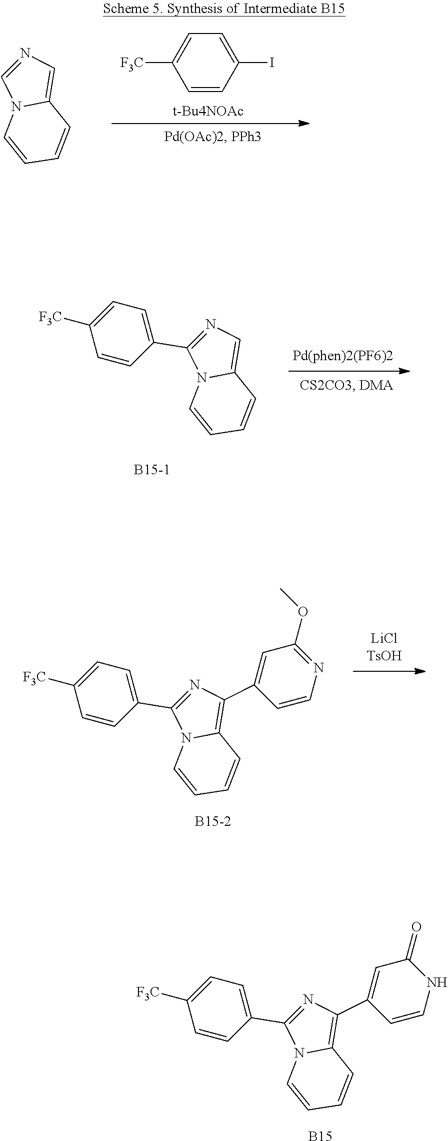

Step 1. To a solution of imidazo[1,5-a]pyridine (236 mg, 1.998 mmol) and 4-iodo-1-(trifluoromethyl)benzene (543 mg, 2.00 mmol) in toluene (2.5 mL) was added Pd(OAc)₂ (22.4 mg, 0.100 mmol), PPh₃ (52.4 mg, 0.20 mmol) and t-Bu₄NOAc (1.20 g, 3.99 mmol), and the mixture was stirred at 100° C. for 16 h under N₂ atmosphere. The mixture was diluted with water (30 mL) and the mixture was extracted with EtOAc (20 mL×4). The combined organic layer was washed with brine, dried over anhydrous Na₂SO₄, filtered and concentrated under reduced pressure. The residue was purified by flash column chromatography on silica gel (Gradient elution, 0-20% EtOAc/PE) to afford compound B15-1 (300 mg, 57.3%) as a yellow solid.

LC-MS (ESI) m/z: 263.1 [M+H]⁺.

Step 2. To a solution of compound B15-1 (250 mg, 0.953 mmol) in DMA (7.5 mL) was added Pd(phen)₂(PF₆)₂ (30 mg, 0.040 mmol), 4-iodo-2-methoxypyridine (291 mg, 1.24 mmol) and Cs₂CO₃ (621 mg, 1.91 mmol), and the mixture was stirred at 150° C. for 15 h in a sealed tube under N2 atmosphere. The mixture was diluted with water (50 mL) and the mixture was extracted with EtOAc (30 mL×3). The combined organic layer was washed with brine, dried over anhydrous Na₂SO₄, filtered and concentrated under reduced pressure. The residue was purified by flash column chromatography on silica gel (Gradient elution, 0-20% EtOAc/PE) to afford compound B15-2 (300 mg, 85.20%) as a brown solid.

LC-MS (ESI) m/z: 370.1 [M+H]⁺.

Step 3. To a solution of compound B15-2 (100 mg, 0.271 mmol) in DMF (3 mL) was added LiCl (57.4 mg, 1.35 mmol) and p-Toluenesulfonic acid monohydrate (257 mg, 1.35 mmol), and the mixture was stirred at 130° C. for 1 h under N₂ atmosphere. The mixture was diluted with water (20 mL) and the mixture was extracted with EtOAc (30 mL×3). The combined layer was washed with brine, dried over anhydrous Na₂SO₄, filtered and concentrated under reduced pressure to afford intermediate B15 (80 mg, 83.2%) as a yellow solid.

LC-MS (ESI) m/z: 356.1 [M+H]⁺.

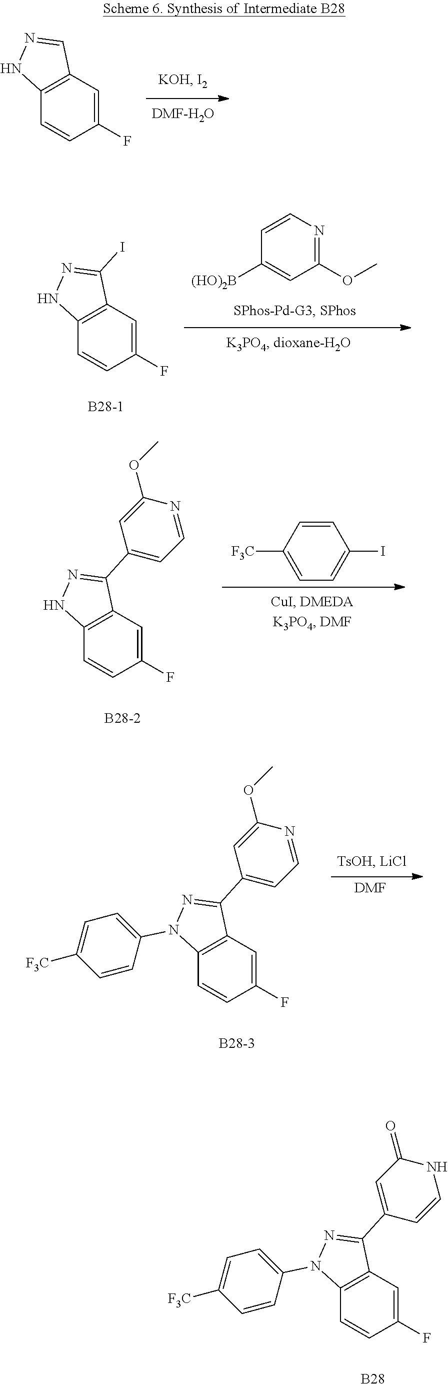

Step 1: To a solution of 5-fluoro-1H-indazole (545 mg, 4.00 mmol) in DMF (10 mL) and water (0.5 mL) was added KOH (1.31 g, 6.00 mmol) followed by 12 (1.52 g, 6.00 mmol) at 20° C. The mixture was stirred for 1 h at 20° C. The reaction was quenched with quenched with aq. Na₂SO₃ and 3N aq. HCl, diluted with water. The mixture was extracted with MTBE twice. The combined organic layer washed with brine, dried over Na₂SO₄, filtered and concentrated under reduced pressure to give compound B28-1 (1.05 g, 100%) as a yellow solid.

LC-MS (ESI) m/z: 262.7 [M+H]⁺.

Step 2: The mixture of compound B28-1 (262 mg, 1.00 mmol) in dioxane (4 mL) and water (1 mL) was added 2-methoxypyridine-4-boronic acid (306 mg, 2.00 mmol), SPhos-Pd-G3 (23 mg, 0.03 mmol), SPhos (12 mg, 0.03 mmol) and K₃PO₄·3H₂O (533 mg, 2.00 mmol) at 20° C. The mixture was stirred for 16 h at 100° C. under nitrogen. The reaction mixture was diluted with water and 3N·aq·HCl (1.5 mL), and extracted with MTBE twice. The combined organic layer washed with brine, dried over Na₂SO₄, filtered and concentrated under reduced pressure. The residual oil was absorbed on silica gel and purified by combi flash (26% EtOAc in Heptane) to give compound B28-2 (119 mg, 49%) as a white solid.

LC-MS (ESI) m/z: 243.9 [M+H]⁺.

Step 3: To a solution of compound B28-2 (119 mg, 0.489 mmol) in dry DMF (2.5 mL) was added CuI (55.9 mg, 0.294 mmol) and DMEDA (52 mg, 63 μL, 0.587 mmol) followed by K₃PO₄ (208 mg, 0.978 mmol). The mixture was heated for 16 h at 130° C. under nitrogen. The reaction was diluted with lots of H₂O and aq. NH₃ (6 mL) and stirred for 20 min. The mixture was filtered and filter cake washed with H₂O. The filter cake was dried at 60° C. to give compound B28-3 (169 mg, 89%) as a light yellow solid.

LC-MS (ESI) m/z: 387.9 [M+H]⁺.

Step 4: To a solution of compound B28-3 (169 mg, 0.436 mmol) in DMF (4 mL) was added LiCl (93 mg, 2.18 mmol) and TsOH·H₂O (415 mg, 2.18 mmol). The mixture was stirred at 120° C. for 12 h. The solution was diluted with water and stirred for 20 min. The mixture was filtered and filter cake was washed with water. The solid was dried at 50° C. to give intermediate B28 (150 mg, 92%) as a light yellow solid.

LC-MS (ESI) m/z: 373.9 [M+H]⁺.

The following intermediates listed below were synthesized according to the procedure of B28:

| Intermediate | | |
|---|---|---|
| No. | Structure | Analytical data |
| B4 | 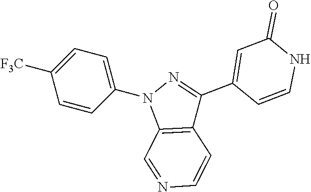 [SMILES: O=c1cc(-c2nn(-c3ccc(C(F)(F)F)cc3)c3cnccc23)cc[nH]1] | LCMS: m/z 357.1 [M + H]⁺. |
| B5 | 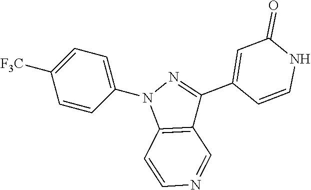 [SMILES: O=c1cc(-c2nn(-c3ccc(C(F)(F)F)cc3)c3ccncc23)cc[nH]1] | LCMS: m/z 357.1 [M + H]⁺. |
| B10 | 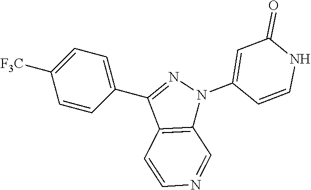 [SMILES: O=c1cc(-n2nc(-c3ccc(C(F)(F)F)cc3)c3ccncc32)cc[nH]1] | LCMS: m/z 357.1 [M + H]⁺. |
| B11 | 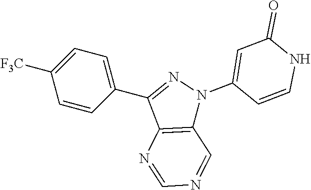 [SMILES: O=c1cc(-n2nc(-c3ccc(C(F)(F)F)cc3)c3ncncc32)cc[nH]1] | LCMS: m/z 358.1 [M + H]⁺. |
| B14 | 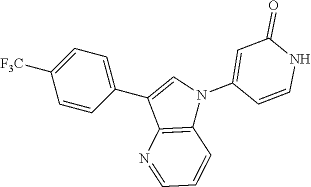 [SMILES: O=c1cc(-n2cc(-c3ccc(C(F)(F)F)cc3)c3ncccc32)cc[nH]1] | LCMS: m/z 356.1 [M + H]⁺. |
| B25 | 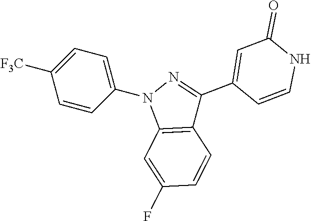 [SMILES: O=c1cc(-c2nn(-c3ccc(C(F)(F)F)cc3)c3cc(F)ccc23)cc[nH]1] | LCMS: m/z 374.1 [M + H]⁺. |
| B26 | 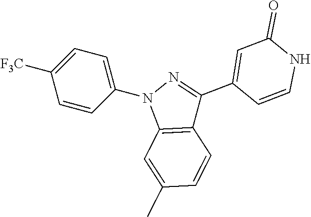 [SMILES: Cc1ccc2c(-c3cc[nH]c(=O)c3)nn(-c3ccc(C(F)(F)F)cc3)c2c1] | LCMS: m/z 370.1 [M + H]⁺. |
| B29 | 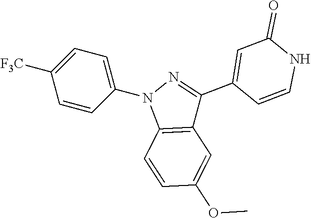 [SMILES: COc1ccc2c(c1)c(-c1cc[nH]c(=O)c1)nn2-c1ccc(C(F)(F)F)cc1] | LCMS: m/z 386.1 [M + H]⁺. |

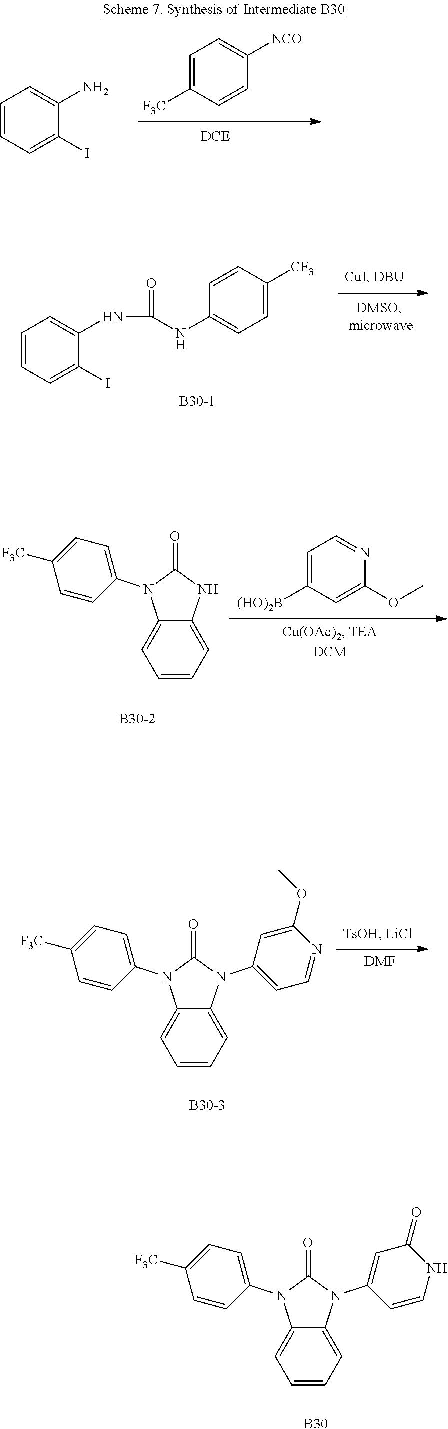

Step 1: To a solution of 4-(trifluoromethyl)phenyl isocyanate (1.12 g, 6.00 mmol) in DCE (30 mL) was added 2-iodoaniline (1.31 g, 6.00 mmol) at 20° C. The mixture was stirred at 70° C. for 16 h. The mixture was cooled to room temperature. The mixture was filtered and the filter cake washed with DCM. The solid was collected to give compound B30-1 (2.15 g, 88%) as a white solid.

LC-MS (ESI) m/z: 356.1 [M+H]⁺.

Step 2: To a solution of B30-1 (2.38 g, 5.85 mmol) in dry DMSO (20 mL) was added CuI (223 mg, 1.17 mmol) and DBU (1.78 g, 11.7 mmol) at 20° C. The mixture was heated at 120° C. for 0.5 h under nitrogen by microwave irradiation. The mixture was cooled to room temperature and poured into H₂O and 3N aq. HCl, stirred for 5 min. The mixture was the filtered and filter cake washed with water. The solid was dried at 60° C. to give compound B30-2 (1.36 g, 83%) as a light yellow solid.

LC-MS (ESI) m/z: 356.1 [M+H]⁺.

Step 3: To a mixture of B30-2 (584 mg, 2.10 mmol), 2-methoxypyridine-4-boronic acid (385 mg, 2.52 mmol) and copper (II) acetate (499 mg, 2.73 mmol) in DCM (14 mL) was add TEA (638 mg, 6.30 mmol). The mixture was stirred for 16 h at 20° C. The mixture was diluted with aq. NH₃ and water, and extracted with DCM twice. The combined organic layer was dried over Na₂SO₄, filtered and concentrated under reduced pressure. The residue was absorbed on silica gel and purified by Combi flash (20% EtOAc in Heptane) to afford compound B30-3 (491 mg, 60%) as a colorless solid

LC-MS (ESI) m/z: 356.1 [M+H]⁺.

Step 4: To a solution of B30-3 (453 mg, 1.18 mmol) in DMF (10 mL) was added LiCl (150 mg, 3.53 mmol) and TsOH·H₂O (671 mg, 3.53 mmol). The mixture was stirred at 20° C. for 17 h. The solution was diluted with water and stirred for 20 min. The mixture was filtered and the filter cake was washed with water. The solid was dried at 60° C. to give intermediate B30 (436 mg, 100%) as a white solid.

LC-MS (ESI) m/z: 371.9 [M+H]⁺

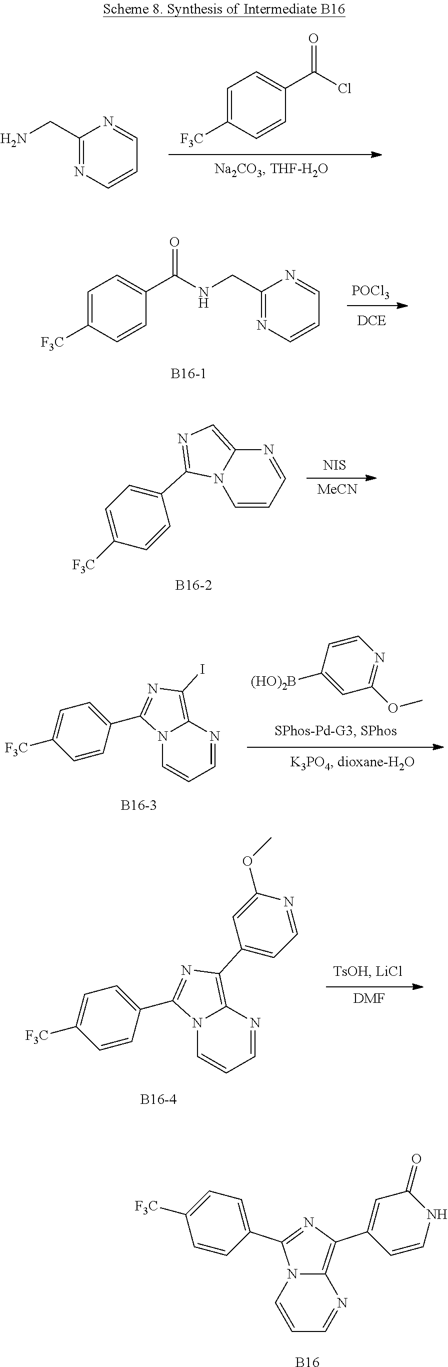

Step 1: To a solution of 2-aminomethyl pyrimidine (546 mg, 5.00 mmol) in THF (15 mL) and water (15 mL) was added Na₂CO₃ (1.06 g, 10.0 mmol) followed by 4-(trifluoromethyl)benzoyl chloride (1.04 g, 5.00 mmol) at 20° C. The mixture was stirred for 1 h. The mixture was diluted with water and extracted with MTBE twice. The combined organic layer washed with brine, dried over Na₂SO₄, filtered and concentrated under reduced pressure to obtain compound B16-1 (1.34 g, 95%) as a light yellow solid.

LC-MS (ESI) m/z: 281.9 [M+H]⁺

Step 2: To a solution of compound B16-1 (703 mg, 2.50 mmol) in DCE (15 mL) was added POCl₃ (4.66 mL, 50 mmol). The mixture was heated for 16 h at 90° C. The solution was cooled to room temperature and slowly poured into water, and then neutralized with solid Na₂CO₃ to pH=8. The mixture was extracted with EA twice. The combined organic layer was dried over Na₂SO₄, filtered and concentrated under reduced pressure. The residue was absorbed on silica gel and purified by Combi flash (60% EtOAc in Heptane) to afford compound B16-2 (419 mg, 64%) as a yellow solid.

LC-MS (ESI) m/z: 263.9 [M+H]⁺

Step 3: To a solution of compound B16-2 (360 mg, 1.37 mmol) in MeCN (10 mL) was added NIS (461.6 mg, 2.05 mmol). The solution was stirred for 1 h at 20° C. The reaction was quenched with aq. Na₂SO₃ solution and diluted with water. The mixture was extracted with EA twice. The combined organic layer dried over Na₂SO₄, filtered and concentrated under reduced pressure. The residual oil was absorbed on silica gel and purified by Combi flash (30% EtOAc in Heptane) to afford compound B16-3 (43 mg, 83%) as a yellow solid.

LC-MS (ESI) m/z: 389.9 [M+H]⁺

Step 4: According to the step 4 of B30, B16 was synthesized. LC-MS (ESI) m/z: 357.1 [M+H]⁺

The following intermediates listed below were synthesized according to the procedure of B16:

| Intermediate | | |
|---|---|---|
| No. | Structure | Analytical data |
| B17 | 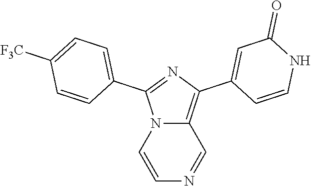 [SMILES: O=c1cc(-c2nc(-c3ccc(C(F)(F)F)cc3)n3ccncc23)cc[nH]1] | LCMS: m/z 357.1 [M + H]⁺. |
| B18 | 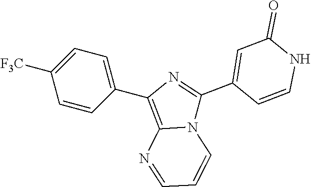 [SMILES: O=c1cc(-c2nc(-c3ccc(C(F)(F)F)cc3)c3ncccn23)cc[nH]1] | LCMS: m/z 357.1 [M + H]⁺. |
| B19 | 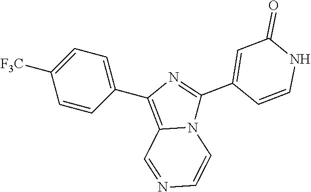 [SMILES: O=c1cc(-c2nc(-c3ccc(C(F)(F)F)cc3)c3cnccn23)cc[nH]1] | LCMS: m/z 357.1 [M + H]⁺. |
| B20 | 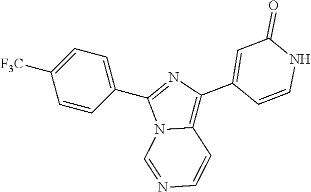 [SMILES: O=c1cc(-c2nc(-c3ccc(C(F)(F)F)cc3)n3cnccc23)cc[nH]1] | LCMS: m/z 357.1 [M + H]⁺. |
| B21 | 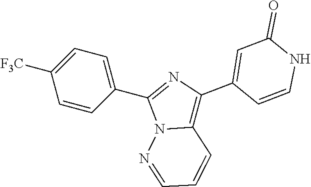 [SMILES: O=c1cc(-c2nc(-c3ccc(C(F)(F)F)cc3)n3ncccc23)cc[nH]1] | LCMS: m/z 357.1 [M + H]⁺. |
| B23 | 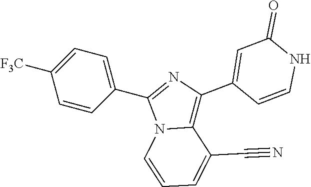 [SMILES: N#Cc1cccn2c(-c3ccc(C(F)(F)F)cc3)nc(-c3cc[nH]c(=O)c3)c12] | LCMS: m/z 381.1 [M + H]⁺. |

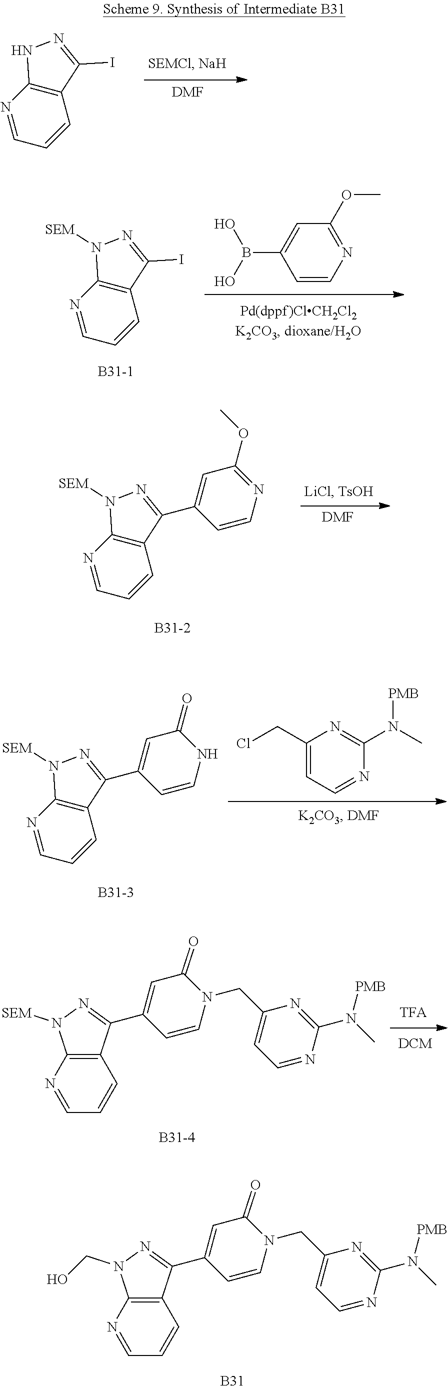

Step 1: To a solution of 3-iodo-1H-pyrazolo[3,4-b]pyridine (10 g, 40.8 mmol) in DMF (100 mL) was added NaH (3.27 g, 81.6 mmol) at 0° C. under N₂. To the mixture was added 2-(Trimethylsilyl)ethoxymethyl Chloride (7.48 g, 44.9 mmol) and the mixture was stirred at 25° C. for overnight. The mixture was diluted with water (300 mL). The mixture was extracted with EtOAc (200 mL×2). The combined layer was washed with brine, dried over anhydrous Na₂SO₄, filtered and concentrated under reduced pressure. The residue was purified by flash column chromatography on silica gel (Gradient elution, 10% EA in Heptane) to afford compound B31-1 (12.0 g, 78.4%) as a white solid.

LC-MS (ESI) m/z: 376.0 [M+H]⁺.

Step 2: To a solution of compound B31-1 (7.0 g, 18.7 mmol) in dioxane (75 mL) was added (2-methoxypyridin-4-yl)bornanediol (3.42 g, 22.4 mmol), K₂CO₃ (5.16 g, 37.3 mmol), Pd(dppf)Cl₂·CH₂Cl₂ (1.52 g, 1.87 mmol) and water (15 mL). The mixture was stirred at 100° C. for 16 h under N₂. The mixture was filtered and washed with EA. The filtrate was concentrated under reduced pressure. The residue was purified by flash column chromatography on silica gel (12% of EA Heptane) to afford compound B31-2 (6.5 g, 97.8%) as yellow oil.

LC-MS (ESI) m/z: 357.2 [M+H]⁺.

Step 3: To a solution of compound B31-2 (490 mg, 1.37 mmol) in DMF (10 mL) were added LiCl (291 mg, 6.87 mmol) and p-Toluenesulfonic acid (1.18 g, 6.87 mmol), and the mixture was stirred at 120° C. for 2 h. The mixture was diluted with water (30 mL). Then the mixture was extracted with EtOAc (40 mL×2). The combined layer was washed with brine, dried over anhydrous Na₂SO₄, filtered and concentrated under reduced pressure. The residue was purified by flash column chromatography on silica gel (Gradient elution, 0-10% MeOH/DCM) to afford compound B31-3 (380 mg, 80.7%) as a white solid.

LC-MS (ESI) m/z: 343.2 [M+H]⁺

Step 4: To a solution of compound B31-3 (350 mg, 1.02 mmol) and 4-(chloromethyl)-2-{[(4-methoxyphenyl)methyl](methyl)amino}pyrimidine (284 mg, 1.02 mmol) in DMF (10 mL) was added K₂CO₃ (282 mg, 2.04 mmol), and the mixture was stirred at 40° C. for 12 h. The mixture was diluted with water (40 mL). The mixture was extracted with EtOAc (40 mL>2). The combined layer was washed with brine, dried over anhydrous Na₂SO₄, filtered and concentrated under reduced pressure. The residue was purified by flash column chromatography on silica gel (Gradient elution, 0-100% EtOAc/PE) to afford compound B31-4 (520 mg, 87.2%) as colorless oil.

LC-MS (ESI) m/z: 584.3 [M+H]⁺

Step 5: A solution of compound B31-4 (500 mg, 0.857 mmol) in TFA (5 mL) and DCM (5 mL) was stirred at 20° C. for 12 h. The mixture was concentrated under reduced pressure. the residue was adjusted pH to 8 with saturated aq. NaHCO₃, then the mixture was extracted with DCM (30 mL>2). The combined layer was washed with brine, dried over anhydrous Na₂SO₄, filtered and concentrated under reduced pressure. The residue was purified by flash column chromatography on silica gel (Gradient elution, 0-10% MeOH/DCM) to afford compound B31 (340 mg, 82.1%) as yellow oil.

LC-MS (ESI) m/z: 484.2 [M+H]⁺.

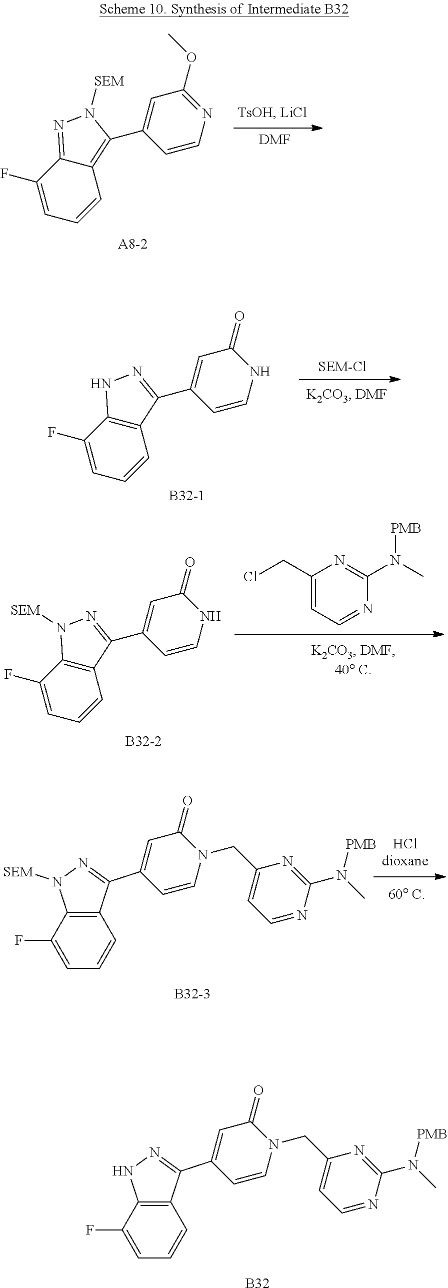

Step 1: To a solution of compound A8-2 (2.30 g, 6.16 mmol) in DMF (50 mL) were added LiCl (1.31 g, 30.8 mmol) and p-Toluenesulfonic acid (5.30 g, 30.8 mmol), and the reaction mixture was stirred at 120° C. for 2 h. The mixture was diluted with water (100 mL). The resulting suspension was filtered, and the filter cake was dried to afford compound B32-1 (1.10 g, 77.9%) as a white solid.

LC-MS (ESI) m/z: 230.1 [M+H]⁺.

Step 2: To a solution of compound B32-1 (800 mg, 3.49 mmol) in DMF (90 mL) were added K₂CO₃ (965 mg, 6.98 mmol) and 1-chloro-5,5-dimethyl-2-oxa-5-silahexane (582 mg, 3.49 mmol), and the mixture was stirred at 40° C. for 1 h. The mixture was diluted with water (160 mL). The mixture was extracted with EtOAc (100 mL>2). The combined layer was washed with brine, dried over anhydrous Na₂SO₄, filtered and concentrated under reduced pressure. The residue was purified by flash column chromatography on silica gel (Gradient elution, 0-10% MeOH/DCM) to afford compound B32-2 (900 mg, 71.7%) as a white solid.

LC-MS (ESI) m/z: 360.1 [M+H]⁺.

Step 3: To a suspension of compound B32-2 (900 mg, 2.50 mmol) in DMF (20 mL) was added 4-(chloromethyl)-2-{[(4-methoxyphenyl)methyl](methyl)amino}pyrimidine (765 mg, 2.75 mmol) and K₂CO₃ (692 mg, 5.01 mmol), and the mixture was stirred at 40° C. for 2 h. The mixture was diluted with water (50 mL). The mixture was extracted with EtOAc (60 mL×2). The combined layer was washed with brine, dried over anhydrous Na₂SO₄, filtered and concentrated under reduced pressure. The residue was purified by flash column chromatography on silica gel (Gradient elution, 0-100% EtOAc/PE) to afford compound B32-3 (1.20 g, 79.8%) as yellow oil.

LC-MS (ESI) m/z: 601.3 [M+H]⁺

Step 4: To a solution of compound B32-3 (1.20 g, 2.00 mmol) in MeOH (20 mL) was added HCl/dioxane (20 mL) (4.0 M), and the mixture was stirred at 60° C. for 12 h. The mixture was concentrated under reduced pressure. The residue was adjusted pH to 8 with saturated aq. NaHCO₃. The mixture was extracted with EtOAc (40 mL×2). The combined layer was washed with brine, dried over anhydrous Na₂SO₄, filtered and concentrated under reduced pressure. The residue was triturated with EtOAc (20 mL), and the resulting suspension was filtered to afford compound B32 (710 mg, 75.6%) as a white solid.

LC-MS (ESI) m/z: 471.2 [M+H]⁺

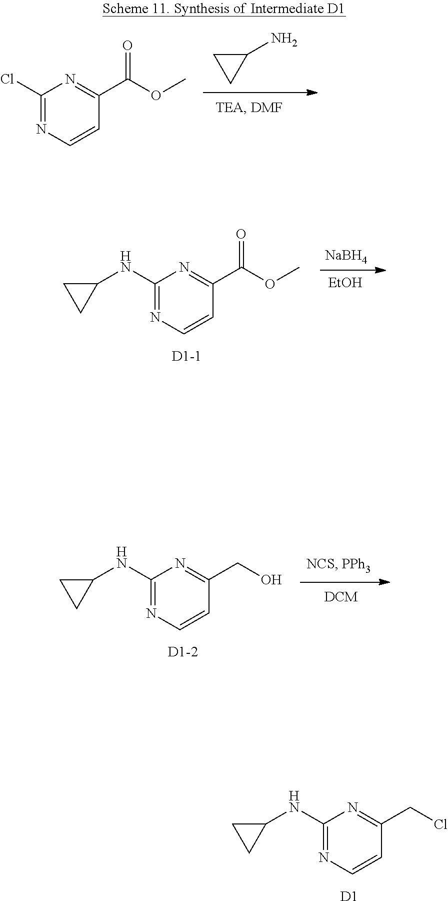

Step 1: To a stirred solution of methyl 2-chloropyrimidine-4-carboxylate (500 mg, 2.90 mmol) and TEA (0.8 mL, 5.80 mmol) in DMF (20 mL) were added cyclopropanamine (0.3 mL, 4.35 mmol), the mixture was stirred at 40° C. for 16 h. The reaction was poured into water, and extracted with DCM. The organic layers were combined and dried over Na₂SO₄. The dried solution was filtered, and the filtrate was concentrated in vacuo to yield a pale yellow solid which was purified by flash chromatography (gradient elution 0-80% EtOAc in hexanes) to afford compound D1-1 (300 mg, 53.59%) as a yellow solid.

LC-MS (ESI) m/z: 194.1 [M+H]⁺.

Step 2: To a solution of compound D1-1 (200 mg, 1.04 mmol) in EtOH (4 mL) were added NaBH₄ (78 mg, 2.07 mmol), and the mixture was stirred at 20° C. for 12 h. To the mixture was added MeOH (5 mL), and the mixture was stirred at 20° C. for 0.5 h. the reaction mixture was concentrated under reduced pressure. the residue was purified by flash column chromatography on silica gel (Gradient elution, 0-5% MeOH/DCM) to afford compound D1-2 (150 mg, 87.7%) as colorless oil.

LC-MS (ESI) m/z: 166.1 [M+H]⁺.

Step 3: To a solution of compound D1-2 (120 mg, 0.726 mmol) in DCM (5 mL) was added PPh₃ (229 mg, 0.872 mmol) and NCS (116 mg, 0.872 mmol) portionwise at 0° C. The reaction mixture was stirred at 20° C. for 0.5 h. The reaction mixture was evaporated in vacuo to dryness. The residue was purified by flash column chromatography on silica gel (Gradient elution, 0-10% EtOAc/DCM) to afford compound D1 (40 mg, 30.0%) as colorless oil.

LC-MS (EST) m/z: 184.1 [M+H]⁺.

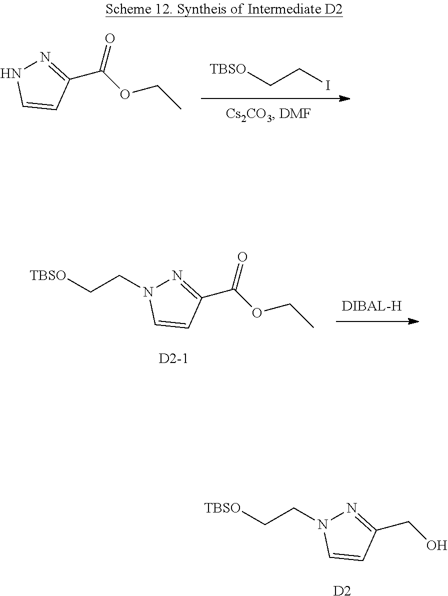

Step 1: To a solution of ethyl 1H-pyrazole-3-carboxylate (1.0 g, 7.14 mmol) in DMF (20 mL) were added 1-iodo-4,4,5,5-tetramethyl-3-oxa-4-silahexane (2.45 g, 8.56 mmol) and Cs₂CO₃ (4.65 g, 14.3 mmol), and the mixture was stirred at 20° C. for 12 h. The mixture was diluted with water (40 mL). The mixture was extracted with PE/EtOAc (1/1, 50 mL>2). The combined layer was washed with brine, dried over anhydrous Na₂SO₄, filtered and concentrated under reduced pressure. The residue was purified by flash column chromatography on silica gel (Gradient elution, 0-10% EtOAc/Heptane) to afford compound D2-1 (1.05 g, 49.30%) as colorless oil.

LC-MS (ESI) m/z: 299.2 [M+H]⁺.

Step 2: To a solution of compound D2-1 (500 mg, 1.68 mmol) in DCM (5 mL) was added Diisobutylaluminum Hydride (10.1 mL, 10.1 mmol) at 0° C., and the mixture was stirred at 0° C. for 0.5 h and stirred at 20° C. for 0.5 h. The reaction was quenched with Potassium sodium tartrate solution (5 mL, 2.0 M) and stirred for 30 min at 20° C. The mixture was extracted with DCM (10 mL×2). The combined layer was washed with brine, dried over anhydrous Na₂SO₄, filtered and concentrated under reduced pressure. The residue was purified by flash column chromatography on silica gel (Gradient elution, 0-50% EtOAc/Heptane) to afford compound D2 (300 mg, 69.8%) as colorless oil.

LC-MS (ESI) m/z: 257.2 [M+H]⁺.

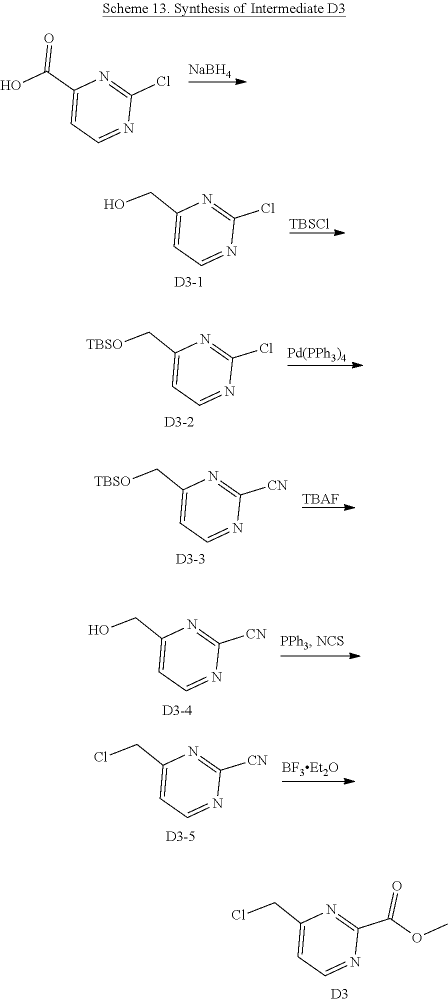

Step 1: To a solution of 2-chloropyrimidine-4-carboxylic acid (2 g, 12.62 mmol) and Et₃N (1.93 mL, 13.88 mmol) in THF (40 mL) was slowly added Isobutyl Chloroformate (1.81 mL, 13.88 mmol). Reaction mixture was stirred at 25° C. for 1 h. The formed precipitate was filtered off and to obtained clear solution was slowly added solution of NaBH₄ (0.95 g, 25.23 mmol) in H₂O (8 mL). Reaction mixture was stirred at 25° C. for 30 min. Then water (100 mL) was added, and the product was extracted with EtOAc (40 mL*3). Combined organic fractions were washed with brine (60 mL) and dried with Na₂SO₄. After evaporation of the solvents under reduced pressure, the residue was purified by combi flash to afford compound D3-1 (860 mg, 5.95 mmol, 47.16%) as white solid.

LC-MS (ESI) m/z: 144.9 [M+H]⁺.

Step 2: To a solution of compound D3-1 (860 mg, 5.95 mmol) and 1H-imidazole (0.60 mL, 8.92 mmol) in DCM (20 mL) was added chlorodimethyl(2-methylprop-2-yl)silane (1.55 mL, 8.92 mmol) at 25° C. The white suspension was stirred at 25° C. for 1 h. TLC showed the reaction was completed. The mixture was extracted with water (40 mL) and DCM (30 mL*2), the combined organic layer was washed with brine (30 mL), dried and filtered. The filtrate was concentrated under reduced pressure. The residue was purified by combi flash (0-10% EtOAc/Petroleum ether) to afford compound D3-2 (1.36 g, 5.256 mmol, 88.33%) as yellow oil.

LC-MS (ESI) m/z: 259.1 [M+H]⁺.

Step 3: A suspension of compound D3-2 (500 mg, 1.93 mmol), Zn(CN)₂ (136.10 mg, 1.16 mmol) and Pd(PPh₃)₄ (223.24 mg, 0.19 mmol) in anhydrous DMF (10 mL) was stirred at 100° C. for 16 h. TLC showed the reaction was completed. The mixture was filtered. The filtrate was extracted with EtOAc (20 mL*3) and water (60 mL). The combined organic layer was washed with brine (60 mL), dried and filtered. The filtrate was concentrated under reduced pressure. The residue was purified by combi flash (0-10% EToAc/Petroleum ether) to afford compound D3-3 (300 mg, 1.20 mmol, 62.27%) as colorless oil.

¹H-NMR (400 MHz, CDCl₃) δ 8.82 (d, J=5.2 Hz, 1H), 7.75 (dt, J=5.2, 1.0 Hz, 1H), 4.81 (s, 2H), 0.96 (s, 9H), 0.14 (s, 6H).

Step 4: To a solution of compound D3-3 (340 mg, 1.36 mmol) in MeOH (2 mL) was added HCl/dioxane (0.5 mL) at 25° C. The yellow solution was stirred at 25° C. for 2 h. TLC showed the reaction was completed. The mixture was adjusted to pH=8 with sat·NaHCO₃ and extracted with EtOAc (10 mL*3). The combined organic layer was washed with brine (15 mL), dried and filtered. The filtrate was concentrated under reduced pressure. The residue was purified by combi flash (0-30% EtOAc/Petroleum ether) to afford compound D3-4 (170 mg, 1.26 mmol, 92.28%) as yellow oil.

Step 5: To a solution of compound D3-4 (120 mg, 0.89 mmol) and PPh₃ (349.38 mg, 1.33 mmol) in DCM (2 mL) was added NCS (177.87 mg, 1.33 mmol) at 25° C. The yellow solution was stirred at 25° C. for 2 h. TLC showed the reaction was completed. The mixture was concentrated under reduced pressure. The residue was purified by combi flash (0-20% EtOAc/Petroleum ether) to afford compound D3-5 (130 mg, 0.85 mmol, 95.33%) as yellow oil.

LC-MS (EST) m/z: 154.1 [M+H]⁺.

Step 6: To a solution of compound D3-5 (110 mg, 0.72 mmol) in MeOH (3 mL) was added BF₃·OEt₂ (3 mL) at 25° C. The brown solution was stirred at 80° C. for 48 h. The starting material was remained, and the desired MS peak was detected. The mixture was adjusted to pH=8 with sat·NaHCO₃ and extracted with EtOAc (10 mL*3). The combined organic layer was washed with brine (20 mL), dried and filtered. The filtrate was concentrated under reduced pressure. The residue was purified by combi flash (0-30% EtOAc/Petroleum ether) to afford compound D3 (35 mg, 0.19 mmol, 26.19%) as yellow oil.

LC-MS (ESI) m/z: 187.1 [M+H]⁺.

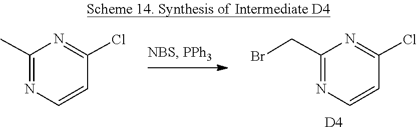

Step 1: A suspension of 4-chloro-2-methylpyrimidine (100 mg, 0.78 mmol), NBS (207.67 mg, 1.17 mmol) and AIBN (63.87 mg, 0.39 mmol) in CCl₄ (5 mL) was stirred at 80° C. for 3 days. The desired MS peak was detected and the starting material was remained. The mixture was concentrated under reduced pressure. The residue was purified by combi flash (0-10% EtOAc/Heptane) to afford compound D4 (30 mg, 0.15 mmol, 18.59%) as colorless oil.

LC-MS (ESI) m/z: 209.1 [M+H]⁺.

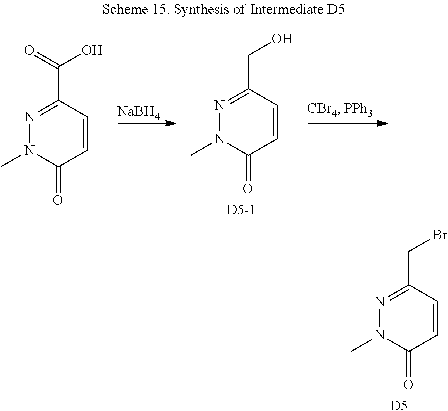

Step 1: To a solution of 1-methyl-6-oxo-1,2-diazine-3-carboxylic acid (200 mg, 1.30 mmol) and Et₃N (0.20 mL, 1.43 mmol) in THF (4 mL) was slowly added Isobutyl Chloroformate (0.19 mL, 1.43 mmol). Reaction mixture was stirred at 25° C. for 1 h. The formed precipitate was filtered off and to obtained clear solution was slowly added solution of NaBH₄ (98.18 mg, 2.60 mmol) in H₂O (1 mL). Reaction mixture was stirred at 25° C. for 30 min. Then water (15 mL) was added, and the product was extracted with EtOAc (10 mL*3). Combined organic fractions were washed with brine (20 mL) and dried with Na₂SO₄. After evaporation of the solvents under reduced pressure, the residue was purified by combi flash to afford compound D5-1 (140 mg, 1.00 mmol, 77.78%) as white solid.

LC-MS (ESI) m/z: 140.9 [M+H]⁺.

Step 2: A solution of compound D5-1 (70 mg, 0.50 mmol), tetrabromomethane (331.30 mg, 1.00 mmol) and PPh₃ (262.03 mg, 1.00 mmol) in DCM (2 mL) was stirred at 40° C. for 2 h. TLC showed the reaction was completed. The mixture was concentrated under reduced pressure. The residue was purified by combi flash (0-25% EtOAc/Petroleum ether) to afford compound D5 (60 mg, 0.30 mmol, 59.16%) as white solid.

LC-MS (ESI) m/z: 205.1 [M+H]⁺.

### Example 1: Synthesis of Compound 1

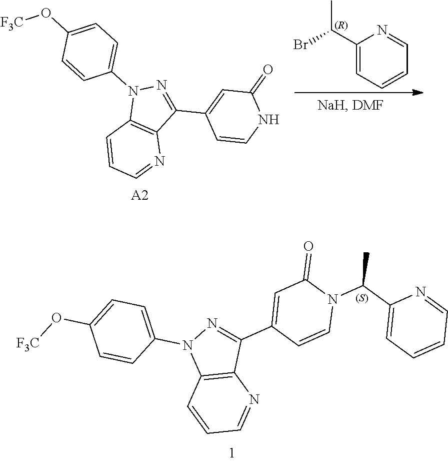

To a solution of compound A2 (50 mg, 0.134 mmol) in DMF (4 mL) were added NaH (10.74 mg, 0.269 mmol, 60% in mineral oil), and the mixture was stirred at 20° C. for 10 min. To the mixture was added 2-[(1R)-1-bromoethyl]pyridine (37 mg, 0.201 mmol), and the mixture was stirred at 20° C. for 3 h. The mixture was diluted with water (20 mL). The mixture was extracted with EtOAc (25 mL×2). The combined organic layer was washed with brine, dried over anhydrous Na₂SO₄, filtered and concentrated under reduced pressure. The residue was purified by flash column chromatography on silica gel (gradient elution, 0-100% EtOAc/PE) to afford compound 1 (32 mg, 43.5% of yield) as an off-white solid.

LC-MS (ESI) m/z: 478.1 [M+H]⁺.

¹H-NMR (400 MHz, CDCl₃) δ 8.74 (dd, J=4.4, 1.6 Hz, 1H), 8.65-8.58 (m, 1H), 8.07 (dd, J=8.8, 1.6 Hz, 1H), 8.01 (d, J=1.6 Hz, 1H), 7.82-7.74 (m, 2H), 7.73-7.60 (m, 2H), 7.47-7.37 (m, 4H), 7.32 (dd, J=7.2, 2.0 Hz, 1H), 7.25-7.18 (m, 1H), 6.55 (q, J=7.2 Hz, 1H), 1.84 (d, J=7.2 Hz, 3H).

The following compounds listed below were synthesized according to the procedure of compound 1:

| | From | | | |
|---|---|---|---|---|
| Compound | Inter- | | | |
| No. | mediate | Structure | Name | Analytical data |
| 1 | A6 | 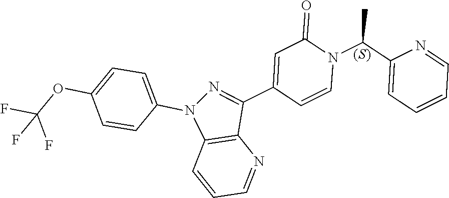 | (S)-1-(1-(pyridin-2-yl)ethyl)- 4-(1-(4- (trifluoromethoxy)phenyl)- 1H-pyrazolo[4,3-b]pyridin-3- yl)pyridin-2(1H)-one | ¹H-NMR (400 MHz, CDCl₃) δ 8.74 (dd, J = 4.4, 1.6 Hz, 1H), 8.65-8.58 (m, 1H), 8.07 (dd, J = 8.8, 1.6 Hz, 1H), 8.01 (d, J = 1.6 Hz, 1H), 7.82-7.74 (m, 2H), 7.73-7.60 (m, 2H), 7.47-7.37 (m, 4H), 7.32 (dd, J = 7.2, 2.0 Hz, 1H), 7.25-7.18 (m, 1H), 6.55 (q, J = 7.2 Hz, 1H), 1.84 (d, J = 7.2 Hz, 3H). LCMS: m/7. 478.1 [M + H]⁺. |
| 2 | A2 | 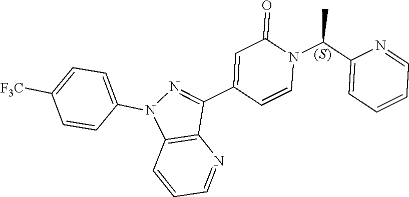 | (S)-1-(1-(pyridin-2-yl)ethyl)- 4-(1-(4- (trifluoromethyl)phenyl)- 1H- pyrazolo[4,3-b]pyridin-3- yl)pyridin-2(1H)-one | ¹H-NMR (400 MHz, CDCl₃) δ 8.75 (dd, J = 4.4, 1.2 Hz, 1H), 8.65-8.57 (m, 1H), 8.13 (dd, J = 8.4, 1.2 Hz, 1H), 8.05-7.99 (m, 1H), 7.91 (d, J = 8.0 Hz, 2H), 7.84 (d, J = 8.4 Hz, 2H), 7.72 (d, J = 7.2 Hz, 1H), 7.66 (td, J = 8.0, 1.6 Hz, 1H), 7.47-7.38 (m, 2H), 7.32 (dd, J = 7.6, 2.0 Hz, 1H), 7.25-7.19 (m, 1H), 6.54 (q, J = 7.2 Hz, 1H), 1.85 (d, J = 7.2 Hz, 3H). LCMS: m/z 462.1 [M + H]⁺. |
| 3 | A2 | 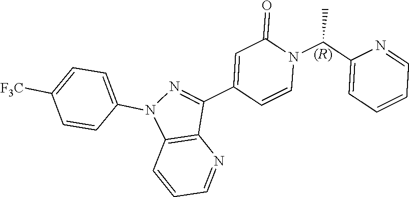 | (R)-1-(1-(pyridin-2-yl)ethyl)- 4-(1-(4- (trifluoromethyl)phenyl)-1H- pyrazolo[4,3-b]pyridin-3- yl)pyridin-2(1H)-one | ¹H-NMR (400 MHz, CDCl₃) δ 8.75 (dd, J = 4.4, 1.6 Hz, 1H), 8.65-8.57 (m, 1H), 8.13 (dd, J = 8.4, 1.2 Hz, 1H), 8.05-7.99 (m, 1H), 7.91 (d, J = 8.0 Hz, 2H), 7.84 (d, J = 8.4 Hz, 2H), 7.72 (d, J = 7.2 Hz, 1H), 7.66 (td, J = 8.0, 1.6 Hz, 1H), 7.47-7.38 (m, 2H), 7.32 (dd, J = 7.6, 2.0 Hz, 1H), 7.25-7.19 (m, 1H), 6.54 (q, J = 7.2 Hz, 1H), 1.85 (d, J = 7.2 Hz, 3H). LCMS: m/z 462.1 [M + H]⁺. |
| 4 | A4 | 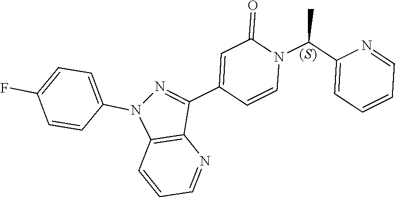 | (S)-4-(1-(4-fluorophenyl)- 1H-pyrazolo[4,3-b]pyridin-3- yl)-1-(1-(pyridin-2- yl)ethyl)pyridin-2(1H)-one | ¹H-NMR (400 MHz, CDCl₃) δ 8.73 (dd, J = 4.4, 1.2 Hz, 1H), 8.64-8.57 (m, 1H), 8.05-7.96 (m, 2H), 7.74-7.63 (m, 4H), 7.43-7.36 (m, 2H), 7.35- 7.25 (m, 3H), 7.24-7.18 (m, 1H), 6.55 (q, J = 7.2 Hz, 1H), 1.84 (d, J = 7.2 Hz, 3H). LCMS: m/z 412.2 [M + H]⁺. |
| 5 | A5 | 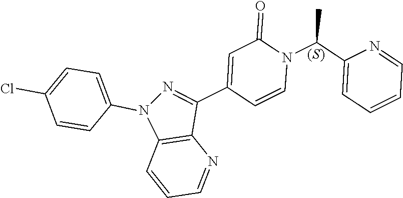 | (S)-4-(1-(4-chlorophenyl)- 1H-pyrazolo[4,3-b]pyridin-3- yl)-1-(1-(pyridin-2- yl)ethyl)pyridin-2(1H)-one | ¹H-NMR (400 MHz, CDCl₃) δ 8.73 (dd, J = 4.4, 1.2 Hz, 1H), 8.65-8.58 (m, 1H), 8.05 (dd, J = 8.8, 1.6 Hz, 1H), 8.00 (d, J = 2.0 Hz, 1H), 7.73-7.62 (m, 4H), 7.58-7.51 (m, 2H), 7.44-7.36 (m, 2H), 7.32 (dd, J = 7.6, 2.0 Hz, 1H), 7.25-7.18 (m, 1H), 6.55 (q, J = 7.2 Hz, 1H), 1.85 (d, J = 7.2 Hz, 3H). LCMS: m/z 428.1 [M + H]⁺. |
| 6 | A3 | 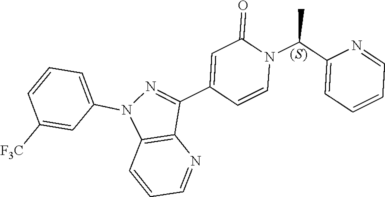 | (S)-1-(1-(pyridin-2-yl)ethyl)- 4-(1-(3- (trifluoromethyl)phenyl)-1H- pyrazolo[4,3-b]pyridin-3- yl)pyridin-2(1H)-one | ¹H-NMR (400 MHz, CDCl₃) δ 8.76 (dd, J = 4.4, 1.2 Hz, 1H), 8.65-8.58 (m, 1H), 8.10 (dd, J = 8.8, 1.2 Hz, 1H), 8.06-8.00 (m, 2H), 7.99-7.93 (m, 1H), 7.76-7.62 (m, 4H), 7.48-7.37 (m, 2H), 7.33 (dd, J = 7.2, 2.0 Hz, 1H), 7.25-7.18 (m, 1H), 6.55 (q, J = 7.2 Hz, 1H), 1.85 (d, J = 7.2 Hz, 3H). LCMS: m/z 462.2 [M + H]⁺. |
| 7 | A2 | 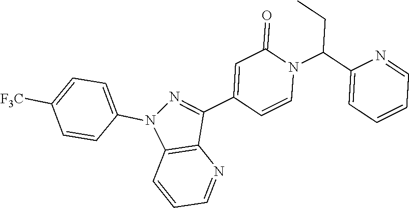 [SMILES: CCC(c1ccccn1)n1ccc(-c2nn(-c3ccc(C(F)(F)F)cc3)c3cccnc23)cc1=O] | 1-(1-(pyridin-2-yl)propyl)-4- (1-(4- (trifluoromethyl)phenyl)-1H- pyrazolo[4,3-b]pyridin-3- yl)pyridin-2(1H)-one | ¹H-NMR (400 MHz, CDCl₃) δ 8.75 (dd, J = 4.4, 1.2 Hz, 1H), 8.65-8.58 (m, 1H), 8.13 (dd, J = 8.4, 1.2 Hz, 1H), 8.00 (d, J = 2.0 Hz, 1H), 7.95-7.80 (m, 5H), 7.65 (td, J = 7.6, 1.6 Hz, 1H), 7.49-7.38 (m, 2H), 7.32 (dd, J = 7.6, 2.0 Hz, 1H), 7.24-7.18 (m, 1H), 6.31 (t, J = 8.0 Hz, 1H), 2.50-2.34 (m, 1H), 2.28-2.12 (m, 1H), 0.97 (t, J = 7.6 Hz, 3H). LCMS: m/z 476.2 [M + H]⁺. |
| 8 | A2 | 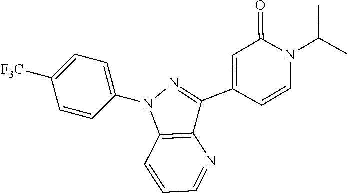 [SMILES: CC(C)n1ccc(-c2nn(-c3ccc(C(F)(F)F)cc3)c3cccnc23)cc1=O] | 1-isopropyl-4-(1-(4- (trifluoromethyl)phenyl)-1H- pyrazolo[4,3-b]pyridin-3- yl)pyridin-2(1H)-one | ¹H-NMR (400 MHz, CDCl₃) δ 8.77 (dd, J = 4.4, 1.2 Hz, 1H), 8.15 (dd, J = 8.8, 1.6 Hz, 1H), 8.05-7.99 (m, 1H), 7.93 (d, J = 8.4 Hz, 2H), 7.85 (d, J = 8.4 Hz, 2H), 7.50-7.42 (m, 2H), 7.41-7.34 (m, 1H), 5.44-5.31 (m, 1H), 1.42 (d, J = 6.8 Hz, 6H). LCMS: m/z 399.1 [M + H]⁺. |
| 9 | B7 | 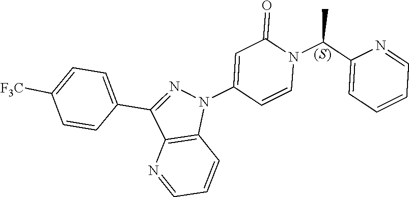 | (S)-1-(1-(pyridin-2-yl)ethyl)- 4-(3-(4- (trifluoromethyl)phenyl)-1H- pyrazolo[4,3-b]pyridin-1- yl)pyridin-2(1H)-one | ¹H-NMR (400 MHz, CDCl₃) δ 8.78 (dd, J = 4.4, 1.2 Hz, 1H), 8.76-8.70 (m, 2H), 8.66-8.59 (m, 1H), 8.29 (dd, J = 8.8, 1.6 Hz, 1H), 7.88 (d, J = 7.6 Hz, 1H), 7.78 (d, J = 8.0 Hz, 2H), 7.69 (td, J = 7.6, 1.6 Hz, 1H), 7.50-7.40 (m, 2H), 7.26-7.20 (m, 1H), 7.01 (dd, J = 7.6, 2.8 Hz, 1H), 6.91 (d, J = 2.4 Hz, 1H), 6.49 (q, J = 7.2 Hz, 1H), 1.85 (d, J = 7.2 Hz, 3H). LCMS: m/z 462.2 [M + H]⁺. |
| 10 | A2 | 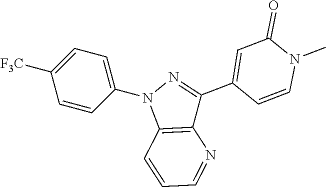 [SMILES: Cn1ccc(-c2nn(-c3ccc(C(F)(F)F)cc3)c3cccnc23)cc1=O] | 1-methyl-4-(1-(4- (trifluoromethyl)phenyl)-1H- pyrazolo[4,3-b]pyridin-3- yl)pyridin-2(1H)-one | ¹H-NMR (400 MHz, CDCl₃) δ 8.77 (dd, J = 4.4, 1.2 Hz, 1H), 8.14 (dd, J = 8.4, 1.2 Hz, 1H), 8.10 (d, J = 2.0 Hz, 1H), 7.93 (d, J = 8.4 Hz, 2H), 7.85 (d, J = 8.4 Hz, 2H), 7.48-7.38 (m, 2H), 7.35 (dd, J = 6.8, 1.6 Hz, 1H), 3.63 (s, 3H). LCMS: m/z 371.1 [M + H]⁺. |
| 11 | A1 | 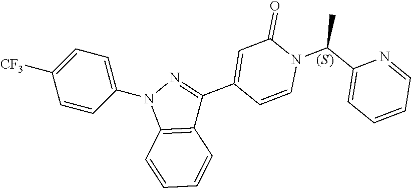 | (S)-1-(1-(pyridin-2-yl)ethyl)- 4-(1-(4- (trifluoromethyl)phenyl)-1H- indazol-3-yl)pyridin-2(1H)- one | ¹H-NMR (400 MHz, CDCl₃) δ 8.63 (dt, J = 4.8, 1.6 Hz, 1H), 8.14 (dt, J = 8.4, 1.0 Hz, 1H), 7.92 (d, J = 8.4 Hz, 2H), 7.82 (d, J = 8.4 Hz, 3H), 7.74 (d, J = 7.2 Hz, 1H), 7.68 (td, J = 7.8, 2.0 Hz, 1H), 7.53 (ddd, J = 8.4, 7.2, 1.2 Hz, 1H), 7.45-7.35 (m, 2H), 7.25-7.21 (m, 1H), 6.97 (dd, J = 7.2, 2.0 Hz, 1H), 6.52 (q, J = 7.2 Hz, 1H), 1.86 (d, J = 7.2 Hz, 3H) LCMS: m/z 461.1 [M + H]⁺ |
| 12 | A1 | 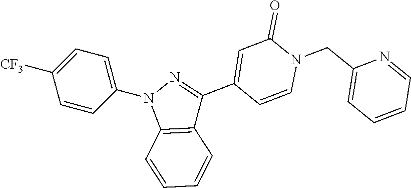 [SMILES: O=c1cc(-c2nn(-c3ccc(C(F)(F)F)cc3)c3ccccc23)ccn1Cc1ccccn1] | 1-(pyridin-2-ylmethyl)-4-(1- (4-(trifluoromethyl)phenyl)- 1H-indazol-3-yl)pyridin-2 (1H)-one | ¹H-NMR (400 MHz, CDCl₃) δ 8.58 (ddd, J = 4.8, 1.6, 0.8 Hz, 1H), 8.15 (dt, J = 8.0, 0.8 Hz, 1H), 7.93 (d, J = 8.4 Hz, 2H), 7.85-7.79 (m, 3H), 7.69 (td, J = 7.6, 1.6 Hz, 1H), 7.65 (d, J = 7.2 Hz, 1H), 7.48- 7.44 (m, 1H), 7.41-7.37 (m, 1H), 7.29 (d, J = 2.0 Hz, 1H), 7.25-7.21 (m, 1H), 7.01 (dd, J = 7.2, 2.0 Hz, 1H), 5.32 (s, 2H). LCMS: m/z 447.1 [M + H]⁺ |
| 13 | A1 | 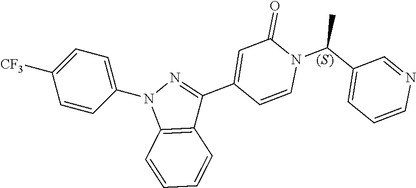 | (S)-1-(1-(pyridin-3-yl)ethyl)- 4-(1-(4- (trifluoromethyl)phenyl)-1H- indazol-3-yl)pyridin-2(1H)- one | ¹H-NMR (400 MHz, CDCl₃) δ 8.69 (d, J = 2.4 Hz, 1H), 8.59 (dd, J = 4.8, 1.6 Hz, 1H), 8.15 (d, J = 8.0 Hz, 1H), 7.92 (d, J = 8.4 Hz, 2H), 7.83 (d, J = 8.4 Hz, 3H), 7.68 (dt, J = 8.0, 2.0 Hz, 1H), 7.55 (ddd, J = 8.4, 7.2, 1.2 Hz, 1H), 7.40 (dd, J = 8.0, 7.2 Hz, 1H), 7.36-7.30 (m, 2H), 7.28 (s, 1H), 6.98 (dd, J = 7.2, 2.0 Hz, 1H), 6.53 (q, J = 7.2 Hz, 1H), 1.85 (d, J = 7.2 Hz, 3H). LCMS: m/z 461.1 [M + H]⁺ |
| 14 | A1 | 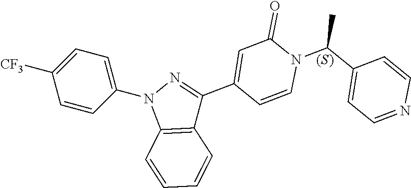 | (S)-1-(1-(pyridin-4-yl)ethyl)- 4-(1-(4- (trifluoromethyl)phenyl)-1H- indazol-3-yl)pyridin-2(1H)- one | ¹H-NMR (400 MHz, CDCl₃) δ 8.63 (s, 2H), 8.16 (d, J = 8.4 Hz, 1H), 7.93 (d, J = 8.4 Hz, 2H), 7.89-7.80 (m, 3H), 7.55 (ddd, J = 8.4, 7.2, 0.8 Hz, 1H), 7.41 (ddd, J = 8.0, 7.2, 0.8 Hz, 1H), 7.33 (d, J = 2.0 Hz, 1H), 7.25-7.21 (m, 2H), 7.01 (dd, J = 7.2, 2.0 Hz, 1H), 6.46 (q, J = 7.2 Hz, 1H), 1.82 (d, J = 7.2 Hz, 3H). LCMS: m/z 461.1 [M + H]⁺ |
| 15 | A1 | 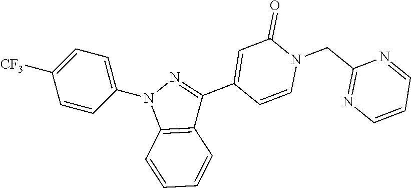 [SMILES: O=c1cc(-c2nn(-c3ccc(C(F)(F)F)cc3)c3ccccc23)ccn1Cc1ncccn1] | 1-(pyrimidin-2-ylmethyl)-4- (1-(4- (trifluoromethyl)phenyl)-1H- indazol-3-yl)pyridin-2(1H)- one | ¹H-NMR (400 MHz, CDCl₃) δ 8.78-8.68 (m, 2H), 8.18 (d, J = 8.4 Hz, 1H), 7.95 (d, J = 8.4 Hz, 2H), 7.84 (d, J = 8.4 Hz, 3H), 7.54 (dd, J = 10.0, 7.2 Hz, 2H), 7.39 (t, J = 7.6 Hz, 1H), 7.34 (d, J = 1.2 Hz, 1H), 7.30-7.22 (m, 1H), 7.07 (dd, J = 7.2, 1.6 Hz, 1H), 5.45 (s, 2H). LCMS: m/z 448.1 [M + H]⁺ |
| 16 | A1 | 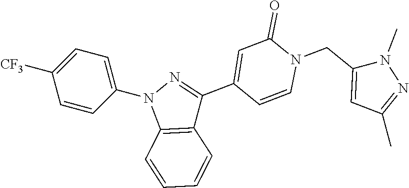 [SMILES: Cc1cc(Cn2ccc(-c3nn(-c4ccc(C(F)(F)F)cc4)c4ccccc34)cc2=O)n(C)n1] | 1-((1,3-dimethyl-1H-pyrazol- 5-yl)methyl)-4-(1-(4- (trifluoromethyl)phenyl)-1H- indazol-3-yl)pyridin-2(1H)- one | ¹H-NMR (400 MHz, CDCl₃) δ 8.14 (d, J = 8.4 Hz, 1H), 7.93 (d, J = 8.4 Hz, 2H), 7.83 (dd, J = 8.8, 1.6 Hz, 3H), 7.55 (ddd, J = 8.4, 7.2, 1.2 Hz, 1H), 7.40 (ddd, J = 8.0, 7.2, 0.8 Hz, 1H), 7.35-7.29 (m, 2H), 7.00 (dd, J = 7.2, 2.0 Hz, 1H), 6.10 (s, 1H), 5.21 (s, 2H), 3.83 (s, 3H), 2.25 (s, 3H). LCMS: m/z 464.1 [M + H]⁺ |
| 17 | A1 | 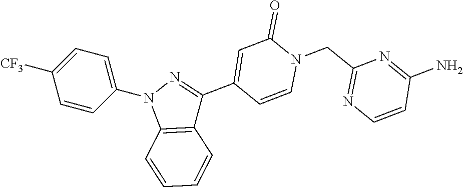 [SMILES: Nc1ccnc(Cn2ccc(-c3nn(-c4ccc(C(F)(F)F)cc4)c4ccccc34)cc2=O)n1] | 1-((4-aminopyrimidin-2- yl)methyl)-4-(1-(4- (trifluoromethyl)phenyl)-1H- indazol-3-yl)pyridin-2(1H)- one | ¹H-NMR (400 MHz, DMSO-d₆) δ 8.23 (d, J = 8.4 Hz, 1H), 8.14 (d, J = 8.4 Hz, 2H), 8.05 (d, J = 8.4 Hz, 1H), 8.02-7.95 (m, 3H), 7.87 (d, J = 7.2 Hz, 1H), 7.70-7.61 (m, 1H), 7.46 (t, J = 7.6 Hz, 1H), 7.06 (d, J = 2.0 Hz, 1H), 6.95 (dd, J = 7.2, 2.0 Hz, 1H), 6.88 (s, 2H), 6.30 (d, J = 6.0 Hz, 1H), 5.06 (s, 2H). LCMS: m/z 463.1 [M + H]+ |
| 18 | A1 | 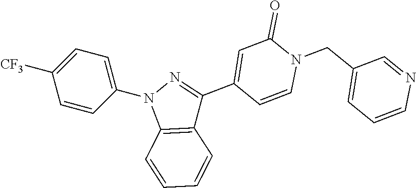 [SMILES: O=c1cc(-c2nn(-c3ccc(C(F)(F)F)cc3)c3ccccc23)ccn1Cc1cccnc1] | 1-(pyridin-3-ylmethyl)-4-(1- (4-(trifluoromethyl)phenyl)- 1H-indazol-3-yl)pyridin- 2(1H)-one | ¹H-NMR (400 MHz, DMSO-d₆) δ 8.66 (d, J = 2.0 Hz, 1H), 8.52 (dd, J = 4.8, 1.6 Hz, 1H), 8.24 (d, J = 8.4 Hz, 1H), 8.14 (d, J = 8.4 Hz, 2H), 8.06 (dd, J = 10.0, 8.0 Hz, 2H), 8.00 (d, J = 8.4 Hz, 2H), 7.79 (dt, J = 8.0, 2.0 Hz, 1H), 7.68-7.60 (m, 1H), 7.50- 7.44 (m, 1H), 7.41 (dd, J = 8.0, 4.8 Hz, 1H), 7.11 (d, J = 2.0 Hz, 1H), 7.00 (dd, J = 7.2, 2.0 Hz, 1H), 5.23 (s, 2H). LCMS: m/z 447.1 [M + H]⁺ |
| 19 | A1 | 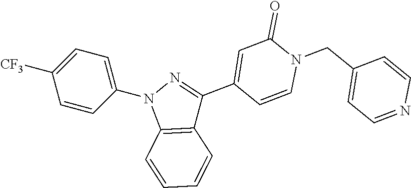 [SMILES: O=c1cc(-c2nn(-c3ccc(C(F)(F)F)cc3)c3ccccc23)ccn1Cc1ccncc1] | 1-(pyridin-4-ylmethyl)-4-(1- (4-(trifluoromethyl)phenyl)- 1H-indazol-3-yl)pyridin- 2(1H)-one | ¹H-NMR (400 MHz, DMSO-d₆) δ 8.56 (dd, J = 8.4, 1.6 Hz 2H), 8.26 (d, J = 8.4 Hz, 1H), 8.14 (d, J = 8.4 Hz, 2H), 8.05 (d, J = 8.4 Hz, 1H), 8.00 (d, J = 7.6 Hz, 3H), 7.65 (t, J = 7.6 Hz. 1H), 7.47 (t, J = 7.6 Hz 1H), 7.29 (d, J = 6.0 Hz. 1H), 7.15 (d, J = 2.0 Hz, 1H), 7.03 (dd, J = 7.2, 2.0 Hz, 1H), 5.24 (s, 2H). LCMS: m/z 447.1 [M + H]⁺ |
| 20 | B1 | 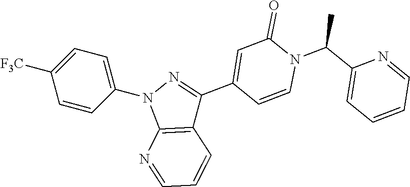 [SMILES: C[C@@H](c1ccccn1)n1ccc(-c2nn(-c3ccc(C(F)(F)F)cc3)c3ncccc23)cc1=O] | (S)-1-(1-(pyridin-2-yl)ethyl)- 4-(1-(4- (trifluoromethyl)phenyl)-1H- pyrazolo[3,4-b]pyridin-3- yl)pyridin-2(1H)-one | ¹H-NMR (400 MHz, DMSO-d₆) δ 8.80 (m, 2H), 8.67 (d, J = 8.6 Hz, 2H), 8.58 (d, J = 4.0 Hz, 1H), 8.00 (d, J = 8.7 Hz, 2H), 7.93 (d, J = 7.3 Hz, 1H), 7.83 (td, J = 7.7, 1.8 Hz, 1H), 7.55 (dd, J = 8.2, 4.6 Hz, 1H), 7.42 (d, J = 7.9 Hz, 1H), 7.34 (dd, J = 6.6, 4.9 Hz, 1H), 7.15 (d, J = 1.9 Hz, 1H), 7.03 (dd, J = 7.3, 2.0 Hz, 1H), 6.25 (d, J = 7.2 Hz, 1H), 1.78 (d, J = 7.2 Hz, 3H). LCMS: m/z 462.1 [M + H]⁺. |
| 22 | B27 | 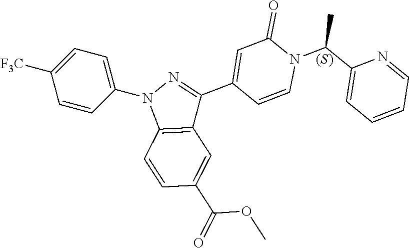 | methyl (S)-3-(2-oxo-1-(1- (pyridin-2-yl)ethyl)-1,2- dihydropyridin-4-yl)-1-(4- (trifluoromethyl)phenyl)-1H- indazole-5-carboxylate | ¹H-NMR (400 MHz, DMSO_d₆) δ 8.72 (s, 1H), 8.59 (dt, J = 5.2, 1.2 Hz, 1H), 8.20-8.10 (m, 3H), 8.03 (d, J = 8.4 Hz, 2H), 7.97 (d, J = 7.2 Hz, 1H), 7.84 (td, J = 7.6, 1.8 Hz, 1H), 7.60 (ddd, J = 18.4, 11.2, 7.2 Hz, 1H), 7.44 (d, J = 8.0 Hz, 1H), 7.35 (ddd, J = 7.6, 4.8, 1.2 Hz, 1H), 7.06 (d, J = 2.0 Hz, 1H), 6.94 (dd, J = 7.2, 2.0 Hz, 1H), 6.26 (q, J = 7.2 Hz, 1H), 3.95 (s, 3H), 1.79 (d, J = 7.2 Hz, 3H). LCMS: m/z 519.1 [M + H]⁺. |
| 26 | B24 | 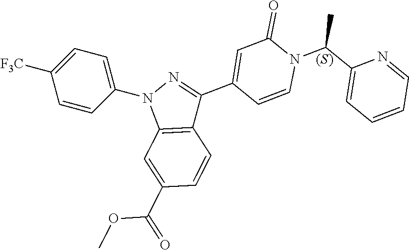 | methyl (S)-3-(2-oxo-1-(1- (pyridin-2-yl)ethyl)-1,2- dihydropyridin-4-yl)-1-(4- (trifluoromethyl)phenyl)-1H- indazole-6-carboxylate | ¹H-NMR (400 MHz, DMSO_d₆) δ 8.60-8.54 (m, 1H), 8.45 (t, J = 1.2 Hz, 1H), 8.36 (d, J = 8.4 Hz, 1H), 8.13 (d, J = 8.4 Hz, 2H), 8.05 (d, J = 8.8 Hz, 2H), 7.97 (dd, J = 8.8, 1.2 Hz, 1H), 7.91 (d, J = 7.2 Hz, 1H), 7.82 (td, J = 7.6, 1.9 Hz, 1H), 7.41 (d, J = 8.0 Hz, 1H), 7.33 (ddd, J = 7.6, 4.8, 1.2 Hz, 1H), 7.09 (d, J = 2.0 Hz, 1H), 6.96 (dd, J = 7.2, 2.0 Hz, 1H), 6.24 (q, J = 7.2 Hz, 1H), 3.92 (s, 3H), 1.77 (d, J = 7.2 Hz, 3H). LCMS: m/z 519.1 [M + H]⁺. |
| 32 | B8 | 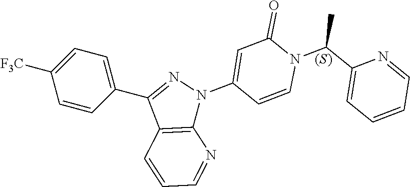 | (S)-1-(1-(pyridin-2-yl)ethyl)- 4-(3-(4- (trifluoromethyl)phenyl)-1H- pyrazolo[3,4-b]pyridin-1- yl)pyridin-2(1H)-one | ¹H-NMR (400 MHz, MeOD) δ 8.74 (dd, J = 4.6, 1.5 Hz, 1H), 8.65 (dd, J = 8.2, 1.5 Hz, 1H), 8.57 (d, J = 4.1 Hz, 1H), 8.30 (d, J = 8.1 Hz, 2H), 7.99 (d, J = 2.4 Hz, 1H), 7.95 (d, J = 7.8 Hz, 1H), 7.88 (d, J = 8.2 Hz, 2H), 7.83-7.80 (m, 2H), 7.52-7.47 (m, 2H), 7.37-7.33 (m, 1H), 6.32 (d, J = 7.2 Hz, 1H), 1.87 (d, J = 7.2 Hz, 3H). LCMS: m/z 462.1 [M + H]⁺. |
| 33 | B28 | 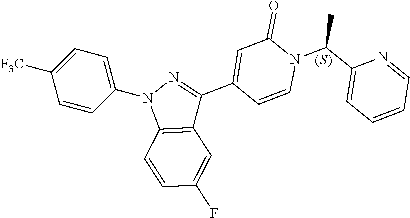 | (S)-4-(5-fluoro-1-(4- (trifluoromethyl)phenyl)-1H- indazol-3-yl)-1-(1-(pyridin- 2-yl)ethyl)pyridin-2(1H)-one | ¹H-NMR (400 MHz, CDCl₃) δ 8.63 (d, J = 4.0 Hz, 1H), 7.93-7.66 (m, 8H), 7.45 (d, J = 7.9 Hz, 1H), 7.34-7.26 (m, 2H), 7.16 (d, J = 1.8 Hz, 1H), 6.92 (dd, J = 7.3, 1.9 Hz, 1H), 6.51 (q, J = 7.1 Hz, 1H), 1.87 (d, J = 7.1 Hz, 3H). LCMS: m/z 478.9 [M + H]⁺. |
| 34 | B29 | 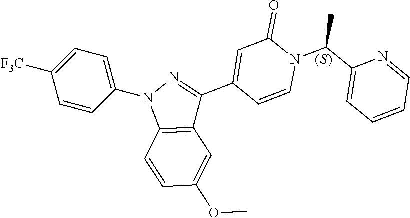 | (S)-4-(5-methoxy-1-(4- (trifluoromethyl)phenyl)-1H- indazol-3-yl)-1-(1-(pyridin- 2-yl)ethyl)pyridin-2(1H)-one | ¹H-NMR (400 MHz, CDCl₃) δ 8.63 (d, J = 4.0 Hz, 1H), 7.90 (d, J = 8.4 Hz, 2H), 7.81 (d, J = 8.6 Hz, 2H), 7.77-7.64 (m, 3H), 7.41 (t, J = 5.5 Hz, 2H), 7.20 (ddd, J = 12.4, 8.4, 2.5 Hz, 3H), 6.95 (dd, J = 7.3, 1.9 Hz, 1H), 6.52 (q, J = 7.1 Hz, 1H), 3.92 (s, 3H), 1.86 (d, J = 7.1 Hz, 3H). LCMS: m/z 490.9 [M + H]⁺. |
| 35 | B15 | 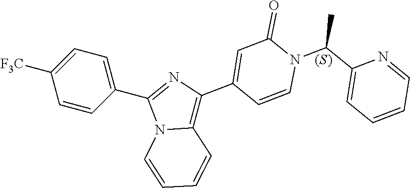 | (S)-1-(1-(pyridin-2-yl)ethyl)- 4-(3-(4- (trifluoromethyl)phenyl) imidazo[1,5-a]pyridin-1- yl)pyridin-2(1H)-one | ¹H-NMR (400 MHz, MeOD) δ 8.54 (dd, J = 20.2, 5.7 Hz, 2H), 8.05 (t, J = 10.3 Hz, 3H), 7.89 (d, J = 8.2 Hz, 2H), 7.82 (dd, J = 4.5, 2.8 Hz, 2H), 7.43 (d, J = 7.9 Hz, 1H), 7.33 (dd, J = 7.1, 5.3 Hz, 1H), 7.15- 7.10 (m, 2H), 7.05 (d, J = 1.8 Hz, 1H), 6.90 (s, 1H), 6.32 (d, J = 7.2 Hz, 1H), 1.85 (d, J = 7.2 Hz, 3H). LCMS: m/z. 461.1 [M + H]⁺. |
| 40 | A7 | 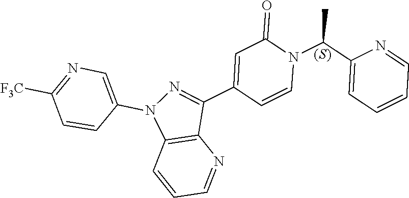 | (S)-1-(1-(pyridin-2-yl)ethyl)- 4-(1-(6- (trifluoromethyl)pyridin-3- yl)-1H-pyrazolo[4,3- b]pyridin-3-yl)pyridin- 2(1H)-one | ¹H-NMR (400 MHz, CDCl₃) δ 9.23 (d, J = 2.4, 1H), 8.80 (dd, J = 4.4, 1.2 Hz, 1H), 8.67-8.59 (m, 1H), 8.33 (dd, J = 8.4, 2.4 Hz, 1H), 8.16 (dd, J = 8.8, 1.6 Hz, 1H), 8.04 (d, J = 1.6 Hz, 1H), 7.93 (d, J = 8.4 Hz, 1H), 7.85-7.70 (m, 2H), 7.56-7.45 (m, 2H), 7.36-7.27 (m, 2H), 6.52 (q, J = 7.2 Hz, 1H), 1.89 (d, J = 7.2 Hz, 3H). LCMS: m/z 463.1 [M + H]⁺. |
| 41 | B7 | 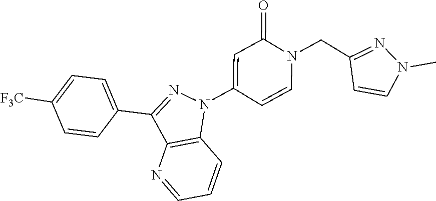 [SMILES: Cn1ccc(Cn2ccc(-n3nc(-c4ccc(C(F)(F)F)cc4)c4ncccc43)cc2=O)n1] | 1-((1-methyl-1H-pyrazol-3- yl)methyl)-4-(3-(4- (trifluoromethyl)phenyl)-1H- pyrazolo[4,3-b]pyridin-1- yl)pyridin-2(1H)-one | LCMS: m/z 451.1 [M + H]⁺. 1H-NMR (400 MHz, CDCl3) δ 8.79 (d, J = 4.2 Hz, 1H), 8.74 (d, J = 8.1 Hz, 2H), 8.34 (d, J = 8.6 Hz, 1H), 7.78 (d, J = 8.2 Hz, 2H), 7.69 (d, J = 7.7 Hz, 1H), 7.50 (dd, J = 8.6, 4.4 Hz, 1H), 7.34 (d, J = 2.0 Hz, 1H), 7.10-6.99 (m, 2H), 6.36 (d, J = 2.2 Hz, 1H), 5.23 (s, 2H), 3.91 (s, 3H). |
| 42 | B7 | 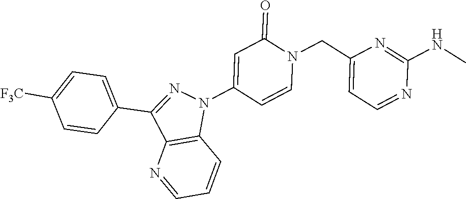 [SMILES: CNc1nccc(Cn2ccc(-n3nc(-c4ccc(C(F)(F)F)cc4)c4ncccc43)cc2=O)n1] | 1-((2- (methylamino)pyrimidin-4- yl)methyl)-4-(3-(4- (trifluoromethyl)phenyl)- 1H-pyrazolo[4,3-b]pyridin- 1-yl)pyridin-2(1H)-one | LCMS: m/z 478.2 [M + H]⁺. 1H-NMR (400 MHz, CDCl3) δ 8.81 (dd, J = 4.4, 1.3 Hz, 1H), 8.75 (d, J = 8.1 Hz, 2H), 8.32 (dd, J = 8.7, 1.3 Hz, 1H), 8.23 (dtd, J = 7.8, 4.2, 3.6, 1.5 Hz, 1H), 7.79 (d, J = 8.2 Hz, 2H), 7.63 (d, J = 7.5 Hz, 1H), 7.51 (dd, J = 8.7, 4.4 Hz, 1H), 7.11 (dd, J = 7.5, 2.5 Hz, 1H), 6.96 (d, J = 2.5 Hz, 1H), 6.59 (d, J = 5.2 Hz, 1H), 5.11 (s, 2H), 2.99 (d, J = 5.0 Hz, 3H). |
| 43 | B7 | 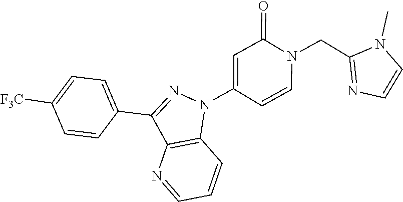 [SMILES: Cn1ccnc1Cn1ccc(-n2nc(-c3ccc(C(F)(F)F)cc3)c3ncccc32)cc1=O] | 1-((1-methyl-1H-imidazol-2- yl)methyl)-4-(3-(4- (trifluoromethyl)phenyl)- 1H-pyrazolo[4,3-b]pyridin- 1-yl)pyridin-2(1H)-one | LCMS: m/z 451.1 [M + H]⁺. 1H-NMR (400 MHz, CDCl3) δ 8.79 (dd, J = 4.4, 1.3 Hz, 1H), 8.76-8.69 (m, 2H), 8.26 (dd, J = 8.7, 1.4 Hz, 1H), 8.10 (d, J = 7.6 Hz, 1H), 7.78 (d, J = 8.2 Hz, 2H), 7.48 (dd, J = 8.7, 4.4 Hz, 1H), 7.12 (d, J = 1.5 Hz, 1H), 7.07 (dd, J = 7.6, 2.5 Hz, 1H), 6.95 (d, J = 1.4 Hz, 1H), 6.92 (d, J = 2.5 Hz, 1H), 5.41 (s, 2H), 3.93 (s, 3H). |
| 44 | B1 | 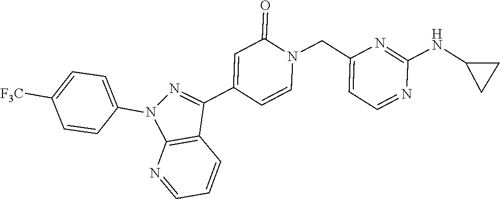 [SMILES: O=c1cc(-c2nn(-c3ccc(C(F)(F)F)cc3)c3ncccc23)ccn1Cc1ccnc(NC2CC2)n1] | 1-((2- (cyclopropylamino)pyrimidin- 4-yl)methyl)-4-(1-(4- (trifluoromethyl)phenyl)- 1H-pyrazolo[3,4-b]pyridin- 3-yl)pyridin-2(1H)-one | LCMS: m/z 504.2 [M + H]⁺. 1H-NMR (400 MHz, DMSO-d6) δ 8.85-8.79 (m, 2H), 8.68 (d, J = 8.5 Hz, 2H), 8.25 (d, J = 4.9 Hz, 1H), 8.00 (d, J = 8.6 Hz, 2H), 7.95 (d, J = 7.0 Hz, 1H), 7.58-7.52 (m, 1H), 7.38 (d, J = 3.6 Hz, 1H), 7.18 (d, J = 1.9 Hz, 1H), 7.08 (dd, J = 7.1, 2.0 Hz, 1H), 6.38 (d, J = 5.1 Hz, 1H), 5.08 (s, 2H), 0.61- 0.59 (m, 2H), 0.47-0.36 (m, 2H). |
| 46 | B7 | 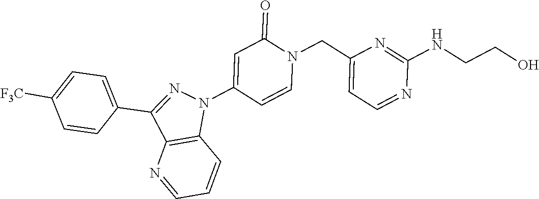 [SMILES: O=c1cc(-n2nc(-c3ccc(C(F)(F)F)cc3)c3ncccc32)ccn1Cc1ccnc(NCCO)n1] | 1-((2-((2- hydroxyethyl)amino)pyrimidin- 4-yl)methyl)-4-(3-(4- (trifluoromethyl)phenyl)- 1H-pyrazolo[4,3-b]pyridin- 1-yl)pyridin-2(1H)-one | LCMS: m/z 508.1 [M + H]⁺. 1H-NMR (400 MHz, DMSO-d6) δ 8.91-8.78 (m, 3H), 8.63 (dd, J = 8.8, 1.3 Hz, 1H), 8.28 (d, J = 5.0 Hz, 1H), 8.03 (d, J = 7.5 Hz, 1H), 7.97 (d, J = 8.3 Hz, 2H), 7.68 (dd, J = 8.8, 4.4 Hz, 1H), 7.09 (dd, J = 7.5, 2.5 Hz, 1H), 6.94 (d, J = 2.5 Hz, 1H), 6.44 (d, J = 5.1 Hz, 1H), 5.11 (s, 2H), 3.68-3.43 (m, 4H), 3.08 (s, 3H). |
| 47 | B7 | 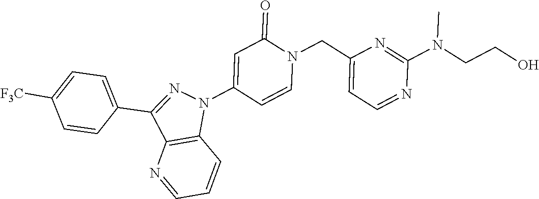 [SMILES: CN(CCO)c1nccc(Cn2ccc(-n3nc(-c4ccc(C(F)(F)F)cc4)c4ncccc43)cc2=O)n1] | 1-((2-((2- hydroxyethyl)(methyl)amino) pyrimidin-4-yl)methyl)-4- (3-(4- (trifluoromethyl)phenyl)- 1H-pyrazolo[4,3-b]pyridin- 1-yl)pyridin-2(1H)-one | LCMS: m/z 522.2 [M + H]⁺. 1H-NMR (400 MHz, DMSO-d6) δ 8.91-8.78 (m, 3H), 8.63 (dd, J = 8.8, 1.3 Hz, 1H), 8.28 (d, J = 5.0 Hz, 1H), 8.03 (d, J = 7.5 Hz, 1H), 7.97 (d, J = 8.3 Hz, 2H), 7.68 (dd, J = 8.8, 4.4 Hz, 1H), 7.09 (dd, J = 7.5, 2.5 Hz, 1H), 6.94 (d, J = 2.5 Hz, 1H), 6.44 (d, J = 5.1 Hz, 1H), 5.11 (s, 2H), 3.68-3.43 (m, 4H), 3.08 (s, 3H). |
| 48 | B7 | 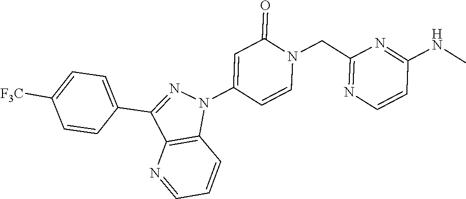 [SMILES: CNc1ccnc(Cn2ccc(-n3nc(-c4ccc(C(F)(F)F)cc4)c4ncccc43)cc2=O)n1] | 1-((4- (methylamino)pyrimidin-2- yl)methyl)-4-(3-(4- (trifluoromethyl)phenyl)- 1H-pyrazolo[4,3-b]pyridin- 1-yl)pyridin-2(1H)-one | LCMS: m/z 478.1 [M + H]⁺. 1H-NMR (400 MHz, DMSO-d6) δ 8.93-8.78 (m, 3H), 8.63 (dd, J = 8.8, 1.3 Hz, 1H), 8.05-7.86 (m, 3H), 7.68 (dd, J = 8.7, 4.4 Hz, 1H), 7.47-7.26 (m, 1H), 7.05 (dd, J = 7.5, 2.5 Hz, 1H), 6.90 (d, J = 2.5 Hz, 1H), 6.33 (d, J = 6.0 Hz, 1H), 5.11 (s, 2H), 2.80- 2.69 (m, 3H). |
| 49 | A17 | 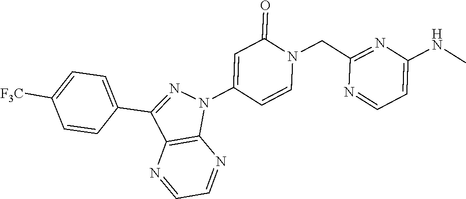 [SMILES: CNc1ccnc(Cn2ccc(-n3nc(-c4ccc(C(F)(F)F)cc4)c4nccnc43)cc2=O)n1] | 1-((4- (methylamino)pyrimidin-2- yl)methyl)-4-(3-(4- (trifluoromethyl)phenyl)- 1H-pyrazolo[3,4-b]pyrazin- 1-yl)pyridin-2(1H)-one | LCMS: m/z. 479.1 [M + H]⁺. 1H-NMR (400 MHz, DMSO-d6) δ 8.96 (d, J = 2.3 Hz, 1H), 8.89 (d, J = 2.3 Hz, 1H), 8.75 (d, J = 7.9 Hz, 2H), 8.03-7.90 (m, 3H), 7.54 (d, J = 2.4 Hz, 1H), 7.46-7.30 (m, 2H), 6.32 (d, J = 6.0 Hz, 1H), 5.09 (s, 2H), 2.74 (d, J = 4.6 Hz, 3H). |
| 50 | A17 | 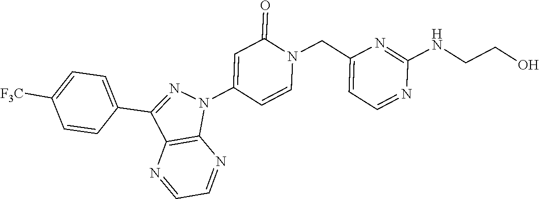 [SMILES: O=c1cc(-n2nc(-c3ccc(C(F)(F)F)cc3)c3nccnc32)ccn1Cc1ccnc(NCCO)n1] | 1-((2-((2- hydroxyethyl)amino)pyrimidin- 4-yl)methyl)-4-(3-(4- (trifluoromethyl)phenyl)- 1H-pyrazolo[3,4-b]pyrazin- 1-yl)pyridin-2(1H)-one | LCMS: m/z 509.1 [M + H]⁺. 1H-NMR (400 MHz, DMSO-d6) δ 8.97 (d, J = 2.3 Hz, 1H), 8.90 (d, J = 2.3 Hz, 1H), 8.75 (d, J = 8.2 Hz, 2H), 8.29 (d, J = 5.7 Hz, 1H), 8.03 (d, J = 7.6 Hz, 1H), 7.99 (d, J = 8.3 Hz, 2H), 7.60 (d, J = 2.5 Hz, 1H), 7.49 (dd, J = 7.5, 2.5 Hz, 1H), 6.61 (d, J = 5.6 Hz, 1H), 5.17 (s, 2H), 3.51-3.44 (m, 2H), 3.38- 3.26 (m, 2H). |
| 51 | A17 | 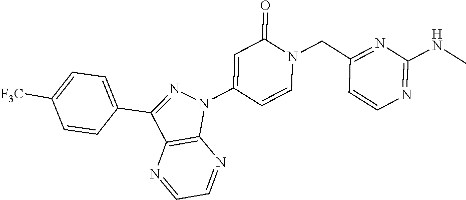 [SMILES: CNc1nccc(Cn2ccc(-n3nc(-c4ccc(C(F)(F)F)cc4)c4nccnc43)cc2=O)n1] | 1-((2- (methylamino)pyrimidin-4- yl)methyl)-4-(3-(4- (trifluoromethyl)phenyl)- 1H-pyrazolo[3,4-b]pyrazin- 1-yl)pyridin-2(1H)-one | LCMS: m/z 479.1 [M + H]⁺. 1H-NMR (400 MHz, DMSO-d6) δ 8.97 (d, J = 2.3 Hz, 1H), 8.90 (d, J = 2.3 Hz, 1H), 8.76 (d, J = 8.0 Hz, 2H), 8.28-8.17 (m, 1H), 8.06-7.96 (m, 3H), 7.60 (d, J = 2.5 Hz, 1H), 7.47 (dd, J = 7.5, 2.5 Hz, 1H), 7.08 (s, 1H), 6.33 (d, J = 5.0 Hz, 1H), 5.06 (s, 2H), 2.74 (d, J = 4.7 Hz, 3H). |
| 52 | A17 | 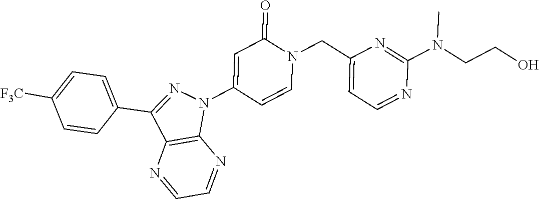 [SMILES: CN(CCO)c1nccc(Cn2ccc(-n3nc(-c4ccc(C(F)(F)F)cc4)c4nccnc43)cc2=O)n1] | 1-((2-((2- hydroxyethyl)(methyl)amino) pyrimidin-4-yl)methyl)-4- (3-(4- (trifluoromethyl)phenyl)- 1H-pyrazolo[3,4-b]pyrazin- 1-yl)pyridin-2(1H)-one | LCMS: m/z 523.2 [M + H]⁺. 1H-NMR (400 MHz, DMSO-d6) δ 8.98 (d, J = 2.3 Hz, 1H), 8.91 (d, J = 2.3 Hz, 1H), 8.78 (d, J = 8.1 Hz, 2H), 8.27 (d, J = 4.9 Hz, 1H), 8.07-7.96 (m, 3H), 7.61 (d, J = 2.5 Hz, 1H), 7.48 (dd, J = 7.6, 2.5 Hz, 1H), 6.42 (d, J = 5.0 Hz, 1H), 5.09 (s, 2H), 3.58- 3.48 (m, 4H), 3.07 (s, 3H). |
| 53 | B25 | 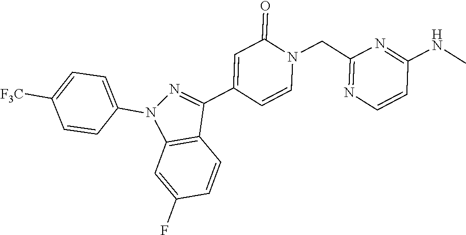 [SMILES: CNc1ccnc(Cn2ccc(-c3nn(-c4ccc(C(F)(F)F)cc4)c4cc(F)ccc34)cc2=O)n1] | 4-(6-fluoro-1-(4- (trifluoromethyl)phenyl)- 1H-indazol-3-yl)-1-((4- (methylamino)pyrimidin-2- yl)methyl)pyridin-2(1H)- one | LCMS: m/z 495.1 [M + H]⁺. 1H-NMR (400 MHz, DMSO-d6) δ 8.28 (dd, J = 9.0, 5.1 Hz, 1H), 8.14 (d, J = 8.4 Hz, 2H), 7.98 (d, J = 8.6 Hz, 2H), 7.93-7.85 (m, 2H), 7.45-7.28 (m, 2H), 7.05 (d, J = 1.9 Hz, 1H), 6.93 (dd, J = 7.1, 2.0 Hz, 1H), 6.33 (d, J = 6.0 Hz, 1H), 5.09 (s, 2H), 2.74 (d, J = 4.7 Hz, 3H). |
| 54 | B28 | 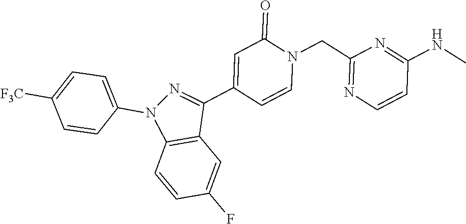 [SMILES: CNc1ccnc(Cn2ccc(-c3nn(-c4ccc(C(F)(F)F)cc4)c4ccc(F)cc34)cc2=O)n1] | 4-(5-fluoro-1-(4- (trifluoromethyl)phenyl)- 1H-indazol-3-yl)-1-((4- (methylamino)pyrimidin-2- yl)methyl)pyridin-2(1H)- one | LCMS: m/z 495.1 [M + H]⁺. 1H-NMR (400 MHz, DMSO-d6) δ 8.14 (d, J = 8.4 Hz, 2H), 8.08 (dd, J = 9.3, 4.2 Hz, 1H), 8.04 (dd, J = 9.2, 2.4 Hz, 1H), 8.00 (d, J = 8.6 Hz, 2H), 7.88 (d, J = 7.2 Hz, 1H), 7.55 (td, J = 9.0, 2.4 Hz, 1H), 7.43- 7.31 (m, 1H), 7.05 (d, J = 1.9 Hz, 1H), 6.95 (dd, J = 7.1, 2.0 Hz, 1H), 6.33 (d, J = 6.0 Hz, 1H), 5.09 (s, 2H), 2.74 (d, J = 4.7 Hz, 3H). |
| 55 | A1 | 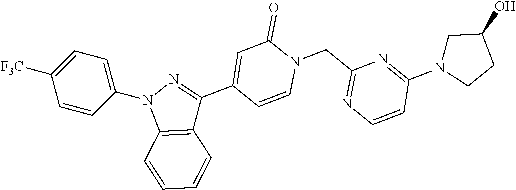 [SMILES: O=c1cc(-c2nn(-c3ccc(C(F)(F)F)cc3)c3ccccc23)ccn1Cc1nccc(N2CC[C@H](O)C2)n1] | (S)-1-((4-(3- hydroxypyrrolidin-1- yl)pyrimidin-2-yl)methyl)- 4-(1-(4- (trifluoromethyl)phenyl)- 1H-indazol-3-yl)pyridin- 2(1H)-one | LCMS: m/z 533.2 [M + H]⁺. 1H-NMR (400 MHz, DMSO-d6) δ 8.24 (d, J = 8.3 Hz, 1H), 8.15 (d, J = 8.4 Hz, 2H), 8.10-8.04 (m, 2H), 8.00 (d, J = 8.5 Hz, 2H), 7.89 (d, J = 7.2 Hz, 1H), 7.70-7.61 (m, 1H), 7.51-7.43 (m, 1H), 7.07 (d, J = 1.9 Hz, 1H), 6.96 (dd, J = 7.0, 2.0 Hz, 1H), 6.41-6.32 (m, 1H), 5.12 (s, 2H), 5.00 (d, J = 38.5 Hz, 1H), 4.34 (d, J = 35.7 Hz, 1H), 3.47-3.38 (m, 3H), 2.02-1.77 (m, 2H). |
| 56 | B7 | 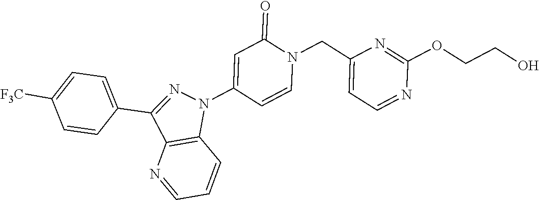 [SMILES: O=c1cc(-n2nc(-c3ccc(C(F)(F)F)cc3)c3ncccc32)ccn1Cc1ccnc(OCCO)n1] | 1-((2-(2- hydroxyethoxy)pyrimidin-4- yl)methyl)-4-(3-(4- (trifluoromethyl)phenyl)- 1H-pyrazolo[4,3-b]pyridin- 1-yl)pyridin-2(1H)-one | LCMS: m/z 509.2 [M + H]⁺. 1H-NMR (400 MHz, DMSO-d6) δ 8.89-8.80 (m, 3H), 8.65 (dd, J = 8.8, 1.3 Hz, 1H), 8.56 (d, J = 5.1 Hz, 1H), 8.07 (d, J = 7.5 Hz, 1H), 7.97 (d, J = 8.3 Hz, 2H), 7.69 (dd, J = 8.8, 4.4 Hz, 1H), 7.13 (dd, J = 7.5, 2.5 Hz, 1H), 7.04 (d, J = 5.1 Hz, 1H), 6.95 (d, J = 2.5 Hz, 1H), 5.25 (s, 2H), 4.86 (t, J = 5.5 Hz, 1H), 4.26 (t, J = 5.6 Hz, 2H), 3.66 (q, J = 5.3 Hz, 2H). |
| 80 | B13 | 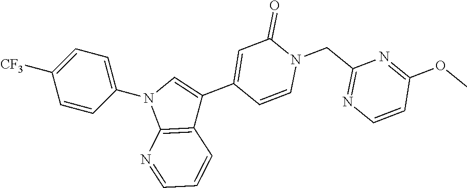 [SMILES: COc1ccnc(Cn2ccc(-c3cn(-c4ccc(C(F)(F)F)cc4)c4ncccc34)cc2=O)n1] | 1-((4-methoxypyrimidin-2- yl)methyl)-4-(1-(4- (trifluoromethyl)phenyl)- 1 H-pyrrolo[2,3-b]pyridin-3- yl)pyridin-2(1H)-one | ¹H-NMR (400 MHz, DMSO-d6) δ 8.76 (s, 1H), 8.60-8.49 (m, 1H), 8.48-8.40 (m, 2H), 8.32 (d, J = 8.4 Hz, 2H), 7.97 (d, J = 8.4 Hz, 2H), 7.93-7.78 (m, 1H), 7.40 (dd, J = 8.0, 4.8 Hz, 1H), 6.98-6.76 (m, 3H), 5.23 (s, 2H), 3.86 (s, 3H). LCMS: m/z 478.1 [M + H]⁺ |
| 83 | A1 | 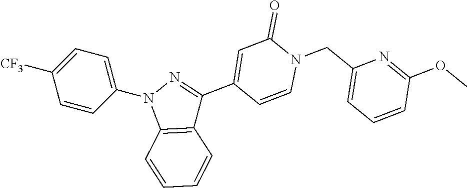 [SMILES: COc1cccc(Cn2ccc(-c3nn(-c4ccc(C(F)(F)F)cc4)c4ccccc34)cc2=O)n1] | 1-((6-methoxypyridin-2- yl)methyl)-4-(1-(4- (trifluoromethyl)phenyl)- 1 H-indazol-3-yl)pyridin- 2(1H)-one | ¹H-NMR (400 MHz, DMSO-d6) δ 8.25 (d, J = 8.4 Hz, 1H), 8.14 (d, J = 8.4 Hz, 2H), 8.05 (d, J = 8.4 Hz, 1H), 7.99 (dd, J = 8.0, 6.4 Hz, 3H), 7.74-7.61 (m, 2H), 7.51-7.41 (m, 1H), 7.11 (d, J = 2.0 Hz, 1H), 7.02 (dd, J = 7.2, 2.0 Hz, 1H), 6.82 (d, J = 7.2 Hz, 1H), 6.73 (d, J = 8.4 Hz, 1H), 5.20 (s, 2H), 3.79 (s, 3H). LCMS: m/z 477.1 [M + H]⁺ |
| 88 | B1 | 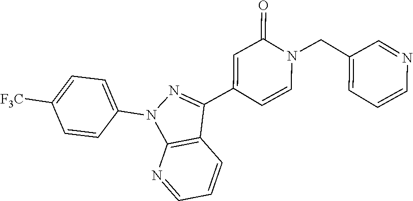 [SMILES: O=c1cc(-c2nn(-c3ccc(C(F)(F)F)cc3)c3ncccc23)ccn1Cc1cccnc1] | 1-(pyridin-3-ylmethyl)-4-(1- (4-(trifluoromethyl)phenyl)- 1H-pyrazolo[3,4-b]pyridin- 3-yl)pyridin-2(1H)-one | ¹H-NMR (400 MHz, CDCl3) δ 8.66-8.64 (m, 2H), 8.52 (d, J= 8.6 Hz, 3H), 8.41 (dd, J = 8.2, 1.3 Hz, 1H), 7.80 (d, J = 7.9 Hz, 1H), 7.74 (d, J = 8.7 Hz, 2H), 7.43 (d, J = 7.1 Hz, 1H), 7.32 (dd, J = 8.2, 4.5 Hz, 2H), 7.27 (s, 1H), 7.00 (dd, J = 7.1, 1.8 Hz, 1H), 5.19 (s, 2H). LCMS: m/z 448.1 [M + H]⁺. |
| 89 | A1 | 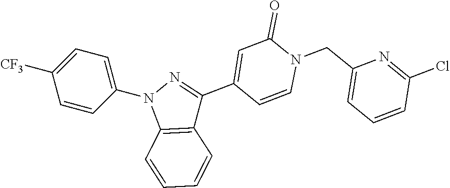 [SMILES: O=c1cc(-c2nn(-c3ccc(C(F)(F)F)cc3)c3ccccc23)ccn1Cc1cccc(Cl)n1] | 1-((6-chloropyridin-2- yl)methyl)-4-(1-(4- (trifluoromethyl)phenyl)- 1H-indazol-3-yl)pyridin- 2(1H)-one | ¹H-NMR (400 MHz, DMSO-d6) δ 8.23 (d, J = 8.4 Hz, 1H), 8.12 (d, J = 8.4 Hz, 2H), 8.03 (d, J = 8.4 Hz, 1H), 8.00-7.94 (m, 3H), 7.87 (t, J = 8.0 Hz, 1H), 7.63 (ddd, J = 8.4, 7.2, 1.2 Hz, 1H), 7.45 (dd, J = 8.0, 5.6 Hz, 2H), 7.30 (d, J = 7.6 Hz, 1H), 7.09 (d, J = 1.6 Hz, 1H), 7.02 (dd, J = 7.2, 2.0 Hz, 1H), 5.26 (s, 2H). LCMS: m/z 481.1 [M + H]⁺ |
| 96 | A1 | 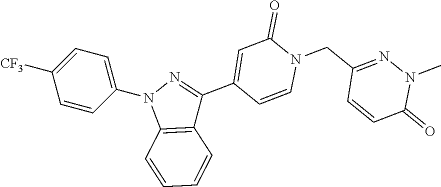 [SMILES: Cn1nc(Cn2ccc(-c3nn(-c4ccc(C(F)(F)F)cc4)c4ccccc34)cc2=O)ccc1=O] | 2-methyl-6-((2-oxo-4-(1-(4- (trifluoromethyl)phenyl)- 1H-indazol-3-yl)pyridin- 1(2H)-yl)methyl)pyridazin- 3(2H)-one | LCMS: m/z 478.1 [M + H]⁺ |
| 104 | A20 | 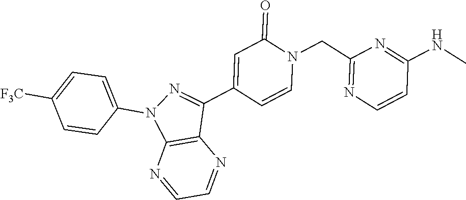 [SMILES: CNc1ccnc(Cn2ccc(-c3nn(-c4ccc(C(F)(F)F)cc4)c4nccnc34)cc2=O)n1] | 1-((4- (methylamino)pyrimidin-2- yl)methyl)-4-(1-(4- (trifluoromethyl)phenyl)- 1H-pyrazolo[3,4-b]pyrazin- 3-yl)pyridin-2(1H)-one | ¹H-NMR (400 MHz, DMSO) δ 8.96 (d, J = 2.2 Hz, 1H), 8.89 (d, J = 2.2 Hz, 1H), 8.62 (d, J = 8.6 Hz, 2H), 8.03 (d, J = 8.7 Hz, 2H), 7.93 (d, J = 7.1 Hz, 1H), 7.73 (d, J = 1.4 Hz, 1H), 7.41 (s, 1H), 7.22 (dd, J = 7.1, 1.8 Hz, 1H), 6.33 (d, J = 6.0 Hz, 1H), 5.10 (s, 2H), 2.73 (d, J = 4.6 Hz, 3H). LCMS: m/z 479.1 [M + H]+. |
| 105 | A20 | 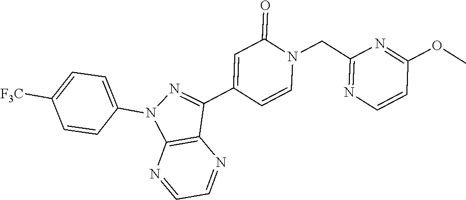 [SMILES: COc1ccnc(Cn2ccc(-c3nn(-c4ccc(C(F)(F)F)cc4)c4nccnc34)cc2=O)n1] | 1-((4-methoxypyrimidin-2- yl)methyl)-4-(1-(4- (trifluoromethyl)phenyl)- 1H-pyrazolo[3,4-b]pyrazin- 3-yl)pyridin-2(1H)-one | ¹H-NMR (400 MHz, DMSO) δ 8.97 (d, J = 2.2 Hz, 1H), 8.90 (d, J = 2.2 Hz, 1H), 8.63 (d, J = 8.5 Hz, 2H), 8.46 (d, J = 5.8 Hz, 1H), 8.05 (d, J = 7.2 Hz, 2H), 8.00 (d, J = 7.2 Hz, 1H), 7.77 (d, J = 1.7 Hz, 1H), 7.27 (dd, J = 7.1, 1.9 Hz, 1H), 6.85 (d, J = 5.8 Hz, 1H), 5.29 (s, 2H), 3.85 (s, 3H). LCMS: m/z 480.1 [M + H]+. |
| 110 | B1 | 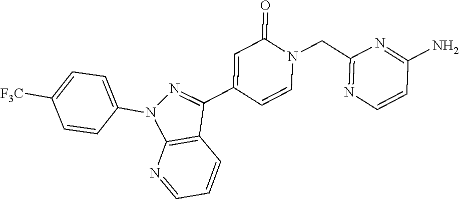 [SMILES: Nc1ccnc(Cn2ccc(-c3nn(-c4ccc(C(F)(F)F)cc4)c4ncccc34)cc2=O)n1] | 1-((4-aminopyrimidin-2- yl)methyl)-4-(1-(4- (trifluoromethyl)phenyl)- 1H-pyrazolo[3,4-b]pyridin- 3-yl)pyridin-2(1H)-one | ¹H-NMR (400 MHz, DMSO) δ 8.83-8.80 (m, 2H), 8.69 (d, J = 8.6 Hz, 2H), 7.99 (dd, J = 9.2, 7.5 Hz, 3H), 7.91 (d, J = 7.1 Hz, 1H), 7.55 (dd, J = 7.5, 5.3 Hz, 1H), 7.14 (d, J = 1.6 Hz, 1H), 7.02 (dd, J = 7.0, 1.8 Hz, 1H), 6.90 (s, 2H), 6.30 (d, J = 5.8 Hz, 1H), 5.07 (s, 2H). LCMS: m/z 464.1 [M + H]+. |
| 118 | B7 | 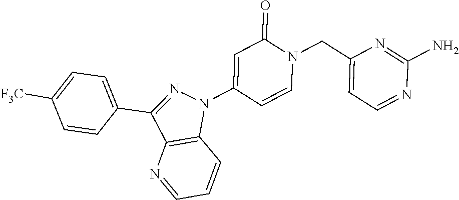 [SMILES: Nc1nccc(Cn2ccc(-n3nc(-c4ccc(C(F)(F)F)cc4)c4ncccc43)cc2=O)n1] | 1-((2-aminopyrimidin-4- yl)methyl)-4-(3-(4- (trifluoromethyl)phenyl)- 1H-pyrazolo[4,3-b]pyridin- 1-yl)pyridin-2(1H)-one | LCMS: m/z. 464.1 [M + H]⁺. 1H-NMR (400 MHz, CDCl3) δ 8.81 (dd, J = 4.4, 1.3 Hz, 1H), 8.75 (d, J = 8.1 Hz, 2H), 8.32 (dd, J = 8.8, 1.3 Hz, 1H), 8.25 (d, J = 5.4 Hz, 1H), 7.79 (d, J = 8.2 Hz, 2H), 7.60 (d, J = 7.5 Hz, 1H), 7.51 (dd, J = 8.7, 4.4 Hz, 1H), 7.12 (dd, J = 7.6, 2.4 Hz, 1H), 6.96 (d, J = 2.4 Hz, 1H), 6.71 (d, J = 5.3 Hz, 1H), 5.68 (s, 2H), 5.11 (s, 2H). |
| 119 | B7 | 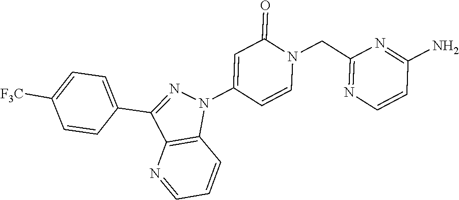 [SMILES: Nc1ccnc(Cn2ccc(-n3nc(-c4ccc(C(F)(F)F)cc4)c4ncccc43)cc2=O)n1] | 1-((4-aminopyrimidin-2- yl)methyl)-4-(3-(4- (trifluoromethyl)phenyl)- 1H-pyrazolo[4,3-b]pyridin- 1-yl)pyridin-2(1H)-one | LCMS: m/z 464.1 [M + H]⁺. 1H-NMR (400 MHz, DMSO-d6) δ 8.90-8.77 (m, 3H), 8.63 (dd, J = 8.8, 1.3 Hz, 1H), 8.07-7.92 (m, 4H), 7.72-7.61 (m, 2H), 7.04 (dd, J = 7.5, 2.5 Hz, 1H), 6.92-6.86 (m, 2H), 6.30 (d, J = 5.9 Hz, 1H), 5.07 (s, 2H). |
| 120 | B7 | 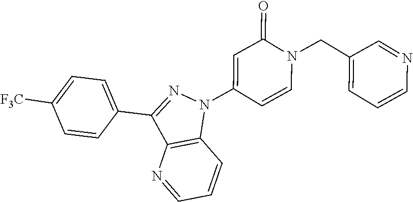 [SMILES: O=c1cc(-n2nc(-c3ccc(C(F)(F)F)cc3)c3ncccc32)ccn1Cc1cccnc1] | 1-(pyridin-3-ylmethyl)-4-(3- (4-(trifluoromethyl)phenyl)- 1H-pyrazolo[4,3-b]pyridin- 1-yl)pyridin-2(1H)-one | LCMS: m/z 464.1 [M + H]⁺. 1H NMR (400 MHz, Chloroform-d) δ 8.80 (dd, J = 4.5, 1.3 Hz, 1H), 8.74 (d, J = 8.1 Hz, 2H), 8.69 (d, J = 2.3 Hz, 1H), 8.60 (dd, J = 4.9, 1.7 Hz, 1H), 8.31 (dd, J = 8.8, 1.4 Hz, 1H), 7.85-7.72 (m, 3H), 7.57- 7.46 (m, 2H), 7.35 (dd, J = 7.9, 4.9 Hz, 1H), 7.08 (dd, J = 7.6, 2.4 Hz, 1H), 6.98 (d, J = 2.4 Hz, 1H), 5.24 (s, 2H). |
| 121 | B7 | 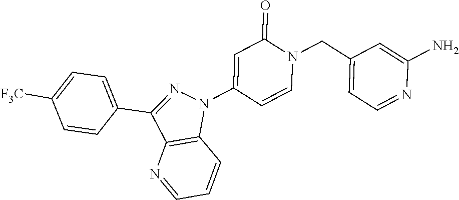 [SMILES: Nc1cc(Cn2ccc(-n3nc(-c4ccc(C(F)(F)F)cc4)c4ncccc43)cc2=O)ccn1] | 1-((2-aminopyridin-4- yl)methyl)-4-(3-(4- (trifluoromethyl)phenyl)- 1H-pyrazolo[4,3-b]pyridin- 1-yl)pyridin-2(1H)-one | LCMS: m/z 463.1 [M + H]⁺. 1H-NMR (400 MHz, CDCl3) δ 8.80 (d, J = 4.2 Hz, 1H), 8.74 (d, J = 8.1 Hz, 2H), 8.31 (d, J = 8.8 Hz, 1H), 7.95-7.87 (m, 1H), 7.79 (d, J = 8.2 Hz, 2H), 7.55-7.42 (m, 2H), 7.12 (d, J = 7.3 Hz, 1H), 7.02- 6.95 (m, 1H), 6.65-6.56 (m, 1H), 6.53-6.44 (m, 1H), 5.83 (s, 2H), 5.12 (s, 2H). |
| 124 | B7 | 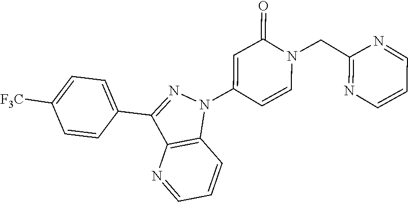 [SMILES: O=c1cc(-n2nc(-c3ccc(C(F)(F)F)cc3)c3ncccc32)ccn1Cc1ncccn1] | 1-(pyrimidin-2-ylmethyl)-4- (3-(4- (trifluoromethyl)phenyl)- 1H-pyrazolo[4,3-b]pyridin- 1-yl)pyridin-2(1H)-one | LCMS: m/z 449.1 [M + H]⁺. 1H-NMR (400 MHz, CDCl3) δ 8.84-8.68 (m, 5H), 8.36 (d, J = 8.5 Hz, 1H), 7.79 (d, J = 8.2 Hz, 2H), 7.63 (d, J = 7.3 Hz, 1H), 7.50 (dd, J = 8.6, 4.4 Hz, 1H), 7.24 (d, J = 4.9 Hz, 1H), 7.19-7.11 (m, 1H), 7.04 (d, J = 2.1 Hz, 1H), 5.46 (s, 2H). |
| 145 | A10 | 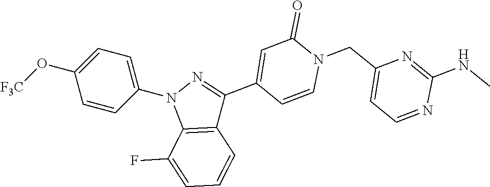 [SMILES: CNc1nccc(Cn2ccc(-c3nn(-c4ccc(OC(F)(F)F)cc4)c4c(F)cccc34)cc2=O)n1] | 4-(7-fluoro-1-(4- (trifluoromethoxy)phenyl)- 1H-indazol-3-yl)-1-((2- (methylamino)pyrimidin-4- yl)methyl)pyridin-2(1H)- one | LCMS: m/z 511.1 [M + H]⁺. 1H-NMR (400 MHz, DMSO-d6) δ 8.29-8.17 (m, 1H), 8.05 (d, J = 8.0 Hz, 1H), 7.96-7.84 (m, 3H), 7.61 (d, J = 8.5 Hz, 2H), 7.50-7.34 (m, 2H), 7.16- 7.02 (m, 2H), 6.94 (dd, J = 7.0, 2.0 Hz, 1H), 6.33 (d, J = 5.0 Hz, 1H), 5.05 (s, 2H), 2.74 (d, J = 4.2 Hz, 3H). |
| 146 | A28 | 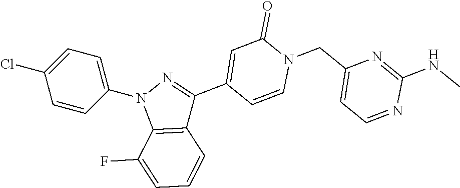 [SMILES: CNc1nccc(Cn2ccc(-c3nn(-c4ccc(Cl)cc4)c4c(F)cccc34)cc2=O)n1] | 4-(1-(4-chlorophenyl)-7- fluoro-1H-indazol-3-yl)-1- ((2- (methylamino)pyrimidin-4- yl)methyl)pyridin-2(1H)- one | LCMS: m/z 461.1 [M + H]⁺. 1H-NMR (400 MHz, DMSO-d6) δ 8.25-8.19 (m, 1H), 8.04 (d, J = 7.9 Hz, 1H), 7.90 (d, J = 7.1 Hz, 1H), 7.79 (d, J = 8.8 Hz, 2H), 7.67 (d, J = 8.8 Hz, 2H), 7.44-7.35 (m, 2H), 7.16-7.10 (m, 1H), 7.06 (d, J = 1.9 Hz, 1H), 6.94 (dd, J = 7.1, 2.0 Hz, 1H), 6.33 (d, J = 4.9 Hz, 1H), 5.06 (s, 2H), 2.78-2.71 (m, 3H). |

### Example 2: Synthesis of Compound 27

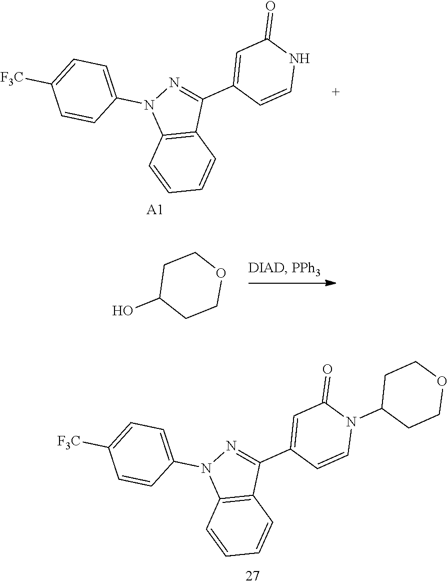

To a solution of compound A1 (50 mg, 0.141 mmol), tetrahydropyran-4-ol (21.6 mg, 0.211 mmol) and PPh3 (55.5 mg, 0.211 mmol) in anhydrous THF (1 mL) was added ethyl [(1E)-(ethoxycarbonyl)diazenyl]methanoate (0.033 mL, 0.211 mmol) at 25° C. The yellow solution was stirred at 25° C. for 15 min. The mixture was concentrated under reduced pressure. The residue was purified by combi flash (0-50% EtOAc/PE) and prep-HPLC (HCOOH) to afford compound 27 (5 mg, 8.07%) as white solid.

¹H-NMR (400 MHz, DMSO_d₆) δ 8.22 (d, J=8.4 Hz, 1H), 8.13 (d, J=8.4 Hz, 2H), 8.04 (d, =8.4 Hz, 1H), 7.99 (d, =8.4 Hz, 2H), 7.93 (d, J=7.2 Hz, 1H), 7.64 (ddd, J=8.4, 7.2, 1.2 Hz, 1H), 7.45 (t, J=7.6 Hz, 1H), 7.07 (d, J=2.0 Hz, 1H), 6.95 (dd, J=7.2, 2.0 Hz, 1H), 4.98 (tt, J=12.0, 4.0 Hz, 1H), 4.01 (dd, J=11.2, 4.2 Hz, 2H), 3.50 (td, J=11.6, 2.0 Hz, 2H), 1.95 (qd, J=12.0, 4.4 Hz, 2H), 1.82-1.71 (m, 2H).

LC-MS (ESI) m/z: 440.1 [M+H]⁺.

The following compounds listed below were synthesized according to the procedure of compound 27:

| Com- | From | | | |
|---|---|---|---|---|
| pound | Inter- | | | |
| No. | mediate | Structure | Name | Analytical data |
| 36 | A1 | 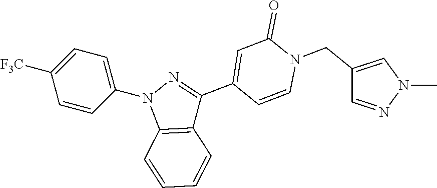 [SMILES: Cn1cc(Cn2ccc(-c3nn(-c4ccc(C(F)(F)F)cc4)c4ccccc34)cc2=O)cn1] | 1-((1-methyl-1H-pyrazol-4- yl)methyl)-4-(1-(4- (trifluoromethyl)phenyl)-1H- indazol-3-yl)pyridin-2(1H)-one | ¹H-NMR (400 MHz, DMSO_d₆) δ 8.22 (d, J = 8.4 Hz, 1H), 8.13 (d, J = 8.4 Hz, 2H), 8.04 (d, J = 8.4 Hz, 1H), 8.00 (d, J = 8.4 Hz, 2H), 7.94 (d, J = 7.2 Hz, 1H), 7.75 (s, 1H), 7.64 (ddd, J = 8.4, 7.2, 1.2 Hz, 1H), 7.49 (s, 1H), 7.48-7.42 (m, 1H), 7.06 (d, J = 2.0 Hz, 1H), 6.92 (dd, J = 7.2, 2.0 Hz, 1H), 4.99 (s, 2H), 3.80 (s, 3H). LCMS: m/z 450.1 [M + H]⁺. |
| 37 | A1 | 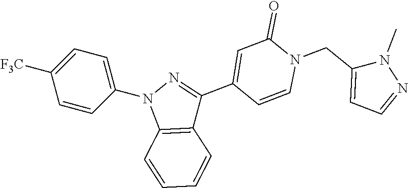 [SMILES: Cn1nccc1Cn1ccc(-c2nn(-c3ccc(C(F)(F)F)cc3)c3ccccc23)cc1=O] | 1-((1-methyl-1 H-pyrazol-5- yl)methyl)-4-(1-(4- (trifluoromethyl)phenyl)-1H- indazol-3-yl)pyridin-2(1H)-one | ¹H-NMR (400 MHz, DMSO_d₆) δ 8.24 (dd, J = 8.4, 1.2 Hz, 1H), 8.13 (d, J = 8.8 Hz, 2H), 8.04 (dd, J = 8.4, 0.8 Hz, 1H), 8.00 (d, J = 8.4 Hz, 2H), 7.92 (d, J = 7.2 Hz, 1H), 7.65 (ddd, J = 8.4, 7.2, 1.2 Hz, 1H), 7.46 (ddd, J = 8.0, 7.2, 0.8 Hz, 1H), 7.36 (d, J = 2.0 Hz, 1H), 7.12 (d, J = 2.0 Hz, 1H), 7.00 (dd, J = 7.2, 2.0 Hz, 1H), 6.22 (d, J = 2.0 Hz, 1H), 5.27 (s, 2H), 3.92 (s, 3H). LCMS: m/z 450.1 [M + H]⁺. |
| 38 | A1 | 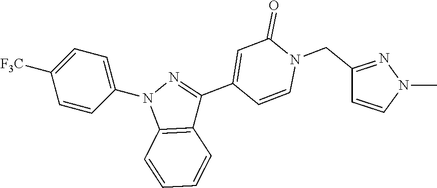 [SMILES: Cn1ccc(Cn2ccc(-c3nn(-c4ccc(C(F)(F)F)cc4)c4ccccc34)cc2=O)n1] | 1-((1-methyl-1H-pyrazol-3- yl)methyl)-4-(1-(4- (trifluoromethyl)phenyl)-1H- indazol-3-yl)pyridin-2(1H)-one | ¹H-NMR (400 MHz, DMSO_d₆) δ 8.22 (dt, J = 8.4, 0.8 Hz, 1H), 8.12 (d, J = 8.8 Hz, 2H), 8.04 (dd, J = 8.8, 0.8 Hz, 1H), 7.99 (d, J = 8.8 Hz, 2H), 7.85 (d, J = 7.2 Hz, 1H), 7.66- 7.61 (m, 2H), 7.45 (ddd, J = 8.0, 7.2, 0.8 Hz, 1H), 7.07 (d, J = 2.0 Hz, 1H), 6.93 (dd, J = 7.2, 2.0 Hz, 1H), 6.19 (d, J = 2.0 Hz, 1H), 5.10 (s, 2H), 3.80 (s, 3H). LCMS: m/z 450.1 [M + H]⁺. |
| 39 | A1 | 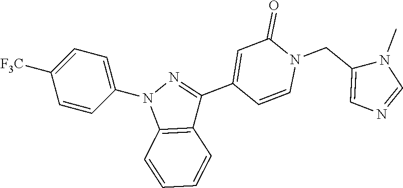 [SMILES: Cn1cncc1Cn1ccc(-c2nn(-c3ccc(C(F)(F)F)cc3)c3ccccc23)cc1=O] | 1-((1-methyl-1H-imidazol-5- yl)methyl)-4-(1-(4- (trifluoromethyl)phenyl)-1H- indazol-3-yl)pyridin-2(1H)-one | ¹H-NMR (400 MHz, DMSO_d₆) δ 8.40 (dd, J = 5.6, 0.8 Hz, 1H), 8.29 (dt, J = 8.4, 1.2 Hz, 1H), 8.18-8.11 (m, 2H), 8.05 (dd, J = 8.8, 0.8 Hz, 1H), 7.99 (d, J = 8.4 Hz, 2H), 7.72 (dd, J = 5.6, 1.2 Hz, 1H), 7.67 (d, J = 2.8 Hz, 1H), 7.65-7.61 (m, 1H), 7.49-7.46 (m, 1H), 7.46-7.42 (m, 1H), 7.07 (d, J = 1.2 Hz, 1H), 5.43 (s, 2H), 3.71 (s, 3H). LCMS: m/z 450.1 [M + H]⁺. |
| 60 | A1 | 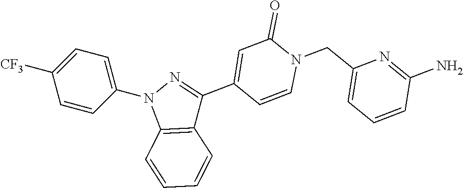 [SMILES: Nc1cccc(Cn2ccc(-c3nn(-c4ccc(C(F)(F)F)cc4)c4ccccc34)cc2=O)n1] | 1-((6-aminopyridin-2- yl)methyl)-4-(1-(4- (trifluoromethyl)phenyl)-1H- indazol-3-yl)pyridin-2(1H)-one | ¹H-NMR (400 MHz, DMSO-d6) δ 8.24 (dt, J = 8.4, 1.2 Hz, 1H), 8.13 (d, J = 8.4 Hz, 2H), 8.04 (d, J = 8.4 Hz, 1H), 7.99 (d, J = 8.4 Hz, 2H), 7.87 (d, J = 7.2 Hz, 1H), 7.64 (ddd, J = 8.4, 7.2, 1.2 Hz, 1H), 7.46 (ddd, J = 8.0, 7.2, 0.8 Hz, 1H), 7.33 (dd, J = 8.0, 7.2 Hz, 1H), 7.09 (d, J = 2.0 Hz, 1H), 6.97 (dd, J = 7.2, 2.0 Hz, 1H), 6.36-6.30 (m, 1H), 6.27 (d, J = 7.2 Hz, 1H), 5.97 (s, 2H), 5.01 (s, 2H). LCMS: m/7. 462.1 [M + H]⁺. |
| 61 | A1 | 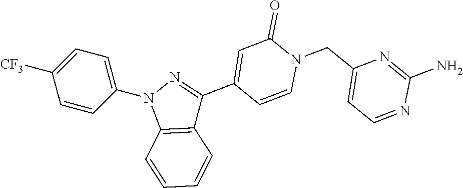 [SMILES: Nc1nccc(Cn2ccc(-c3nn(-c4ccc(C(F)(F)F)cc4)c4ccccc34)cc2=O)n1] | 1-((2-aminopyrimidin-4- yl)methyl)-4-(1-(4- (trifluoromethyl)phenyl)-1H- indazol-3-yl)pyridin-2(1H)-one | ¹H-NMR (400 MHz, DMSO-d6) δ 8.25 (d, J = 8.4 Hz, 1H), 8.20-8.11 (m, 3H), 8.05 (d, J = 8.8 Hz, 1H), 8.00 (d, J = 8.8 Hz, 2H), 7.91 (d, J = 7.2 Hz, 1H), 7.69-7.59 (m, 1H), 7.46 (t, J = 7.6 Hz, 1H), 7.10 (d, J = 2.0 Hz, 1H), 7.00 (dd, J = 7.2, 2.0 Hz, 1H), 6.64 (s, 2H), 6.30 (d, J = 4.8 Hz, 1H), 5.03 (s, 2H). LCMS: m/z 463.1 [M + H]⁺. |
| 62 | A1 | 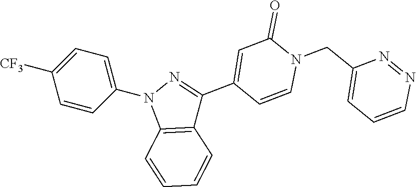 [SMILES: O=c1cc(-c2nn(-c3ccc(C(F)(F)F)cc3)c3ccccc23)ccn1Cc1cccnn1] | 1-(pyridazin-3-ylmethyl)-4-(1- (4-(trifluoromethyl)phenyl)- 1H-indazol-3-yl)pyridin-2(1H)- one | ¹H-NMR (400 MHz, DMSO-d6) δ 8.73 (d, J = 1.2 Hz, 1H), 8.67-8.55 (m, 2H), 8.24 (d, J = 8.4 Hz, 1H), 8.14 (d, J = 8.4 Hz, 2H), 8.10-7.97 (m, 4H), 7.65 (ddd, J = 8.4, 7.2, 1.2 Hz, 1H), 7.46 (t, J = 7.6 Hz, 1H), 7.08 (d, J = 2.0 Hz, 1H), 7.02 (dd, J = 7.2, 2.0 Hz, 1H), 5.35 (s, 2H). LCMS: m/z 448.1 [M + H]⁺. |
| 63 | A1 | 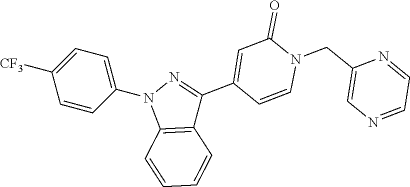 [SMILES: O=c1cc(-c2nn(-c3ccc(C(F)(F)F)cc3)c3ccccc23)ccn1Cc1cnccn1] | 1-(pyrazin-2-ylmethyl)-4-(1-(4- (trifluoromethyl)phenyl)-1H- indazol-3-yl)pyridin-2(1H)-one | ¹H-NMR (400 MHz, DMSO-d6) δ 8.73 (d, J = 1.2 Hz, 1H), 8.67-8.55 (m, 2H), 8.24 (d, J = 8.4 Hz, 1H), 8.14 (d, J = 8.4 Hz, 2H), 8.10-7.97 (m, 4H), 7.65 (ddd, J = 8.4, 7.2, 1.2 Hz, 1H), 7.46 (t, J = 7.6 Hz, 1H), 7.08 (d, J = 2.0 Hz, 1H), 7.02 (dd, J = 7.2, 2.0 Hz, 1H), 5.35 (s, 2H). LCMS: m/z 448.1 [M + H]⁺. |
| 64 | B13 | 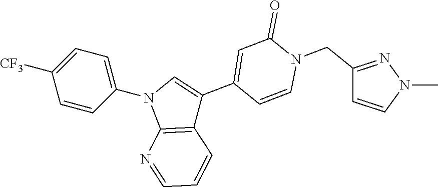 [SMILES: Cn1ccc(Cn2ccc(-c3cn(-c4ccc(C(F)(F)F)cc4)c4ncccc34)cc2=O)n1] | 1-((1-methyl-1H-pyrazol-3- yl)methyl)-4-(1-(4- (trifluoromethyl)phenyl)-1H- pyrrolo[2,3-b]pyridin-3- yl)pyridin-2(1H)-one | ¹H-NMR (400 MHz, DMSO-d6) δ 8.73 (s, 1H), 8.52- 8.43 (m, 2H), 8.32 (d, J = 8.4 Hz, 2H), 7.98 (d, J = 8.4 Hz, 2H), 7.78 (d, J = 7.2 Hz, 1H), 7.64 (d, J = 2.0 Hz, 1H), 7.41 (dd, J = 8.0, 4.8 Hz, 1H), 6.87 (d, J = 2.0 Hz, 1H), 6.81 (dd, J = 7.2, 2.0 Hz, 1H), 6.18 (d, J = 2.0 Hz, 1H), 5.07 (s, 2H), 3.81 (s, 3H). LCMS: m/z 450.1 [M + H]⁺. |
| 65 | A1 | 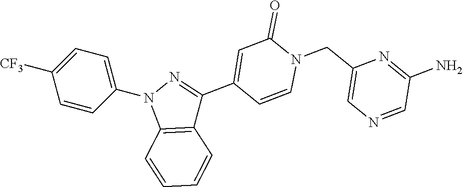 [SMILES: Nc1cncc(Cn2ccc(-c3nn(-c4ccc(C(F)(F)F)cc4)c4ccccc34)cc2=O)n1] | 1-((6-aminopyrazin-2- yl)methyl)-4-(1-(4- (trifluoromethyl)phenyl)-1H- indazol-3-yl)pyridin-2(1H)-one | ¹H-NMR (400 MHz, DMSO-d6) δ 8.24 (d, J = 8.4 Hz, 1H), 8.13 (d, J = 8.4 Hz, 2H), 8.05 (s, 1H), 7.99 (d, J = 8.4 Hz, 2H), 7.91 (d, J = 7.2 Hz, 1H), 7.77 (s, 1H), 7.67-7.62 (m, 1H), 7.60 (s, 1H), 7.50-7.42 (m, 1H), 7.08 (d, J = 2.0 Hz, 1H), 6.98 (dd, J = 7.2, 2.0 Hz, 1H), 6.48 (s, 2H), 5.05 (s, 2H). LCMS: m/z 463.1 [M + H]⁺. |
| 66 | A1 | 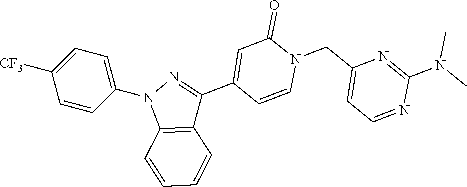 [SMILES: CN(C)c1nccc(Cn2ccc(-c3nn(-c4ccc(C(F)(F)F)cc4)c4ccccc34)cc2=O)n1] | 1-((2- (dimethylamino)pyrimidin-4- yl)methyl)-4-(1-(4- (trifluoromethyl)phenyl)-1H- indazol-3-yl)pyridin-2(1H)-one | ¹H-NMR (400 MHz, DMSO-d6) δ8.29 (d, J = 5.2 Hz, 1H), 8.25 (d, J = 8.4 Hz, 1H), 8.14 (d, J = 8.4 Hz, 2H), 8.05 (d, J = 8.4 Hz, 1H), 8.00 (d, J = 8.4 Hz, 2H), 7.93 (d, J = 7.2 Hz, 1H), 7.72-7.59 (m, 1H), 7.47 (t, J = 7.6 Hz, 1H), 7.11 (d, J = 2.0 Hz, 1H), 7.02 (dd, J = 7.2, 2.0 Hz, 1H), 6.40 (d, J = 5.2 Hz, 1H), 5.10 (s, 2H), 3.05 (s, 6H). LCMS: m/z 491.1 [M + H]⁺. |
| 67 | A | 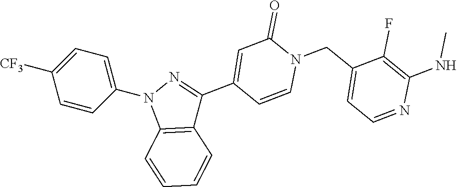 [SMILES: CNc1nccc(Cn2ccc(-c3nn(-c4ccc(C(F)(F)F)cc4)c4ccccc34)cc2=O)c1F] | 1-((3-fluoro-2- (methylamino)pyridin-4- yl)methyl)-4-(1-(4- (trifluoromethyl)phenyl)-1H- indazol-3-yl)pyridin-2(1H)-one | ¹H-NMR (400 MHz, DMSO-d6) δ 8.25 (d, J = 8.4 Hz, 1H), 8.14 (d, J = 8.4 Hz, 2H), 8.06 (d, J = 8.4 Hz, 1H), 8.00 (d, J = 8.4 Hz, 2H), 7.94 (d, J = 7.2 Hz, 1H), 7.77 (d, J = 5.2 Hz, 1H), 7.66 (dd, J = 8.4, 7.2 Hz, 1H), 7.47 (t, J = 7.6 Hz, 1H), 7.11 (d, J = 2.0 Hz, 1H), 7.02 (dd, J = 7.2, 2.0 Hz, 1H), 6.69 (d, J = 5.2 Hz, 1H), 6.22 (t, J = 5.2 Hz, 1H), 5.19 (s, 2H), 2.84 (d, J = 4.8 Hz, 3H). LCMS: m/z. 494.1 [M + H]⁺. |
| 68 | A1 | 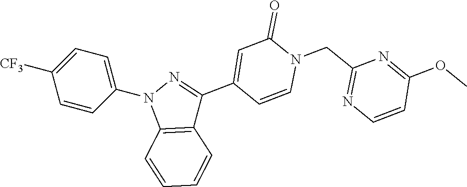 [SMILES: COc1ccnc(Cn2ccc(-c3nn(-c4ccc(C(F)(F)F)cc4)c4ccccc34)cc2=O)n1] | 1-((4-methoxypyrimidin-2- yl)methyl)-4-(1-(4- (trifluoromethyl)phenyl)-1H- indazol-3-yl)pyridin-2(1H)-one | ¹H-NMR (400 MHz, DMSO-d6) δ 8.47 (d, J = 5.6 Hz, 1H), 8.26 (dt, J = 8.4, 1.0 Hz, 1H), 8.15 (d, J = 8.4 Hz, 2H), 8.10-7.92 (m, 4H), 7.66 (ddd, J = 8.4, 6.8, 0.8 Hz, 1H), 7.47 (ddd, J = 8.0, 6.8, 0.8 Hz, 1H), 7.10 (d, J = 2.0 Hz, 1H), 7.01 (dd, J = 7.2, 2.0 Hz, 1H), 6.86 (d, J = 5.6 Hz, 1H), 5.29 (s, 2H), 3.87 (s, 3H). LCMS: m/z 478.1 [M + H]⁺. |
| 76 | B1 | 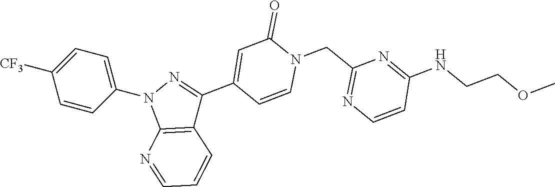 [SMILES: COCCNc1ccnc(Cn2ccc(-c3nn(-c4ccc(C(F)(F)F)cc4)c4ncccc34)cc2=O)n1] | 1-((4-((2- methoxyethyl)amino)pyrimidin- 2-yl)methyl)-4-(1-(4- (trifluoromethyl)phenyl)-1H- pyrazolo[3,4-b]pyridin-3- yl)pyridin-2(1H)-one | ¹H-NMR (400 MHz, DMSO-d6) δ 8.89-8.74 (m, 2H), 8.69 (d, J = 8.4 Hz, 2H), 8.01 (d, J = 8.8 Hz, 2H), 7.96 (br.s, 1H), 7.92 (d, J = 7.2 Hz, 1H), 7.56 (dd, J = 8.0, 4.8 Hz, 2H), 7.15 (d, J = 2.0 Hz, 1H), 7.03 (dd, J = 7.2, 2.0 Hz, 1H), 6.38 (d, J = 6.0 Hz, 1H), 5.10 (s, 2H), 3.72-3.62 (m, 4H), 3.18 (s, 3H). LCMS: m/z 522.1 [M + H]⁺. |
| 97 | B1 | 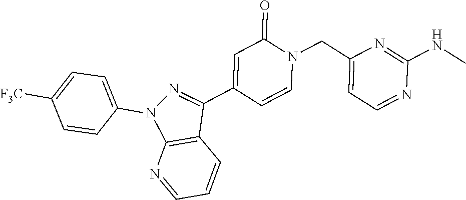 [SMILES: CNc1nccc(Cn2ccc(-c3nn(-c4ccc(C(F)(F)F)cc4)c4ncccc34)cc2=O)n1] | 1-((2-(methylamino)pyrimidin- 4-yl)methyl)-4-(1-(4- (trifluoromethyl)phenyl)-1H- pyrazolo[3,4-b]pyridin-3- yl)pyridin-2(1H)-one | ¹H-NMR (400 MHz, DMSO) δ 8.83-8.81 (m, 2H), 8.69 (d, J = 8.5 Hz, 2H), 8.23 (s, 1H), 8.02-7.97 (m, 3H), 7.57 (s, 1H), 7.20-7.07 (m, 3H), 6.35 (s, 1H), 5.08 (s, 2H), 2.75 (d, J = 4.0 Hz, 3H). LCMS: m/z 478.1 [M + H]+. |
| 98 | B1 | 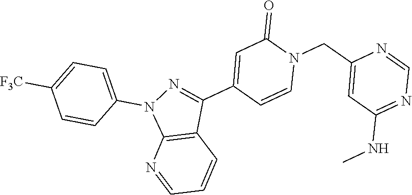 [SMILES: CNc1cc(Cn2ccc(-c3nn(-c4ccc(C(F)(F)F)cc4)c4ncccc34)cc2=O)ncn1] | 1-((6-(methylamino)pyrimidin- 4-yl)methyl)-4-(1-(4- (trifluoromethyl)phenyl)-1H- pyrazolo[3,4-b]pyridin-3- yl)pyridin-2(1H)-one | ¹H-NMR (400 MHz, DMSO) δ 8.82 (d, J = 6.0 Hz, 2H), 8.69 (d, J = 8.5 Hz, 2H), 8.40 (s, 1H), 8.01 (d, J = 8.8 Hz, 2H), 7.96 (d, J = 7.1 Hz, 1H), 7.58-7.54 (m, 2H), 7.19 (s, 1H), 7.09 (d, J = 7.1 Hz, 1H), 6.21 (s, 1H), 5.04 (s, 2H), 2.79 (s, 3H). LCMS: m/z 478.1 [M + H]+. |
| 99 | A20 | 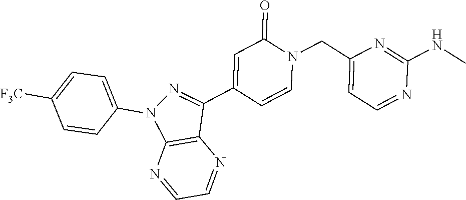 [SMILES: CNc1nccc(Cn2ccc(-c3nn(-c4ccc(C(F)(F)F)cc4)c4nccnc34)cc2=O)n1] | 1-((2-(methylamino)pyrimidin- 4-yl)methyl)-4-(1-(4- (trifluoromethyl)phenyl)-1H- pyrazolo[3,4-b]pyrazin-3- yl)pyridin-2(1H)-one | ¹H-NMR (400 MHz, DMSO) δ 8.96 (d, J = 2.1 Hz, 1H), 8.89 (d, J = 2.1 Hz, 1H), 8.62 (d, J = 8.6 Hz, 2H), 8.22 (s, 1H), 8.04 (d, J = 8.7 Hz, 2H), 7.97 (d, J = 7.0 Hz, 1H), 7.77 (s, 1H), 7.27 (d, J = 7.0 Hz, 1H), 7.08 (s, 1H), 6.33 (d, J = 4.8 Hz, 1H), 5.07 (s, 2H), 2.74 (d, J = 3.9 Hz, 3H). LCMS: m/z 479.1 [M + H]+. |
| 109 | B1 | 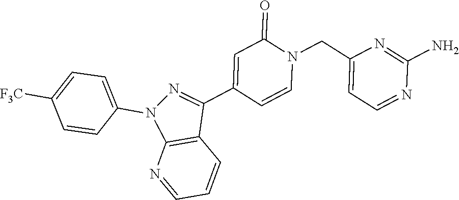 [SMILES: Nc1nccc(Cn2ccc(-c3nn(-c4ccc(C(F)(F)F)cc4)c4ncccc34)cc2=O)n1] | 1-((2-aminopyrimidin-4- yl)methyl)-4-(1-(4- (trifluoromethyl)phenyl)-1H- pyrazolo[3,4-b]pyridin-3- yl)pyridin-2(1H)-one | ¹H-NMR (400 MHz, DMSO) δ 8.83-8.81 (m, 2H), 8.69 (d, J = 8.6 Hz, 2H), 8.18 (d, J = 5.1 Hz, 1H), 7.98 (dd, J = 21.4, 7.9 Hz, 3H), 7.55 (dd, J = 7.7, 5.1 Hz, 1H), 7.19 (d, J = 1.7 Hz, 1H), 7.08 (dd, J = 7.1, 1.9 Hz, 1H), 6.68 (s, 2H), 6.32 (d, J = 5.1 Hz, 1H), 5.05 (s, 2H). LCMS: m/z 464.1 [M + H]+. |
| 111 | B1 | 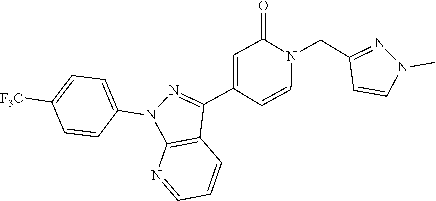 [SMILES: Cn1ccc(Cn2ccc(-c3nn(-c4ccc(C(F)(F)F)cc4)c4ncccc34)cc2=O)n1] | 1-((1-methyl-1H-pyrazol-3- yl)methyl)-4-(1-(4- (trifluoromethyl)phenyl)-1H- pyrazolo[3,4-b]pyridin-3- yl)pyridin-2(1H)-one | ¹H-NMR (400 MHz, CDCl3) δ 8.73 (dd, J = 4.5, 1.4 Hz, 1H), 8.61 (d, J = 8.5 Hz, 2H), 8.52 (dd, J = 8.2, 1.4 Hz, 1H), 7.82 (d, J = 8.7 Hz, 2H), 7.68-7.63 (m, 1H), 7.49 (d, J = 4.9 Hz, 1H), 7.35 (d, J = 2.4 Hz, 2H), 7.06 (dd, J = 7.1, 1.7 Hz, 1H), 6.37 (d, J = 2.2 Hz, 1H), 5.25 (s, 2H), 3.93 (s, 3H). LCMS: m/z 450.1 [M + H]+. |
| 112 | B1 | 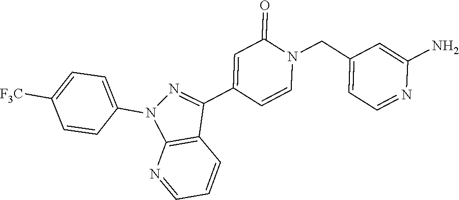 [SMILES: Nc1cc(Cn2ccc(-c3nn(-c4ccc(C(F)(F)F)cc4)c4ncccc34)cc2=O)ccn1] | 1-((2-aminopyridin-4- yl)methyl)-4-(1-(4- (trifluoromethyl)phenyl)-1H- pyrazolo[3,4-b]pyridin-3- yl)pyridin-2(1H)-one | ¹H-NMR (400 MHz, CDCl3) δ 8.65 (dd, J = 8.2, 1.4 Hz, 1H), 8.53 (d, J = 8.5 Hz, 2H), 8.41 (dd, J = 8.2, 1.4 Hz, 1H), 7.93 (d, J = 5.5 Hz, 1H), 7.74 (d, J = 8.6 Hz, 2H), 7.36-7.31 (m, 2H), 7.21 (s, 1H), 7.02 (dd, J = 7.1, 1.8 Hz, 1H), 6.53 (d, J = 5.6 Hz, 1H), 6.40 (s, 1H), 5.05 (s, 2H), 4.56 (s, 2H). LCMS: m/z 463.1 [M + H]+. |
| 115 | B1 | 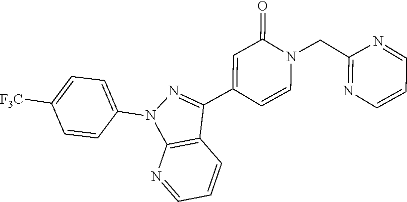 [SMILES: O=c1cc(-c2nn(-c3ccc(C(F)(F)F)cc3)c3ncccc23)ccn1Cc1ncccn1] | 1-(pyrimidin-2-ylmethyl)-4-(1- (4-(trifluoromethyl)phenyl)- 1H-pyrazolo[3,4-b]pyridin-3- yl)pyridin-2(1H)-one | ¹H-NMR (400 MHz, CDCl3) δ 8.76-8.72 (m, 3H), 8.63 (d, J = 8.5 Hz, 2H), 8.53 (dd, J = 8.2, 1.5 Hz, 1H), 7.83 (d, J = 8.6 Hz, 2H), 7.59 (d, J = 7.1 Hz, 1H), 7.40 (dd, J = 8.2, 4.5 Hz, 1H), 7.34 (d, J = 1.6 Hz, 1H), 7.26 (t, J = 4.9 Hz, 1H), 7.15 (dd, J = 7.1, 1.8 Hz, 1H), 5.50 (d, J = 16.2 Hz, 2H). LCMS: m/z. 449.1 [M + H]+. |

### Example 3: Synthesis of Compound 21, 23, 24 and 25

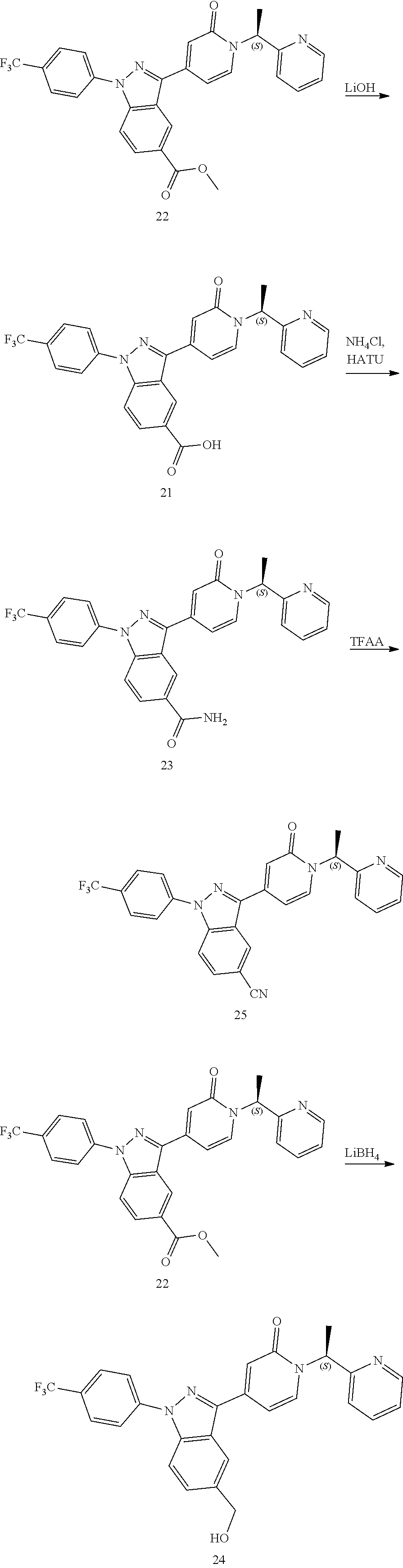

Step 1: To a suspension of compound 22 (200 mg, 0.386 mmol) in THF (3 mL) and H₂O (3 mL) was added LiOH·H₂O (80.98 mg, 1.930 mmol). The yellow solution was stirred at 25° C. for 12 h. The mixture was adjusted to pH=4 with critic acid and extracted with EtOAc (10 mL*3). The combined organic layer was washed with brine, dried and filtered. The filtrate was concentrated under reduced pressure. The residue was purified by combi flash (0-10% MeOH/DCM) to afford compound 21 (170 mg, 87.4%) as a yellow solid.

LC-MS (ESI) m/z: 505.1 [M+H]⁺.

¹H-NMR (400 MHz, DMSO_d₆) δ 8.69 (s, 1H), 8.58 (d, J=4.8 Hz, 1H), 8.22-8.10 (m, 3H), 8.07 (d, J=8.8 Hz, 1H), 8.02 (d, J=8.4 Hz, 2H), 7.95 (d, J=7.2 Hz, 1H), 7.83 (t, J=7.6 Hz, 1H), 7.42 (d, J=8.0 Hz, 1H), 7.34 (dd, J=7.6, 4.8 Hz, 1H), 7.05 (d, J=2.0 Hz, 1H), 6.95 (dd, J=7.2, 2.0 Hz, 1H), 6.25 (q, J=7.2 Hz, 1H), 1.78 (d, J=7.2 Hz, 3H).

Step 2: To a suspension of compound 21 (120 mg, 0.238 mmol), DMAP (145.30 mg, 1.189 mmol) and NH₄Cl (0.025 mL, 0.714 mmol) in DMF (2 mL) was added EDCI (91.20 mg, 0.476 mmol). The brown suspension was stirred at 25° C. for 12 h. The mixture was poured into water (50 mL) and extracted with EtOAc (15 mL*3). The combined organic layer was washed with brine (20 mL), dried and filtered. The filtrate was concentrated under reduced pressure. The residue was purified by combi flash (0-7% MeOH/DCM) to afford compound 23 (100 mg, 83.5%) as a yellow solid.

LC-MS (EST) m/z: 504.1 [M+H]⁺.

¹H-NMR (400 MHz, DMSO_d₆) δ 8.73 (d, J=1.2 Hz, 1H), 8.59 (dt, J=4.8, 1.6 Hz, 1H), 8.42 (s, 1H), 8.16-8.12 (m, 3H), 8.07 (d, J=8.8 Hz, 1H), 8.02 (d, J=8.4 Hz, 2H), 7.92 (d, J=7.2 Hz, 1H), 7.83 (td, J=7.6, 2.0 Hz, 1H), 7.51 (s, 1H), 7.43 (d, J=8.0 Hz, 1H), 7.35 (ddd, J=7.6, 4.8, 1.2 Hz, 1H), 7.25 (d, J=2.0 Hz, 1H), 6.99 (dd, J=7.2, 2.0 Hz, 1H), 6.27 (q, J=7.2 Hz, 1H), 1.79 (d, J=7.2 Hz, 3H).

Step 3: To a solution of compound 23 (30 mg, 0.060 mmol) and TEA (0.037 mL, 0.268 mmol) in anhydrous THF (1 mL) was added TFAA (0.017 mL, 0.119 mmol) at 25° C. The green solution was stirred at 25° C. for 1 h. The mixture was concentrated under reduced pressure. The residue was purified by combi flash (0-70% EtOAc/PE) to afford compound 25 (15 mg, 51.85%) as a yellow solid.

LC-MS (ESI) m/z: 486.1 [M+H]⁺.

¹H-NMR (400 MHz, DMSO_d₆) δ 8.89 (s, 1H), 8.63-8.55 (m, 1H), 8.14 (dd, J=10.8, 8.4 Hz, 3H), 8.02 (d, J=8.4 Hz, 2H), 7.96 (dd, J=8.8, 1.6 Hz, 1H), 7.91 (d, J=7.2 Hz, 1H), 7.83 (td, J=7.6, 2.0 Hz, 1H), 7.42 (d, J=8.0 Hz, 1H), 7.39-7.28 (m, 1H), 7.18 (d, J=2.0 Hz, 1H), 6.97 (dd, J=7.2, 2.0 Hz, 1H), 6.25 (q, J=7.2 Hz, 1H), 1.78 (d, J=7.2 Hz, 3H).

Step 4: To a solution of compound 22 (20 mg, 0.039 mmol) in THF (1 mL) was added LiBH₄ (0.84 mg, 0.039 mmol) at 25° C. The colorless solution was stirred at 25° C. for 1 h. The mixture was quenched with sat·NH₄Cl (10 mL) and extracted with EtOAc (5 mL*3). The combined organic layer was washed with brine, dried and filtered. The filtrate was concentrated under reduced pressure. The residue was purified by combi flash (0-4% MeOH/DCM) to afford compound 24 (13 mg, 68.7%) as a white solid.

LC-MS (ESI) m/z: 491.1 [M+H]⁺.

¹H-NMR (400 MHz, DMSO_d₆) δ 8.57 (ddd, J=4.8, 2.0, 0.8 Hz, 1H), 8.13-8.10 (m, 2H), 7.99 (dd, J=8.8, 7.2 Hz, 3H), 7.89 (d, J=7.2 Hz, 1H), 7.81 (td, J=7.6, 2.0 Hz, 1H), 7.58 (dd, J=8.8, 1.6 Hz, 1H), 7.42-7.38 (m, 1H), 7.33 (ddd, J=7.6, 4.8, 1.2 Hz, 1H), 7.07 (d, J=2.0 Hz, 1H), 6.95 (dd, J=7.2, 2.0 Hz, 1H), 6.52 (s, 1H), 6.24 (q, J=7.2 Hz, 1H), 5.39 (t, J=5.6 Hz, 1H), 4.69 (d, J=5.6 Hz, 2H), 1.77 (d, J=7.2 Hz, 3H).

The following compounds listed below were synthesized according to the procedure of compound 25:

| Com- | From | | | |
|---|---|---|---|---|
| pound | Inter- | | | |
| No. | mediate | Structure | Name | Analytical data |
| 29 | 226 | 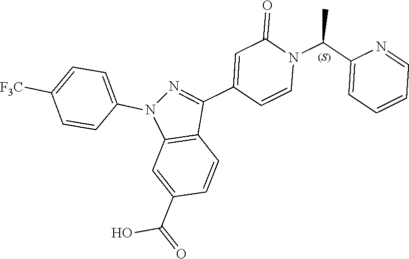 | (S)-3-(2-oxo-1-(1- (pyridin-2-yl)ethyl)- 1,2-dihydropyridin-4- yl)-1-(4- (trifluoromethyl)phenyl)- 1H-indazole-6- carboxylic acid | ¹H-NMR (400 MHz, DMSO_d₆) δ 13.42 (brs, 1H), 8.58 (dt, J = 4.8, 1.2 Hz, 1H), 8.46 (t, J = 1.2 Hz, 1H), 8.34 (d, J = 8.4 Hz, 1H), 8.14 (d, J = 8.4 Hz, 2H), 8.06 (d, J = 8.4 Hz, 2H), 7.98 (dd, J = 8.4, 1.2 Hz, 1H), 7.92 (d, J = 7.2 Hz, 1H), 7.83 (td, J = 7.6, 1.6 Hz, 1H), 7.42 (d, J = 8.0 Hz, 1H), 7.34 (ddd, J = 7.6, 4.8, 1.2 Hz, 1H), 7.10 (d, J = 2.0 Hz, 1H), 6.98 (dd, J = 7.2, 2.0 Hz, 1H), 6.25 (q, J = 7.2 Hz, 1H), 1.78 (d, J = 7.2 Hz, 3H). LCMS: m/z 505.1 [M + H]⁺. |
| 30 | 29 | 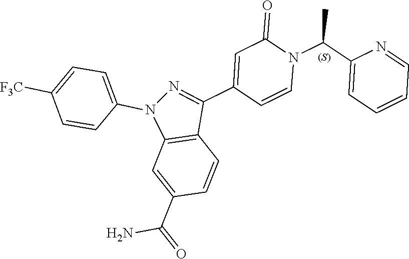 | (S)-3-(2-oxo-1-(1- (pyridin-2-yl)ethyl)- 1,2-dihydropyridin-4- yl)-1-(4- (trifluoromethyl)phenyl)- 1H-indazole-6- carboxamide | ¹H-NMR (400 MHz, DMSO_d₆) δ 8.58 (ddd, J = 4.8, 1.6, 1.2 Hz, 1H), 8.44 (t, J = 1.2 Hz, 1H), 8.34-8.24 (m, 2H), 8.16 (d, J = 8.8 Hz, 2H), 8.03 (d, J = 8.4 Hz, 2H), 7.97- 7.88 (m, 2H), 7.82 (td, J = 7.6, 2.0 Hz, 1H), 7.60 (s, 1H), 7.41 (dt, J = 8.0, 1.2 Hz, 1H), 7.34 (ddd, J = 7.6, 4.8, 1.2 Hz, 1H), 7.10 (d, J = 2.0 Hz, 1H), 6.98 (dd, J = 7.2, 2.0 Hz, 1H), 6.25 (q, J = 7.2 Hz, 1H), 1.77 (d, J = 7.2 Hz, 3H). LCMS: m/z 504.1 [M + H]⁺. |
| 31 | 30 | 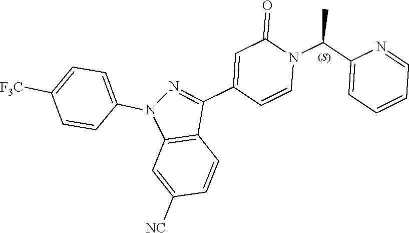 | (S)-3-(2-oxo-1-(1- (pyridin-2-yl)ethyl)- 1,2-dihydropyridin-4- yl)-1-(4- (trifluoromethyl)phenyl)- 1H-indazole-6- carbonitrile | ¹H-NMR (400 MHz, DMSO_d₆) δ 8.64 (d, J = 1.2 Hz, 1H), 8.58 (dt, J = 4.8, 1.6 Hz, 1H), 8.41 (d, J = 8.4 Hz, 1H), 8.16 (d, J = 8.4 Hz, 2H), 8.00 (d, J = 8.4 Hz, 2H), 7.92 (d, J = 7.2 Hz, 1H), 7.83 (td, J = 7.6, 1.6 Hz, 1H), 7.76 (dd, J = 8.4, 1.2 Hz, 1H), 7.42 (d, J = 7.6 Hz, 1H), 7.34 (ddd, J = 7.6, 4.8, 1.2 Hz, 1H), 7.08 (d, J = 2.0 Hz, 1H), 6.95 (dd, J = 7.2, 2.0 Hz, 1H), 6.32- 6.19 (q, J = 7.2 Hz, 1H), 1.78 (d, J = 7.2 Hz, 3H). LCMS: m/z 486.1 [M + H]⁺. |
| 28 | 26 | 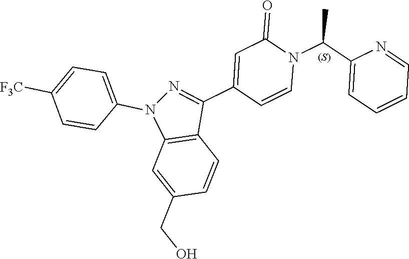 | (S)-4-(6- (hydroxymethyl)-1-(4- (trifluoromethyl)phenyl)- 1H-indazol-3-yl)-1- (1-(pyridin-2- yl)ethyl)pyridin- 2(1H)-one | ¹H-NMR (400 MHz, DMSO_d₆) δ 8.58 (ddd, J = 4.8, 2.0, 0.8 Hz, 1H), 8.16 (d, J = 8.4 Hz, 1H), 8.10 (d, J = 8.4 Hz, 2H), 8.02 (d, J = 8.4 Hz, 2H), 7.94 (t, J = 0.8 Hz, 1H), 7.89 (d, J = 7.2 Hz, 1H), 7.82 (td, J = 7.6, 2.0 Hz, 1H), 7.45- 7.38 (m, 2H), 7.34 (ddd, J = 7.6, 4.8, 1.2 Hz, 1H), 7.07 (d, J = 2.0 Hz, 1H), 6.97 (dd, J = 7.2, 2.0 Hz, 1H), 6.25 (q, J = 7.2 Hz, 1H), 5.44 (t, J = 5.6 Hz, 1H), 4.72 (d, J = 5.6 Hz, 2H), 1.77 (d, J = 7.2 Hz, 3H). LCMS: m/z 491.1 [M + H]⁺. |

### Example 4: Synthesis of Compound 45

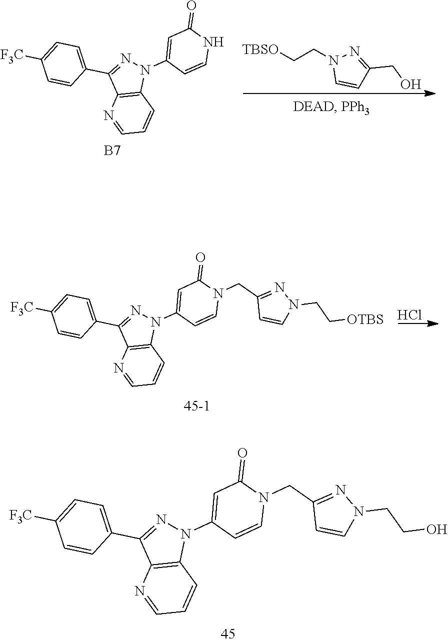

Step 1: To a stirred mixture of compound B7 (50 mg, 0.140 mmol) and compound D2 (44 mg, 0.154 mmol) in DMF (1 mL) were added PPh₃ (55 mg, 0.210 mmol) and Diethyl azodicarboxylate (37 mg, 0.210 mmol), and the mixture was stirred at 20° C. for 12 h. The mixture was diluted with water (10 mL). The mixture was extracted with EtOAc (10 mL×2). The combined layer was washed with brine, dried over anhydrous Na₂SO₄, tittered and concentrated under reduced pressure. The residue was purified by flash column chromatography on silica gel (Gradient elution, 0-50% EtOAc/DCM) to afford compound 45-1 (80 mg, impure) as colorless oil.

LC-MS (ESI) m/z: 595.2 [M+H]⁺.

Step 2: To a solution of compound 45-1 (80 mg, 0.135 mmol) in MeOH (2 mL) was added HCl/dioxane (1 mL, 4.0 M), and the mixture was stirred at 20′CC for 1 h. The reaction mixture was concentrated under reduced pressure. The residue was diluted with saturated aq. NaHCO₃ (10 mL), and the resulting mixture was extracted with DCM (20 mL). The combined layer was washed with brine, dried over anhydrous Na₂SO₄, filtered and concentrated under reduced pressure. The residue was purified by flash column chromatography on silica gel (Gradient elution, 0-10% MeOH/DCM) to afford compound 45 (28 mg, 43.3%) as a white solid.

LC-MS (ESI) m/z: 481.2 [M+H]⁺.

¹H-NMR (400 MHz, CDCl₃) δ 8.83-8.77 (m, 1H), 8.73 (d, J=8.1 Hz, 2H), 8.37 (ddd, J=10.3, 5.5, 1.8 Hz, 1H), 7.78 (d, J=8.1 Hz, 2H), 7.76-7.68 (m, 1H), 7.55-7.42 (m, 2H), 7.11 (dh, J=7.8, 2.6 Hz, 2H), 6.41 (td, J=6.5, 5.9, 2.6 Hz, 1H), 5.26 (s, 2H), 4.34-4.20 (m, 2H), 4.09-3.94 (m, 2H).

### Example 5: Synthesis of Compound 57

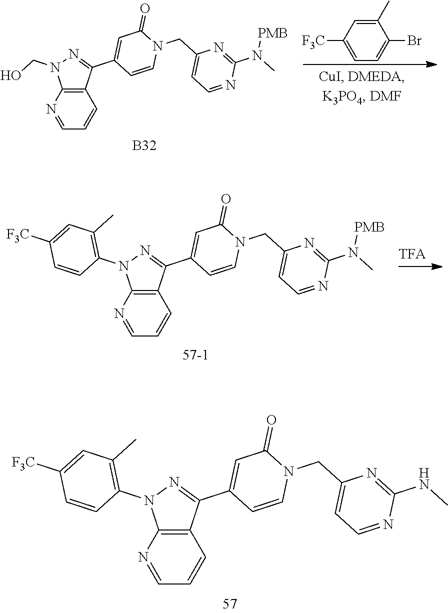

Step 1: To a solution of 1-bromo-2-methyl-4-(trifluoromethyl)benzene (20 mg, 0.083 mmol) and compound B32 (40 mg, 0.083 mmol) in DMF (1 mL) were added CuI (9 mg, 0.050 mmol), N,N′-DiMethylethylenediaMine (9 mg, 0.099 mmol) and K₃PO₄ (35 mg, 0.165 mmol), and the mixture was stirred at 130° C. for 12 h under N₂ atmosphere. The mixture was diluted with water (5 mL). The mixture was extracted with EtOAc (5 mL×2). The combined layer was washed with brine, dried over anhydrous Na₂SO₄, filtered and concentrated under reduced pressure. The residue was purified by flash column chromatography on silica gel (Gradient elution, 0-100% EtOAc/PE) to afford compound 57-1 (40 mg, 79.1%) as a white solid.

LC-MS (ESI) m/z: 612.2 [M+H]⁺.

Step 2: A solution of compound 57-1 (40 mg, 0.065 mmol) in TFA (1 mL) was stirred at 60° C. for 12 h. The mixture was concentrated under reduced pressure. the residue was adjusted pH to 8 with saturated aq. NaHCO₃, then the mixture was extracted with DCM (10 mL×2). The combined layer was washed with brine, dried over anhydrous Na₂SO₄, filtered and concentrated under reduced pressure. The residue was purified by prep-HPLC (formic acid as additive) to afford compound 57 (13 mg, 40.5%) as a white solid.

LC-MS (ESI) m/z: 492.2 [M+H]⁺.

¹H-NMR (400 MHz, DMSO_d6) δ 8.81 (dd, J=8.2, 1.5 Hz, 1H), 8.67 (dd, J=4.5, 1.5 Hz, 1H), 8.26-8.17 (m, 1H), 7.97-7.87 (m, 2H), 7.86-7.77 (m, 2H), 7.49 (dd, J=8.2, 4.5 Hz, 1H), 7.16 (d, J=2.0 Hz, 1H), 7.12-7.03 (m, 1H), 7.00 (dd, J=7.1, 2.0 Hz, 1H), 6.36-6.27 (m, 1H), 5.06 (s, 2H), 2.74 (d, J=4.7 Hz, 3H), 2.28 (s, 3H).

The following compounds listed below were synthesized according to the procedure of compound 57:

| Com- | From | | | |
|---|---|---|---|---|
| pound | Inter- | | | |
| No. | mediate | Structure | Name | Analytical data |
| 58 | B31 | 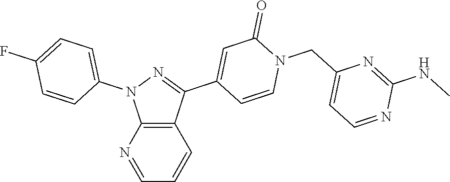 [SMILES: CNc1nccc(Cn2ccc(-c3nn(-c4ccc(F)cc4)c4ncccc34)cc2=O)n1] | 4-(1-(4- fluorophenyl)- 1H-pyrazolo[3,4- b]pyridin- 3-yl)-1-((2- (methylamino) pyrimidin- 4-yl)methyl) pyridin- 2(1H)-one | ¹H-NMR (400 MHz, DMSO-d6) δ 8.85-8.73 (m, 2H), 8.35 (dd, J = 9.2, 4.9 Hz, 2H), 8.27-8.17 (m, 1H), 7.93 (d, J = 7.1 Hz, 1H), 7.55-7.43 (m, 3H), 7.16 (d, J = 1.9 Hz, 1H), 7.13-7.03 (m, 2H), 6.37-6.27 (m, 1H), 5.06 (s, 2H), 2.74 (d, J = 4.7 Hz, 3H). LCMS: m/z 428.2 [M + H]⁺. |
| 59 | B31 | 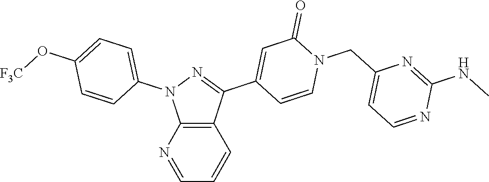 [SMILES: CNc1nccc(Cn2ccc(-c3nn(-c4ccc(OC(F)(F)F)cc4)c4ncccc34)cc2=O)n1] | 1-((2- (methylamino) pyrimidin- 4-yl)methyl)- 4-(1-(4- (trifluoromethoxy) phenyl)- 1H-pyrazolo[3,4- b]pyridin-3-yl) pyridin- 2(1H)-one | ¹H-NMR (400 MHz, DMSO-d6) δ 8.86-8.74 (m, 2H), 8.50 (d, J = 9.2 Hz, 2H), 8.22 (s, 1H), 7.94 (d, J = 7.2 Hz, 1H), 7.71-7.60 (m, 2H), 7.53 (dd, J = 8.2, 4.5 Hz, 1H), 7.17 (d, J = 2.0 Hz, 1H), 7.14-7.04 (m, 2H), 6.33 (s, 1H), 5.07 (s, 2H), 2.74 (d, J = 4.5 Hz, 3H). LCMS: m/z 494.0 [M + H]⁺. |
| 144 | B31 | 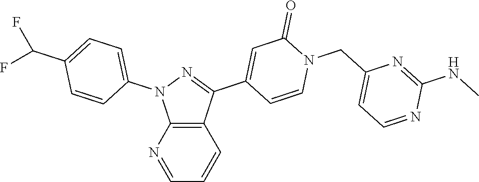 [SMILES: CNc1nccc(Cn2ccc(-c3nn(-c4ccc(C(F)F)cc4)c4ncccc34)cc2=O)n1] | 4-(1-(4- (difluoromethyl) phenyl)- 1H-pyrazolo[3,4- b]pyridin-3- yl)-1-((2- (methylamino) pyrimidin- 4-yl)methyl) pyridin- 2(1H)-one | ¹H-NMR (400 MHz, DMSO_d₆) δ 8.85-8.76 (m, 2H), 8.56 (d, J = 8.4 Hz, 2H), 8.28-8.16 (m, 1H), 7.94 (d, J = 7.1 Hz, 1H), 7.83 (d, J = 8.4 Hz, 2H), 7.53 (dd, J = 8.1, 4.7 Hz, 1H), 7.18 (d, J = 2.0 Hz, 1H), 7.14 (t, J = 56.0 Hz, 1H), 7.12-7.04 (m, 2H), 6.33 (m, 1H), 5.07 (s, 2H), 2.74 (d, J = 4.8 Hz, 3H). LCMS: m/z 460.2 [M + H]⁺. |

### Example 6: Synthesis of Compound 71

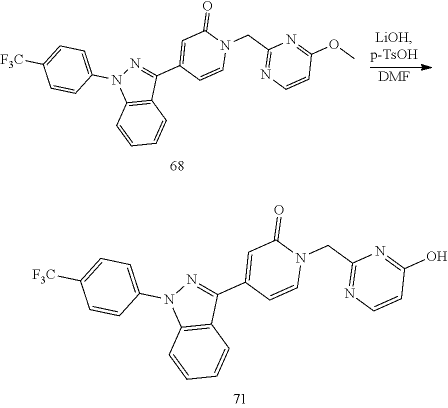

Step 1: To a solution of compound 68 (70 mg, 0.15 mmol) and LiCl (31.07 mg, 0.73 mmol) in DMF (2 mL) was added p-TsOH (139.44 mg, 0.73 mmol) at 20° C. The yellow suspension was stirred at 120° C. for 2 h. TLC showed the reaction was completed. The mixture was cooled to room temperature and poured into water (15 mL) and stirred for 30 min. The white suspension was filtered. The filter cake was triturated with MTBE (20 mL) and filtered. The filter cake was dried under vacuum to afford compound 71 (60 mg, 0.13 mmol, 88.31%) as white solid.

LC-MS (ESI) nm/z: 464.1 [M+H]⁺.

¹H-NMR (400 MHz, DMSO_d₆) δ 12.86 (s, 1H), 8.28-8.23 (m, 1H), 8.15 (d, J=8.4 Hz, 2H), 8.06 (d, J=8.4 Hz, 1H), 8.01 (d, J=8.4 Hz, 2H), 7.90 (d, J=7.2 Hz, 1H), 7.82 (d, J=6.8 Hz, 1H), 7.66 (ddd, J=8.4, 6.8, 1.2 Hz, 1H), 7.52-7.42 (m, 1H), 7.08 (d, J=2.0 Hz, 1H), 7.00 (dd, J=7.2, 2.0 Hz, 1H), 6.24 (s, 1H), 5.08 (s, 2H).

The following compounds listed below were synthesized according to the procedure of compound 71:

| Com- | From | | | |
|---|---|---|---|---|
| pound | Interme | | | |
| No. | diate | Structure | Name | Analytical data |
| 100 | A20 | 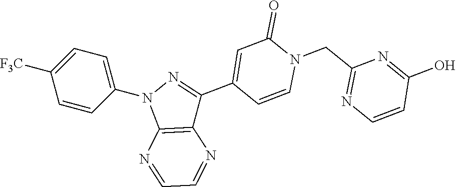 [SMILES: O=c1cc(-c2nn(-c3ccc(C(F)(F)F)cc3)c3nccnc23)ccn1Cc1nccc(O)n1] | 1-((4-hydroxypyrimidin- 2-yl)methyl)-4-(1-(4- (trifluoromethyl)phenyl)- 1H-pyrazolo[3,4- b]pyrazin-3-yl)pyridin- 2(1H)-one | ¹H-NMR (400 MHz, DMSO) δ 12.86 (s, 1H), 8.97 (d, J = 2.2 Hz, 1H), 8.90 (d, J = 2.2 Hz, 1H), 8.62 (d, J = 8.5 Hz, 2H), 8.04 (d, J = 8.7 Hz, 2H), 7.94 (d, J = 7.1 Hz, 1H), 7.74 (d, J = 1.6 Hz, 2H), 7.25 (dd, J = 7.1, 1.8 Hz, 1H), 6.23 (s, 1H), 5.08 (s, 2H). LCMS: m/z 466.1 [M + H]+. |
| 101 | B1 | 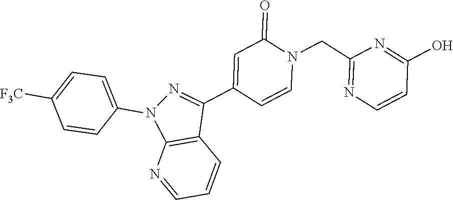 [SMILES: O=c1cc(-c2nn(-c3ccc(C(F)(F)F)cc3)c3ncccc23)ccn1Cc1nccc(O)n1] | 1-((4-hydroxypyrimidin- 2-yl)methyl)-4-(1-(4- (trifluoromethyl)phenyl)- 1H-pyrazolo[3,4- b]pyridin-3-yl)pyridin- 2(1H)-one | ¹H-NMR (400 MHz, DMSO) δ 8.82 (d, J = 6.2 Hz, 2H), 8.69 (d, J = 8.6 Hz, 2H), 8.01 (d, J = 8.5 Hz, 2H), 7.93 (s, 1H), 7.84 (d, J = 6.4 Hz, 1H), 7.56 (t, J = 6.8 Hz, 1H), 7.16 (s, 1H), 7.08 (d, J = 6.9 Hz, 1H), 6.26 (d, J = 6.5 Hz, 1H), 5.10 (s, 2H). LCMS: m/z 465.1 [M + H]+. |

### Example 7: Synthesis of Compound 72

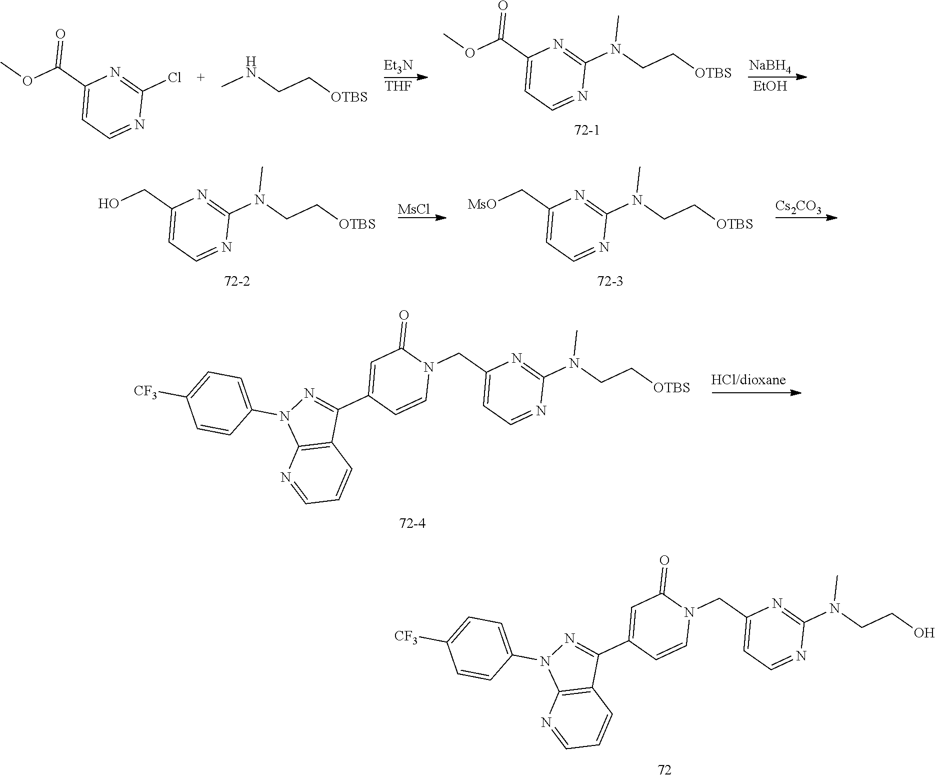

Step 1: To a solution of methyl 2-chloropyrimidine-4-carboxylate (500 mg, 2.90 mmol) and Et₃N (1.21 mL, 8.69 mmol) in THF (4 mL) was added 2,2,3,3-tetramethyl-7-aza-4-oxa-3-silaoctane (548.68 mg, 2.90 mmol) dropwise at 25° C. The yellow solution was stirred at 25° C. for 12 h. The desired MS peak was detected by LCMS. The mixture was concentrated under reduced pressure. The residue was purified by combi flash (0-10% EtOAc/Petroleum ether) to afford compound 72-1 (900 mg, 2.77 mmol, 95.44%) as yellow oil.

LC-MS (ESI) m/z: 326.1 [M+H]⁺.

Step 2: To a solution of compound 72-1 (600 mg, 1.84 mmol) in EtOH (10 mL) were added NaBH₄ (139.45 mg, 3.67 mmol) at 25° C., and the mixture was stirred at 25° C. for 2 h. The reaction was quenched with MeOH and stirred for 30 min at 25° C. The mixture was concentrated under reduced pressure. The residue was purified by flash column chromatography on silica gel (Gradient elution, 0-20% EtOAc/Petroleum ether) to afford compound 72-2 (300 mg, 1.01 mmol, 54.71%) as colorless oil.

LC-MS (ESI) m/z: 298.1 [M+H]+.

Step 3: To a solution of compound 72-2 (60 mg, 0.20 mmol) and DIPEA (0.07 mL, 0.40 mmol) in DCM (1 mL) was added MsCl (0.02 mL, 0.22 mmol) at 25° C. The yellow solution was stirred at 25° C. for 0.5 h. TLC showed the reaction was completed. The mixture was extracted with DCM (5 mL*3) and water (10 mL). The combined organic layer was dried over anhydrous sodium sulfate and filtered. The filtrate was concentrated under reduced pressure to afford compound 72-3 (76 mg, 0.20 mmol, 100%) as yellow oil, which was used for next step directly.

LC-MS (ESI) m/z: 376.1 [M+H]⁺.

Step 4: A suspension of compound B1 (50 mg, 0.14 mmol), compound 72-3 (52.70 mg, 0.14 mmol) and Cs₂CO₃ (91.44 mg, 0.28 mmol) in DMF (2 mL) was stirred at 80° C. for 12 h. TLC showed the reaction was completed. The mixture was extracted with EtOAc (10 mL*3) and water (20 mL), the combined organic layer was washed with brine (20 mL), dried and filtered. The filtrate was concentrated under reduced pressure. The residue was purified by combi flash (0-5% MeOH/DCM) to afford compound 72-4 (50 mg, 0.08 mmol, 56.04%) as yellow solid.

LC-MS (ESI) m/z: 636.1 [M+H]⁺.

Step 5: A solution of compound 72-4 (50 mg, 0.08 mmol) in HCl/dioxane (0.5 mL) and MeOH (2 mL) was stirred at 25° C. for 1 h. The desired MS peak was detected by LCMS. The mixture was adjusted to pH=8 with sat·NaHCO₃ and extracted with EtOAc (10 mL*3), the combined organic layer was washed with brine (15 mL), dried and filtered. The filtrate was concentrated under reduced pressure. The residue was purified by combi flash (0-7% MeOH/DCM) to afford compound 72 (20 mg, 0.04 mmol, 48.76%) as yellow solid.

LC-MS (ESI) m/z: 522.1 [M+H]⁺.

¹H-NMR (400 MHz, DMSO_d₆) δ 8.84-8.78 (m, 2H), 8.68 (d, J=8.8 Hz, 2H), 8.27 (d, J=5.2 Hz, 1H), 8.00 (d, J=8.8 Hz, 2H), 7.94 (d, J=7.2 Hz, 1H), 7.60-7.52 (m, 1H), 7.17 (d, J=1.6 Hz, 1H), 7.07 (d, J=7.2 Hz, 1H), 6.41 (d, J=5.2 Hz, 1H), 5.10 (s, 2H), 4.60 (brs, 1H), 3.62-3.42 (m, 4H), 3.07 (s, 3H).

The following compounds listed below were synthesized according to the procedure of compound 72:

| Com- | From | | | |
|---|---|---|---|---|
| pound | Inter- | | | |
| No. | mediate | Structure | Name | Analytical data |
| 73 | B1 | 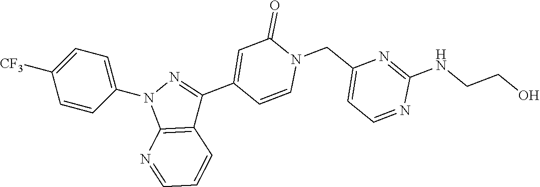 [SMILES: O=c1cc(-c2nn(-c3ccc(C(F)(F)F)cc3)c3ncccc23)ccn1Cc1ccnc(NCCO)n1] | 1-((2-((2- hydroxyethyl)amino) pyrimidin- 4-yl)methyl)-4-(1-(4- (trifluoromethyl)phenyl)- 1H-pyrazolo[3,4-b]pyridin- 3-yl)pyridin-2(1H)-one | ¹H NMR (400 MHz, DMSO-d6) δ 8.81 (d, J = 6.4 Hz, 2H), 8.68 (d, J = 8.4 Hz, 2H), 8.21 (d, J = 5.2 Hz, 1H), 8.00 (d, J = 8.4 Hz, 2H), 7.60-7.52 (m, 1H), 7.18 (d, J = 2.0 Hz, 1H), 7.07 (dd, J = 7.2, 2.0 Hz, 2H), 6.36 (d, J = 5.2 Hz, 1H), 5.07 (s, 2H), 4.63 (br.s, 1H), 3.55-3.43 (m, 4H). LCMS: m/z 508.1 [M + H]⁺. |
| 74 | B1 | 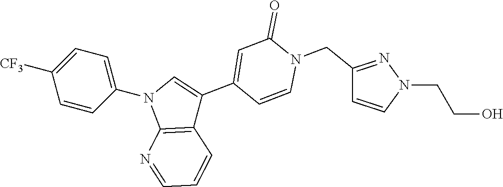 [SMILES: O=c1cc(-c2cn(-c3ccc(C(F)(F)F)cc3)c3ncccc23)ccn1Cc1ccn(CCO)n1] | 1-((1-(2-hydroxyethyl)-1H- pyrazol-3-yl)methyl)-4-(1- (4-(trifluoromethyl)phenyl)- 1H-pyrazolo[3,4-b]pyridin- 3-yl)pyridin-2(1H)-one | ¹H NMR (400 MHz, DMSO-d6) δ 8.88-8.77 (m, 2H), 8.67 (d, J = 8.8 Hz, 2H), 8.00 (d, J = 8.8 Hz, 2H), 7.89 (d, J = 7.2 Hz, 1H), 7.66 (d, J = 2.0 Hz, 1H), 7.55 (dd, J = 8.4, 4.8 Hz, 1H), 7.16 (d, J = 2.0 Hz, 1H), 7.01 (dd, J = 7.2, 2.0 Hz, 1H), 5.13 (s, 2H), 4.89 (t, J = 5.2 Hz, 1H), 4.11 (t, J = 5.6 Hz, 2H), 3.72 (q, J = 5.6 Hz, 2H). LCMS: m/z 481.1 [M + H]⁺. |
| 75 | B1 | 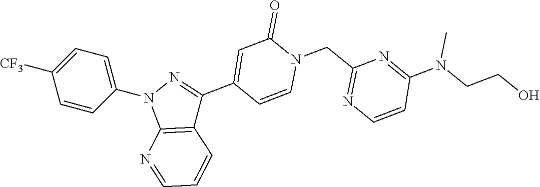 [SMILES: CN(CCO)c1ccnc(Cn2ccc(-c3nn(-c4ccc(C(F)(F)F)cc4)c4ncccc34)cc2=O)n1] | 1-((4-((2- hydroxyethyl)(methyl)amino) pyrimidin-2-yl)methyl)-4- (1-(4- (trifluoromethyl)phenyl)- 1H-pyrazolo[3,4-b]pyridin- 3-yl)pyridin-2(1H)-one | ¹H NMR (400 MHz, DMSO) δ 8.88-8.79 (m, 2H), 8.70 (d, J = 8.4 Hz, 2H), 8.09 (s, 1H), 8.02 (d, J = 8.4 Hz, 2H), 7.93 (d, J = 7.2 Hz, 1H), 7.44-7.38 (m, 1H), 7.15 (d, J = 2.0 Hz, 1H), 7.04 (d, J = 7.2 Hz, 1H), 6.55 (br.s, 1H), 5.13 (s, 2H), 3.59-3.39 (m, 4H), 3.02 (s, 3H). LCMS: m/z 522.1 [M + H]⁺. |
| 102 | A20 | 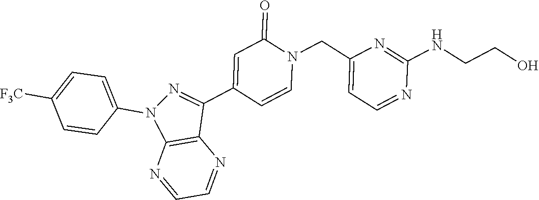 [SMILES: O=c1cc(-c2nn(-c3ccc(C(F)(F)F)cc3)c3nccnc23)ccn1Cc1ccnc(NCCO)n1] | 1-((2-((2- hydroxyethyl)amino) pyrimidin- 4-yl)methyl)-4-(1-(4- (trifluoromethyl)phenyl)- 1H-pyrazolo[3,4-b]pyrazin- 3-yl)pyridin-2(1H)-one | ¹H NMR (400 MHz, DMSO) δ 8.97 (d, J = 2.2 Hz, 1H), 8.90 (d, J = 2.2 Hz, 1H), 8.63 (d, J = 8.5 Hz, 2H), 8.22 (d, J = 5.0 Hz, 1H), 8.04 (d, J = 8.7 Hz, 2H), 7.96 (d, J = 7.1 Hz, 1H), 7.78 (d, J = 1.5 Hz, 1H), 7.27 (dd, J = 7.1, 1.8 Hz, 1H), 7.11 (s, 1H), 6.37 (d, J = 5.1 Hz, 1H), 5.07 (s, 2H), 3.50-3.43 (m, 4H). LCMS: m/z 509.1 [M + H]+. |
| 106 | A20 | 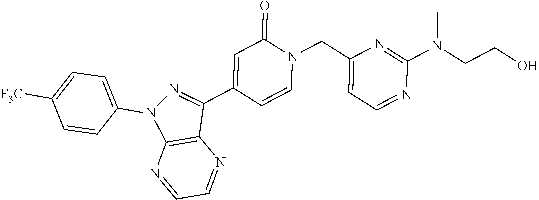 [SMILES: CN(CCO)c1nccc(Cn2ccc(-c3nn(-c4ccc(C(F)(F)F)cc4)c4nccnc34)cc2=O)n1] | 1-((2-((2- hydroxyethyl)(methyl)amino) pyrimidin-4-yl)methyl)-4- (1-(4- (trifluoromethyl)phenyl)- 1H-pyrazolo[3,4-b]pyrazin- 3-yl)pyridin-2(1H)-one | ¹H NMR (400 MHz, DMSO) δ 8.96 (d, J = 2.1 Hz, 1H), 8.89 (d, J = 2.2 Hz, 1H), 8.62 (d, J = 8.6 Hz, 2H), 8.27 (d, J = 5.0 Hz, 1H), 8.03 (d, J = 8.7 Hz, 2H), 7.96 (d, J = 7.1 Hz, 1H), 7.78 (s, 1H), 7.26 (d, J = 7.0 Hz, 1H), 6.42 (d, J = 5.0 Hz, 1H), 5.10 (s, 2H), 4.60 (s, 1H), 3.52 (d, J = 23.5 Hz, 4H), 3.07 (s, 3H). LCMS: m/z 523.4 [M + H]+. |
| 107 | A20 | 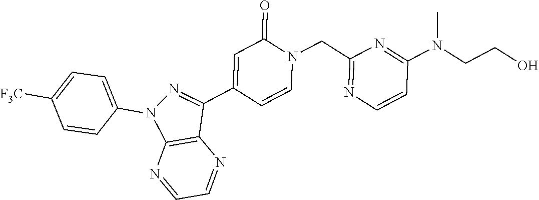 [SMILES: CN(CCO)c1ccnc(Cn2ccc(-c3nn(-c4ccc(C(F)(F)F)cc4)c4nccnc34)cc2=O)n1] | 1-((4-((2- hydroxyethyl)(methyl)amino) pyrimidin-2-yl)methyl)-4- (1-(4- (trifluoromethyl)phenyl)- 1H-pyrazolo[3,4-b]pyrazin- 3-yl)pyridin-2(1H)-one | ¹H NMR (400 MHz, DMSO) δ 8.96 (d, J = 2.2 Hz, 1H), 8.89 (d, J = 2.2 Hz, 1H), 8.62 (d, J = 8.5 Hz, 2H), 8.07 (dd, J = 32.1, 15.0 Hz, 3H), 7.93 (d, J = 7.1 Hz, 1H), 7.74 (d, J = 1.7 Hz, 1H), 7.22 (dd, J = 7.1, 1.8 Hz, 1H), 6.56 (s, 1H), 5.12 (s, 2H), 4.66 (s, 1H), 3.48 (s, 4H), 3.00 (s, 3H). LCMS: m/z 523.4 [M + H]+. |
| 138 | A25 | 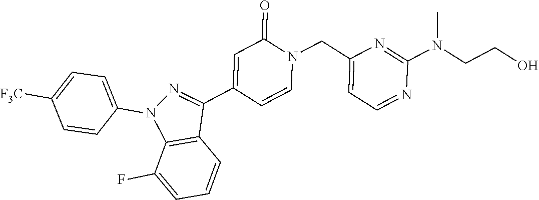 [SMILES: CN(CCO)c1nccc(Cn2ccc(-c3nn(-c4ccc(C(F)(F)F)cc4)c4c(F)cccc34)cc2=O)n1] | 4-(7-fluoro-1-(4- (trifluoromethyl)phenyl)- 1H-indazol-3-yl)-1-((2-((2- hydroxyethyl)(methyl)amino) pyrimidin-4- yl)methyl)pyridin-2(1H)- one | ¹H NMR (400 MHz, DMSO) δ 8.30 (d, J = 5.2 Hz, 1H), 8.06 (d, J = 8.1 Hz, 1H), 7.99 (d, J = 9.2 Hz, 4H), 7.94 (d, J = 7.1 Hz, 1H), 7.51-7.21 (m, 3H), 7.08 (s, 1H), 6.96 (d, J = 7.1 Hz, 1H), 6.53 (s, 1H), 5.15 (s,2H), 3.51-3.44 (m, 4H), 3.10 (s, 3H). LCMS: m/z 539.1 [M + H]+. |
| 139 | A26 | 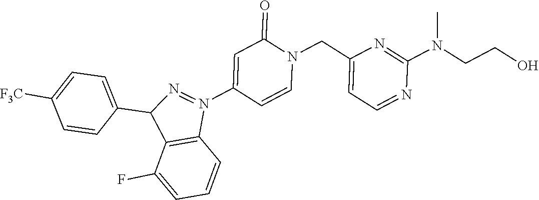 | 4-(4-fluoro-3-(4- (trifluoromethyl)phenyl)- 1H-indazol-1-yl)-1-((2-((2- hydroxyethyl)(methyl)amino) pyrimidin-4- yl)methyl)pyridin-2(1H)- one | ¹H NMR (400 MHz, DMSO) δ 8.90 (s, 1H), 8.32 (d, J = 5.2 Hz, 1H), 8.17 (d, J = 7.7 Hz,2H), 8.16-7.93 (m, 4H), 7.69-7.64 (m, 1H), 7.27 (dd, J = 11.1, 7.9 Hz, 1H), 7.03- 7.00 (m, 1H), 6.88 (d, J = 2.2 Hz, 1H), 6.62 (s, 1H), 5.20 (s, 2H), 3.64-3.44 (m, 4H), 3.13 (s, 3H). LCMS: m/z 539.1 [M + H]+. |

### Example 8: Synthesis of Compound 77

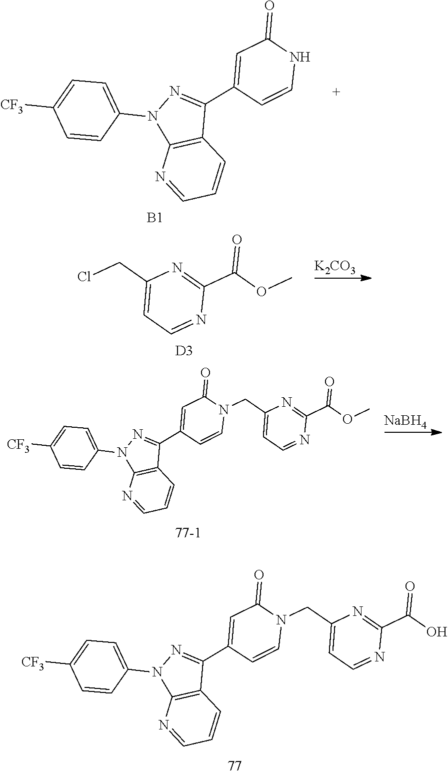

Step 1: A suspension of Compound B1 (35 mg, 0.1 mmol), compound D3 (21.99 mg, 0.12 mmol) and K₂CO₃ (27.15 mg, 0.20 mmol) in DMF (1 mL) was stirred at 40° C. for 12 h. The desired MS peak was detected by LCMS. The mixture was extracted with DCM/MeOH (10/1, 10 mL*3) and water (15 mL), washed with brine (15 mL), dried and filtered. The filtrate was concentrated under reduced pressure. The residue was purified by combi flash (0-25% EtOAc/DCM) to afford compound 77-1 (30 mg, 0.06 mmol, 60.30%) as yellow solid.

LC-MS (ESI) m/z: 505.1 [M−H]⁻.

Step 2: A suspension of compound 77-1 (20 mg, 0.04 mmol) and NaBH₄ (4.5 mg, 0.12 mmol) in EtOH (2 mL) was stirred at 25° C. for 12 h. The desired MS peak was detected by LCMS. The mixture was concentrated under reduced pressure. The residue was purified by combi flash (0-6% MeOH/DCM) and further purified by prep-HPLC (HCOOH) to afford compound 77 (3 mg, 0.006 mmol, 15.88%) as white solid.

LC-MS (ESI) m/z: 479.1 [M+H]⁺.

¹H-NMR (400 MHz, DMSO_d₆) δ 8.86-8.79 (m, 2H), 8.74-8.68 (m, 3H), 8.04-7.99 (m, 3H), 7.55 (dd, J=8.0, 4.8 Hz, 1H), 7.20 (d, J=2.0 Hz, 1H), 7.18-7.08 (m, 2H), 5.28-5.25 (m, 3H), 4.57 (d, J=6.0 Hz, 2H).

### Example 9: Synthesis of Compound 128

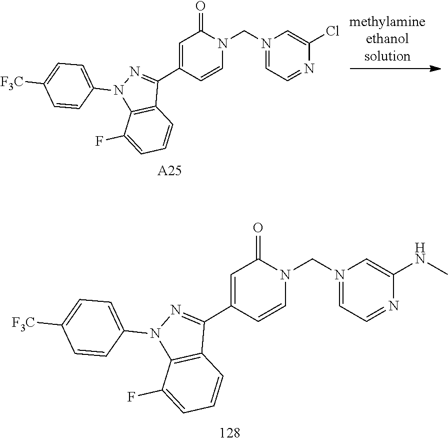

To a solution of compound A25 (370 mg, 0.74 mmol) in methylamine ethanol solution (5 mL) was stirred at 25° C. for 8 h. The mixture was diluted with water and extracted with EtOAc twice. The combined organic layer was washed with brine, dried over anhydrous Na₂SO₄, filtered and concentrated under reduced pressure. The residue was purified by flash column chromatography on silica gel (Gradient elution, 5% MeOH in DCM) to give compound 128 (345 mg, 0.70 mmol, 94.26%) as a white solid.

¹H-NMR (400 MHz, DMSO) δ 8.22 (s, 1H), 8.07 (d, J=8.1 Hz, 1H), 8.03-7.97 (m, 4H), 7.92 (d, J=7.0 Hz, 1H), 7.53-7.40 (m, 2H), 7.08 (d, J=1.7 Hz, 2H), 6.95 (dd, J=7.1, 1.8 Hz, 1H), 6.33 (d, J=4.6 Hz, 1H), 5.07 (s, 2H), 2.74 (d, J=4.2 Hz, 3H).

LCMS: m/z 495.1 [M+H]⁺.

The following compounds listed below were synthesized according to the procedure of compound 128:

| | From | | | |
|---|---|---|---|---|
| Compound | Inter- | | | |
| No. | mediate | Structure | Name | Analytical data |
| 69 | A1 | 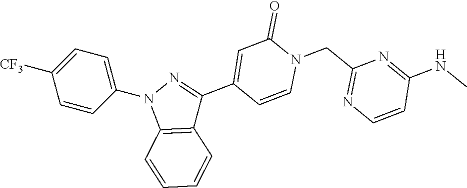 [SMILES: CNc1ccnc(Cn2ccc(-c3nn(-c4ccc(C(F)(F)F)cc4)c4ccccc34)cc2=O)n1] | 1-((4- (methylamino)pyrimidin- 2-yl)methyl)-4-(1-(4- (trifluoromethyl)phenyl)- 1H-indazol-3-yl)pyridin- 2(1H)-one | ¹H NMR (400 MHz, DMSO-d6) δ 8.26 (d, J = 8.4 Hz, 1H), 8.15 (d, J = 8.4 Hz, 2H), 8.07 (d, J = 8.4 Hz, 1H), 8.02 (d, J = 8.4 Hz, 2H), 7.97 (d, J = 7.2 Hz, 1H), 7.67 (dd, J = 8.8, 7.2 Hz, 1H), 7.51-7.45 (m, 1H), 7.15-7.05 (m, 2H), 6.77-6.67 (m, 1H), 6.66- 6.58 (m, 1H), 5.25 (s, 2H), 2.81 (s, 3H). LCMS: m/z 477.1 [M + H]⁺. |
| 70 | B1 | 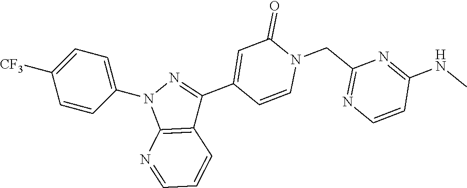 [SMILES: CNc1ccnc(Cn2ccc(-c3nn(-c4ccc(C(F)(F)F)cc4)c4ncccc34)cc2=O)n1] | 1-((4- (methylamino)pyrimidin- 2-yl)methyl)-4-(1-(4- (trifluoromethyl)phenyl)- 1H-pyrazolo[3,4- b]pyridin-3-yl)pyridin- 2(1H)-one | ¹H NMR (400 MHz, DMSO-d6) δ 8.90-8.78 (m, 2H), 8.70 (d, J = 8.4 Hz, 2H), 8.01 (d, J = 8.4 Hz, 2H), 7.93 (d, J = 7.2 Hz, 1H), 7.61-7.52 (m, 1H), 7.49-7.41 (m, 1H), 7.15 (d, J = 2.0 Hz, 1H), 7.04 (dd, J = 7.2, 2.0 Hz, 1H), 6.34 (d, J = 6.0 Hz, 1H), 5.11 (s, 2H), 2.75 (s, 3H). LCMS: m/z 478.1 [M + H]⁺. |
| 78 | A1 | 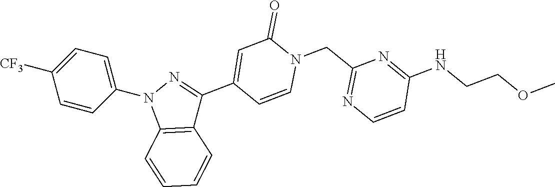 [SMILES: COCCNc1ccnc(Cn2ccc(-c3nn(-c4ccc(C(F)(F)F)cc4)c4ccccc34)cc2=O)n1] | 1-((4-((2- methoxyethyl)amino) pyrimidin-2-yl)methyl)-4-(1- (4- (trifluoromethyl)phenyl)- 1H-indazol-3-yl)pyridin- 2(1H)-one | ¹H NMR (400 MHz, DMSO-d6) δ 8.24 (d, J = 8.4 Hz, 1H), 8.14 (d, J = 8.4 Hz, 2H), 8.05 (d, J = 8.4 Hz, 1H), 8.00 (d, J = 8.4 Hz, 2H), 7.95 (brs, 1H), 7.88 (d, J = 7.2 Hz, 1H), 7.65 (ddd, J = 8.4, 7.2, 1.2 Hz, 1H), 7.52 (brs, 1H), 7.47 (dd, J = 8.0, 7.2 Hz, 1H), 7.06 (d, J = 2.0 Hz, 1H), 6.95 (dd, J = 7.2, 2.0 Hz, 1H), 6.38 (d, J = 6.0 Hz, 1H), 5.09 (s, 2H), 3.42- 3.32 (m, 4H), 3.18 (s, 3H). LCMS: m/z 521.1 [M + H]⁺. |
| 79 | A1 | 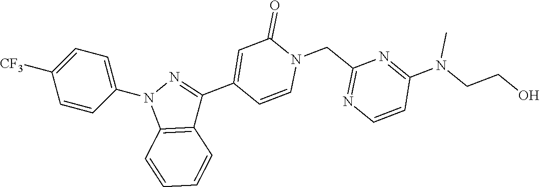 [SMILES: CN(CCO)c1ccnc(Cn2ccc(-c3nn(-c4ccc(C(F)(F)F)cc4)c4ccccc34)cc2=O)n1] | 1-((4-((2- hydroxyethyl)(methyl) amino)pyrimidin-2- yl)methyl)-4-(1-(4- (trifluoromethyl)phenyl)- 1H-indazol-3-yl)pyridin- 2(1H)-one | ¹H NMR (400 MHz, DMSO-d6) δ 8.23 (d, J = 8.4 Hz, 1H), 8.14 (d, J = 8.4 Hz, 2H), 8.09 (d, J = 6.4 Hz, 1H), 8.05 (d, J = 8.4 Hz, 1H), 8.00 (d, J = 8.4 Hz, 2H), 7.89 (d, J = 7.2 Hz, 1H), 7.65 (ddd, J = 8.4, 7.2, 1.2 Hz, 1H), 7.46 (dd, J = 8.0, 7.2 Hz, 1H), 7.06 (d, J = 2.0 Hz, 1H), 6.96 (dd, J = 7.2, 2.0 Hz, 1H), 6.54 (s, 1H), 5.12 (s, 2H), 4.66 (br.s, 1H), 3.48 (s, 4H), 3.01 (s, 3H). LCMS: m/z 521.1 [M + H]⁺ |
| 81 | A1 | 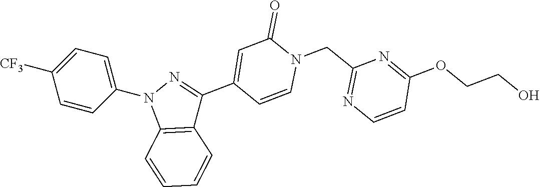 [SMILES: O=c1cc(-c2nn(-c3ccc(C(F)(F)F)cc3)c3ccccc23)ccn1Cc1nccc(OCCO)n1] | 1-((4-(2- hydroxyethoxy)pyrimidin- 2-yl)methyl)-4-(1-(4- (trifluoromethyl)phenyl)- 1H-indazol-3-yl)pyridin- 2(1H)-one | ¹H NMR (400 MHz, DMSO-d6) δ 8.46 (d, J = 6.0 Hz, 1H), 8.24 (d, J = 8.4 Hz, 1H), 8.14 (d, J = 8.4 Hz, 2H), 8.05 (d, J = 8.4 Hz, 1H), 8.00 (d, J = 8.4 Hz, 2H), 7.94 (d, J = 7.2 Hz, 1H), 7.65 (t, J = 7.6 Hz, 1H), 7.46 (t, J = 7.6 Hz, 1H), 7.08 (d, J = 2.0 Hz, 1H), 6.99 (dd, J = 7.2, 2.0 Hz, 1H), 6.83 (d, J = 6.0 Hz, 1H), 5.27 (s, 2H), 4.86 (t, J = 5.6 Hz, 1H), 4.29- 4.21 (m, 2H), 3.65 (q, J = 5.2 Hz, 2H). LCMS: m/z 508.1 [M + H]⁺ |
| 82 | A1 | 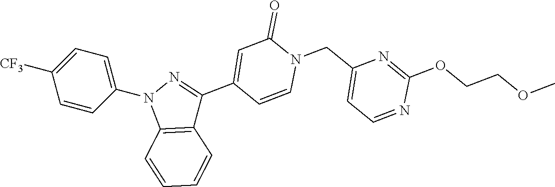 [SMILES: COCCOc1nccc(Cn2ccc(-c3nn(-c4ccc(C(F)(F)F)cc4)c4ccccc34)cc2=O)n1] | 1-((2-(2- methoxyethoxy)pyrimidin- 4-yl)methyl)-4-(1-(4- (trifluoromethyl)phenyl)- 1H-indazol-3-yl)pyridin- 2(1H)-one | ¹H NMR (400 MHz, DMSO-d6) δ 8.47 (d, J = 6.0 Hz, 1H), 8.24 (d, J = 8.4 Hz, 1H), 8.14 (d, J = 8.4 Hz, 2H), 8.05 (d, J = 8.8 Hz, 1H), 8.00 (d, J = 8.8 Hz, 2H), 7.94 (d, J = 7.2 Hz, 1H), 7.65 (ddd, J = 8.4, 7.0, 1.1 Hz, 1H), 7.46 (t, J = 7.6 Hz, 1H), 7.08 (d, J = 2.0 Hz, 1H), 7.00 (dd, J = 7.2, 2.0 Hz, 1H), 6.85 (d, J = 6.0 Hz, 1H), 5.27 (s, 2H), 4.40-4.33 (m, 2H), 3.64- 3.56 (m, 2H), 3.22 (s, 3H). LCMS: m/z 522.1 [M + H]⁺ |
| 84 | A1 | 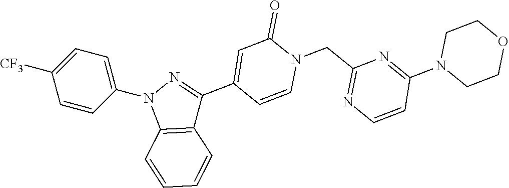 [SMILES: O=c1cc(-c2nn(-c3ccc(C(F)(F)F)cc3)c3ccccc23)ccn1Cc1nccc(N2CCOCC2)n1] | 1-((4- morpholinopyrimidin-2- yl)methyl)-4-(1-(4- (trifluoromethyl)phenyl)- 1H-indazol-3-yl)pyridin- 2(1H)-one | ¹H NMR (400 MHz, DMSO-d6) δ 8.23 (dt, J = 8.4, 1.0 Hz, 1H), 8.19-8.11 (m, 3H), 8.06 (dd, J = 8.8, 0.8 Hz, 1H), 8.00 (d, J = 8.8 Hz, 2H), 7.90 (d, J = 7.2 Hz, 1H), 7.65 (ddd, J = 8.4, 7.2, 0.8 Hz, 1H), 7.47 (ddd, J = 8.0, 6.8, 0.8 Hz, 1H), 7.07 (d, J = 2.0 Hz, 1H), 6.97 (dd, J = 7.2, 2.0 Hz, 1H), 6.73 (d, J = 6.4 Hz, 1H), 5.14 (s, 2H), 3.63 (dd, J = 5.6, 3.6 Hz, 4H), 3.55 (q, J = 4.8, 3.6 Hz, 4H). LCMS: m/z 533.1 [M + H]⁺ |
| 85 | A1 | 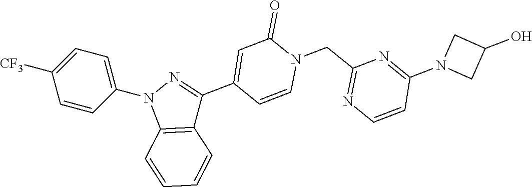 [SMILES: O=c1cc(-c2nn(-c3ccc(C(F)(F)F)cc3)c3ccccc23)ccn1Cc1nccc(N2CC(O)C2)n1] | 1-((4-(3-hydroxyazetidin- 1-yl)pyrimidin-2- yl)methyl)-4-(1-(4- (trifluoromethyl)phenyl)- 1H-indazol-3-yl)pyridin- 2(1H)-one | 1H NMR (400 MHz, DMSO-d6) δ 8.24 (dt, J = 8.4, 1.2 Hz, 1H), 8.18-8.11 (m, 2H), 8.11-8.04 (m, 2H), 8.00 (d, J = 8.4 Hz, 2H), 7.65 (ddd, J = 8.4, 6.8, 1.2 Hz, 1H), 7.47 (ddd, J = 8.0, 6.8, 1.2 Hz, 1H), 6.96 (dd, J = 7.2, 2.0 Hz, 1H), 6.29 (d, J = 6.0 Hz, 1H), 5.83 (d, J = 6.4 Hz, 1H), 5.11 (s, 2H), 4.58 (q, J = 5.2 Hz, 1H), 4.20 (dd, J = 9.6, 6.8 Hz, 2H), 3.79-3.67 (m, 2H). LCMS: m/z 519.1 [M + H]⁺ |
| 86 | A1 | 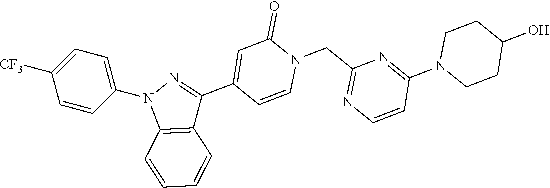 [SMILES: O=c1cc(-c2nn(-c3ccc(C(F)(F)F)cc3)c3ccccc23)ccn1Cc1nccc(N2CCC(O)CC2)n1] | 1-((4-(4-hydroxypiperidin- 1-yl)pyrimidin-2- yl)methyl)-4-(1-(4- (trifluoromethyl)phenyl)- 1H-indazol-3-yl)pyridin- 2(1H)-one | ¹H NMR (400 MHz, DMSO-d6) δ 8.23 (d, J = 8.4 Hz, 1H), 8.15 (d, J = 8.4 Hz, 2H), 8.10 (d, J = 6.4 Hz, 1H), 8.06 (d, J = 8.8 Hz, 1H), 8.00 (d, J = 8.4 Hz, 2H), 7.90 (d, J = 7.2 Hz, 1H), 7.68-7.63 (m, 1H), 7.47 (t, J = 7.6 Hz, 1H), 7.07 (d, J = 2.0 Hz, 1H), 6.97 (dd, J = 7.2, 2.0 Hz, 1H), 6.72 (d, J = 6.4 Hz, 1H), 5.12 (s, 2H), 4.74 (s, 1H), 4.02-3.92 (m, 2H), 3.76-3.66 (m, 1H), 3.23-3.13 (m, 2H), 1.77-1.64 (m, 2H), 1.34-1.24 (m, 2H). LCMS: m/z 547.1 [M + H]⁺ |
| 87 | A1 | 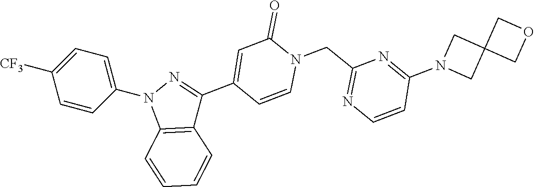 [SMILES: O=c1cc(-c2nn(-c3ccc(C(F)(F)F)cc3)c3ccccc23)ccn1Cc1nccc(N2CC3(COC3)C2)n1] | 1-((4-(2-oxa-6- azaspiro[3.3]heptan-6- yl)pyrimidin-2-yl)methyl)- 4-(1-(4- (trifluoromethyl)phenyl)- 1H-indazol-3-yl)pyridin- 2(1H)-one | 1H NMR (400 MHz, DMSO-d6) δ 8.24 (dt, J = 8.4, 1.0 Hz, 1H), 8.18-8.11 (m, 2H), 8.09 (d, J = 6.0 Hz, 1H), 8.05 (dd, J = 8.8, 1.0 Hz, 1H), 8.00 (d, J = 8.8 Hz, 2H), 7.87 (d, J = 7.2 Hz, 1H), 7.65 (ddd, J = 8.4, 7.2, 0.8 Hz, 1H), 7.47 (ddd, J = 8.0, 7.2, 0.8 Hz, 1H), 7.06 (d, J = 2.0 Hz, 1H), 6.96 (dd, J = 7.2, 2.0 Hz, 1H), 6.28 (d, J = 6.0 Hz, 1H), 5.11 (s, 2H), 4.70 (s, 4H), 4.17 (s, 4H). LCMS: m/z 545.1 [M + H]⁺ |
| 90 | A1 | 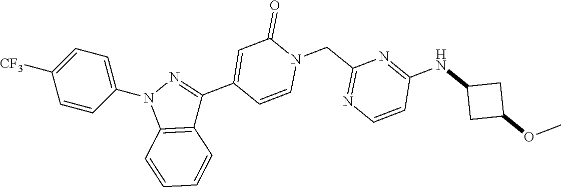 | 1-((4-(((1s,3s)-3- methoxycyclobutyl)amino) pyrimidin-2-yl)methyl)- 4-(1-(4- (trifluoromethyl)phenyl)- 1H-indazol-3-yl)pyridin- 2(1H)-one | ¹H NMR (400 MHz, DMSO-d6) δ 8.23 (d, J = 8.4 Hz, 1H), 8.14 (d, J = 8.4 Hz, 2H), 8.05 (d, J = 8.4 Hz, 1H), 8.00 (d, J = 8.4 Hz, 2H), 7.87 (d, J = 7.2 Hz, 1H), 7.73-7.57 (m, 2H), 7.46 (t, J = 7.6 Hz, 1H), 7.05 (d, J = 2.0 Hz, 1H), 6.95 (dd, J = 7.2, 2.0 Hz, 1H), 6.28 (d, J = 6.0 Hz, 1H), 5.08 (s, 2H), 3.89-3.79 (m, 1H), 3.38-3.32 (m, 1H), 2.98 (s, 3H), 1.69-1.61 (m, 2H), 1.31-1.19 (m, 2H). LCMS: m/z 547.1 [M + H]⁺ |
| 91 | A1 | 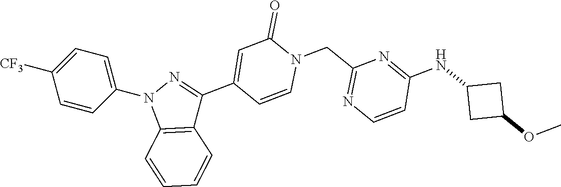 | 1-((4-(((1r,3r)-3- methoxycyclobutyl)amino) pyrimidin-2-yl)methyl)- 4-(1-(4- (trifluoromethyl)phenyl)- 1H-indazol-3-yl)pyridin- 2(1H)-one | ¹H NMR (400 MHz, DMSO-d6) δ 8.25 (d, J = 8.4 Hz, 1H), 8.15 (d, J = 8.4 Hz, 2H), 8.06 (d, J = 8.8 Hz, 1H), 8.01 (d, J = 8.4 Hz, 2H), 7.89 (d, J = 7.2 Hz, 1H), 7.74 (d, J = 6.0 Hz, 1H), 7.66 (ddd, J = 8.4, 7.2, 1.2 Hz, 1H), 7.48 (dd, J = 8.4, 7.2 Hz, 1H), 7.07 (d, J = 2.0 Hz, 1H), 6.97 (dd, J = 7.2, 2.0 Hz, 1H), 6.30 (s, 1H), 5.10 (s, 2H), 4.27-4.19 (m, 1H), 3.94-3.84 (m, 1H), 2.96 (s, 3H), 2.17-2.04 (m, 4H). LCMS: m/z 547.1 [M + H]⁺ |
| 92 | A1 | 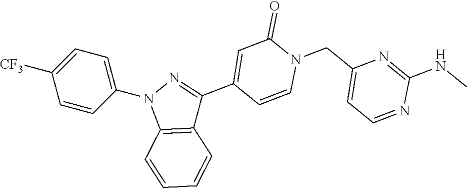 [SMILES: CNc1nccc(Cn2ccc(-c3nn(-c4ccc(C(F)(F)F)cc4)c4ccccc34)cc2=O)n1] | 1-((2- (methylamino)pyrimidin- 4-yl)methyl)-4-(1-(4- (trifluoromethyl)phenyl)- 1H-indazol-3-yl)pyridin- 2(1H)-one | ¹H NMR (400 MHz, DMSO-d6) δ 8.23 (d, J = 8.4 Hz, 2H), 8.13 (d, J = 8.4 Hz, 2H), 8.04 (d, J = 8.4 Hz, 1H), 7.99 (d, J = 8.4 Hz, 2H), 7.92 (d, J = 7.2 Hz, 1H), 7.64 (dd, J = 8.8, 6.8 Hz, 1H), 7.46 (t, J = 7.6 Hz, 1H), 7.35 (br.s, 1H), 7.11 (d, J = 2.0 Hz, 1H), 7.02 (dd, J = 7.2, 2.0 Hz, 1H), 6.42 (d, J = 5.2 Hz, 1H), 5.09 (s, 2H), 2.76 (s, 3H). LCMS: m/z 477.1 [M + H]⁺ |
| 93 | A1 | 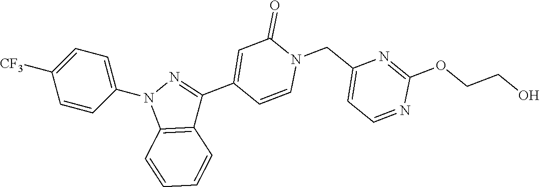 [SMILES: O=c1cc(-c2nn(-c3ccc(C(F)(F)F)cc3)c3ccccc23)ccn1Cc1ccnc(OCCO)n1] | 1-((2-(2- hydroxyethoxy)pyrimidin- 4-yl)methyl)-4-(1-(4- (trifluoromethyl)phenyl)- 1H-indazol-3-yl)pyridin- 2(1H)-one | ¹H NMR (400 MHz, DMSO-d6) δ 8.56 (d, J = 5.2 Hz, 1H), 8.29-8.25 (m, 1H), 8.16 (d, J = 8.4 Hz, 2H), 8.09-8.05 (m, 1H), 8.01 (d, J = 8.4 Hz, 2H), 7.97 (d, J = 7.2 Hz, 1H), 7.66 (ddd, J = 8.4, 7.2, 1.2 Hz, 1H), 7.53-7.44 (m, 1H), 7.12 (d, J = 2.0 Hz, 1H), 7.07-7.00 (m, 2H), 5.24 (s, 2H), 4.88 (t, J = 5.6 Hz, 1H), 4.34-4.21 (m, 2H), 3.67 (q, J = 5.2 Hz, 2H). LCMS: m/z 508.1 [M + H]⁺ |
| 94 | A1 | 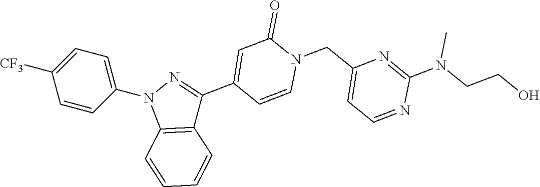 [SMILES: CN(CCO)c1nccc(Cn2ccc(-c3nn(-c4ccc(C(F)(F)F)cc4)c4ccccc34)cc2=O)n1] | 1-((2-((2- hydroxyethyl)(methyl)amino) pyrimidin-4- yl)methyl)-4-(1-(4- (trifluoromethyl)phenyl)- 1H-indazol-3-yl)pyridin- 2(1H)-one | ¹H NMR (400 MHz, DMSO-d6) δ 8.28 (d, J = 5.2 Hz, 1H), 8.25 (d, J = 8.4 Hz, 1H), 8.15 (d, J = 8.4 Hz, 2H), 8.06 (d, J = 8.8 Hz, 1H), 8.01 (d, J = 8.4 Hz, 2H), 7.92 (d, J = 7.2 Hz, 1H), 7.70-7.62 (m, 1H), 7.47 (dd, J = 8.0, 7.2 Hz, 1H), 7.10 (d, J = 2.0 Hz, 1H), 7.04-6.97 (m, 1H), 6.42 (d, J = 4.8 Hz, 1H), 5.10 (s, 2H), 4.62 (brs, 1H), 3.58-3.48 (m, 4H), 3.08 (s, 3H). LCMS: m/z 521.1 [M + H]⁺ |
| 95 | A1 | 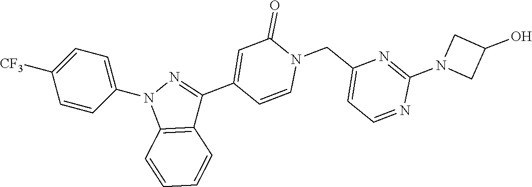 [SMILES: O=c1cc(-c2nn(-c3ccc(C(F)(F)F)cc3)c3ccccc23)ccn1Cc1ccnc(N2CC(O)C2)n1] | 1-((2-(3-hydroxyazetidin- 1-yl)pyrimidin-4- yl)methyl)-4-(1-(4- (trifluoromethyl)phenyl)- 1H-indazol-3-yl)pyridin- 2(1H)-one | ¹H NMR (400 MHz, DMSO-d6) δ 8.30-8.22 (m, 2H), 8.14 (d, J = 8.4 Hz, 2H), 8.05 (d, J = 8.4 Hz, 1H), 7.99 (d, J = 8.4 Hz, 2H), 7.91 (d, J = 7.2 Hz, 1H), 7.64 (ddd, J = 8.4, 7.2, 1.2 Hz, 1H), 7.46 (dd, J = 8.0, 7.2 Hz, 1H), 7.11 (d, J = 2.0 Hz, 1H), 7.01 (dd, J = 7.2, 2.0 Hz, 1H), 6.39 (d, J = 5.2 Hz, 1H), 5.67 (brs, 1H), 5.08 (s, 2H), 4.23-4.10 (m, 2H), 3.72 (dd, J = 9.6, 4.4 Hz, 2H). LCMS: m/z 519.1 [M + H]⁺ |
| 125 | B15 | 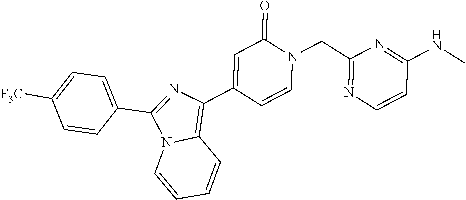 [SMILES: CNc1ccnc(Cn2ccc(-c3nc(-c4ccc(C(F)(F)F)cc4)n4ccccc34)cc2=O)n1] | 1-((4- (methylamino)pyrimidin- 2-yl)methyl)-4-(3-(4- (trifluoromethyl)phenyl) imidazo[1,5-a]pyridin-1- yl)pyridin-2(1H)-one | ¹H-NMR (400 MHz, DMSO) δ 8.66 (d, J = 7.2 Hz, 1H), 8.15 (dd, J = 8.7, 3.8 Hz, 3H), 7.95 (d, J = 8.3Hz, 2H), 7.76 (d, J = 7.2 Hz, 1H), 7.38 (s, 1H), 7.19 (dd, J = 9.1, 6.3 Hz, 1H), 6.96-6.93 (m, 2H), 6.88 (d, J =1.8 Hz, 1H), 6.32 (d, J = 6.0 Hz, 1H), 5.05 (s, 2H), 2.74 (d, J = 4.4 Hz, 3H). LCMS: m/z 477.1 [M + H]+. |
| 126 | B1 | 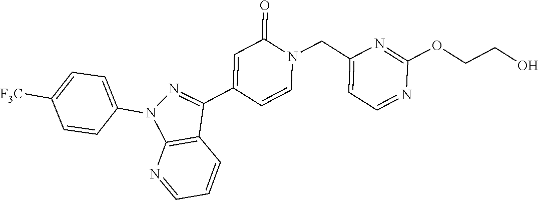 [SMILES: O=c1cc(-c2nn(-c3ccc(C(F)(F)F)cc3)c3ncccc23)ccn1Cc1ccnc(OCCO)n1] | 1-((2-(2- hydroxyethoxy)pyrimidin- 4-yl)methyl)-4-(1-(4- (trifluoromethyl)phenyl)- 1H-pyrazolo[3,4- b]pyridin-3-yl)pyridin- 2(1H)-one | ¹H-NMR (400 MHz, DMSO) δ 8.81 (s, 2H), 8.69 (d, J = 8.5 Hz, 2H), 8.56 (d, J = 5.1 Hz, 1H), 8.00 (dd, J = 7.8, 5.3 Hz, 3H), 7.56 (dd, J = 7.9, 4.8 Hz, 1H), 7.19 (d, J = 1.8 Hz, 1H), 7.11 (dd, J = 7.1, 1.9 Hz, 1H), 7.03 (d, J = 5.1 Hz, 1H), 5.24 (s, 2H), 4.87 (t, J = 5.6 Hz, 1H), 4.26 (t, J = 5.2 Hz, 2H), 3.67 (dd, J = 10.2, 5.4 Hz, 2H). LCMS: m/z 509.4 [M + H]+. |
| 127 | B15 | 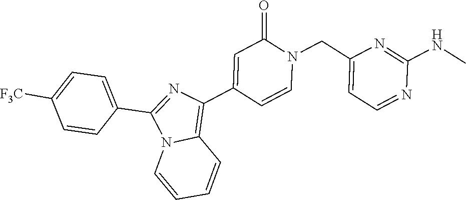 [SMILES: CNc1nccc(Cn2ccc(-c3nc(-c4ccc(C(F)(F)F)cc4)n4ccccc34)cc2=O)n1] | 1-((2- (methylamino)pyrimidin- 4-yl)methyl)-4-(3-(4- (trifluoromethyl)phenyl) imidazo[1,5-a]pyridin-1- yl)pyridin-2(1H)-one | ¹H-NMR (400 MHz, DMSO) δ 8.66 (d, J = 7.2 Hz, 1H), 8.26-8.14 (m, 4H), 7.95 (d, J = 8.3 Hz, 2H), 7.80 (d, J = 7.2 Hz, 1H), 7.20 (dd, J = 9.5, 6.4 Hz, 1H), 7.09 (s, 1H), 7.02-6.92 (m, 3H), 6.28 (d, J = 4.3 Hz, 1H), 5.01 (s, 2H), 2.76 (d, J = 4.7 Hz, 3H). LCMS: m/z 477.1 [M + H]+. |
| 128 | A25 | 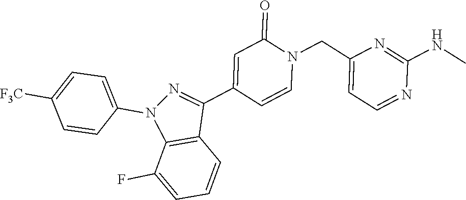 [SMILES: CNc1nccc(Cn2ccc(-c3nn(-c4ccc(C(F)(F)F)cc4)c4c(F)cccc34)cc2=O)n1] | 4-(7-fluoro-1-(4- (trifluoromethyl)phenyl)- 1H-indazol-3-yl)-1-((2- (methylamino)pyrimidin- 4-yl)methyl)pyridin- 2(1H)-one | ¹H-NMR (400 MHz, DMSO) δ 8.22 (s, 1H), 8.07 (d, J = 8.1 Hz, 1H), 8.03-7.97 (m, 4H), 7.92 (d, J = 7.0 Hz, 1H), 7.53-7.40 (m, 2H), 7.08 (d, J = 1.7 Hz, 2H), 6.95 (dd, J = 7.1, 1.8 Hz, 1H), 6.33 (d, J = 4.6 Hz, 1H), 5.07 (s, 2H), 2.74 (d, J = 4.2 Hz, 3H). LCMS: m/z 495.1 [M + H]+. |
| 129 | A8 | 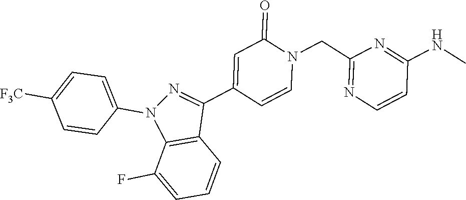 [SMILES: CNc1ccnc(Cn2ccc(-c3nn(-c4ccc(C(F)(F)F)cc4)c4c(F)cccc34)cc2=O)n1] | 4-(7-fluoro-1-(4- (trifluoromethyl)phenyl)- 1H-indazol-3-yl)-1-((4- (methylamino)pyrimidin- 2-yl)methyl)pyridin- 2(1H)-one | ¹H-NMR (400 MHz, DMSO) δ 8.06 (d, J = 8.1 Hz, 1H), 8.04-7.96 (m, 5H), 7.90 (d, J = 7.1 Hz, 1H), 7.46-7.39 (m, 3H), 7.07 (s, 1H), 6.99 (dd, J = 7.1, 1.6 Hz, 1H), 6.35 (d, J = 6.1 Hz, 1H), 5.10 (s, 2H), 2.74 (d, J = 4.6 Hz, 3H). LCMS: m/z 495.1 [M + H]+. |
| 132 | B15 | 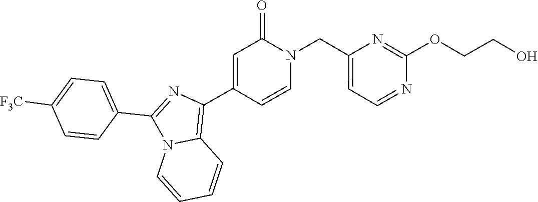 [SMILES: O=c1cc(-c2nc(-c3ccc(C(F)(F)F)cc3)n3ccccc23)ccn1Cc1ccnc(OCCO)n1] | 1-((2-(2- hydroxyethoxy)pyrimidin- 4-yl)methyl)-4-(3-(4- (trifluoromethyl)phenyl) imidazo[1,5-a]pyridin-1- yl)pyridin-2(1H)-one | ¹H-NMR (400 MHz, MeOD) δ 8.53 (dd, J = 14.5, 6.2 Hz, 2H), 8.08 (t, J = 7.7 Hz, 3H), 7.91 (d, J = 8.2Hz, 2H), 7.81 (d, J = 7.1 Hz, 1H), 7.23-7.19 (m, 2H), 7.08 (d, J = 1.8 Hz, 1H), 7.01 (d, J = 5.1 Hz, 1H), 6.93 (t, J = 6.4 Hz, 1H), 5.27 (s, 2H), 4.37 (t, J = 4.8 Hz, 2H), 3.81 (t, J = 4.8 Hz, 2H). LCMS: m/z 508.1 [M + H]+. |
| 133 | A25 | 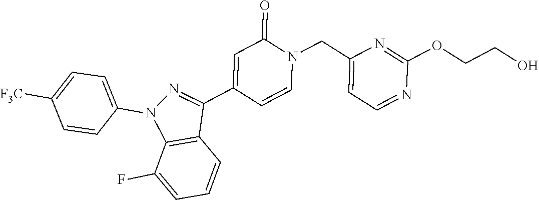 [SMILES: O=c1cc(-c2nn(-c3ccc(C(F)(F)F)cc3)c3c(F)cccc23)ccn1Cc1ccnc(OCCO)n1] | 4-(7-fluoro-1-(4- (trifluoromethyl)phenyl)- 1H-indazol-3-yl)-1-((2-(2- hydroxyethoxy)pyrimidin- 4-yl)methyl)pyridin- 2(1H)-one | ¹H-NMR (400 MHz, DMSO) δ 8.55 (d, J = 5.1 Hz, 1H), 8.08 (d, J = 7.9 Hz, 1H), 7.98 (dd, J = 16.0, 5.5 Hz, 5H), 7.48-7.42 (m, 2H), 7.08 (d, J = 1.8 Hz, 1H), 7.02 (d, J = 5.1 Hz, 1H), 6.98 (dd, J = 7.1, 1.9 Hz, 1H), 5.23 (s, 2H), 4.86 (t, J = 5.6 Hz, 1H), 4.25 (t, J = 4.8 Hz, 2H), 3.66 (dd, J = 10.1, 5.4 Hz, 2H). LCMS: m/z 526.1 [M + H]+. |
| 134 | A25 | 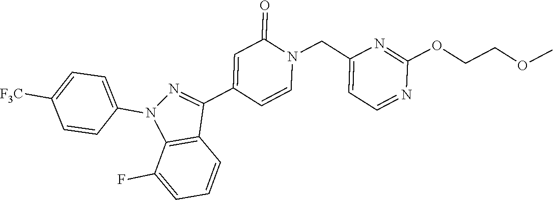 [SMILES: COCCOc1nccc(Cn2ccc(-c3nn(-c4ccc(C(F)(F)F)cc4)c4c(F)cccc34)cc2=O)n1] | 4-(7-fluoro-1-(4- (trifluoromethyl)phenyl)- 1H-indazol-3-yl)-1-((2-(2- methoxyethoxy)pyrimidin- 4-yl)methyl)pyridin- 2(1H)-one | ¹H-NMR (400 MHz, DMSO) δ 8.56 (d, J = 5.1 Hz, 1H), 8.08 (d, J = 7.9 Hz, 1H), 8.00 (s, 5H), 7.51- 7.45 (m, 2H), 7.10 (dd, J = 11.7, 4.8 Hz, 1H), 7.04 (d, J = 5.1 Hz, 1H), 6.97 (d, J = 2.4 Hz, 1H), 5.23 (s, 2H), 4.36 (dd, J = 5.3, 4.0 Hz, 2H), 3.61 (dd, J = 5.3, 4.0 Hz, 2H), 3.24 (s, 3H). LCMS: m/z 540.1 [M + H]+. |
| 135 | A26 | 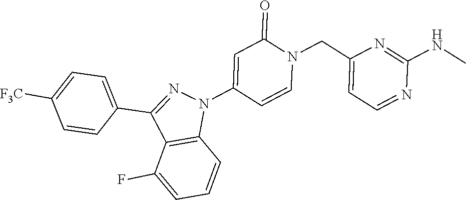 [SMILES: CNc1nccc(Cn2ccc(-n3nc(-c4ccc(C(F)(F)F)cc4)c4c(F)cccc43)cc2=O)n1] | 4-(4-fluoro-3-(4- (trifluoromethyl)phenyl)- 1H-indazol-1-yl)-1-((2- (methylamino)pyrimidin- 4-yl)methyl)pyridin- 2(1H)-one | ¹H-NMR (400 MHz, DMSO) δ 8.27-8.15 (m, 3H), 8.01-7.97 (m, 4H), 7.67 (td, J = 8.2, 5.1 Hz, 1H), 7.27 (dd, J =11.2, 7.8 Hz, 1H), 7.10 (s, 1H), 7.00 (dd, J = 7.4, 2.4 Hz, 1H), 6.89 (d, J = 2.4 Hz, 1H), 6.36 (d, J = 4.7 Hz, 1H), 5.08 (s, 2H), 2.75 (d, J = 4.3 Hz, 3H). LCMS: m/z 495.1 [M + H]+. |
| 136 | A26 | 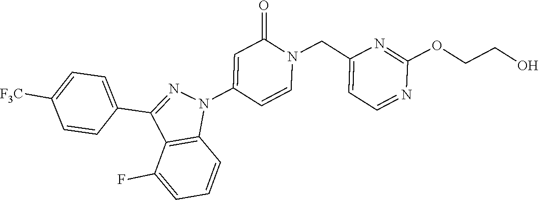 [SMILES: O=c1cc(-n2nc(-c3ccc(C(F)(F)F)cc3)c3c(F)cccc32)ccn1Cc1ccnc(OCCO)n1] | 4-(4-fluoro-3-(4- (trifluoromethyl)phenyl)- 1H-indazol-1-yl)-1-((2-(2- hydroxyethoxy)pyrimidin- 4-yl)methyl)pyridin- 2(1H)-one | ¹H-NMR (400 MHz, DMSO) δ 8.56 (d, J = 5.1 Hz, 1H), 8.17 (d, J = 7.6 Hz, 2H), 8.06 (d, J = 7.5 Hz, 1H), 7.95 (t, J = 8.2 Hz, 3H), 7.67 (td, J = 8.2, 5.1 Hz, 1H), 7.27 (dd, J = 11.2, 7.9 Hz, 1H), 7.04 (dd, J = 9.6, 6.4 Hz, 2H), 6.89 (d, J = 2.4 Hz, 1H), 5.25 (s, 2H), 4.87 (s, 1H), 4.26 (t, J = 4.8 Hz, 2H), 3.67 (d, J = 4.8 Hz, 2H). LCMS: m/z 526.1 [M + H]+. |
| 137 | A26 | 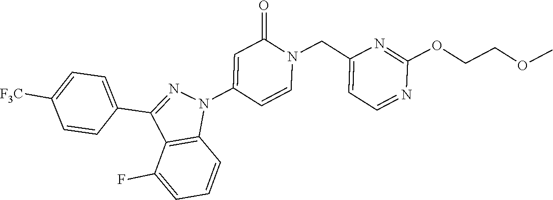 [SMILES: COCCOc1nccc(Cn2ccc(-n3nc(-c4ccc(C(F)(F)F)cc4)c4c(F)cccc43)cc2=O)n1] | 4-(4-fluoro-3-(4- (trifluoromethyl)phenyl)- 1H-indazol-1-yl)-1-((2-(2- methoxyethoxy)pyrimidin- 4-yl)methyl)pyridin- 2(1H)-one | ¹H-NMR (400 MHz, DMSO) δ 8.56 (d, J = 5.1 Hz, 1H), 8.17 (d, J = 7.5 Hz, 2H), 8.06 (d, J = 7.5 Hz, 1H), 7.96 (t, J = 8.9 Hz, 3H), 7.67 (td, J = 8.2, 5.1 Hz, 1H), 7.27 (dd, J = 11.2, 7.8 Hz, 1H), 7.06-7.01 (m, 2H), 6.89 (d, J = 2.4 Hz, 1H), 5.25 (s, 2H), 4.36 (t, J = 4.8 Hz, 2H), 3.61 (t, J = 4.8 Hz, 3H), 3.24 (s, 3H). LCMS: m/z 540.1 [M + H]+. |
| 140 | B28 | 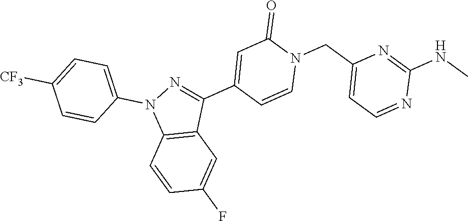 [SMILES: CNc1nccc(Cn2ccc(-c3nn(-c4ccc(C(F)(F)F)cc4)c4ccc(F)cc34)cc2=O)n1] | 4-(5-fluoro-1-(4- (trifluoromethyl)phenyl)- 1H-indazol-3-yl)-1-((2- (methylamino)pyrimidin- 4-yl)methyl)pyridin- 2(1H)-one | ¹H NMR (400 MHz, DMSO-d6) δ 8.22 (s, 1H), 8.13 (d, J = 8.4 Hz, 2H), 8.06 (td, J = 9.2, 8.8, 3.2 Hz, 2H), 8.00 (d, J = 8.4 Hz, 2H), 7.91 (d, J = 7.2 Hz, 1H), 7.54 (td, J = 9.2, 2.4 Hz, 1H), 7.09 (d, J = 2.0 Hz, 2H), 7.00 (dd, J = 7.2, 2.0 Hz, 1H), 6.32 (d, J = 4.8 Hz, 1H), 5.06 (s, 2H), 2.75 (d, J = 4.8 Hz, 3H). LCMS: m/z 495.1 [M + H]⁺ |
| 141 | B25 | 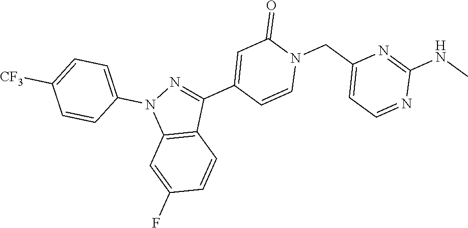 [SMILES: CNc1nccc(Cn2ccc(-c3nn(-c4ccc(C(F)(F)F)cc4)c4cc(F)ccc34)cc2=O)n1] | 4-(6-fluoro-1-(4- (trifluoromethyl)phenyl)- 1H-indazol-3-yl)-1-((2- (methylamino)pyrimidin- 4-yl)methyl)pyridin- 2(1H)-one | ¹H NMR (400 MHz, DMSO-d6) δ 8.30 (dd, J = 9.2, 5.2 Hz, 1H), 8.22 (s, 1H), 8.14 (d, J = 8.4 Hz, 2H), 7.99 (d, J = 8.4 Hz, 2H), 7.95-7.86 (m, 2H), 7.34 (td, J = 9.2, 2.0 Hz, 1H), 7.10 (d, J = 2.0 Hz, 2H), 6.98 (dd, J = 7.2, 2.0 Hz, 1H), 6.33 (d, J = 5.2 Hz, 1H), 5.07 (s, 2H), 2.75 (d, J = 4.8 Hz, 3H). LCMS: m/z 495.1 [M + H]⁺ |
| 142 | A24 | 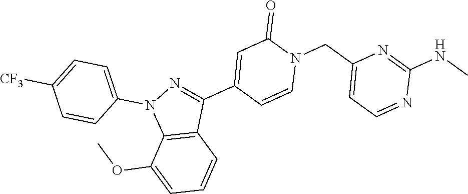 [SMILES: CNc1nccc(Cn2ccc(-c3nn(-c4ccc(C(F)(F)F)cc4)c4c(OC)cccc34)cc2=O)n1] | 4-(7-methoxy-1-(4- (trifluoromethyl)phenyl)- 1H-indazol-3-yl)-1-((2- (methylamino)pyrimidin- 4-yl)methyl)pyridin- 2(1H)-one | ¹H NMR (400 MHz, DMSO-d6) δ 8.22 (s, 1H), 7.90 (q, J = 8.8 Hz, 5H), 7.76 (d, J = 8.0 Hz, 1H), 7.38 (t, J = 8.0 Hz, 1H), 7.15 (d, J = 7.6 Hz, 1H), 7.10 (s, 1H), 7.04 (d, J = 2.0 Hz, 1H), 6.94 (dd, J = 7.2, 2.0 Hz, 1H), 6.33 (d, J = 4.8 Hz, 1H), 5.06 (s, 2H), 3.85 (s, 3H), 2.75 (d, J = 4.4 Hz, 3H). LCMS: m/z 507.1 [M + H]⁺ |
| 143 | A21 | 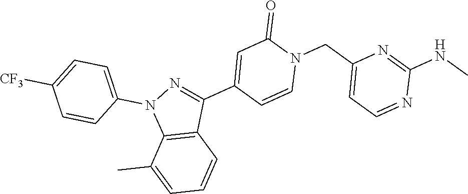 [SMILES: CNc1nccc(Cn2ccc(-c3nn(-c4ccc(C(F)(F)F)cc4)c4c(C)cccc34)cc2=O)n1] | 4-(7-methyl-1-(4- (trifluoromethyl)phenyl)- 1H-indazol-3-yl)-1-((2- (methylamino)pyrimidin- 4-yl)methyl)pyridin- 2(1H)-one | ¹H NMR (400 MHz, DMSO-d6) δ 8.22 (s, 1H), 8.12- 8.03 (m, 1H), 7.98 (d, J = 8.3 Hz, 2H), 7.93-7.85 (m, 3H), 7.42-7.27 (m, 2H), 7.09 (s, 1H), 7.04 (d, J = 1.9 Hz, 1H), 6.92 (dd, J = 7.1, 2.0 Hz, 1H), 6.32 (d, J = 5.0 Hz, 1H), 5.05 (s, 2H), 2.75 (d, J = 4.8 Hz, 3H), 2.14 (s, 3H). LCMS: m/z 491.1 [M + H]⁺ |
| 148 | A22 | 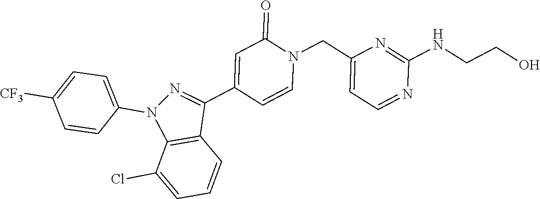 [SMILES: O=c1cc(-c2nn(-c3ccc(C(F)(F)F)cc3)c3c(Cl)cccc23)ccn1Cc1ccnc(NCCO)n1] | 4-(7-chloro-1-(4- (trifluoromethyl)phenyl)- 1H-indazol-3-yl)-1-((2- ((2hydroxyethyl)amino) pyrimidin-4- yl)methyl)pyridin-2(1H)- one | ¹H NMR (400 MHz, DMSO-d6) δ 8.54 (d, J = 5.2 Hz, 1H), 8.24 (d, J = 8.0 Hz, 1H), 8.03-7.88 (m, 5H), 7.69 (d, J = 7.6 Hz, 1H), 7.42 (t, J = 8.0 Hz, 1H), 7.06 (d, J = 2.0 Hz, 1H), 7.01 (d, J = 5.2 Hz, 1H), 6.93 (dd, J = 7.2, 2.0 Hz, 1H), 5.22 (s, 2H), 4.86 (s, 1H), 4.24 (t, J = 5.2 Hz, 2H), 3.65 (d, J = 5.2 Hz, 2H). LCMS: m/z 542.1 [M + H]⁺ |
| 149 | A13 | 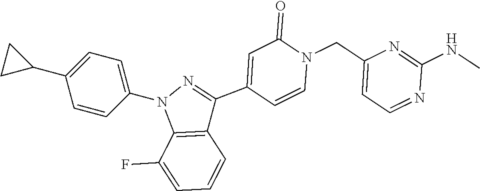 [SMILES: CNc1nccc(Cn2ccc(-c3nn(-c4ccc(C5CC5)cc4)c4c(F)cccc34)cc2=O)n1] | 4-(1-(4- cyclopropylphenyl)-7- fluoro-1H-indazol-3-yl)-1- ((2- (methylamino)pyrimidin- 4-yl)methyl)pyridin- 2(1H)-one | ¹H NMR (400 MHz, DMSO-d6) δ 8.20 (s, 1H), 7.99 (dd, J = 7.2, 1.6 Hz, 1H), 7.87 (d, J = 7.2 Hz, 1H), 7.55 (dd, J = 8.4, 2.8 Hz, 2H), 7.42-7.32 (m, 2H), 7.30-7.22 (m, 2H), 7.04 (d, J = 2.0 Hz, 2H), 6.93 (dd, J = 7.2, 2.0 Hz, 1H), 6.32 (d, J = 5.2 Hz, 1H), 5.04 (s, 2H), 2.73 (d, J = 4.8 Hz, 3H), 2.02 (tt, J = 8.4, 5.2 Hz, 1H), 1.07-0.97 (m, 2H), 0.79-0.68 (m, 2H). LCMS: m/z 467.1 [M + H]⁺ |
| 150 | A10 | 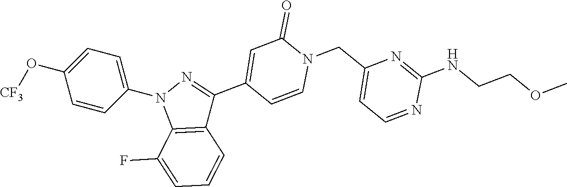 [SMILES: COCCNc1nccc(Cn2ccc(-c3nn(-c4ccc(OC(F)(F)F)cc4)c4c(F)cccc34)cc2=O)n1] | 4-(7-fluoro-1-(4- (trifluoromethoxy)phenyl)- 1H-indazol-3-yl)-1-((2- ((2- methoxyethyl)amino) pyrimidin-4- yl)methyl)pyridin-2(1H)- one | ¹H NMR (400 MHz, DMSO-d6) δ 8.55 (d, J = 5.2 Hz, 1H), 8.09-8.04 (m, 1H), 7.95 (d, J = 7.2 Hz, 1H), 7.90 (dd, J = 9.2, 2.8 Hz, 2H), 7.63-7.59 (m, 2H), 7.47-7.37 (m, 2H), 7.07 (d, J = 2.0 Hz, 1H), 7.03 (d, J = 5.2 Hz, 1H), 6.97 (dd, J = 7.2, 2.0 Hz, 1H), 5.22 (s, 2H), 4.41-4.29 (m, 2H), 3.64-3.56 (m, 2H), 3.24 (s, 3H). LCMS: m/z 556.1 [M + H]⁺ |
| 151 | A10 | 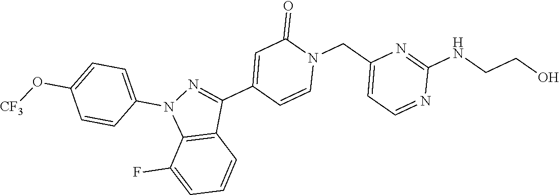 [SMILES: O=c1cc(-c2nn(-c3ccc(OC(F)(F)F)cc3)c3c(F)cccc23)ccn1Cc1ccnc(NCCO)n1] | 4-(7-fluoro-1-(4- (trifluoromethoxy)phenyl)- 1H-indazol-3-yl)-1-((2- ((2- hydroxyethyl)amino) pyrimidin-4- yl)methyl)pyridin-2(1H)- one | ¹H NMR (400 MHz, DMSO-d6) δ 8.55 (d, J = 5.2 Hz, 1H), 8.06 (dd, J = 8.4, 0.8 Hz, 1H), 7.97-7.84 (m, 3H), 7.67-7.58 (m, 2H), 7.53-7.34 (m, 2H), 7.07 (d, J = 2.0 Hz, 1H), 7.02 (d, J = 5.2 Hz, 1H), 6.97 (dd, J = 7.2, 2.0 Hz, 1H), 5.23 (s, 2H), 4.87 (t, J = 5.6 Hz, 1H), 4.25 (t, J = 5.2 Hz, 2H), 3.66 (q, J = 5.2 Hz, 2H). LCMS: m/z 542.1 [M + H]⁺ |
| 158 | A27 | 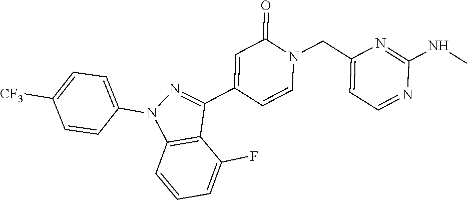 [SMILES: CNc1nccc(Cn2ccc(-c3nn(-c4ccc(C(F)(F)F)cc4)c4cccc(F)c34)cc2=O)n1] | 4-(1-(4-((difluoro-13- methyl)-12- fluoranyl)phenyl)-4- fluoro-1H-indazol-3-yl)-1- ((2- (methylamino)pyrimidin- 4-yl)methyl)pyridin- 2(1H)-one | ¹H NMR (400 MHz, DMSO) δ 8.23 (s, 1H), 8.13 (d, J = 8.5 Hz, 2H), 8.02 (d, J = 8.5 Hz, 2H), 7.90 (d, J = 7.0 Hz, 1H), 7.85 (d, J = 8.6 Hz, 1H), 7.64 (td, J = 8.0, 5.0 Hz, 1H), 7.26 (dd, J = 11.4, 7.8 Hz, 1H), 7.10 (s, 1H), 7.03 (s, 1H), 6.91 (d, J = 6.8 Hz, 1H), 6.33 (s, 1H), 5.06 (s, 2H), 2.75 (d, J = 4.1 Hz, 3H). LCMS: m/z 495.2 [M + H]+. |
| 147 | A26 | 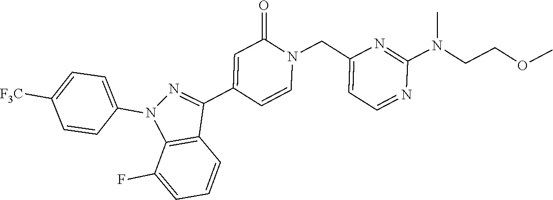 [SMILES: COCCN(C)c1nccc(Cn2ccc(-c3nn(-c4ccc(C(F)(F)F)cc4)c4c(F)cccc34)cc2=O)n1] | 4-(7-fluoro-1-(4- (trifluoromethyl)phenyl)- 1H-indazol-3-yl)-1-((2- ((2- methoxyethyl)(methyl) amino)pyrimidin-4- yl)methyl)pyridin-2(1H)- one | ¹H NMR (400 MHz, DMSO) δ 8.29 (d, J = 5.0 Hz, 1H), 8.06 (d, J = 8.0 Hz, 1H), 8.03-7.96 (m, 4H), 7.92 (d, J = 7.1 Hz, 1H), 7.54-7.46 (m, 1H), 7.45- 7.39 (m, 1H), 7.07 (d, J = 1.6 Hz, 1H), 6.90-6.97 (m, 1H), 6.47 (s, 1H), 5.11 (s, 2H), 3.76-3.56 (m, 2H), 3.36-3.48 (m, 2H), 3.15 (s, 3H), 3.05 (s, 3H). LCMS: m/z 553.2 [M + H]+. |
| 153 | A27 | 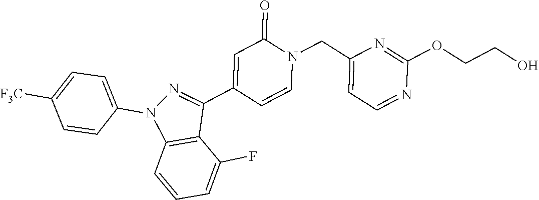 [SMILES: O=c1cc(-c2nn(-c3ccc(C(F)(F)F)cc3)c3cccc(F)c23)ccn1Cc1ccnc(OCCO)n1] | 4-(4-fluoro-1-(4- (trifluoromethyl)phenyl)- 1H-indazol-3-yl)-1-((2-(2- hydroxyethoxy)pyrimidin- 4-yl)methyl)pyridin- 2(1H)-one | ¹H NMR (400 MHz, DMSO) δ 8.70 (d, J = 5.1 Hz, 1H), 8.27 (d, J = 8.5 Hz, 2H), 8.15 (d, J = 8.6 Hz, 2H), 8.08 (d, J = 7.1 Hz, 1H), 7.99 (d, J = 8.6 Hz, 1H), 7.81-7.75 (m, 1H), 7.42-7.37 (m, 1H), 7.16 (d, J = 4.9 Hz, 2H), 7.06 (d, J = 7.1 Hz, 1H), 5.36 (s, 2H), 4.40 (t, J = 4.8 Hz, 2H), 3.81 (t, J =4.8 Hz, 2H), 3.53 (s, 1H). LCMS: m/z. 526.1 [M + H]+. |
| 154 | A27 | 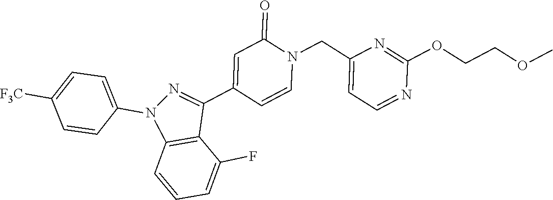 [SMILES: COCCOc1nccc(Cn2ccc(-c3nn(-c4ccc(C(F)(F)F)cc4)c4cccc(F)c34)cc2=O)n1] | 4-(4-fluoro-1-(4- (trifluoromethyl)phenyl)- 1H-indazol-3-yl)-1-((2-(2- methoxyethoxy)pyrimidin- 4-yl)methyl)pyridin- 2(1H)-one | ¹H NMR (400 MHz, DMSO) δ 8.56 (d, J = 5.1 Hz, 1H), 8.14 (d, J = 8.5 Hz, 2H), 8.02 (d, J = 8.6 Hz, 2H), 7.94 (d, J = 7.1 Hz, 1H), 7.86 (d, J = 8.6 Hz, 1H), 7.66-7.61 (m, 1H), 7.27 (dd, J = 11.5, 7.8 Hz, 1H), 7.03 (d, J = 5.0 Hz, 2H), 6.93 (d, J = 7.1 Hz, 1H), 5.22 (s, 2H), 4.36 (t, J = 4.8 Hz, 2H), 3.61 (t, J = 4.8 Hz, 2H), 3.22 (s, 3H). LCMS: m/z 540.1 [M + H]+. |
| 155 | A27 | 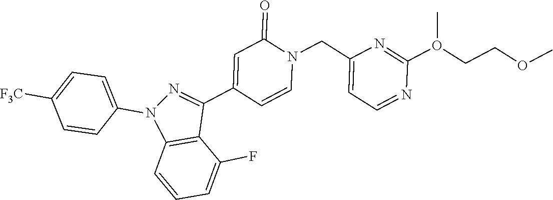 | 4-(4-fluoro-1-(4- (trifluoromethyl)phenyl)- 1H-indazol-3-yl)-1-((2- ((2- methoxyethyl)(methyl)amino) pyrimidin-4- yl)methyl)pyridin-2(1H)- one | ¹H NMR (400 MHz, DMSO) δ 8.29 (d, J = 5.0 Hz, 1H), 8.13 (d, J = 8.4 Hz, 2H), 8.02 (d, J = 8.6 Hz, 2H), 7.90 (d, J = 7.1 Hz, 1H), 7.85 (d, J = 8.6 Hz, 1H), 7.67-7.61 (m, 1H), 7.26 (dd, J = 11.4, 7.7 Hz, 1H), 7.02 (s, 1H), 6.89 (d, J = 7.1 Hz, 1H), 6.46 (s, 1H), 5.10 (s, 2H), 3.75-3.60 (m, 2H), 3.47-3.38 (m, 2H), 3.16 (s, 3H), 3.06 (s, 3H). LCMS: m/z 553.1 [M + H]+. |
| 157 | A25 | 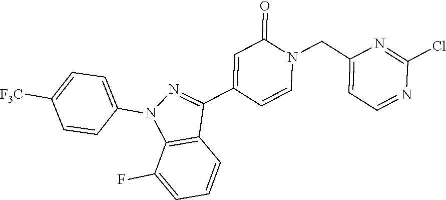 [SMILES: O=c1cc(-c2nn(-c3ccc(C(F)(F)F)cc3)c3c(F)cccc23)ccn1Cc1ccnc(Cl)n1] | 1-((2-chloropyrimidin-4- yl)methyl)-4-(7-fluoro-1- (4- (trifluoromethyl)phenyl)- 1H-indazol-3-yl)pyridin- 2(1H)-one | ¹H NMR (400 MHz, DMSO) δ 8.75 (d, J = 5.1 Hz, 1H), 8.07 (d, J = 8.1 Hz, 1H), 7.92-8.03 (m, 5H), 7.36-7.54 (m, 3H), 7.09 (d, J = 1.7 Hz, 1H), 7.00 (dd, J = 7.1, 1.9 Hz, 1H), 5.30 (s, 2H). |

### Example 10: Synthesis of Compound 131

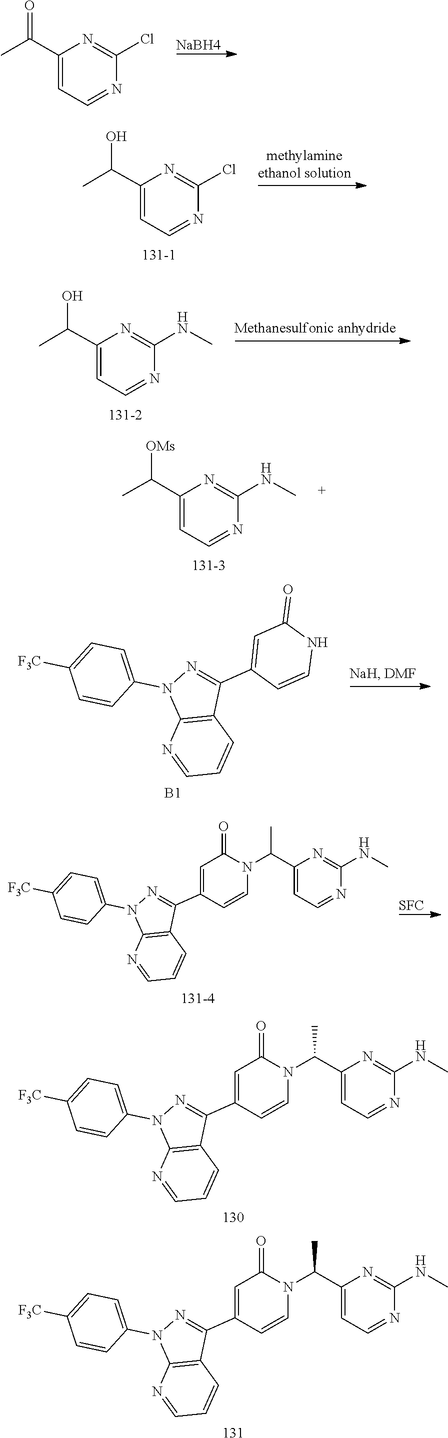

Step 1: To a solution of 1-(2-chloropyrimidin-4-yl)ethan-1-one (900 mg, 5.75 mmol) in THF (5 mL) was added Water (5 mL) and NaBH₄ (326 mg, 8.62 mmol) at 0° C. The mixture was stirred at 25° C. for 2 h. The mixture was diluted with water and extracted with EtOAc twice. The combined organic layer was washed with brine, dried over anhydrous Na₂SO₄, filtered and concentrated under reduced pressure. The residue was purified by flash column chromatography on silica gel (Gradient elution, 20% EtOAc in Heptane) to give compound 131-1 (350 mg, 2.21 mmol, 38.39%) as a colorless oil.

LCMS: m/z 159.0 [M+H]⁺.

Step 2: To a solution of compound 131-1 (350 mg, 2.22 mmol) in methylamine ethanol solution (5 mL) was stirred at 25° C. for 14 h. The mixture was diluted with water and extracted with EtOA twice. The combined organic layer was washed with brine, dried over anhydrous Na₂SO₄, filtered and concentrated under reduced pressure. The residue was purified by flash column chromatography on silica gel (Gradient elution, 35% EtOAc in Heptane) to give compound 131-2 (300 mg, 88.5% yield) as a colorless oil.

LCMS: m/z 154.1 [M+H]⁺.

Step 3: To a solution of compound 131-2 (70 mg, 0.46 mmol) in DCM (3 mL) was added Methanesulfonic anhydride (103 mg, 0.59 mmol) and TEA (92 mg, 0.91 mmol). The mixture was stirred at 23° C. for 2 h. The mixture was concentrated under reduced pressure. The residue was used the next step directly.

LCMS: m/z 232.1 [M+H]⁺.

Step 4: To a solution of compound B1 (75 mg, 0.21 mmol) in DMF (3 mL) was added sodium hydrogen (15 mg, 0.63 mmol) at 25° C. under N₂ atmosphere. The mixture was stirred at 25° C. for 0.5 h. To the mixture was added compound 131-3 (49 mg, 0.21 mmol), and the mixture was stirred at 50° C. for 18 h. The mixture was quenched with ammonium chloride aqueous solution and extracted with EtOAc twice. The combined organic layer was washed with brine, dried over anhydrous Na₂SO₄ and filtered. The filtrate was concentrated under reduced pressure. The residue was purified by flash column chromatography on silica gel (4% MeOH in DCM) to give compound 131-4 (50 mg, 0.102 mmol, 48.33%) as a white solid.

LCMS: m/z 492.1 [M+H]⁺.

The compound 131-4 (50 mg) was separated by SFC to give compound 130 (15 mg, 30% yield) and compound 131 (17 mg, 34% yield).

(compound 130) ¹H-NMR (400 MHz, MeOD) δ 8.70 (dd, J=4.5, 1.5 Hz, 1H), 8.66 (d, J=8.6 Hz, 2H), 8.58 (dd, J=8.2, 1.5 Hz, 1H), 8.22 (d, J=5.1 Hz, 1H), 7.92 (d, J=7.0 Hz, 1H), 7.84 (d, J=8.7 Hz, 2H), 7.45 (dd, J=8.2, 4.5 Hz, 1H), 7.23-7.18 (m, 2H), 6.58 (d, J=5.2 Hz, 1H), 6.09 (q, J=7.2 Hz, 1H), 2.88 (s, 3H), 1.81 (d, J=7.2 Hz, 3H).

LCMS: m/z 492.1 [M+H]⁺.

(compound 131) ¹H-NMR (400 MHz, MeOD) δ 8.73-8.71 (m, 3H), 8.60 (dd, J=8.2, 1.5 Hz, 1H), 8.22 (d, J=5.1 Hz, 1H), 7.93 (d, J=6.9 Hz, 1H), 7.87 (s, 2H), 7.47 (dd, J=8.2, 4.5 Hz, 1H), 7.24 (s, 2H), 6.58 (d, J=5.2 Hz, 1H), 6.09 (q, J=7.1 Hz, 1H), 2.88 (s, 3H), 1.82 (d, J=7.2 Hz, 3H).

LCMS: m/z 492.1 [M+H]⁺.

The following compounds listed below were synthesized according to the procedure of compound 131:

| Com- | From | | | |
|---|---|---|---|---|
| pound | Inter- | | | |
| No. | mediate | Structure | Name | Analytical data |
| 130 | B1 | 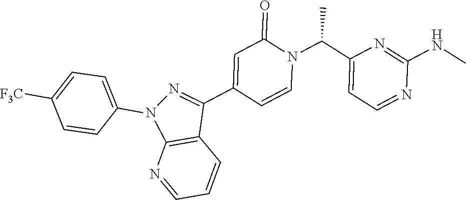 [SMILES: CNc1nccc([C@@H](C)n2ccc(-c3nn(-c4ccc(C(F)(F)F)cc4)c4ncccc34)cc2=O)n1] | (R)-1-(1-(2- (methylamino) pyrimidin-4- yl)ethyl)-4-(1-(4- (trifluoromethyl) phenyl)-1H- pyrazolo[3,4-b] pyridin-3- yl)pyridin- 2(1H)-one | ¹H-NMR (400 MHz, MeOD) δ 8.70 (dd, J = 4.5, 1.5 Hz, 1H), 8.66 (d, J = 8.6 Hz, 2H), 8.58 (dd, J = 8.2, 1.5 Hz, 1H), 8.22 (d, J = 5.1 Hz, 1H), 7.92 (d, J = 7.0 Hz, 1H), 7.84 (d, J = 8.7 Hz, 2H), 7.45 (dd, J = 8.2, 4.5 Hz, 1H), 7.23 -7.18 (m, 2H), 6.58 (d, J = 5.2 Hz, 1H), 6.09 (q, J = 7.2 Hz, 1H), 2.88 (s, 3H), 1.81 (d, J = 7.2 Hz, 3H). LCMS: m/z 492.1 [M + H]+. |
| 131 | B1 | 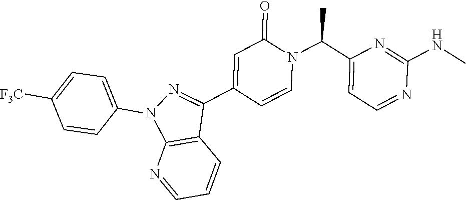 [SMILES: CNc1nccc([C@H](C)n2ccc(-c3nn(-c4ccc(C(F)(F)F)cc4)c4ncccc34)cc2=O)n1] | (S)-1-(1-(2- (methylamino) pyrimidin-4- yl)ethyl)-4-(1-(4- (trifluoromethyl) phenyl)-1H- pyrazolo[3,4-b] pyridin-3- yl)pyridin- 2(1H)-one | ¹H-NMR (400 MHz, MeOD) δ 8.73-8.71 (m, 3H), 8.60 (dd, J = 8.2, 1.5 Hz, 1H), 8.22 (d, J = 5.1 Hz, 1H), 7.93 (d, J = 6.9 Hz, 1H), 7.87 (s, 2H), 7.47 (dd, J = 8.2, 4.5 Hz, 1H), 7.24 (s, 2H), 6.58 (d, J = 5.2 Hz, 1H), 6.09 (q, J = 7.1 Hz, 1H), 2.88 (s, 3H), 1.82 (d, J = 7.2 Hz, 3H). LCMS: m/z 492.1 [M + H]+. |

### Example 11: Synthesis of Compound 152

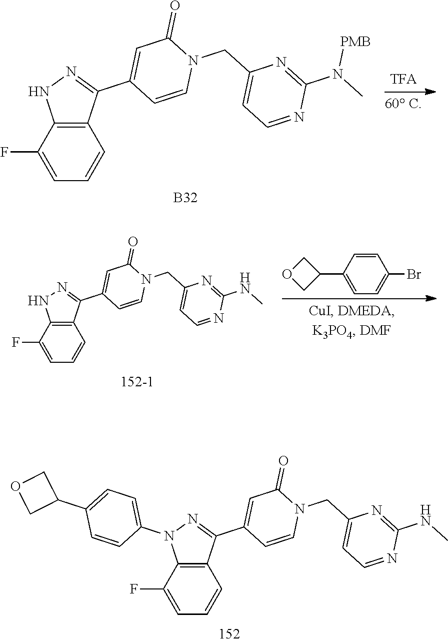

Step 1: A solution of compound B32 (400 mg, 0.850 mmol) in TFA (8 mL) was stirred at 60° C. for 12 h. The mixture was concentrated under reduced pressure. the residue was adjusted pH to 8 with saturated aq. NaHCO₃. To the mixture was added EtOAc (10 mL), and then the suspension was filtered. The filter cake was dried to afford compound 152-1 (290 mg, 97.4%) as a white solid.

LCMS: m/z 351.1 [M+H]⁺.

Step 2: To a solution of compound 145-1 (50 mg, 0.143 mmol) and 3-(4-bromophenyl)oxetane (33.5 mg, 0.157 mmol) in DMF (3 mL) were added CuI (16.3 mg, 0.086 mmol), N,N′-DiMethylethylenediaMine (15.1 mg, 0.171 mmol) and K₃PO₄ (60.6 mg, 0.285 mmol), and the mixture was stirred at 120° C. for 12 h under N₂ atmosphere. The mixture was diluted with water (6 mL). The mixture was extracted with EtOAc (8 mL>2). The combined layer was washed with brine, dried over anhydrous Na₂SO₄, filtered and concentrated under reduced pressure. The residue was purified by prep-HPLC (formic acid as additive) to afford compound 152 (31 mg, 45.0%) as an off-white solid.

LCMS: m/z 483.2 [M+H]⁺.

¹H NMR (400 MHz, DMSO-d6) δ 8.28-8.16 (m, 1H), 8.03 (d, J=7.9 Hz, 1H), 7.89 (d, J=7.1 Hz, 1H), 7.72 (dd, J=8.6, 2.9 Hz, 2H), 7.62 (d, J=8.5 Hz, 2H), 7.47-7.33 (m, 2H), 7.16-7.01 (m, 4H), 6.94 (dd, J=7.1, 2.0 Hz, 1H), 6.32 (d, J=5.0 Hz, 1H), 5.05 (s, 2H), 5.00 (dd, J=8.3, 5.9 Hz, 2H), 4.69 (t, J=6.3 Hz, 2H), 4.45-4.31 (m, 1H), 2.74 (d, J=4.7 Hz, 3H).

### Example 12: Synthesis of Compound 156

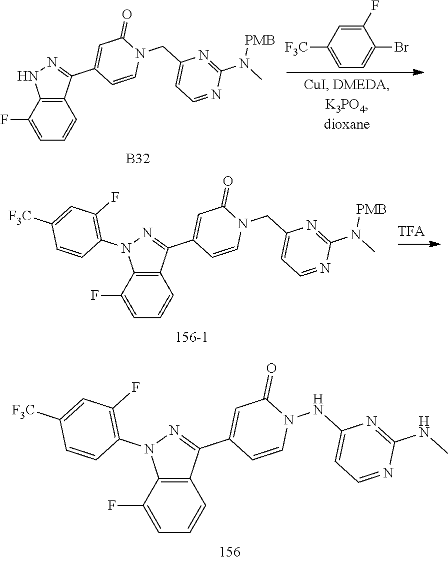

Step 1: To a solution of compound B32 (100 mg, 0.213 mmol) and 1-bromo-2-fluoro-4-(trifluoromethyl)benzene (56.8 mg, 0.234 mmol) in dioxane (4 mL) were added CuI (24.3 mg, 0.128 mmol), N,N′-DiMethylethylenediaMine (22.5 mg, 0.255 mmol) and K₃PO₄ (90.2 mg, 0.425 mmol), and the mixture was stirred at 100° C. for 12 h under N₂ atmosphere. The mixture was diluted with water (10 mL). The mixture was extracted with EtOAc (15 mL>2). The combined layer was washed with brine, dried over anhydrous Na₂SO₄, filtered and concentrated under reduced pressure. The residue was purified by flash column chromatography on silica gel (Gradient elution, 0-67% EtOAc/PE) to afford compound 156-1 (40 mg, 46.9% of purity, 14.0% yield) as a yellow solid.

LCMS: m/z 633.2 [M+H]⁺.

Step 2: A solution of compound 146-1 (40 mg, 0.032 mmol) in TFA (3 mL) was stirred at 60° C. for 12 h. The mixture was concentrated under reduced pressure, and the residue was adjusted pH to 8 with saturated aq. NaHCO₃. The resulting mixture was extracted with DCM (10 mL>2). The combined layer was washed with brine, dried over anhydrous Na₂SO₄, filtered and concentrated under reduced pressure. The residue was purified by prep-HPLC (formic acid as additive) to afford compound 156 (4 mg, 26.3%) as a white solid.

LCMS: m/z 513.1 [M+H]⁺.

¹H NMR (400 MHz, DMSO-d6) δ 8.27-8.18 (m, 1H), 8.18-8.03 (m, 3H), 7.98-7.84 (m, 2H), 7.53-7.39 (m, 2H), 7.16-7.03 (m, 2H), 6.92 (dd, J=7.1, 2.0 Hz, 1H), 6.33 (d, J=4.3 Hz, 1H), 5.06 (s, 2H), 2.74 (d, J=4.8 Hz, 3H).

### Example 4: TR-FRET Assay for TEAD1(210-426) Binding to YAP(50-100)

Compounds were performed 3 fold serial dilution, 12 data points, added to 384 well white plate. The final DMSO concentration is 1%, duplicate for each concentration. Then His-TEAD protein was added to a final concentration of 10 nM, and allowed to preincubate for 30 minutes at room temperature. Assay buffer was added as background control. The TEAD1(210-426) protein sequence used here is named SEQ ID NO:1

(SEQ ID NO: 1)
RSIGTTKLRL VEFSAFLEQQ RDPDSYNKHL FVHIGHANHS
YSDPLLESVD IRQIYDKFPE KKGGLKELFG KGPQNAFFLV
KFWADLNCNI QDDAGAFYGV TSQYESSENM TVTCSTKVCS
FGKQVVEKVE TEYARFENGR FVYRINRSPM CEYMINFIHK
LKHLPEKYMM NSVLENFTIL LVVTNRDTQE TLLCMACVFE
VSNSEHGAQH HIYRLVKD

YAP-Biotin peptide was added to a final concentration of 50 nM, mixed well, incubate 10 minutes at room temperature. The YAP(50-100) peptide sequence used here is named SEQ ID NO: 2

(SEQ ID NO: 2)
AGHQIVHVRG DSETDLEALF NAVMNPKTAN VPQTVPMRLR
KLPDSFFKPP E

The Europium-labeled anti-6×His antibody and APC labeled streptavidin were diluted according to the instructions, this detection solution was mixed 1:1 with reaction solution, incubated 1 hour at room temperature.

TR-FRET measurements were made on an Envision (Perkin Elmer), using an excitation wavelength of 320 nm and emissions read at 620 nm and 665 nm. The ratio of the 665 nm to the 620 nm multiply 10000 provides a normalized TR-FRET signal that represents the extent of binding.

The inhibition (%) was calculated for each compound using the following formula % inhibition=(1−(sample−background control)/(DMSO control−background control))*100%. Half inhibition concentration IC₅₀ value was calculated by XLfit software. The activity of the compounds were listed in below.

| Compound No. | IC₅₀ |
|---|---|
| 1 | A |
| 2 | A |
| 3 | B |
| 4 | B |
| 5 | A |
| 6 | B |
| 7 | A |
| 8 | B |
| 9 | A |
| 10 | C |
| 11 | A |
| 12 | A |
| 13 | A |
| 14 | A |
| 15 | A |
| 16 | B |
| 17 | A |
| 18 | A |
| 19 | A |
| 20 | A |
| 21 | B |
| 22 | B |
| 23 | B |
| 24 | A |
| 25 | B |
| 26 | B |
| 27 | C |
| 28 | A |
| 29 | C |
| 30 | C |
| 31 | C |
| 32 | A |
| 33 | A |
| 34 | A |
| 35 | A |
| 36 | B |
| 37 | A |
| 38 | A |
| 39 | C |
| 40 | C |
| 41 | A |
| 42 | A |
| 43 | B |
| 44 | A |
| 45 | A |
| 46 | A |
| 47 | A |
| 48 | A |
| 49 | A |
| 50 | A |
| 51 | A |
| 52 | A |
| 53 | A |
| 54 | A |
| 55 | A |
| 56 | A |
| 57 | A |
| 58 | A |
| 59 | A |
| 60 | A |
| 61 | A |
| 62 | A |
| 63 | A |
| 64 | A |
| 65 | A |
| 66 | A |
| 67 | A |
| 68 | A |
| 69 | A |
| 70 | A |
| 71 | A |
| 72 | A |
| 73 | A |
| 74 | A |
| 75 | A |
| 76 | A |
| 77 | A |
| 78 | A |
| 79 | A |
| 80 | A |
| 81 | A |
| 82 | A |
| 83 | A |
| 84 | A |
| 85 | A |
| 86 | A |
| 87 | A |
| 88 | A |
| 89 | A |
| 90 | B |
| 91 | A |
| 92 | A |
| 93 | A |
| 94 | A |
| 95 | A |
| 96 | A |
| 97 | A |
| 98 | A |
| 99 | A |
| 100 | A |
| 101 | A |
| 102 | A |
| 103 | A |
| 104 | B |
| 105 | B |
| 106 | A |
| 107 | A |
| 108 | NA |
| 109 | A |
| 110 | A |
| 111 | A |
| 112 | A |
| 113 | NA |
| 114 | NA |
| 115 | A |
| 116 | NA |
| 117 | NA |
| 118 | A |
| 119 | A |
| 120 | A |
| 121 | A |
| 122 | NA |
| 123 | NA |
| 124 | NA |
| 125 | A |
| 126 | A |
| 127 | A |
| 128 | A |
| 129 | A |
| 130 | B |
| 131 | A |
| 132 | A |
| 133 | A |
| 134 | A |
| 135 | A |
| 136 | A |
| 137 | A |
| 138 | B |
| 139 | A |
| 140 | A |
| 141 | A |
| 142 | B |
| 143 | B |
| 144 | A |
| 145 | A |
| 146 | A |
| 147 | A |
| 148 | A |
| 149 | A |
| 150 | A |
| 151 | A |
| 152 | A |
| 153 | A |
| 154 | A |
| 155 | B |
| 156 | A |
| 157 | A |
| 158 | A |

A: IC₅₀ ≤ 0.1 uM;
B: IC₅₀ is in the range of 0.1 uM to 1 uM;
Group C: IC₅₀ > 1 uM

### Example 5: Proliferation Assay

The effect of compound on YAP driven cancer cells proliferation was assessed in MSTO-211H (ATCC, CRL-2081TM) or NCI-H2052 (ATCC, CRL-5915TM) cells using CellTiter-Glo Luminescent Cell Viability Assay (Promega, G7570).

Briefly, individual cell lines were seeded in 96-well cell culture plate at the density of 1,500 cells per well and cultured in RPMII1640 (Gibco-A10491-01)+10% FBS (Gibco-10099-141C)+1% Penicillin-Streptomycin (5,000 U/mL, Gibco-15070-063) overnight at 37° C. with 5% CO₂ before adding the compounds.

Following incubation, serial dilutions of compounds dissolved in DMSO were added to each well (3-fold dilution with 9 concentration points). The final concentration of DMSO present in this assay is below 0.5% and make duplication for each compound treated well.

Following 96 hours treatment, compound mediated cell growth inhibition was measured using CellTiter-Go Luminescent Cell Viability Assay (Promega, G7570). Add soul CellTiter-Glo in each assay well, mix and incubate at room temperature for 10 min. Read signal using EnVision® Multilabel Plate Reader (Perkin Elmer).

For data analysis, cell proliferation inhibition was calculated using the following formula:

Inhibition %=(1−(Compound treated well CTG signal/DMSO well CTG signal))*100.

IC₅₀ values were calculated using GraphPad Prism software using a four-parameter fit model.

The activity of the compounds are listed below.

| Compound No. | MSTO-211H IC₅₀ |
|---|---|
| 1 | A |
| 2 | A |
| 3 | B |
| 4 | B |
| 5 | B |
| 6 | B |
| 7 | B |
| 8 | A |
| 9 | A |
| 11 | A |
| 12 | B |
| 13 | A |
| 14 | A |
| 15 | A |
| 16 | A |
| 17 | A |
| 18 | A |
| 19 | A |
| 20 | A |
| 24 | A |
| 28 | A |
| 29 | A |
| 30 | A |
| 31 | A |
| 33 | A |
| 34 | A |
| 37 | A |
| 38 | A |
| 39 | A |
| 40 | A |
| 41 | A |
| 42 | A |
| 43 | A |
| 44 | A |
| 45 | A |
| 46 | A |
| 47 | A |
| 48 | A |
| 49 | A |
| 50 | A |
| 51 | A |
| 52 | A |
| 53 | A |
| 54 | A |
| 55 | A |
| 56 | A |
| 57 | A |
| 58 | A |
| 59 | A |
| 60 | A |
| 61 | A |
| 62 | A |
| 63 | A |
| 64 | A |
| 65 | A |
| 66 | A |
| 67 | A |
| 68 | A |
| 69 | A |
| 70 | A |
| 71 | A |
| 72 | A |
| 73 | A |
| 74 | A |
| 75 | A |
| 76 | A |
| 77 | A |
| 78 | A |
| 79 | A |
| 80 | A |
| 81 | A |
| 82 | A |
| 83 | A |
| 84 | A |
| 85 | A |
| 86 | A |
| 87 | A |
| 88 | A |
| 89 | A |
| 90 | A |
| 91 | A |
| 92 | A |
| 93 | A |
| 94 | A |
| 95 | A |
| 96 | B |
| 97 | A |
| 98 | A |
| 99 | A |
| 100 | A |
| 101 | A |
| 102 | A |
| 103 | A |
| 104 | A |
| 105 | A |
| 106 | A |
| 107 | A |
| 109 | A |
| 110 | A |
| 111 | A |
| 112 | A |
| 115 | A |
| 118 | A |
| 119 | A |
| 120 | A |
| 121 | A |
| 125 | A |
| 126 | A |
| 127 | A |
| 128 | A |
| 129 | A |
| 130 | A |
| 131 | A |
| 132 | A |
| 133 | A |
| 134 | A |
| 135 | A |
| 136 | A |
| 137 | A |
| 138 | A |
| 139 | A |
| 140 | A |
| 141 | A |
| 142 | A |
| 143 | A |
| 144 | A |
| 145 | A |
| 146 | A |
| 147 | A |
| 148 | A |
| 149 | A |
| 150 | A |
| 151 | A |
| 152 | A |
| 153 | A |
| 154 | A |
| 155 | A |
| 156 | A |
| 157 | A |
| 158 | A |

A: IC₅₀ ≤ 1 uM;
B: IC₅₀ is in the range of 1 uM to 10 uM;
Group C: IC₅₀ > 10 uM

The examples and embodiments described herein are for illustrative purposes only and various modifications or changes suggested to persons skilled in the art are to be included within the spirit and purview of this application and scope of the appended claims.

1-87. (canceled)

88. A compound of Formula (I), or a pharmaceutically acceptable salt or solvate thereof: 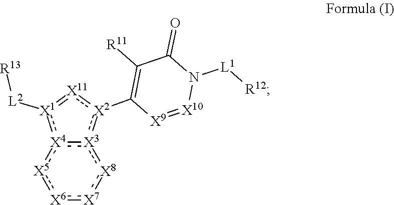
wherein:
X¹ is C, C(R¹), or N;
X² is C, C(R²), or N;
X³ is C, C(R³), or N;
X⁴ is C, C(R⁴), or N;
X⁵ is C(R⁵), C(R⁵)(R^{5a}), N(R^{5b}), or N;
X⁶ is C(R⁶), C(R⁶)(R^{6a}), N(R^{6b}), or N;
X⁷ is C(R⁷), C(R⁷)(R^{7a}), N(R^{7b}), or N;
X⁸ is C(R⁸), C(R⁸)(R^{8a}), N(R^{8b}), or N;
X⁹ is C(R⁹) or N;
X¹⁰ is C(R¹⁰) or N;
X¹¹ is C(R¹⁶), C(R¹⁶)(R^{16a}), C(O), N(R^{16b}), or N;
L¹ and L² are independently selected from a bond and C₁₋₆alkylene optionally substituted with one, two, or three groups selected from R^{14a};
R¹, R², R³, and R⁴ are independently selected from hydrogen and C₁₋₆alkyl;
R⁵, R^{5a}, R⁶, R^{6a}, R⁷, R^{7a}, R⁸, R^{8a}, R¹⁶, and R^{16a} are independently selected from hydrogen, halogen, —CN, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR²⁰, —SR²⁰, —SF₅, —N(R²⁰)(R^{21a}), —C(O)OR²⁰, —OC(O)N(R²⁰)(R²¹), —N(R²²)C(O)N(R²⁰)(R²¹), —N(R²²)C(O)OR²³, —N(R²²)S(O)₂R²³, —C(O)R²³, —S(O)R²³, —OC(O)R²³, —C(O)N(R²⁰)(R²¹), —C(O)C(O)N(R²⁰)(R²¹), —N(R²²)C(O)R²³, —S(O)₂R²³, —S(O)₂N(R²⁰)(R²¹)—, —N═S(═O)(R²³)₂, —S(═O)(═NH)N(R²⁰)(R²¹), —S(═O)(═NH)C(R²⁰)(R²¹), —S(═O)(═NR²³)R²³, —CH₂C(O)N(R²⁰)(R²¹), —CH₂N(R²²)C(O)R²³, —CH₂S(O)₂R²³, and —CH₂S(O)₂N(R²⁰)(R²¹), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three groups selected from R^{14b};
R^{5b}, R^{6b}, R^{7b}, R^{8b}, and R^{16b} are independently selected from hydrogen, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —C(O)OR²⁰, —OC(O)N(R²⁰)(R²¹), —C(O)R²³, —C(O)N(R²⁰)(R²¹), and —S(O)₂R²³, wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three groups selected from R^{14c};
R⁹, R¹⁰, and R¹¹ are independently selected from hydrogen, halogen, and C₁₋₆alkyl;
R¹² is selected from C₁₋₆alkyl, C₁₋₆alkoxyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl, wherein C₁₋₆alkyl, C₁₋₆alkoxyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three groups selected from R^{15a};
R¹³ is selected from C₁₋₆alkyl, C₁₋₆alkoxyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl, wherein C₁₋₆alkyl, C₁₋₆alkoxyl, C₂₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three groups selected from R^{15b};
each R^{14A}, R¹⁴, and R^{14c} are each independently selected from halogen, oxo, —CN, C₁₋₆alkyl, C₁₋₆haloalkyl, C₂₋₆alkenyl, C₂₋₆alkynyl, C₃₋₁₀cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR²⁰, —SR²⁰, —SF₅, —N(R²⁰)(R²¹), —C(O)OR²⁰, —OC(O)N(R²⁰)(R²¹), —N(R²²)C(O)N(R²⁰)(R²¹), —N(R²²)C(O)OR²³, —N(R²²)S(O)₂R²³, —C(O)R²³, —S(O)R²³, —OC(O)R²³, —C(O)N(R²⁰)(R²¹), —C(O)C(O)N(R²⁰)(R²¹), —N(R²²)C(O)R²³, —S(O)₂R²³, —S(O)₂N(R²⁰)(R²¹)—, —N═S(═O)(R²³)₂, —S(═O)(═NH)N(R²⁰)(R²¹), —S(═O)(═NH)C(R²⁰)(R²¹), —S(═O)(═NR²³)R²³, —CH₂C(O)N(R²⁰)(R²¹), —CH₂N(R²²)C(O)R²³, —CH₂S(O)₂R²³, —CH₂S(O)₂N(R²⁰)(R²¹), wherein C₁₋₆alkyl, C₁₋₆haloalkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²⁰, —SR²⁰, —SF₅, —N(R²⁰)(R²¹), —C(O)OR²⁰, —OC(O)N(R²⁰)(R²¹), —N(R²²)C(O)N(R²⁰)(R²¹), —N(R²²)C(O)OR²³, —N(R²²)S(O)₂R²³, —C(O)R²³, —S(O)R²³, —OC(O)R²³, —C(O)N(R²⁰)(R²¹), —C(O)C(O)N(R²⁰)(R²¹), —N(R²²)C(O)R²³, —S(O)₂R²³, —S(O)₂N(R²⁰)(R²¹)—, —N═S(═O)(R²³)₂, —S(═O)(═NH)N(R²⁰)(R²¹), —S(═O)(═NH)C(R²⁰)(R²¹), —S(═O)(═NR²³)R²³, —CH₂C(O)N(R²⁰)(R²¹), —CH₂N(R²²)C(O)R²³, —CH₂S(O)₂R²³, and —CH₂S(O)₂N(R²⁰)(R²¹);
each R^{15a} is independently selected from halogen, oxo, —CN, C₁₋₆alkyl, C₁₋₆haloalkyl, C₂₋₆alkenyl, C₂₋₆alkynyl, C₃₋₁₀cycloalkyl, —CH₂—C₃₋₆cycloalkyl, —NH—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR²⁰, —SR²⁰, —SF₅, —N(R²⁰)(R²¹), —C(O)OR²⁰, —OC(O)N(R²⁰)(R²¹), —N(R²²)C(O)N(R²⁰)(R²¹), —N(R²²)C(O)OR²³, —N(R²²)S(O)₂R²³, —C(O)R²³, —S(O)R²³, —OC(O)R²³, —C(O)N(R²⁰)(R²¹), —C(O)C(O)N(R²⁰)(R²¹), —N(R²²)C(O)R²³, —S(O)₂R₂₃, —S(O)₂N(R²⁰)(R²¹)—, —N═S(═O)(R²³)₂, —S(═O)(═NH)N(R²⁰)(R²¹), —S(═O)(═NH)C(R²⁰)(R²¹), —S(═O)(═NR²³)R²³, —CH₂C(O)N(R²⁰)(R²¹), —CH₂N(R²²)C(O)R²³, —CH₂S(O)₂R²³, —CH₂S(O)₂N(R²⁰)(R²¹), wherein C₁₋₆alkyl, C₁₋₆haloalkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²⁰, —SR²⁰, —SF₅, —N(R²⁰)(R²¹), —C(O)OR²⁰, —OC(O)N(R²⁰)(R²¹), —N(R²²)C(O)N(R²⁰)(R²¹), —N(R²²)C(O)OR²³, —N(R²²)S(O)₂R²³, —C(O)R²³, —S(O)R²³, —OC(O)R²³, —C(O)N(R²⁰)(R²¹), —C(O)C(O)N(R²⁰)(R²¹), —N(R²²)C(O)R²³, —S(O)₂R²³, —S(O)₂N(R²⁰)(R²¹)—, —N═S(═O)(R²³)₂, —S(═O)(═NH)N(R²⁰)(R²¹), —S(═O)(═NH)C(R²⁰)(R²¹), —S(═O)(═NR²³)R²³, —CH₂C(O)N(R²⁰)(R²¹), —CH₂N(R²²)C(O)R²³, —CH₂S(O)₂R²³, and —CH₂S(O)₂N(R²⁰)(R²¹); or
two R^{15a} are combined to form a C₃₋₆cycloalkyl or C₂₋₉heterocycloalkyl, wherein C₃₋₆cycloalkyl and C₂₋₉heterocycloalkyl are optionally substituted with one, two, or three groups independently selected from halogen, oxo, C₁₋₆alkyl, C₁₋₆haloalkyl, and C₁₋₆alkoxy;
each R^{15b} is independently selected from halogen, oxo, —CN, C₁₋₆alkyl, C₁₋₆haloalkyl, C₂₋₆alkenyl, C₂₋₆alkynyl, C₃₋₁₀cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR²⁰, —SR²⁰, —SF₅, —N(R²⁰)(R²¹), —C(O)OR²⁰, —OC(O)N(R²⁰)(R²¹), —N(R²²)C(O)N(R²⁰)(R²¹), —N(R²²)C(O)OR²³, —N(R²²)S(O)₂R²³, —C(O)R²³, —S(O)R²³, —OC(O)R²³, —C(O)N(R²⁰)(R²¹), —C(O)C(O)N(R²⁰)(R²¹), —N(R²²)C(O)R²³, —S(O)₂R²³, —S(O)₂N(R²⁰)(R²¹)—, —N═S(═O)(R²³)₂, —S(═O)(═NH)N(R²⁰)(R²¹), —S(═O)(═NH)C(R²⁰)(R²¹), —S(═O)(═NR²³)R²³,
—CH₂C(O) N(R²⁰)(R²¹), —CH₂N(R²²)C(O)R²³, —CH₂S(O)₂R²³, —CH₂S(O)₂N(R²⁰)(R²¹), wherein C₁₋₆alkyl, C₁₋₆haloalkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²⁰, —SR²⁰, —SF₅, —N(R²⁰)(R²¹), —C(O)OR²⁰, —OC(O)N(R²⁰)(R²¹), —N(R²²)C(O)N(R²⁰)(R²¹), —N(R²²)C(O)OR²³, —N(R²²)S(O)₂R²³, —C(O)R²³, —S(O)R²³, —OC(O)R²³, —C(O)N(R²⁰)(R²¹), —C(O)C(O)N(R²⁰)(R²¹), —N(R²²)C(O)R²³, —S(O)₂R²³, —S(O)₂N(R²⁰)(R²¹)—, —N═S(═O)(R²³)₂, —S(═O)(═NH)N(R²⁰)(R²¹), —S(═O)(═NH)C(R²⁰)(R²¹), —S(═O)(═NR²³)R²³, —CH₂C(O)N(R²⁰)(R²¹), —CH₂N(R²²)C(O)R²³, —CH₂S(O)₂R²³, and —CH₂S(O)₂N(R²⁰)(R²¹); or
two R^{15b} are combined to form a C₃₋₆cycloalkyl or C₂₋₉heterocycloalkyl, wherein C₃₋₆cycloalkyl and C₂₋₉heterocycloalkyl are optionally substituted with one, two, or three groups independently selected from halogen, oxo, C₁₋₆alkyl, C₁₋₆haloalkyl, and C₁₋₆alkoxy;
each R²⁰ is independently selected from hydrogen, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R²¹ is independently selected from hydrogen, C₁₋₆alkyl, and C₁₋₆haloalkyl; or R²⁰ and R²¹, together with the nitrogen to which they are attached, form a C₂₋₉heterocycloalkyl;
each R^{21a} is independently selected from C₁₋₆alkyl and C₁₋₆haloalkyl;
each R²² is independently selected from hydrogen, C₁₋₆alkyl, and C₁₋₆haloalkyl;
each R²³ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl; and [symbol]
indicates a single or double bond such that all valences are satisfied.

89. The compound of claim 88, or a pharmaceutically acceptable salt or solvate thereof, wherein R¹² is selected from C₂₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl, wherein C₂₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three groups selected from R^{15a}; or R¹² is C₁₋₉heteroaryl optionally substituted with one, two, or three groups selected from R^{15a}; or R¹² is C₁₋₉heteroaryl optionally substituted with one, two, or three groups selected from R^{15a}, wherein C₁₋₉heteroaryl is selected from pyridyl, pyrimidinyl, pyrazinyl, pyridazinyl, thienyl, furanyl, pyrrolyl, oxazolyl, thiazolyl, pyrazolyl, indazolyl, and imidazolyl; or R¹² is C₂₋₉heterocycloalkyl optionally substituted with one, two, or three groups selected from R^{15a}.

90. The compound of claim 88, or a pharmaceutically acceptable salt or solvate thereof, wherein R¹³ is selected from C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl, wherein C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three groups selected from R^{15b}; or R¹³ is selected from C₃₋₁₀cycloalkyl, C₂₋₉heterocycloalkyl, C₆₋₁₀aryl, and C₁₋₉heteroaryl, wherein C₃₋₁₀cycloalkyl, C₂₋₉heterocycloalkyl, C₆₋₁₀aryl, and C₁₋₉heteroaryl are optionally substituted with one, two, or three groups selected from R^{15b}; or R¹³ is C₆₋₁₀aryl optionally substituted with one, two, or three groups selected from R^{15b}; or R¹³ is phenyl substituted with one, two, or three groups selected from R^{15b}; or R¹³ is C₁₋₉heteroaryl optionally substituted with one, two, or three groups selected from R^{15b}; or R¹³ is C₁₋₉heteroaryl optionally substituted with one, two, or three groups selected from R^{15b}, wherein C₁₋₉heteroaryl is selected from pyridyl, pyrimidinyl, pyrazinyl, pyridazinyl, thienyl, furanyl, pyrrolyl, oxazolyl, thiazolyl, pyrazolyl, indazolyl, and imidazolyl; or R¹³ is C₃₋₁₀cycloalkyl optionally substituted with one, two, or three groups selected from R^{15b}; or R¹³ is C₂₋₉heterocycloalkyl optionally substituted with one, two, or three groups selected from R^{15b}.

91. The compound of claim 88, or a pharmaceutically acceptable salt or solvate thereof, wherein each R^{14a}, R^{14b}, and R^{14c} are each independently selected from halogen, oxo, —CN, C₁₋₆alkyl, C₁₋₆haloalkyl, C₂₋₆alkenyl, C₂₋₆alkynyl, C₃₋₁₀cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR²⁰, —SRO, —SF₅, —N(R²⁰)(R²¹), —C(O)OR²⁰, —OC(O)N(R²⁰)(R²¹), —N(R²²)C(O)N(R²⁰)(R²¹), —N(R²²)C(O)OR²³, —N(R²²)S(O)₂R²³, —C(O)R²³, —S(O)R²³, —OC(O)R²³, —C(O)N(R²⁰)(R²¹), —C(O)C(O)N(R²⁰)(R²¹), —N(R²²)C(O)R²³, —S(O)₂R²³, —S(O)₂N(R²⁰)(R²¹)—, —N═S(═O)(R²³)₂, —S(═O)(═NH)N(R²⁰)(R²¹), —S(═O)(═NH)C(R²⁰)(R²¹), —S(═O)(═NR²³)R²³, —CH₂C(O)N(R²⁰)(R²¹), —CH₂N(R²²)C(O)R²³, —CH₂S(O)₂R²³, —CH₂S(O)₂N(R²⁰)(R²¹), wherein C₁₋₆alkyl, C₁₋₆haloalkyl, C₂₋₆alkenyl, C₂₋₆alkynyl, C₃₋₆cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²⁰, —SR²⁰, —SF₅, —N(R²⁰)(R²¹), —C(O)OR²⁰, —OC(O)N(R²⁰)(R²¹), —N(R²²)C(O)N(R²⁰)(R²¹), —N(R²²)C(O)OR²³, —N(R²²)S(O)₂R²³, —C(O)R²³, —S(O)R²³, —OC(O)R²³, —C(O)N(R²⁰)(R²¹), —C(O)C(O)N(R²⁰)(R²¹), —N(R²²)C(O)R²³, —S(O)₂R²³, —S(O)₂N(R²⁰)(R²¹)—, —N═S(═O)(R²³)₂, —S(═O)(═NH)N(R²⁰)(R²¹), —S(═O)(═NH)C(R²⁰)(R²¹), —S(═O)(═NR²³)R²³, —CH₂C(O)N(R²⁰)(R²¹), —CH₂N(R²²)C(O)R²³, —CH₂S(O)₂R²³, and —CH₂S(O)₂N(R²⁰)(R²¹).

92. The compound of claim 88, or a pharmaceutically acceptable salt or solvate thereof, wherein each R^{15a} is independently selected from halogen, oxo, —CN, C₁₋₆alkyl, C₁₋₆haloalkyl, C₂₋₆alkenyl, C₂₋₆alkynyl, C₃₋₁₀cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR²⁰, —SR²⁰, —SF₅, —N(R²⁰)(R²¹), —C(O)OR²⁰, —OC(O)N(R²⁰)(R²¹), —N(R²²)C(O)N(R²⁰)(R²¹), —N(R²²)C(O)OR²³, —N(R²²)S(O)₂R²³, —C(O)R²³, —S(O)R²³, —OC(O)R²³, —C(O)N(R²⁰)(R²¹), —C(O)C(O)N(R²⁰)(R²¹), —N(R²²)C(O)R²³, —S(O)₂R²³, —S(O)₂N(R²⁰)(R²¹)—, —N═S(═O)(R²³)₂, —S(═O)(═NH)N(R²⁰)(R²¹), —S(═O)(═NH)C(R²⁰)(R²¹), —S(═O)(═NR²³)R²³, —CH₂C(O)N(R²⁰)(R²¹), —CH₂N(R²²)C(O)R²³, —CH₂S(O)₂R²³, —CH₂S(O)₂N(R²⁰)(R²¹), wherein C₁₋₆alkyl, C₁₋₆haloalkyl, C₂₋₆alkenyl, C₂₋₆alkynyl, C₃₋₆cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²⁰, —SR²⁰, —SF₅, —N(R²⁰)(R²¹), —C(O)OR²⁰, —OC(O)N(R²⁰)(R²¹), —N(R²²)C(O)N(R²⁰)(R²¹), —N(R²²)C(O)OR²³, —N(R²²)S(O)₂R²³, —C(O)R²³, —S(O)R²³, —OC(O)R²³, —C(O)N(R²⁰)(R²¹), —C(O)C(O)N(R²⁰)(R²¹), —N(R²²)C(O)R²³, —S(O)₂R²³, —S(O)₂N(R²⁰)(R²¹)—, —N═S(═O)(R²³)₂, —S(═O)(═NH)N(R²⁰)(R²¹), —S(═O)(═NH)C(R²⁰)(R²¹), —S(═O)(═NR²³)R²³, —CH₂C(O)N(R²⁰)(R²¹), —CH₂N(R²²)C(O)R²³, —CH₂S(O)₂R²³, and —CH₂S(O)₂N(R²⁰)(R²¹); or two R^{15a} are combined to form a C₃₋₆cycloalkyl or C₂₋₉heterocycloalkyl, wherein C₃₋₆cycloalkyl and C₂₋₉heterocycloalkyl are optionally substituted with one, two, or three groups independently selected from halogen, oxo, C₁₋₆alkyl, C₁₋₆haloalkyl, and C₁₋₆alkoxy; or each R^{15a} is independently selected from —C₃₋₇heterocycloalkyl, —NH—C₃₋₆cycloalkyl, —OR²⁰, and —N(R²⁰)(R²¹), wherein —C₃₋₇heterocycloalkyl and —NH—C₃₋₆cycloalkyl, are optionally substituted with one, two, or three groups independently selected from halogen, C₁₋₆alkyl, and —OR²⁰; or each R^{15a} is independently selected from halogen, —CN, C₁₋₆alkyl, and C₁₋₆haloalkyl, —OR²⁰, and —N(R²⁰)(R²¹); or each R^{15a} is independently selected from —OR²⁰, and —N(R²⁰)(R²¹); or each R^{15a} is independently selected from —NH₂, —NH(CH₃), —N(CH₃)₂, —N(CH₃)(CH₂)₂OH, —NH(CH₂)OCH₃, 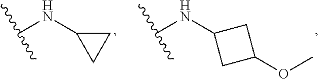
—OH, —OCH₃, —OCH₂CH₂OH, and —OCH₂CH₂OCH₃.

93. The compound of claim 88, or a pharmaceutically acceptable salt or solvate thereof, wherein each R^{15b} is independently selected from halogen, oxo, —CN, C₁₋₆alkyl, C₁₋₆haloalkyl, C₂₋₆alkenyl, C₂₋₆alkynyl, C₃₋₁₀cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR²⁰, —SR²⁰, —SF₅, —N(R²⁰)(R²¹), —C(O)OR²⁰, —OC(O)N(R²⁰)(R²¹), —N(R²²)C(O)N(R²⁰)(R²¹), —N(R²²)C(O)OR²³, —N(R²²)S(O)₂R²³, —C(O)R²³, —S(O)R²³, —OC(O)R²³, —C(O)N(R²⁰)(R²¹), —C(O)C(O)N(R²⁰)(R²¹), —N(R²²)C(O)R²³, —S(O)₂R²³, —S(O)₂N(R²⁰)(R²¹)—, —N═S(═O)(R²³)₂, —S(═O)(═NH)N(R²⁰)(R²¹), —S(═O)(═NH)C(R²⁰)(R²¹), —S(═O)(═NR²³)R²³, —CH₂C(O) N(R²⁰)(R²¹), —CH₂N(R²²)C(O)R²³, —CH₂S(O)₂R²³, —CH₂S(O)₂N(R²⁰)(R²¹), wherein C₁₋₆alkyl, C₁₋₆haloalkyl, C₂₋₆alkenyl, C₂₋₆alkynyl, C₃₋₆cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²⁰, —SR²⁰, —SF₅, —N(R²⁰)(R²¹), —C(O)OR²⁰, —OC(O)N(R²⁰)(R²¹), —N(R²²)C(O)N(R²⁰)(R²¹), —N(R²³)C(O)OR²³, —N(R²²)S(O)₂R²³, —C(O)R²³, —S(O)R²³, —OC(O)R²³, —C(O)N(R²⁰)(R²¹), —C(O)C(O)N(R²⁰)(R²¹), —N(R²²)C(O)R²³, —S(O)₂R²³, —S(O)₂N(R²⁰)(R²¹)—, —N═S(═O)(R²³)₂, —S(═O)(═NH)N(R²⁰)(R²¹), —S(═O)(═NH)C(R²⁰)(R²¹), —S(═O)(═NR²³)R²³, —CH₂C(O)N(R²⁰)(R²¹), —CH₂N(R²²)C(O)R²³, —CH₂S(O)₂R²³, and —CH₂S(O)₂N(R²⁰)(R²¹); or two R^{15b} are combined to form a C₃₋₆cycloalkyl or C₂₋₉heterocycloalkyl, wherein C₃₋₆cycloalkyl and C₂₋₉heterocycloalkyl are optionally substituted with one, two, or three groups independently selected from halogen, oxo, C₁₋₆alkyl, C₁₋₆haloalkyl, and C₁₋₆alkoxy; or each R^{15b} is independently selected from halogen, C₁₋₆alkyl, C₁₋₆haloalkyl, and —OR²⁰, wherein R²⁰ is selected from C₁₋₆alkyl and C₁₋₆haloalkyl.

94. The compound of claim 88, or a pharmaceutically acceptable salt or solvate thereof, wherein X⁹ is C(R⁹) or hydrogen.

95. The compound of claim 88, or a pharmaceutically acceptable salt or solvate thereof, wherein X¹⁰ is C(R¹⁰) or hydrogen.

96. The compound of claim 88, or a pharmaceutically acceptable salt or solvate thereof, wherein R¹¹ is hydrogen, N, C(R¹⁶), C(H) or C(O).

97. The compound of claim 88, or a pharmaceutically acceptable salt or solvate thereof, wherein X¹ is N and X² is C, or X¹ is C and X² is N.

98. The compound of claim 88, or a pharmaceutically acceptable salt or solvate thereof, wherein X³ is C and X⁴ is C, or X³ is N and X⁴ is C, or X³ is C and X⁴ is N.

99. The compound of claim 88, or a pharmaceutically acceptable salt or solvate thereof, wherein X⁵ is C(R⁵) or N; X⁶ is C(R⁶) or N; X⁷ is C(R⁷) or N; and X⁸ is C(R⁸) or N.

100. The compound of claim 88, or a pharmaceutically acceptable salt or solvate thereof, having the structure of Formula (Ia), Formula (Ib), Formula (Ic), Formula (Id), Formula (Ie), Formula (If), Formula (Ig), Formula (Ih), Formula (Ii), Formula (Ij), Formula (Ik), Formula (II), Formula (Im), Formula (In), Formula (Iq), Formula (Ir), Formula (It), Formula (Iu), Formula (Iv), Formula (Iw), Formula (Ix) or Formula (Iy):

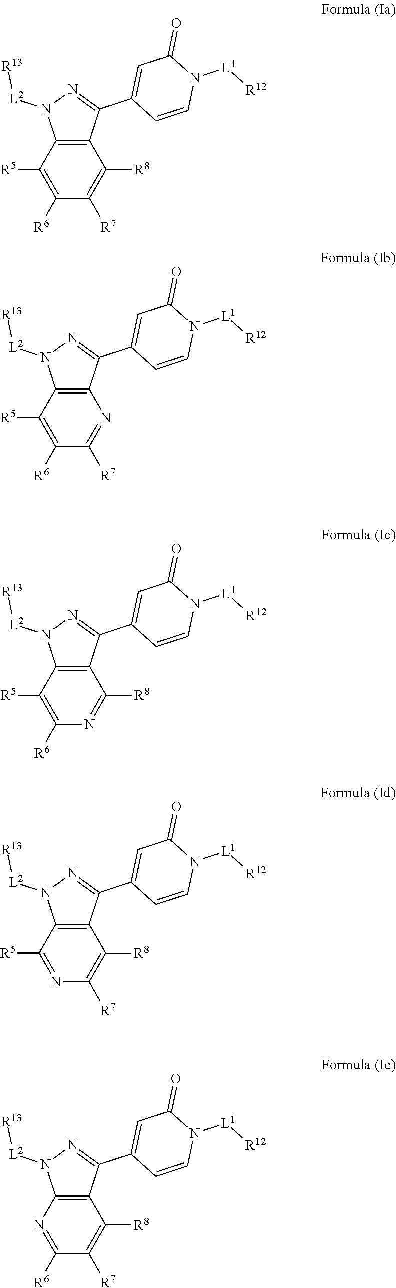

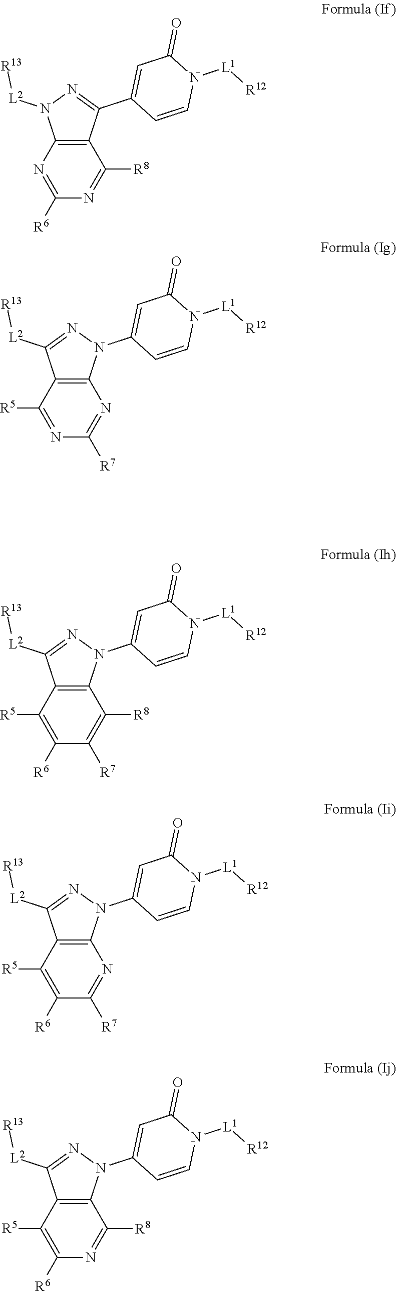

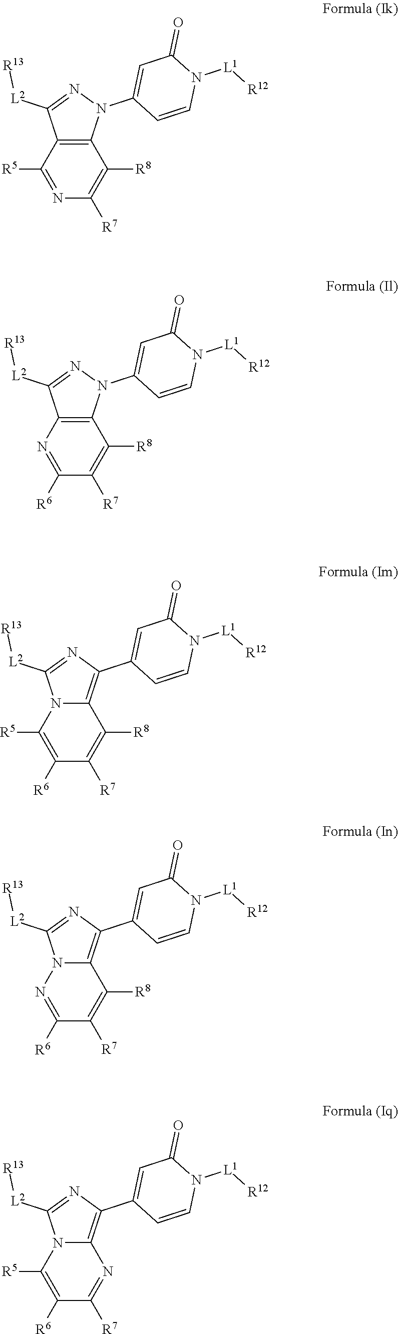

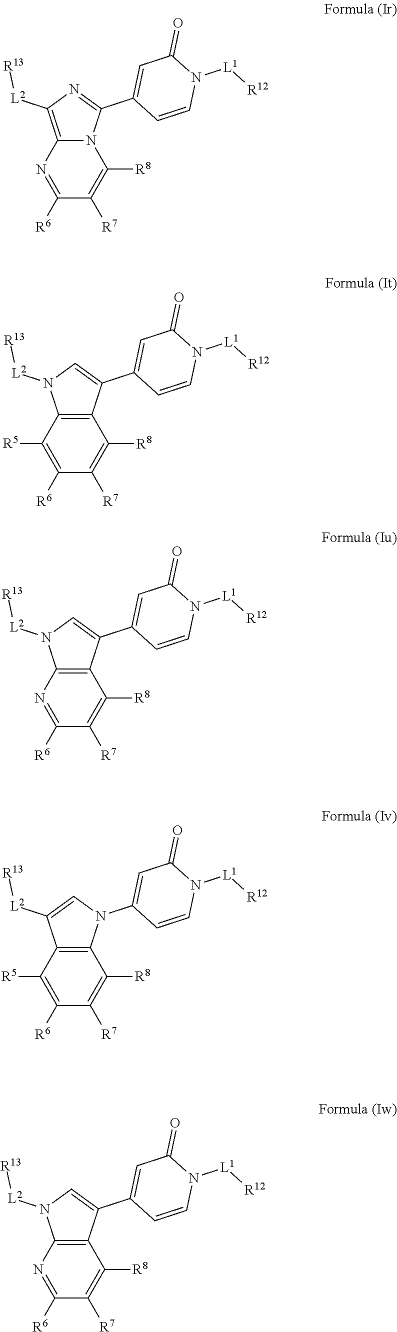

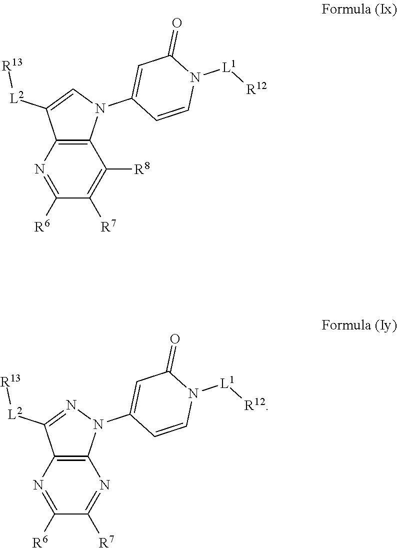

101. The compound of claim 88, or a pharmaceutically acceptable salt or solvate thereof, wherein R⁵ is selected from hydrogen, halogen, —CN, C₁₋₆alkyl, C₁₋₆haloalkyl, —OR²⁰, —C(O)OR²⁰, —C(O)R²³, —C(O)N(R²⁰)(R²¹), —S(O)₂R²³, and —S(O)₂N(R²⁰)(R²¹)—, wherein C₁₋₆alkyl is optionally substituted with one, two, or three groups selected from R^{14b}; or R⁵ is selected from hydrogen, halogen, —CN, C₁₋₆alkyl, —OR²⁰, and —C(O)N(R²⁰)(R²¹), wherein C₁₋₆alkyl is optionally substituted with one, two, or three groups selected from R^{14b}, and wherein R²⁰ is independently selected from hydrogen and C₁₋₆alkyl; or R is hydrogen, F or Cl.

102. The compound of claim 88 or a pharmaceutically acceptable salt or solvate thereof, wherein R⁶ is selected from hydrogen, halogen, —CN, C₁₋₆alkyl, C₁₋₆haloalkyl, —OR²⁰, —C(O)OR²⁰, —C(O)R²³, —C(O)N(R²⁰)(R²¹), —S(O)₂R²³, and —S(O)₂N(R²⁰)(R²¹)—, wherein C₁₋₆alkyl is optionally substituted with one, two, or three groups selected from R^{14b}; or R⁶ is selected from hydrogen, halogen, —CN, C₁₋₆alkyl, —OR²⁰, and —C(O)N(R²⁰)(R²¹), wherein C₁₋₆alkyl is optionally substituted with one, two, or three groups selected from R^{14b}, and wherein R²⁰ is independently selected from hydrogen and C₁₋₆alkyl; or R⁶ is hydrogen.

103. The compound of claim 88, or a pharmaceutically acceptable salt or solvate thereof, wherein R⁷ is selected from hydrogen, halogen, —CN, C₁₋₆alkyl, C₁₋₆haloalkyl, —OR²⁰, —C(O)OR²⁰, —C(O)R²³, —C(O)N(R²⁰)(R²¹), —S(O)₂R²³, and —S(O)₂N(R²⁰)(R²¹)—, wherein C₁₋₆alkyl is optionally substituted with one, two, or three groups selected from R^{14b}; or R⁷ is selected from hydrogen, halogen, —CN, C₁₋₆alkyl, —OR²⁰, and —C(O)N(R²⁰)(R²¹), wherein C₁₋₆alkyl is optionally substituted with one, two, or three groups selected from R^{14b}, and wherein R²⁰ is independently selected from hydrogen and C₁₋₆alkyl; or R⁷ is hydrogen.

104. The compound of claim 88, or a pharmaceutically acceptable salt or solvate thereof, wherein R⁸ is selected from hydrogen, halogen, —CN, C₁₋₆alkyl, C₁₋₆haloalkyl, —OR²⁰, —C(O)OR²⁰, —C(O)R²³, —C(O)N(R²⁰)(R²¹), —S(O)₂R²³, and —S(O)₂N(R²⁰)(R²¹)—, wherein C₁₋₆alkyl is optionally substituted with one, two, or three groups selected from R^{14b}; or R⁷ is selected from hydrogen, halogen, —CN, C₁₋₆alkyl, —OR²⁰, and —C(O)N(R²⁰)(R²¹), wherein C₁₋₆alkyl is optionally substituted with one, two, or three groups selected from R^{14b}, and wherein R²⁰ is independently selected from hydrogen and C₁₋₆alkyl; or R⁷ is hydrogen.

105. The compound of claim 88, or a pharmaceutically acceptable salt or solvate thereof, wherein L² is a bond; or L² is C₁₋₆alkylene optionally substituted with one, two, or three groups selected from R^{14a}.

106. The compound of claim 88, or a pharmaceutically acceptable salt or solvate thereof, wherein L¹ is C₁₋₆alkylene optionally substituted with one, two, or three groups selected from R^{14a}; or L¹ is unsubstituted C₁₋₆alkylene or a bond.

107. The compound of claim 88, or a pharmaceutically acceptable salt or solvate thereof, wherein R²⁰ and R²¹ together with the nitrogen to which they are attached, form azetidinyl, pyrrolidinyl, morpholino, 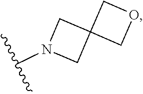 [SMILES: *N1CC2(COC2)C1]
or piperidinyl, wherein azetidinyl, pyrrolidinyl, morpholino, 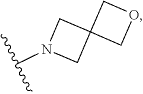 [SMILES: *N1CC2(COC2)C1]
and piperidinyl are optionally substituted with hydroxy, or C₁₋₆alkyl.

108. A compound selected from: 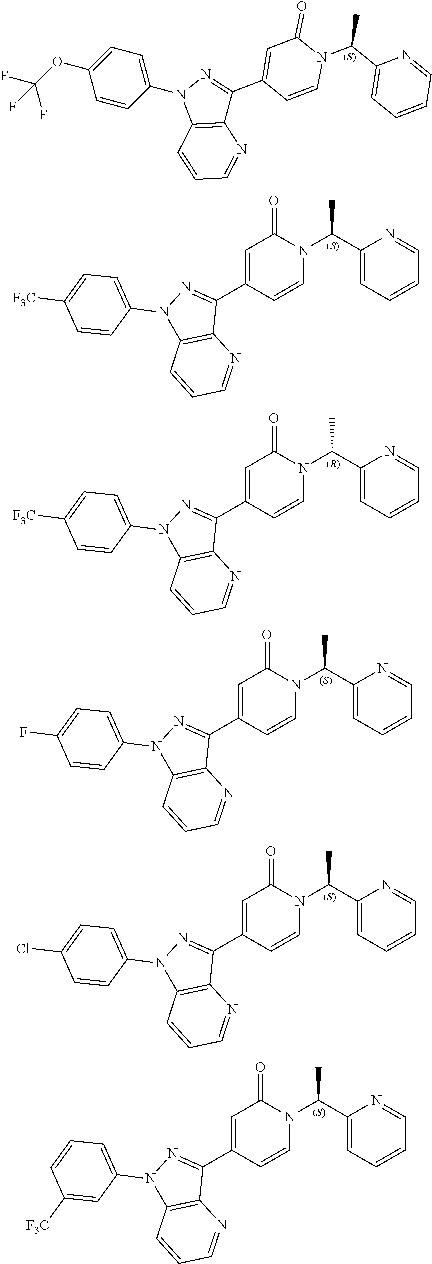 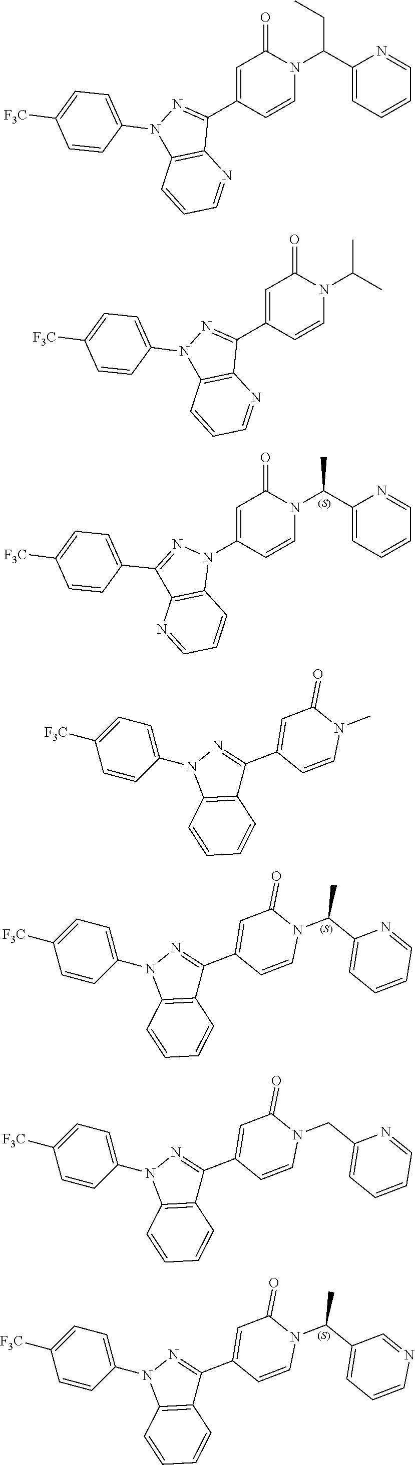 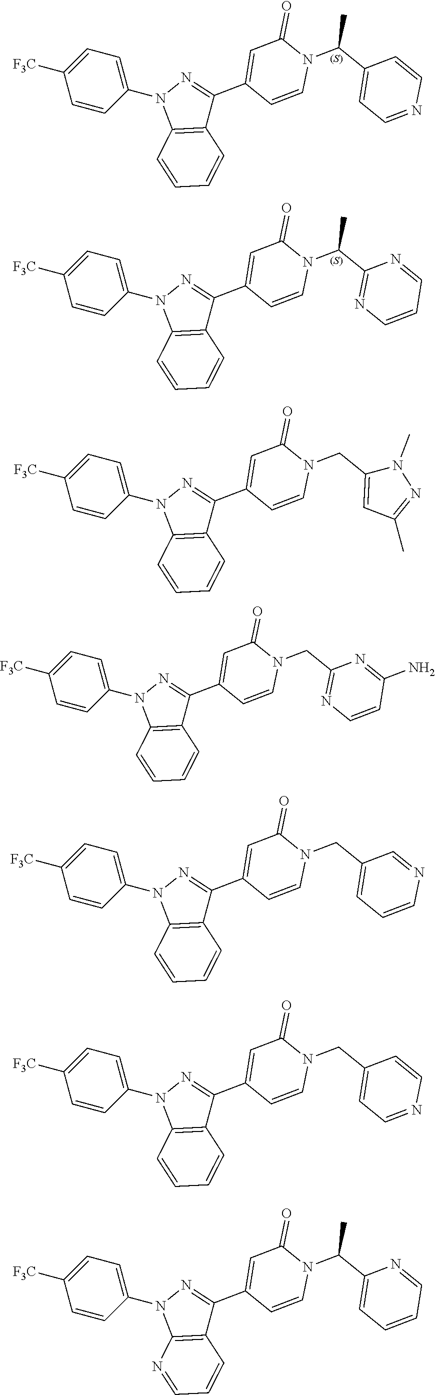 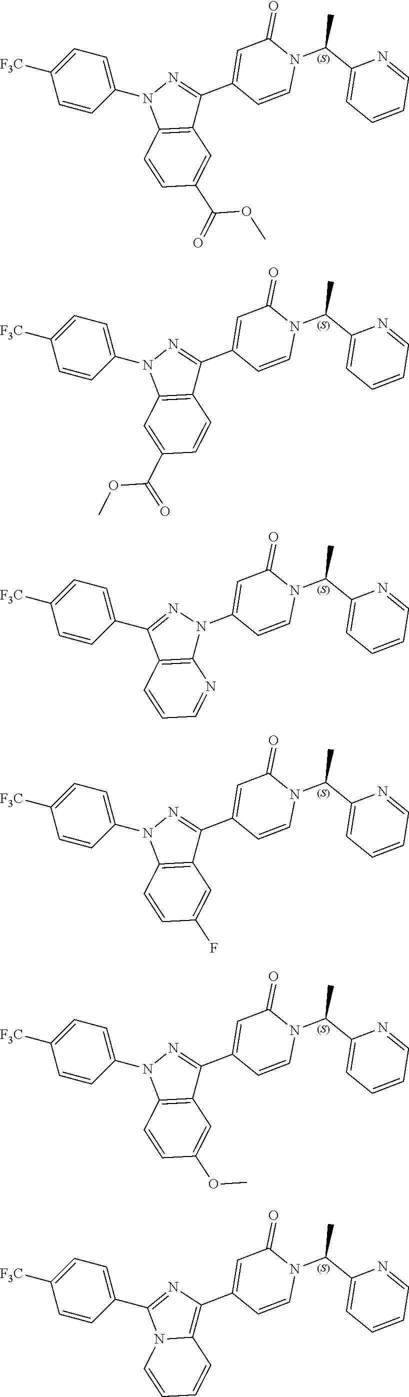 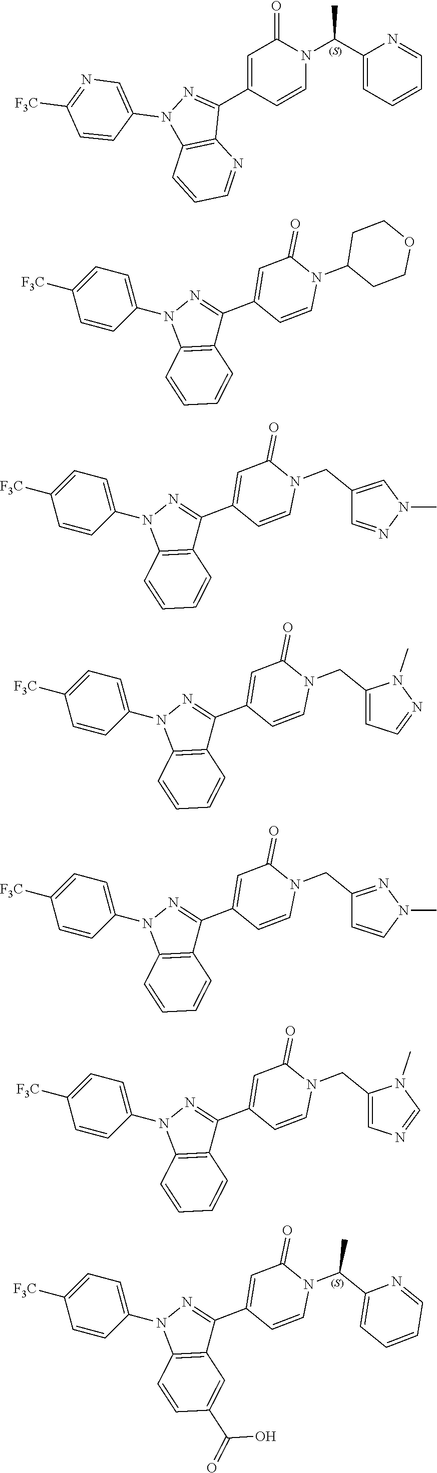 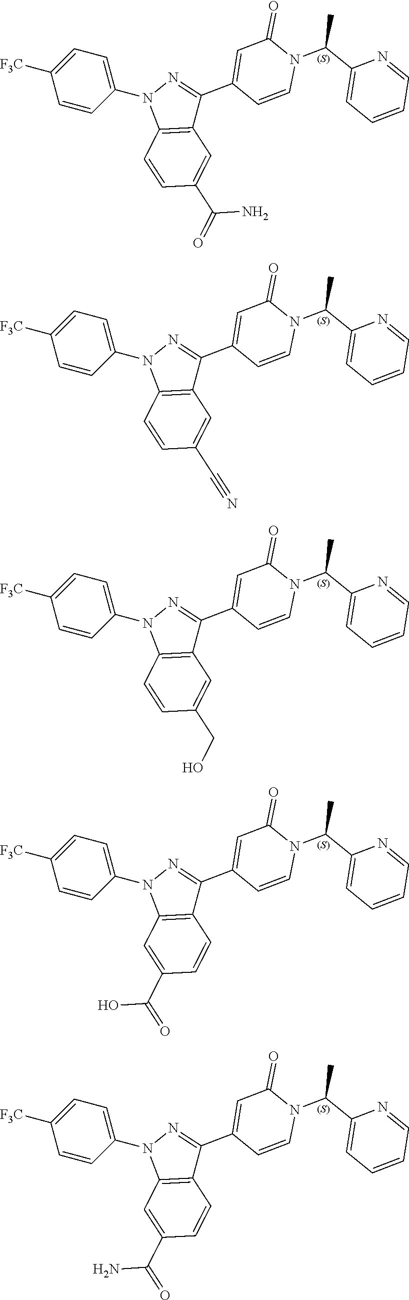 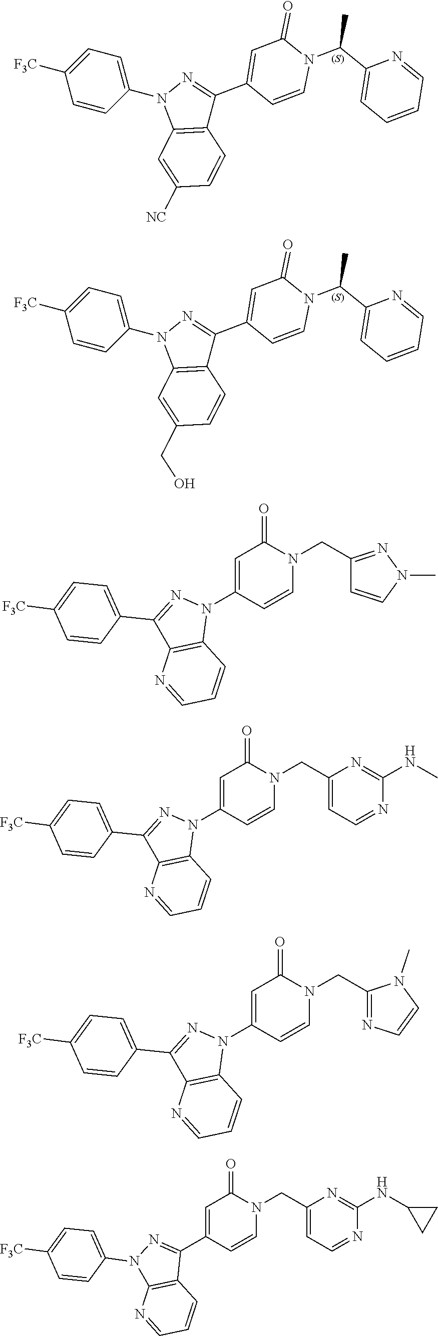 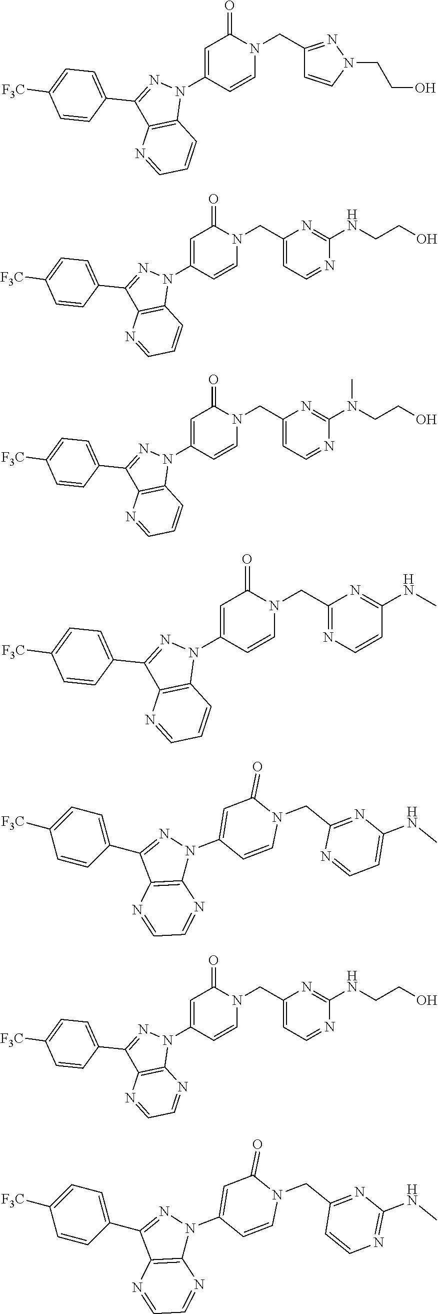 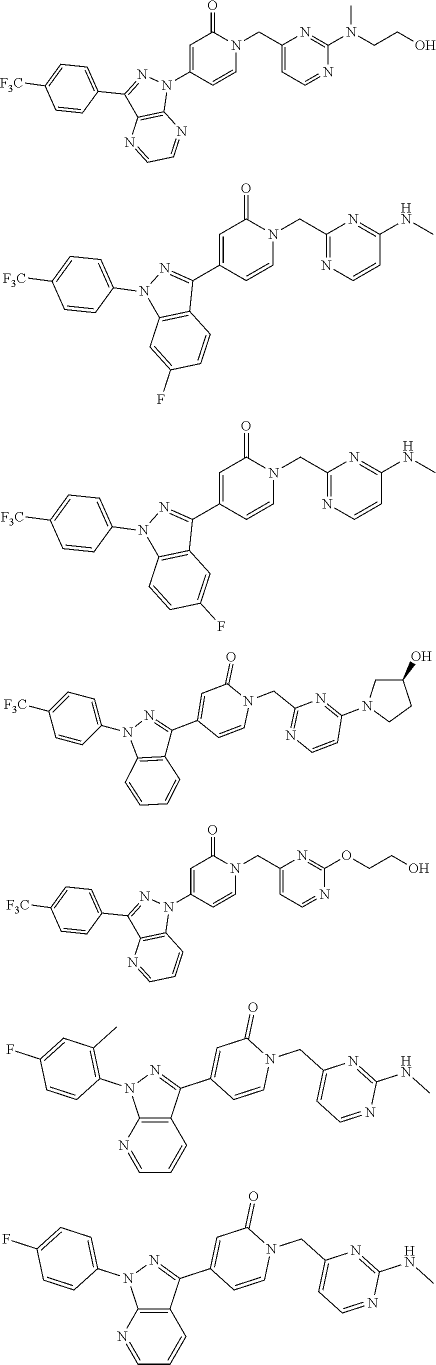 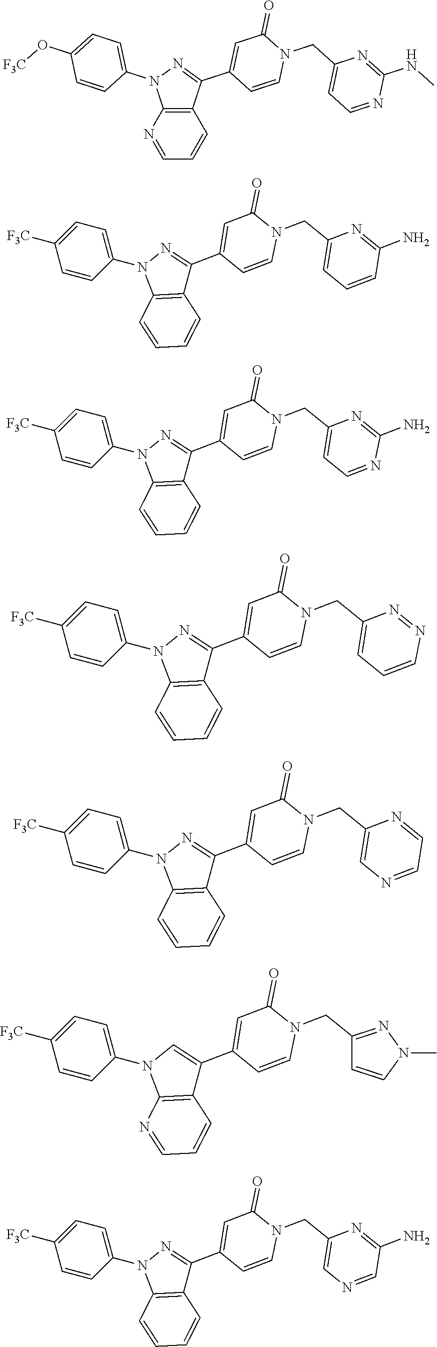 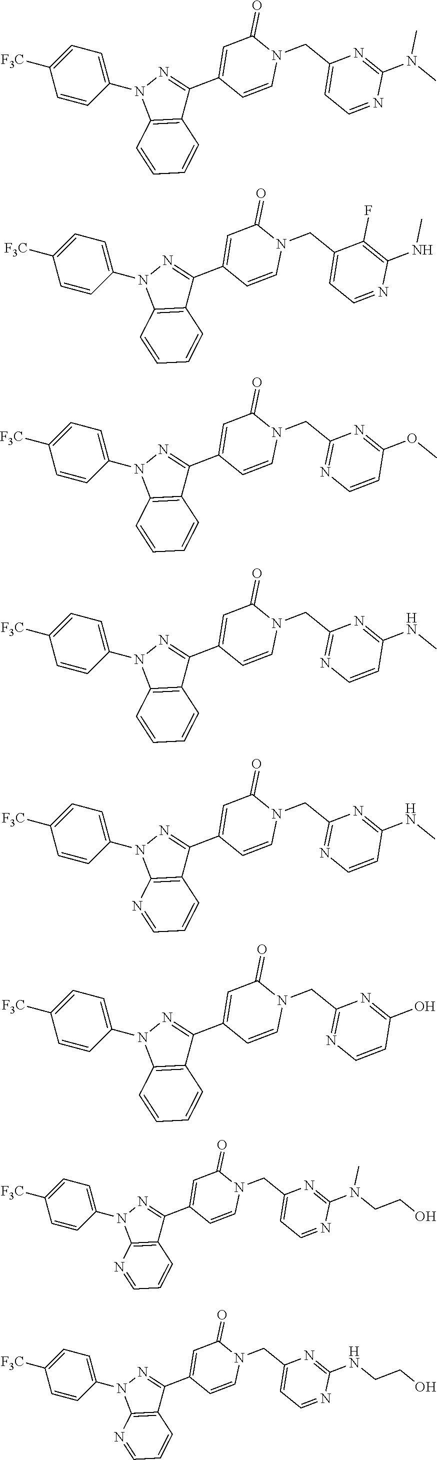 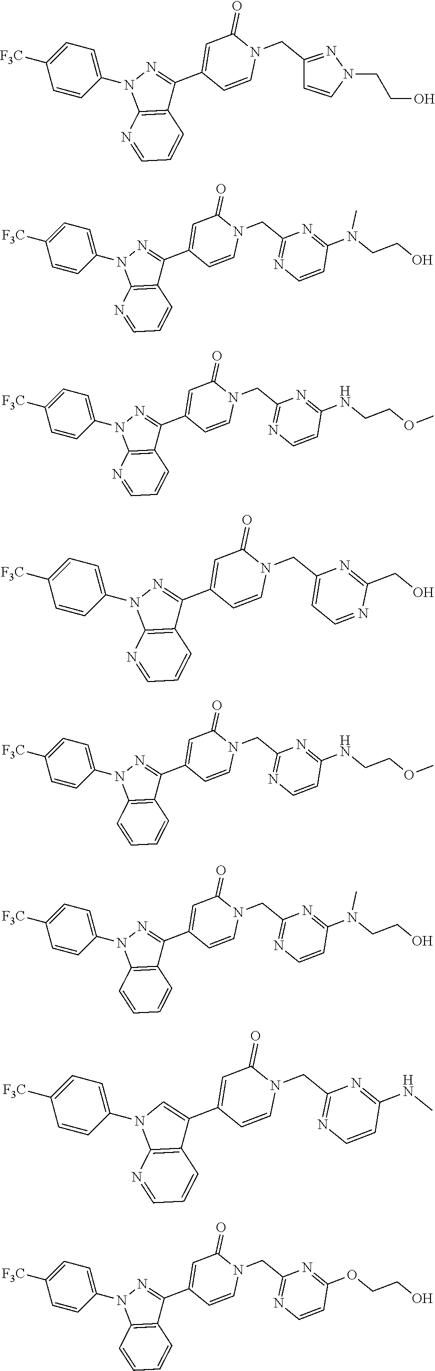 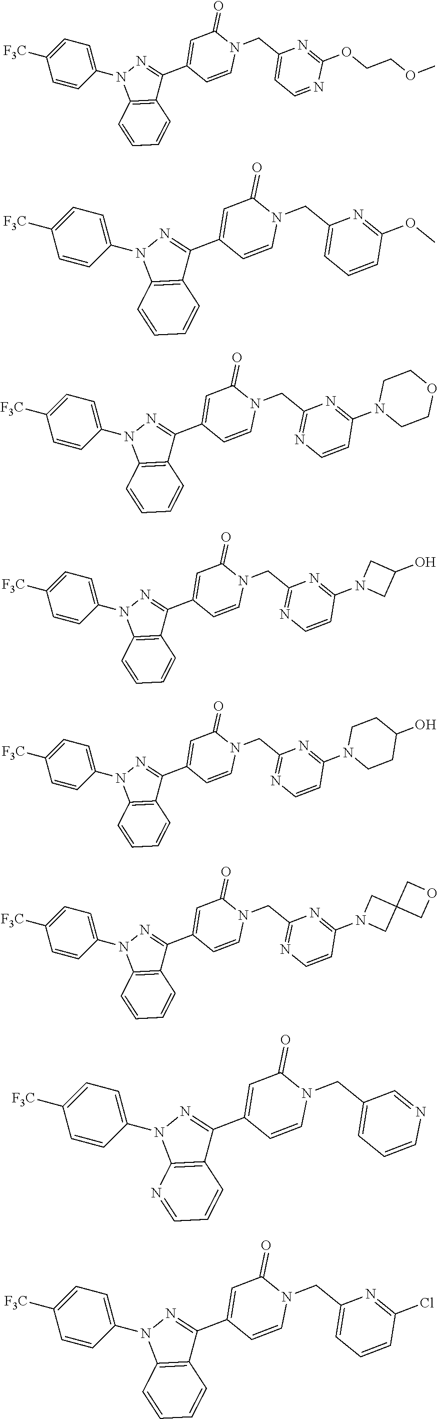 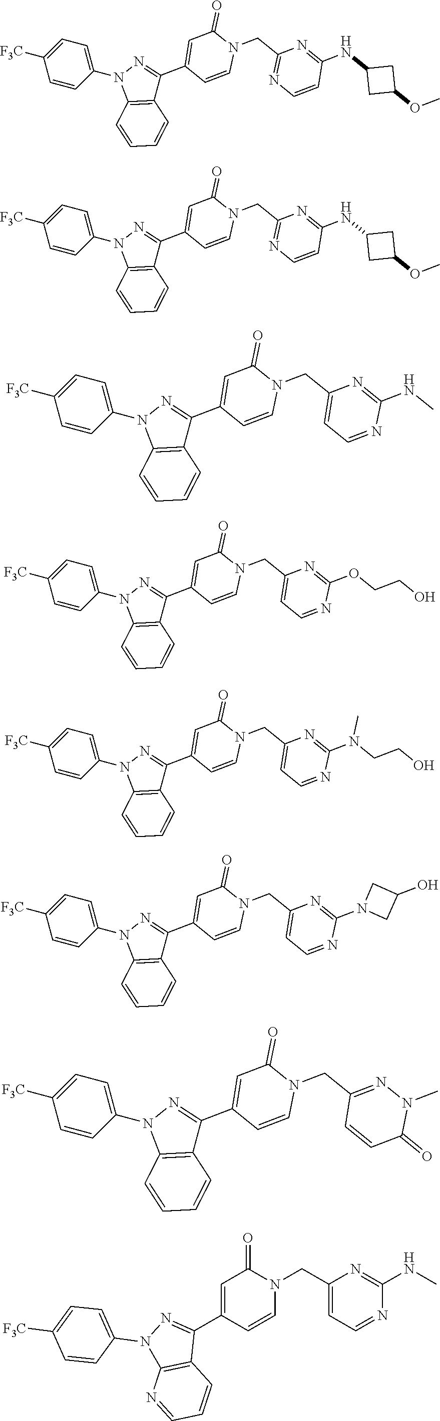 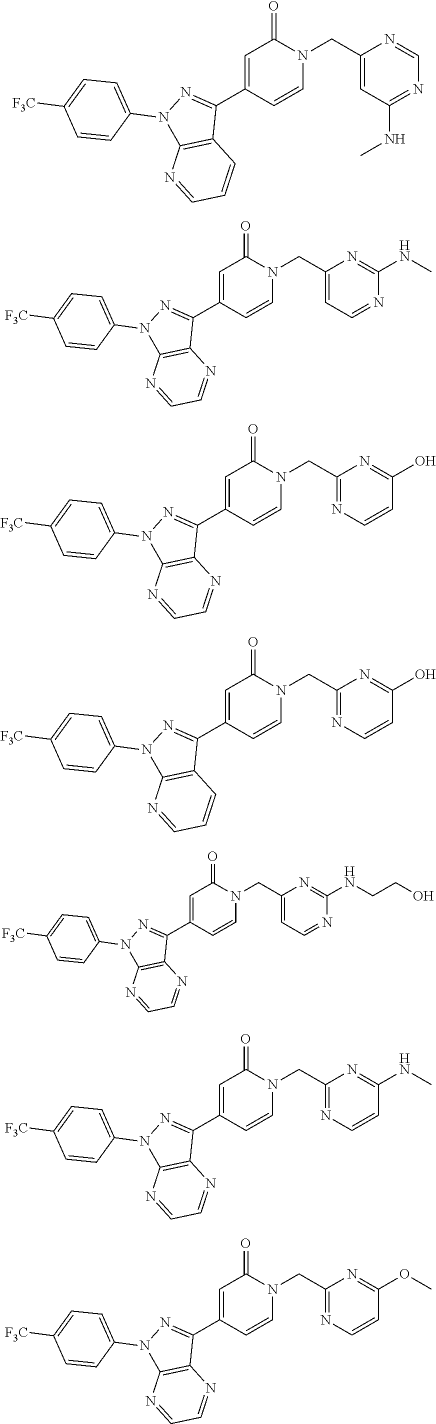 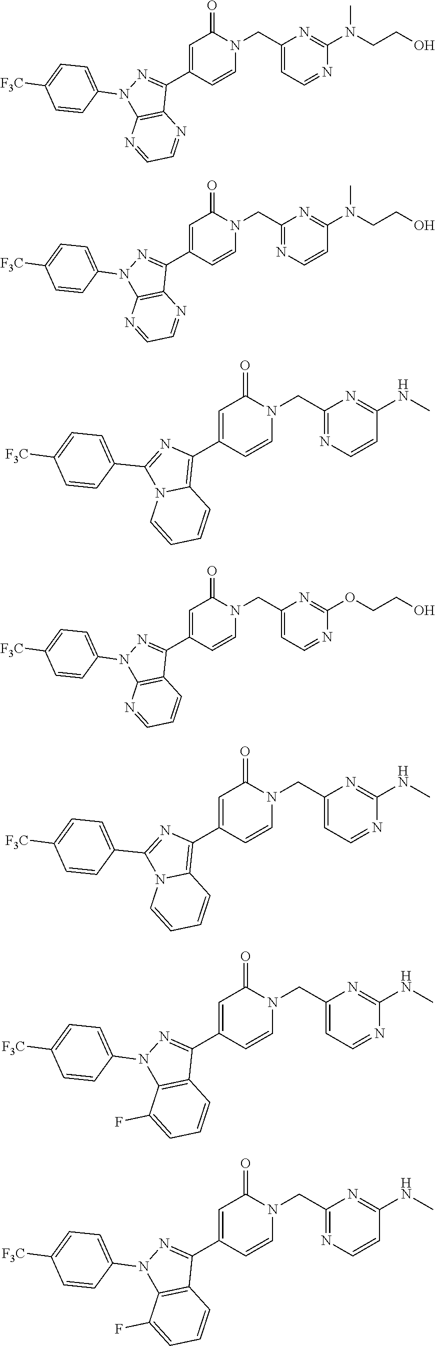 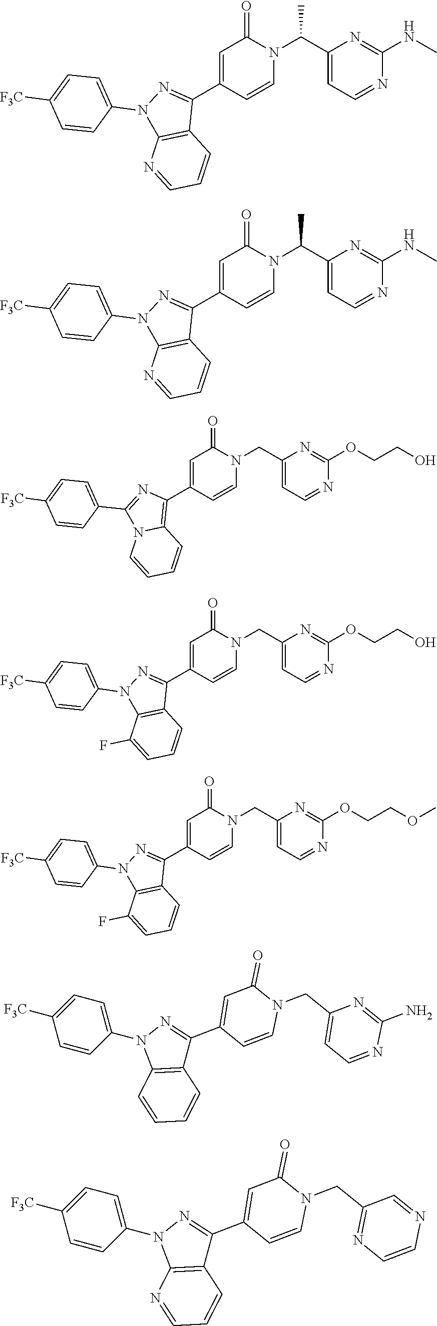 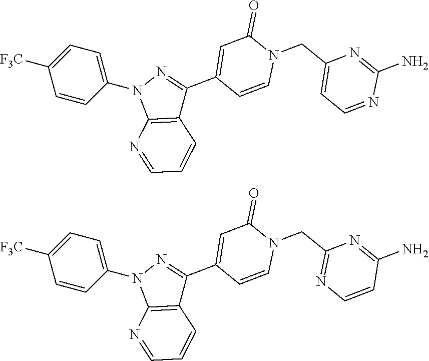 [SMILES: Nc1nccc(Cn2ccc(-c3nn(-c4ccc(C(F)(F)F)cc4)c4ncccc34)cc2=O)n1 ; Nc1ccnc(Cn2ccc(-c3nn(-c4ccc(C(F)(F)F)cc4)c4ncccc34)cc2=O)n1] 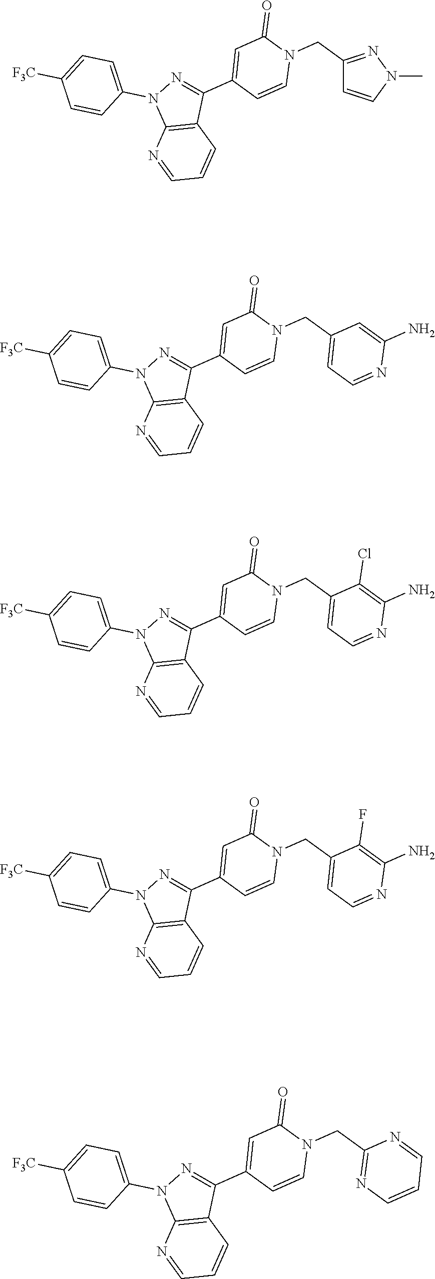 [SMILES: Cn1ccc(Cn2ccc(-c3nn(-c4ccc(C(F)(F)F)cc4)c4ncccc34)cc2=O)n1 ; Nc1cc(Cn2ccc(-c3nn(-c4ccc(C(F)(F)F)cc4)c4ncccc34)cc2=O)ccn1 ; Nc1nccc(Cn2ccc(-c3nn(-c4ccc(C(F)(F)F)cc4)c4ncccc34)cc2=O)c1Cl ; Nc1nccc(Cn2ccc(-c3nn(-c4ccc(C(F)(F)F)cc4)c4ncccc34)cc2=O)c1F ; O=c1cc(-c2nn(-c3ccc(C(F)(F)F)cc3)c3ncccc23)ccn1Cc1ncccn1] 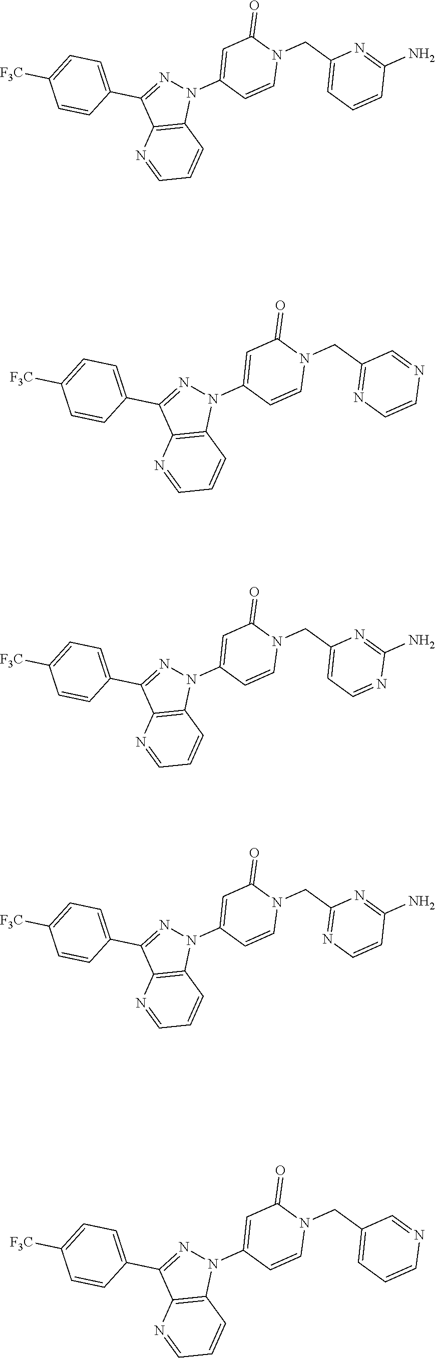 [SMILES: Nc1cccc(Cn2ccc(-n3nc(-c4ccc(C(F)(F)F)cc4)c4ncccc43)cc2=O)n1 ; O=c1cc(-n2nc(-c3ccc(C(F)(F)F)cc3)c3ncccc32)ccn1Cc1cnccn1 ; Nc1nccc(Cn2ccc(-n3nc(-c4ccc(C(F)(F)F)cc4)c4ncccc43)cc2=O)n1 ; Nc1ccnc(Cn2ccc(-n3nc(-c4ccc(C(F)(F)F)cc4)c4ncccc43)cc2=O)n1 ; O=c1cc(-n2nc(-c3ccc(C(F)(F)F)cc3)c3ncccc32)ccn1Cc1cccnc1] 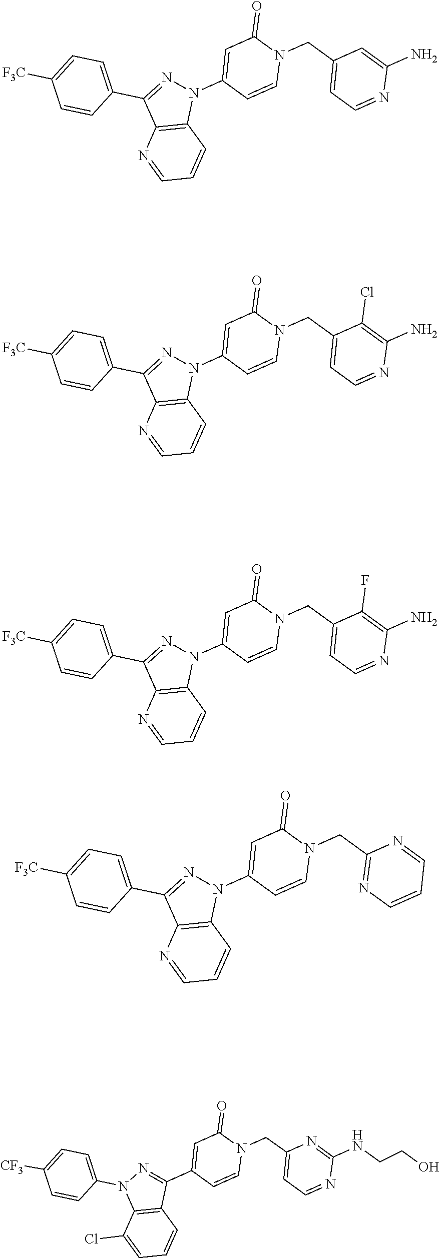 [SMILES: Nc1cc(Cn2ccc(-n3nc(-c4ccc(C(F)(F)F)cc4)c4ncccc43)cc2=O)ccn1 ; Nc1nccc(Cn2ccc(-n3nc(-c4ccc(C(F)(F)F)cc4)c4ncccc43)cc2=O)c1Cl ; Nc1nccc(Cn2ccc(-n3nc(-c4ccc(C(F)(F)F)cc4)c4ncccc43)cc2=O)c1F ; O=c1cc(-n2nc(-c3ccc(C(F)(F)F)cc3)c3ncccc32)ccn1Cc1ncccn1 ; O=c1cc(-c2nn(-c3ccc(C(F)(F)F)cc3)c3c(Cl)cccc23)ccn1Cc1ccnc(NCCO)n1] 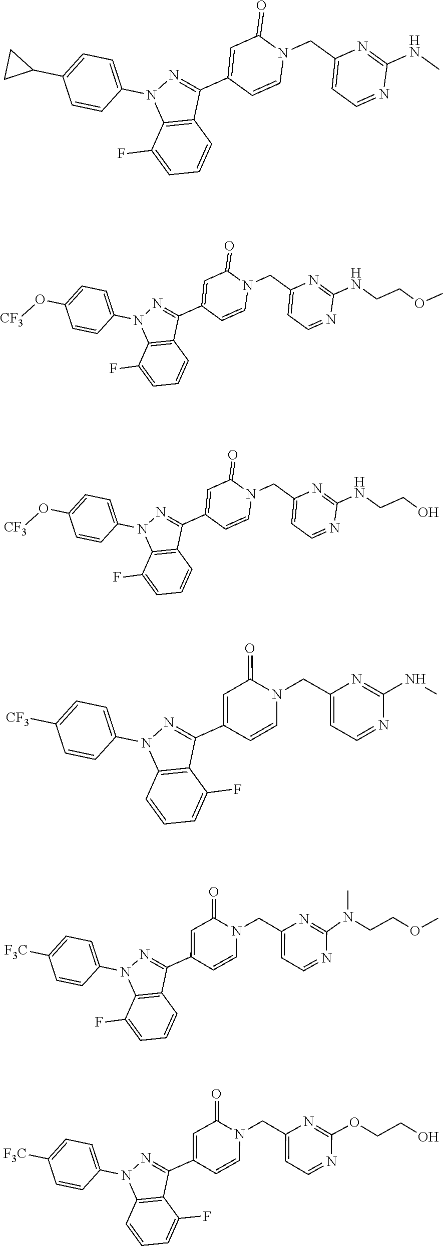 [SMILES: CNc1nccc(Cn2ccc(-c3nn(-c4ccc(C5CC5)cc4)c4c(F)cccc34)cc2=O)n1 ; COCCNc1nccc(Cn2ccc(-c3nn(-c4ccc(OC(F)(F)F)cc4)c4c(F)cccc34)cc2=O)n1 ; O=c1cc(-c2nn(-c3ccc(OC(F)(F)F)cc3)c3c(F)cccc23)ccn1Cc1ccnc(NCCO)n1 ; CNc1nccc(Cn2ccc(-c3nn(-c4ccc(C(F)(F)F)cc4)c4cccc(F)c34)cc2=O)n1 ; COCCN(C)c1nccc(Cn2ccc(-c3nn(-c4ccc(C(F)(F)F)cc4)c4c(F)cccc34)cc2=O)n1 ; O=c1cc(-c2nn(-c3ccc(C(F)(F)F)cc3)c3cccc(F)c23)ccn1Cc1ccnc(OCCO)n1] 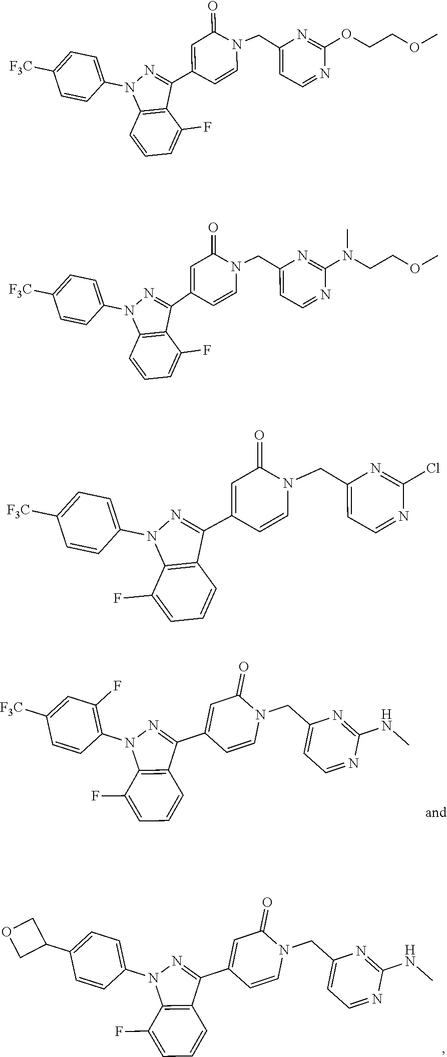 [SMILES: COCCOc1nccc(Cn2ccc(-c3nn(-c4ccc(C(F)(F)F)cc4)c4cccc(F)c34)cc2=O)n1 ; COCCN(C)c1nccc(Cn2ccc(-c3nn(-c4ccc(C(F)(F)F)cc4)c4cccc(F)c34)cc2=O)n1 ; O=c1cc(-c2nn(-c3ccc(C(F)(F)F)cc3)c3c(F)cccc23)ccn1Cc1ccnc(Cl)n1 ; CNc1nccc(Cn2ccc(-c3nn(-c4ccc(C(F)(F)F)cc4F)c4c(F)cccc34)cc2=O)n1 ; CNc1nccc(Cn2ccc(-c3nn(-c4ccc(C5COC5)cc4)c4c(F)cccc34)cc2=O)n1]
or a pharmaceutically acceptable salt or solvate thereof.

109. A pharmaceutical composition comprising a compound of claim 88, or a pharmaceutically acceptable salt or solvate thereof, and a pharmaceutically acceptable excipient.

110. A method of inhibition of the Hippo pathway in a subject in need thereof or YAP-TEAD and/or TAZ-TEAD interaction in a subject in need thereof, treating a disease or condition affected by the inhibition of YAP-TEAD and/or TAZ-TEAD interaction in a subject in need thereof, treating cancer in a subject in need thereof, treating cardiovascular disease in a subject in need thereof or treating fibrosis in a subject in need thereof, comprising administering to the subject a therapeutically effective amount of a compound of claim 88, or a pharmaceutically acceptable salt or solvate thereof.

111. The method of claim 88, wherein the cancer is selected from breast cancer, lung cancer, liver cancer, ovarian cancer, squamous cancer, renal cancer, gastric cancer, medulloblastoma, colon cancer, and pancreatic cancer; the fibrosis is liver fibrosis, kidney fibrosis and lung fibrosis.
